(19) United States
(12) Patent Application Publication
(10) Pub. No.: US 2026/0366033 A1
(43) Pub. Date: Nov. 19, 2026

(54) CIS-REGULATORY ELEMENTS OF TRANSLATION AND METHODS USING SAME

(71) Applicant: Yale University, New Haven, CT (US)

(72) Inventors: Antonio Giraldez, New Haven, CT (US); Ethan Strayer, New Haven, CT (US); Srikar Gopinath, New Haven, CT (US)

(21) Appl. No.: 19/488,150

(22) PCT Filed: May 25, 2024
(86) PCT No.: PCT/US2024/031165

§ 371 Date: Nov. 26, 2025

Related U.S. Application Data
(60) Provisional application No. 63/504,628, filed on May 26, 2023.

(51) Int. Cl. C12N 15/67

(52) U.S. Cl. CPC C12N 15/67; C12N 2840/10

(57) ABSTRACT

Repressive Described herein is a non-natural mRNA molecule, which includes a non-natural cisregulatory element that modulates the translational efficiency of the mRNA molecule. Also described herein is a method for modulating mRNA translational efficiency.

### CROSS-REFERENCE TO RELATED APPLICATIONS

The present application claims priority under 35 U.S.C. § 119 (e) to U.S. Provisional Patent Application No. 63/504,628, filed May 26, 2023, which is incorporated herein by reference in its entirety.

### STATEMENT REGARDING FEDERALLY SPONSORED RESEARCH

This invention was made with government support under GM122580 awarded by National Institutes of Health. The government has certain rights in the invention.

### SEQUENCE LISTING

The XML text file named “047162-7468WO1 (02270)_Seq Listing.xml” created on May 25, 2024, comprising 497,445 bytes, is hereby incorporated by reference in its entirety.

### BACKGROUND

The identification of cis-regulatory elements of translation (i.e., specific sequences in mRNA molecules that can modulate translational efficiency of the mRNA molecules—i.e., the rate at which mRNA translates into proteins) would allow the engineering of mRNA molecules that have specific translational efficiencies. For example, high translational efficiency is desirable for mRNA-based vaccines and large-scale protein productions, and decreased translational efficiency is desirable for the delivery of therapeutic nucleic acids which produce proteins that are toxic at higher dosages.

However, both knowledge on the sequences of cis-regulatory elements and methods of identifying such cis-regulatory elements are unfortunately lacking. Therefore, there is a need for novel methods of identifying cis-regulatory elements of translation, as well as for sequences of specific cis-regulatory elements in various species or cell types. The present study addresses this need.

### SUMMARY

In some aspects, the present invention is directed to the following non-limiting embodiments:

Non-Natural mRNA Molecule

In some aspects, the present invention is directed to an mRNA molecule.

In some embodiments, the mRNA molecule comprises a cis-regulatory element selected from the group consisting of SEQ ID NOs: 1-2353 and 2384, wherein the cis-regulatory element does not naturally exist in the mRNA molecule at a location of the cis-regulatory element.

In some embodiments, the cis-regulatory element is a cis-regulatory element for the translational machinery of a mammal, optionally a human.

In some embodiments, the cis-regulatory element comprises at least one sequence selected from the group consisting of SEQ ID NOs: 1-1156 and 2384.

In some embodiments, the cis-regulatory element is a cis-regulatory element for the translational machinery of a fish, optionally a zebrafish.

In some embodiments, the cis-regulatory element comprises at least one sequence selected from the group consisting of SEQ ID NOs: 1157-2353.

In some embodiments, the mRNA molecule further comprises a modified nucleobase that does not naturally exist at the location of the modified nucleobase.

In some embodiments, the modified nucleobase comprises at least one of N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, and 4-acetylcytidine.

In some embodiments, the mRNA molecule encodes a therapeutic peptide or a therapeutic protein.

In some embodiments, the therapeutic peptide or the therapeutic protein comprises a vaccine.

Method of Modulating a Translational Efficiency of mRNA

In some aspects, the present invention is directed to a method of modulating a translational efficiency of a messenger RNA (mRNA) molecule in a cell.

In some embodiments, the method comprises: modifying the mRNA molecule to comprise a cis-regulatory element selected from the group consisting of SEQ ID NOs: 1-2353 and 2384.

In some embodiments, modifying the mRNA molecule comprises modifying a sequence of a DNA molecule encoding the mRNA molecule.

In some embodiments, the cis-regulatory element is a cis-regulatory element for the translational machinery of a mammal, optionally a human.

In some embodiments, the cis-regulatory element comprises at least one sequence selected from the group consisting of SEQ ID NOs: 1-1156 and 2384.

In some embodiments, the cis-regulatory element is a cis-regulatory element for the translational machinery of a fish, optionally a zebrafish.

In some embodiments, the cis-regulatory element comprises at least one sequence selected from the group consisting of SEQ ID NOs: 1157-2353.

In some embodiments, the mRNA molecule is further modified to comprise a modified nucleobase.

In some embodiments, the modified nucleobase comprises at least one of N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

In some embodiments, the cis-regulatory element activates the translation of the mRNA molecule, and wherein a translational efficiency of the mRNA molecule is increased by about 50% or more in comparison to an mRNA molecule lacking the cis-regulatory element but otherwise has the same sequence.

In some embodiments, the cis-regulatory element represses the translation of the mRNA molecule, and wherein a translational efficiency of the mRNA molecule is decreased by about 30% or more in comparison to an mRNA molecule lacking the cis-regulatory element but otherwise has the same sequence.

In some embodiments, the cis-regulatory element activates or represses the translation of the mRNA molecule in the cell at a certain cellular stage.

In some embodiments, the cis-regulatory element is located in a 5′ untranslated region (5′-UTR), at a region including the start codon of the main open reading frame, within the open reading frame, or in a 3′ untranslated region (3′-UTR).

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-IC demonstrate that NaP-TRAP is able to translation through immunocapture, in accordance with some embodiments. FIG. 1A: In NaP-TRAP reporters contain an N-terminal FLAG-tag. Ribosomes translating in-frame express the 3×FLAG peptide in their nascent chains. Translation is measured through immunocapture, using anti-FLAG magnetic beads. The immunoprecipitation efficiency of a reporter correlates with mean number of active ribosomes translating the reporter in-frame. FIG. 1B: NaP-TRAP captures translation repression by a morpholino at 2 hpf. FIG. 1C: NaP-TRAP captures miR430 mediated translation repression at 4 hpf.

FIGS. 2A-2G demonstrate that NaP-TRAP is able to measure the effect of Kozak strength on translation, in accordance with some embodiments. FIG. 2A: Construction of a library containing six random nucleotides upstream and one nucleotide downstream of the start codon of 3×FLAG-GFP. The NaP-TRAP was performed at 6 hpf. Sequence logos for the top and bottom 10% of reporters were generated. FIGS. 2B-2D: A random forest regressor was utilized to predict translation. Reporter Kozak sequences were one-hot encoded. All nucleotide positions were supplied to the model. The model's prediction explained 86% of the variance of the test set (SPR). The permuted feature importance on the trained RFM was performed. Nucleotide positions in red correlate negatively with translation, whereas positions in blue correlate positively with translation (logistic regression). FIG. 2E: The translation measurements were compared at 6 hpf to an in silico derived Kozak score based on frequency of Kozak sequences in the transcriptome. Translation measurement and Kozak scores correlated weakly (SPR 0.3). FIGS. 2F-2G: The translation of ten reporters with a Kozak score was measured between 295-305 using luciferase-based assay. The luciferase and NaP-TRAP translation values correlated well. The sequences shown in FIGS. 2A and 2F are the following:

(SEQ ID NO: 2354)
NNNNNNAUGN
(SEQ ID NO: 2355)
GCCRCCAUGG
(SEQ ID NO: 2356)
GCAAACAUGG
(SEQ ID NO: 2357)
UUUUGCAUGG
(SEQ ID NO: 2358)
GUAUUCAUGU
(SEQ ID NO: 2359)
UUUGUCAUGU
(SEQ ID NO: 2360)
AUACCAAUGA

FIGS. 3A-3K show an example of using NaP-TRAP to predict the translation of maternal 5′ UTRs, in accordance with some embodiments. FIG. 3A: A 11,088 reporter synthetic oligo library was designed from the 5′ UTRs of 1,750 maternally-supplied transcripts. Endogenous 5′ UTRs were tiled every 25 nucleotides with 124 nucleotide segments. Spike-ins were employed added at Trizol extraction step to normalize translation across experiments. FIG. 3B: The present study observed an increase in translation of 1.8-fold between 2 and 6 hpf. FIG. 3C: The random forest regression model was trained using kmer counts (1-6) and features characterizing uAUGs. Features were filtered prior to training based on their correlation to translation. FIGS. 3D-3G: A random forest regression machine learning model was performed to predict translation at 2 and 6 hpf (FIGS. 3D-3E). Given the prominent role of uORFs the analysis was repeated on reporters without upstream start codons (FIGS. 3F-3G).

FIGS. 4A-4J demonstrate that NaP-TRAP captures cis-regulatory elements encoded in the 5′ UTRs of maternally supplied transcripts, in accordance with some embodiments. FIG. 4A: The present study measured translation at 2 hpf and 6 hpf using NaP-TRAP. Reporters were divided into four groups based on their translation rank at both time-points (repressed, active, early activated, and late activated/orange, blue, green and pink.). FIGS. 4B-4E: The fold-change enrichment or depletion was calculated for each tetramer in each group relative to all reporters. A hyper-geometric test was employed to calculate significance (Bonferroni corrected p-value threshold). 4F: The translation of repeats of each tetramer (256 reporters total) at 2 and 6 hpf was measured. FIGS. 4G-4J: The reporters were color based on the tetramer enrichment of each group in the maternal 5′ UTR library.

FIGS. 5A-5H shows examples of the investigation of developmentally dynamic cis-regulatory elements, in accordance with some embodiments. FIG. 5A: Maternal 5′ UTR library translation values at 2 and 6 hpf. Reporters containing miR430 seeds labeled in red (GCACUUA, AGCACUU). FIG. 5B: Dual luciferase assay measuring the inhibitory effect of miR430 binding sites in the 5′ UTR. 5×mir430-nanoluc and 5×mir430-shuffled-nanoluc were injected in wildtype and miR430 knockout embryos. FIG. 5C: Effect of 5′ UTR miR430 seeds on mRNA half-life. FIG. 5D: Rank plot comparing features driving translation at 2 and 6 hpf for reporter mRNAs with no uORFs. FIG. 5E: Cartoon illustration NaP-TRAP experiment to measure the effect of ZGA on the dynamic role of C-richness. FIGS. 5F-5H: NaP-TRAP experiments measuring U-rich, C-rich and U>C and C>U rich reporters at 2 hpf and 6 hpf.

FIGS. 6A-6G shows the adaption of the NaP-TRAP to human cell lines, in accordance with some embodiments. FIG. 6A: HEK293T were transfected using lipofectamine message max and performed NaP-TRAP at 12 hours post transfection. FIG. 6B: The distribution of translation values in HEK293T cells. FIG. 6C: Pentamers enriched in the bottom 10% of reporters based on translation values. FIG. 6D: Pentamers enriched in the top 10% of reporters based on translation values. FIG. 6E: The effect of uORF number on translation in HEK293T cells. FIG. 6F: Random Forest regression model predicts translation, explaining 79% of the variation observed in the test set. FIG. 6G: Permuted feature importance was performed on the Random Forest model.

FIGS. 7A-7J: Random Forest regression models predict translation for sv40 (FIGS. 7A and 7C), pA (FIGS. 7B and 7D) and human (FIG. 7E) NaP-TRAP 5′ UTR maternal libraries, in accordance with some embodiments. FIGS. 7F-7J: permuted feature importance was performed on random forest regression models: sv40 (FIGS. 7F and 7H), pA (FIGS. 7G and 7I) and human (FIG. 7J) NaP-TRAP 5′ UTR maternal libraries.

FIGS. 8A-8C demonstrate that NaP-TRAP is able to measure translation through immunocapture, in accordance with some embodiments. FIG. 8A: Schematic detailing the NaP-TRAP method. FLAG-tagged nascent chain complexes of reporter mRNAs are enriched via an anti-FLAG immunoprecipitation. Translation is measured as a ratio of reporter reads in the pulldown relative to the input. FIG. 8B: NaP-TRAP derived translation at 6 hpf (left, N=3) and immunofluorescence measurements at 24 hpf (right, −MO N=14, +MO N=9) in the presence or absence of a translation blocking morpholino (unpaired t-test: **p<0.005, ****p<0.0001). FIG. 8C: NaP-TRAP derived translation values for 3×FLAG-GFP-3×miR-430 (red) and 3×FLAG-GFP-3×miR-204 (black) at 2 hpf (N=4) and 4.3 hpf (N=4) (unpaired t-test: **p<0.01, ***p<0.005). Schematic representation of repression of translation exercised by miR-430, but not miR-204 at 4.3 hpf.

FIGS. 9A-9G demonstrate that NaP-TRAP is able to measure the effect of Kozak strength on translation, in accordance with some embodiments. FIG. 9A: A schematic detailing the Kozak library containing six random nucleotides upstream and one nucleotide downstream of the start codon of 3×FLAG-GFP (top left). Histogram of NaP-TRAP translation values at 6 hpf (bottom left). Sequence logos for the top and bottom 10% of reporters based on translation (right). FIGS. 9B-9C: Cartoon of random forest regression model feature generation through the one-hot-encoding of reporter sequences (FIG. 9B). Scatterplot comparing the model's prediction to the experimentally derived translation values of a test set of reporters (FIG. 9C; Pearson's R) (N=785 reporters). FIG. 9D: Permuted feature importance derived from random forest regression model. Nucleotide positions in purple correlate negatively with translation, whereas positions in blue correlate positively with translation (Spearman Rank Correlation Coefficient). FIG. 9E: Comparison of translation measurements at 6 hpf and an in silico derived Kozak score based on the frequency of Kozak sequences in the transcriptome (Pearson's R) (N=2,617 reporters). FIGS. 9F-9G: The translation of four reporters with a Kozak score between 295-305 was measured using a dual luciferase-based assay (FIG. 9F). Plot comparing relative luciferase activity (Nano luciferase/Firefly luciferase), and NaP-TRAP translation values (FIG. 9G) (Pearson's R, N=3). The sequences shown in FIGS. 9A and 9F are the following:

(SEQ ID NO: 2354)
NNNNNNAUGN
(SEQ ID NO: 2355)
GCCRCCAUGG
(SEQ ID NO: 2356)
GCAAACAUGG
(SEQ ID NO: 2357)
UUUUGCAUGG
(SEQ ID NO: 2358)
GUAUUCAUGU
(SEQ ID NO: 2359)
UUUGUCAUGU
(SEQ ID NO: 2360)
AUACCAAUGA

FIGS. 10A-10I show an example of using NaP-TRAP to predict the translation of maternal 5′ UTRs, in accordance with some embodiments. FIG. 10A: A schematic detailing global changes in translation during the maternal-to-zygotic transition in the developing zebrafish embryo (top left). The 5′ UTR library was generated by tiling the 5′ UTRs of zebrafish (bottom left). The NaP-TRAP workflow with the addition of spike-ins at the RNA extraction step (right). FIG. 10B: Comparison of translation values between 2 and 6 hpf of the 5′ UTR library (Mann-Whitney p<10-100, N=8,529 reporters). FIG. 10C: Schematic detailing random forest regression model feature selection (k-mer counts 1-6 nt and features characterizing uAUGs). FIGS. 10D-10I: Scatterplot comparing each model's prediction to the experimentally derived translation values of a test set of reporters (N=2,559 reporters) (FIGS. 10D-10E; Pearson's R). The permuted feature importance of features (top 12) with the greatest effect on model performance (blue refers to a positive correlation, purple refers to a negative correlation; Spearman Rank Correlation Coefficient) (FIGS. 10F-10G). The correlation of selected features with translation at the indicated timepoints (FIGS. 10H-10I).

FIGS. 11A-11H demonstrate that NaP-TRAP is able to capture general and dynamic 5′ UTRs cis-regulation, in accordance with some embodiments. FIG. 11A: The translation of the 5′ UTR library at 2 hpf and 6 hpf using NaP-TRAP (Pearson's R, N=503 8,529 reporters). Reporters were divided into four groups based on their translation rank at both time-points: repressed, active, active post ZGA, and repressed post ZGA (orange, blue, pink, and green, respectively). FIGS. 11B-11E: Fold-change enrichment and depletion for all pentamers in each group from FIG. 11A relative to the reporter library (hyper-geometric test to calculate significance; Bonferroni corrected p-value threshold p<5*10-6). FIG. 11F: Schematic detailing the library of all possible tetramer repeats separated by dinucleotide spacers. FIG. 11G: Translation measurements of the validation library at 2 and 6 hpf (Pearson's R, N=195 reporters). Tetrameric reporters were labeled based on whether their repeat was enriched in the reporter groups described above (FIG. 11A). FIG. 11H: Cumulative distribution plot 20 comparing the translation (2hpf/6 hpf) of validation reporters identified as active post ZGA (pink) and repressed post ZGA (green) (Mann-Whitney U-test: p<10-5). The sequences shown in FIG. 11F are the following:

(SEQ ID NO: 2361)
AAAUCAAAAACAAAAAGAAAACGAAAA
(SEQ ID NO: 2362)
AAAGUCAAAGACAAAGAGAAAGCGAAAG
(SEQ ID NO: 2363)
AAAUUCAAAUACAAAUAGAAUCGAAAU
(SEQ ID NO: 2364)
AAACUCAAACACAAACAGAAACCGAAAC

FIGS. 12A-12E demonstrates that zygotic expression of miR-430 inhibits translation of mRNAs with 5′ UTR seed sites, in accordance with some embodiments. FIG. 12A: Cumulative distributions of translation (6 hpf/2 hpf) for reporters containing miR-430 (GCACUUA, GCACUUU, AGCACUU; N=212 reporters) or miR-1 (ACAUUCC, CAUUCCA; N=71 reporters) heptamers, labeled in green and blue respectively. Reporters labeled in gray contain neither seed sequence (N=8,242 reporters) (Mann-Whitney U test, p<10-27 miR-430 reporters vs reporters with no seed; Mann-Whitney U test, p<0.001 miR-1 reporters vs reporters with no seed). FIGS. 12B-12C: The effect of number of complementary bases to miR-430 (B, N=275 reporters) or miR-1 (C, N=178 reporters) on the translation (6 hpf/2 hpf) of reporters with seed sequences (Pearson's R). FIGS. 12D-12E: Schematic detailing dual luciferase assay measuring the inhibitory effect of miR-430 binding sites in the 5′ UTR (FIG. 12D). 4×miR-430-nanoluc and 4×miR-430-shuffled-nanoluc were injected in wild-type and miR-430 knockout embryos. Relative Luciferase Activity (RLU) values were normalized to each reporter (N=3, unpaired t-test) (FIG. 12E).

FIGS. 13A-13F show the dynamic effect of C-richness on translation, in accordance with some embodiments. FIG. 13A: Plot comparing feature rank at 2 and 6 hpf (k-mers≤4). At each time point feature rank was determined by calculating the mean difference in translation between non-uAUG reporters enriched and depleted (top and bottom 20%) in each feature (red: repressive at 2 hpf, blue: active at 2 hpf, purple: features that were repressive at 2 hpf and then active at 6 hpf). Features were only included if there was a significant difference in mean translation at each timepoint (Bonferroni corrected t-test). FIG. 13B: Proposed models explaining how the maternal-to-zygotic transition can alter the effect of cis-regulatory elements in the 5′ UTR. FIGS. 13C-13F: U's in two active (U-rich) reporters were mutated to C's (U>C), whereas C's in two active post ZGA (C-rich) reporters were mutated to U's (C>U). Translation values were measured using NaP-TRAP at 2 and 6 hpf. Wild-type reporters are colored gray, whereas mutant reporters are labeled red (N=3, unpaired t-test).

FIGS. 14A-14J shows the adaptation of NaP-TRAP to human cell lines, in accordance with some embodiments. FIG. 14A: Schematic detailing NaP-TRAP in HEK293T cells. Cells were transfected with the 5′ UTR library using lipid nanoparticles. Translation was measured at 12 hours post transfection. FIG. 14B: The distribution of translation values of the 5′ UTR library in HEK293T cells. The top and bottom 10% of reporters based on their translation values are labeled in orange and blue (repressed and active reporters, respectively; N=7,506 reporters). FIGS. 14C-14D: A differential enrichment analysis identified pentamers enriched and depleted in repressed (FIG. 14C) and active (FIG. 14D) reporters, blue and orange respectively (hypergeometric test with a Bonferroni corrected p-value). FIG. 14E: Venn-diagram comparing the pentamers enriched in active reporters in HEK293T cells and zebrafish embryos at 2 and 6 hpf. FIG. 14F: Translation values at 2 hpf in zebrafish compared to translation values in HEK293T cells (Pearson's R). Reporters were divided into four groups based on their translation rank at each condition: active (blue), repressed (orange), active in zebrafish at 2 hpf (pink) and active in HEK293T cells (green) (N=7,506 reporters). FIGS. 14G-14J: The fold-change enrichment or depletion for all pentamers in each group (active (FIG. 14G), repressed (FIG. 14H), active in zebrafish at 2 hpf (FIG. 14I), and active in HEK293T (FIG. 14J)) relative to the reporter library. Significance was calculated using a hyper-geometric test (Bonferroni corrected p-value threshold).

FIGS. 15A-15F demonstrate that the NaP-TRAP method can be adapted to study the translation of multiple ORFs simultaneously in vivo, in accordance with some embodiments. FIG. 15A: NaP-TRAP is an accessible, versatile, and quantitative method that measures the translation of thousands of reporters simultaneously through the immunocapture of FLAG-tagged nascent peptides. FIG. 15B: Through the over-expression of an HA-tagged RNA binding protein (RBP). NaP-TRAP can be employed in conjunction with an RNA immunoprecipitation experiment to measure the effect of RBP recruitment on translation. FIGS. 15C-15F: NaP-TRAP quantifies translation in a frame-specific manner. Through the incorporation of additional epitope tags in frames 2,3 or ORFs outside of the main open reading frame, the NaP-TRAP method can be utilized to: (1) measure out-of-frame translation in the main ORF 586 (FIG. 15C), (2) detect internal ribosome entry sites (IRES) sequences in an unbiased manner through the use of a bicistronic 587 reporter (FIG. 15D), (3) identify frameshifting elements (FIG. 15E), and (4) quantify stop codon readthrough (FIG. 15F).

FIG. 16 shows the comparison of NaP-TRAP to existing translation based MPRA methods, in accordance with some embodiments. NaP-TRAP was developed to quantify the translation of thousands of reporters simultaneously in a frame-specific manner. In contrast to existing methods, NaP-TRAP can be adapted to dynamic model systems (e.g. the developing zebrafish embryo), and does not require specialized equipment or a large amount of source material to measure translation.

FIGS. 17A-17C exemplify using NaP-TRAP to model and predict Kozak Strength in early embryos, in accordance with some embodiments. FIG. 17A: Plot comparing replicate translation values for the Kozak library (Pearson's R; N=2,712 reporters). FIG. 17B: The correlation coefficient between the top 12 features identified by the permuted feature importance analysis and translation (blue refers to positive correlation, purple refers to negative correlation; Spearman Rank Correlation Coefficient). FIG. 17C: The predicted translation values of all possible Kozak sequences in early zebrafish embryos (N=15,364 Kozak sequences) (Random Forest regression model: FIGS. 9B-9D).

FIGS. 18A-18J demonstrate that NaP-TRAP is able to quantitatively measure the translation of zebrafish 5′ UTRs, in accordance with some embodiments. FIGS. 18A-18B: Comparing the translation values of replicates of the 5′ UTR library with a hard-encoded poly-A at 2 and 6 hpf (Pearson's R) (FIG. 18A: N=9,516 reporters; FIG. 18B: N=8,529 reporters). FIGS. 18C-18D: Plot comparing relative luciferase activity (Nano-luciferase/Firefly luciferase) to NaP-TRAP derived translation values of the zebrafish 5′ UTR library validation reporters at 2 (FIG. 18C, N=3) and 6 hpf (FIG. 18D, N=4) (Pearson's R). FIGS. 18E-18F: Random Forest regression models were trained on reporters without upstream AUGs. The performance of each model was evaluated by predicting the translation values of a test set of reporters (Pearson's R) (N=660 reporters). FIGS. 18G-18I: The permuted feature importance of the top 12 features informing the predictive power of each model (FIGS. 18G and 18H). The spearman rank correlations between the top 12 features and translation at each timepoint (FIGS. 18I and 18J) (blue represents positive correlation, purple represents negative correlation).

FIGS. 19A-19N exemplify using NaP-TRAP to measure the regulatory activity of cis-elements in an environment with reduced translation, in accordance with some embodiments. FIGS. 19A-19B: Replicates of translation values for SV40 zebrafish 5′ UTR library (Pearson's R) (FIG. 19A: N=9,999 reporters; FIG. 19B: N=7,442 reporters). FIG. 19C: The distributions of the translation values for the hard encoded pA (60A) and the sv40 zebrafish 5′ UTR libraries at 2 and 6 hpf (Mann-Whitney U test p<10-100) at 2 and 6 hpf (N=7,427 reporters). FIGS. 19D-19I: Random Forest regression models predict translation at 2 and 6 hpf (N=2,233 reporters) (FIGS. 19D and 19G). The present study utilized a permuted feature importance analysis to identify the top 12 features informing the prediction of the RFM at both timepoints (FIGS. 19E and 19H). The present study plotted the Spearman Rank Correlation of each of the selected features with translation (blue is positive and purple is negative) (FIGS. 19F and 19I). FIG. 19J: NaP-TRAP derived translation values the SV40 zebrafish 5′ UTR library at 2 hpf and 6 hpf. Reporters colored by enrichment group (yellow: repressive, blue: active, pink: active post-ZGA, and green: active pre-ZGA). FIGS. 19K-19N: The enrichment for all pentamers in each group relative to all reporters (hyper-geometric test to calculate significance with Bonferroni corrected p-value threshold).

FIGS. 20A-20E illustrate certain aspects of the NaP-TRAP validation library of tetramer repeats, in accordance with some embodiments. FIGS. 20A-20D: Reporters were labeled based on whether their encoded tetramer repeat was enriched in the reporter groups defined in the differential kmer enrichment analysis performed on the 5′ UTR library (FIGS. 11B-11E) (orange: repressive, blue: active, pink: active post ZGA, green: repressed post ZGA, gray: no group). The validation of the active motifs was limited by the length of the motifs as the dinucleotide spacers created the motifs present in the other groups (FIG. 20B). FIG. 20E: Previously labelled reporters were colored gray if they contained tetramers found in other groups (four or more counts).

FIGS. 21A-21L show the comparison of translation in HEK293T cells and the developing zebrafish embryo, in accordance with some embodiments. FIGS. 21A-21C: Replicates of translation values for the 5′ UTR library in HEK293T cells (Pearson's R) (N=7,584 reporters). FIG. 21D: Venn-diagram comparing the repressive features of HEK293T cells and the zebrafish embryo at 2 and 6 hpf (pentamers enriched in the bottom 10% of reporters). FIG. 21E: NaP-TRAP derived translation values from the zebrafish embryos at 6 hpf and HEK293T cells at 12 hpt. Both libraries had a hard-encoded 60A tail. Reporters colored by enrichment group (orange: repressive, blue: active, pink: active at 6 hpf, and green: active in HEK293T cells) (N=7,506 reporters). FIGS. 21F-21I: The enrichment for all pentamers in each group relative to all pentamers found in the reporter library. Significance was determined using a hyper-geometric test (Bonferroni corrected p-value threshold). FIGS. 21J-21L: Random Forest regression model predicts translation (N=2,252 reporters) (FIG. 21J). The permuted feature importance for features with the greatest effect on the model's predictive power (top 12; FIG. 21K). The correlation of said features with translation (FIG. 21L) (blue is positive correlation, purple is negative correlation, Spearman Rank Correlation Coefficient).

FIG. 22 demonstrates that, in the absence of out-of-frame translation, NaP-TRAP and polysome profiling correlate strongly, in accordance with some embodiments. Specifically, the present study performed polysome profiling in HEK 293T cells using the zebrafish 5′ UTR library. The present study observed a strong correlation between NaP-TRAP derived translation values and mean ribosome load (MRL).

FIGS. 23A-23D demonstrate that NaP-TRAP is able to capture translation in multiple frames simultaneously, in accordance with some embodiments. FIGS. 23A-23B: To measure translation in multiple frames simultaneously, the present study incorporated a FLAG, HA and MYC tag into frames 0, 1+ and 2+ respectively. The present study measured the translation of library consisting of upstream open reading frames in each frame by performing three different pulldowns on each sample using anti-FLAG, anti-HA and anti-MYC beads. FIGS. 23C-23D: The present study observed out-of-frame translation (immunocapture) for reporters that contain oORFs in frames 1+ and 2+. An in-frame overlapping ORF or the presence of the main start codon in frame 0 represses translation in frames 1+ and 2+.

FIG. 24 illustrates certain aspects of a luciferase assay, which shows 10-fold increased luciferase expression with RESA mRNA-V1 5′UTR as compared to mRNA-1273 and BNT16 2b2 5′UTRs, in accordance with some embodiments. Error bars represent SD and statistical significance was calculated using independent t-test (****p-value<0.001).

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Cis-regulatory elements encoded in the sequence and structure of mRNAs control protein expression. Conventional methods of determine translational efficiency of an mRNA molecule and identifying cis-regulatory elements are based on the immunocapture of ribosomes (such as epitope tagged ribosomes) followed by sequencing the mRNA molecules associated with the captured ribosomes. The rationale of such methods is that mRNA molecules having higher translational activity are being translated by more ribosomes, and vice versa. As such, the ribosome load on the mRNA molecules can show the translational activity of the mRNA molecules and reveal the existence, sequence and/or location of such cis-regulatory elements.

In the study described herein (“the present study”), it was discovered that ribosome load is not an entirely reliable indication of the translational efficiency. For example, ribosomes associated with an mRNA molecule can be inactive ribosomes, or translating outside of the main open reading frame of the mRNA molecule. Ribosome load cannot distinguish the efficiency of the translation of the main open reading frame from such non-productive associations.

The present study further developed, in one aspect, a method of solving the above issues with the conventional methods. Rather than capturing the ribosomes, the method of the invention herein captures the translation complex that includes the mRNA, the ribosome, and the partially translated peptide still associated with the mRNA and the ribosome by capturing the partially translated peptide. This way, all the mRNA captured by the method herein are associated with peptides that are being actively translated from the mRNA molecules.

Furthermore, using the novel method herein, cis-regulatory elements of translation were identified in zebrafish and human cells. The cis-regulatory elements, some of which result in high translational efficiency, some of which result in low translational efficiency, some of which increase or decrease translational efficiency as time points advance, can be used to construct DNA or mRNA molecules that have desired translational efficiencies or patterns of translational efficiencies.

Accordingly, in some aspects, the present invention is directed to a method of identifying cis-regulatory elements of mRNA translation.

In some aspects, the present invention is directed to a method of determining translational efficiency of an mRNA molecule.

In some aspects, the present invention is directed to a mRNA molecule including a non-natural cis-regulatory elements, or a DNA molecule encoding the same.

In some aspects, the present invention is directed to a method of modulating translation efficiency of an mRNA molecule or a DNA molecule encoding the same.

In some embodiments, the present invention is directed to a method of constructing mRNA molecules for producing therapeutic proteins/peptides.

### DEFINITIONS

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of +20% or +10%, in certain embodiments±5%, in certain embodiments±1%, in certain embodiments±0.1% from the specified value, as such variations are appropriate to perform the disclosed methods.

Method of Identifying Cis-Regulatory Elements of mRNA Translation

In some aspects, the instant specification is directed to a method of identifying cis-regulatory element of translation.

In some embodiments, the method includes translating a plurality of mRNA molecules, each independently comprising a potential cis-regulatory element, with ribosomes to obtain an initial mixture.

In some embodiments, the method further includes generating from the initial mixture an enriched complex/mixture comprising mRNA molecules being translated, the ribosomes, and partial translation products being produced by the ribosomes from the mRNA molecules according to an affinity of the partial translation products to a binding partner thereof.

In some embodiments, the method further includes sequencing the mRNA molecules present in the enriched complex/mixture.

In some embodiments, the method further includes determining an abundance of each mRNA molecule present in the enriched complex/mixture.

In some embodiments, the method further includes identifying cis-regulatory elements of translation based on the determined abundance of each of the plurality of mRNA molecules.

In some embodiments, each mRNA molecule of the plurality of mRNA molecules comprises a common sequence which translates into a peptide motif recognized by the binding partner. The peptide motif is not limited. One of ordinary skill in the art would understand that, as long as a peptide motif can, either by itself or via modifications, specifically interact with a binding partner and therefore enriched or purified by the affinity to the binding partner, the peptide motif is suitable for the purpose herein. Non-limiting examples of peptide motifs include ALFA-tag, AviTag, C-tag, Calmodulin-tag, polyglutamate tag, polyarginine tag, E-tag, FLAG-tag, HA-tag, His-tag, Myc-tag, NE-tag, SBP-tag, Spot-tag, Strep-tag, T7-tag, TC tag, V5 tag, VSV-tag, Xpress tag, and the like. The peptide sequences of such peptide motifs are well known in the art and, as such, nucleic acid sequences that translate into the peptide motifs can also be determined or designed. In some embodiments, the common sequence is a nature sequence in an mRNA molecule and the peptide sequence thereof can be recognized by, for example, an antibody or an antigen binding fragment thereof.

In some embodiments, the common sequence is to the 3′-end of the start codon. In some embodiments, the common sequence is within about 300 nucleotides from the start codon, such as within about 270 nucleotides, within about 240 nucleotides, within about 210 nucleotides, within about 180 nucleotides, within about 150 nucleotides, within about 120 nucleotides, within about 90 nucleotides, within about 60 nucleotides, within about 30 nucleotides, within about 27 nucleotides, within about 24 nucleotides, within about 21 nucleotides, within about 18 nucleotides, within about 15 nucleotides, within about 12 nucleotides, within about 9 nucleotides, within about 6 nucleotides, within about 3 nucleotides from the start codon. In some embodiments, the common sequence starts right after the start codon or include the start codon. In some embodiments, the start codon is the start codon of the main open reading frame. In some embodiments, the start codon is not the start codon of an upstream open reading frame (uORF) or a downstream open reading frame (dORF).

In some embodiments, the size of the peptide motif is about 100 amino acid residues or less, such as about 90 amino acid residues or less, about 80 amino acid residues or less, about 70 amino acid residues or less, about 60 amino acid residues or less, about 50 amino acid residues or less, about 40 amino acid residues or less, about 30 amino acid residues or less, about 20 amino acid residues or less, about 15 amino acid residues or less, about 12 amino acid residues or less, about 10 amino acid residues or less, or about 8 amino acid residues or less.

In some embodiments, apart from the potential cis-regulatory elements, the plurality of the mRNA molecules has a same nucleotide sequence. In some embodiments, the plurality of the mRNA molecules is engineered from a same nucleic acid molecule, by either inserting the potential cis-regulatory elements into the nucleic acid molecule and/or altering the existing sequences in the nucleic acid molecule to arrive at the potential cis-regulatory elements.

The choice of the binding partner depends on the peptide motif used for enriching the complex, and is not particularly limited. For example, virtually all unique peptide motifs can be targeted by antibodies or antigen binding fragments thereof that recognizes the peptide motif. In some embodiments, the peptide motif is recognized by a non-antibody binding partner. For example, AviTag can be biotinylation by the enzyme BirA, which can then be recognized by biotin binding proteins such as streptavidin. For example, His-Tag can be recognized by nickel (II) or cobalt (II) ions. For example, SBP-Tag can be recognized by streptavidin. One of ordinary skill in the art would know how to select the binding partner based on the choice of the peptide motif for recognition.

In some embodiments, generating the enriched complex/mixture includes immobilizing the binding partner to a substrate, a bead, or the like, contacting the translation mixture with the immobilized binding partner, and at least partially removing the unbound translation mixture from the immobilized binding partner. In some embodiments, the immobilized binding partner and the translation complex captured thereon are further washed by, for example, a buffer solution to remove mRNA molecules not associated with the complex.

In some embodiments, the mRNA molecules present in the enriched complex is sequenced by a high throughput sequencing method. High throughput sequencing methods are described in, for example, Reuter et al. (Mol Cell. 2015 May 21; 58 (4): 586-597).

In some embodiments, the mRNA molecules present in the enriched complex is converted into a cDNA molecule. In some embodiments, mRNA molecules present in the enriched complex are sequenced directly, such as by a nanopore sequencing method.

In some embodiments, the abundance of each mRNA molecule present in the enriched complex is determined by comparing abundance of each mRNA molecule with a predetermined baseline. The predetermined baseline can be, for example, an average or a median of the abundance of each mRNA molecules in the same sample or similar types of samples.

In some embodiments, a potential cis-regulatory element is determined to be cis-regulatory element that enhances translational efficiency if the abundance of mRNA molecules comprising the same is higher than the baseline, such as about 10% higher than the baseline, about 20% higher than the baseline, about 30% higher than the baseline, about 40% higher than the baseline, about 50% higher than the baseline, about 75% higher than the baseline, about 100% higher than the baseline, about 150% higher than the baseline, about 200% higher than the baseline, about 250% higher than the baseline, or about 300% higher than the baseline.

In some embodiments, a potential cis-regulatory element is determined to be cis-regulatory element that suppresses translational efficiency if the abundance of mRNA molecules comprising the same is lower than the baseline, such as less than about 90% of the baseline, less than about 80% of the baseline, less than about 70% of the baseline, less than about 60% of the baseline, less than about 50% of the baseline, less than about 40% of the baseline, or less than about 30% of the baseline.

In some embodiments, the method is configured to identify cis-regulatory elements that have different effects on translation efficiency at different time points.

In some embodiments, the different time points are different time points of cell division, cell differentiation, disease progression, embryo development, aging process, and so forth.

In some embodiments, the method includes translating the plurality of mRNA molecules at a first time point to obtain a first mixture; translating the plurality of mRNA molecules at a second time point after the first time point to obtain a second mixture; performing the enriching step, the sequencing step, the determination step and the identification step to identify cis-regulatory elements of translation that have different effects on translational efficiency at the first time point and the second time point.

In some embodiments, translating the plurality of mRNA molecules comprises introducing the plurality of mRNA molecules into a cell.

In some embodiments, the cell is from a cell line, a primary cell, a cell in a tissue, a cell in an organ, a stem cell, an embryonic cell, a dividing cell, or a differentiating cell.

In some embodiments, the cell is a bacterial cell, a plant cell, a eukaryotic cell, a vertebrate cell, a mammalian cell, a human cell, or the like.

Method of Determining Translational Efficiency of mRNA Molecule

In some embodiments, the instant specification is directed to a method of determining translational efficiency of an mRNA molecule.

In some embodiments, the method includes translating the mRNA molecules with ribosomes to obtain an initial mixture; generating from the initial mixture an enriched complex/mixture comprising the mRNA molecules being translated, the ribosomes, and partial translation products being produced by the ribosomes according to affinity of the partial translation products to a binding partner thereof; determining abundance of the mRNA in the enriched complex/mixture; and comparing the abundance with a predetermined baseline.

In some embodiments, some or all the steps in the method of determining translational efficiency of an mRNA molecule are the same as or similar to those described elsewhere herein, such as in the “Method of Identifying Cis-Regulatory Elements of mRNA Translation” section.

mRNA Molecule or DNA Molecule Encoding the Same

In some aspects, the present invention is directed to an mRNA molecule, or a DNA molecule encoding the same.

In some embodiments, the mRNA molecule includes a non-natural cis-regulatory elements that does not exist in the mRNA molecule naturally. The term “non-natural cis-regulatory elements” does not indicate that the cis-regulatory elements cannot exist in natural mRNA molecules. Rather, if the mRNA molecules herein includes a “non-natural cis-regulatory element” that exist in some natural mRNA molecules, the cis-regulatory element does not exist in the natural mRNA molecule.

In some embodiments, the mRNA molecule includes a modified nucleobase. As used herein, the term “modified nucleobase” refers to nucleobases other than the four common nucleobases adenine (A), cytosine (C), uracil (U), and guanine (G). The term “modified nucleobase” does not exclude naturally occurring nucleobases that are found in non-mRNA molecules. For example, nucleobases like pseudouridine, 5-methylcytosine, N1-methylpseudouridine and 2′-O-methylated exist in nature. These nucleobases are considered to be examples of modified nucleobases herein.

In some embodiments, the cis-regulatory element is in the 5′ untranslated region (5′-UTR). In some embodiments, the 5′-UTR includes an upstream open reading frame (uORF).

In some embodiments, the cis-regulatory element includes some or all the nucleotides of the start codon of the main open reading frame (also referred to herein as the “open reading frame” or the “ORF”), and a portion of the 5′-UTR and/or the main open reading frame.

In some embodiments, the cis-regulatory element is in the main open reading frame.

In some embodiments, the cis-regulatory elements is in the 3′ untranslated region (3′-UTR).

In some embodiments, the cis-regulatory element is a cis-regulatory element that regulates translation in a mammalian cell, such as a human cell.

In some embodiments, the cis-regulatory element is has a nucleotide sequence set forth in at least one of SEQ ID NOs: 1-1156.

In some embodiments, cis-regulatory element is a cis-regulatory element that regulates translation in a fish cell, such as a zebrafish cell.

In some embodiments, the cis-regulatory element is has a nucleotide sequence set forth in at least one of SEQ IDs: 1157-2353.

In some embodiments, the cis-regulatory element activates the translation. In some embodiments, the cis-regulatory element activates the translation at a certain cellular stage, such as a certain stage of a cellular process, a cell division, a cell differentiation, a disease progression, an embryo development, an aging process, and so forth. In some embodiments, the inclusion of the cis-regulatory element increase the translation efficiency by about 10% or more, such as about 20% or more, 30% or more, 50% or more, 75% or more, 100% or more, 150% or more, 200% or more, 300% or more, 400% or more, or 500% or more, when compared to an mRNA molecule that does not include the cis-regulatory element but otherwise have the same sequence.

In some embodiments, the cis-regulatory element represses translation. In some embodiments, the cis-regulatory element represses the translation at a certain cellular stage, such as a certain stage of a cellular process, a cell division, a cell differentiation, a disease progression, an embryo development, an aging process, and so forth. In some embodiments, the inclusion of the cis-regulatory element decreases the translation efficiency by about 10% or more, such as about 20% or more, about 30% or more, about 40% or more, about 50% or more, about 60% or more, about 70% or more, about 80% or more, about 85% or more, about 90% or more, about 95% or more, about 98% or more, or about 99% or more, when compared to an mRNA molecule that does not include the cis-regulatory element but otherwise have the same sequence.

In some embodiments, the increase or decrease of the translation is measured according to the Nascent Peptide Translating Ribosome Affinity Purification (NaP-TRAP) assay according to some embodiments herein.

In some embodiments, the modified nucleobase is the nucleobases of N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

In some embodiments, the mRNA molecule encodes a therapeutic peptide or a therapeutic protein.

In some embodiments, the therapeutic peptide or the therapeutic protein includes a vaccine.

Method of Modulating Translation Efficiency or Constructing mRNA Molecules

In some aspects, the present invention is directed to a method of modulating translational efficiency of an mRNA molecule or a DNA molecule encoding the same.

In some embodiments, the method includes modifying the mRNA molecule, or the DNA molecule encoding the same to include a cis-regulatory element in the mRNA. In some embodiments, the inclusion of the cis-regulatory element increases or decreases the translational efficiency. In some embodiments, the inclusion of the cis-regulatory element increases or decreases the translational efficiency at a certain cellular stage.

In some aspects, the present invention is directed to a method of constructing an mRNA molecule. In some embodiments, the mRNA molecule is an mRNA molecule for producing a therapeutic peptide or a therapeutic protein.

In some embodiments, the method includes incorporating a cis-regulatory element in a template mRNA that encodes the therapeutic peptide or the therapeutic protein.

In some embodiments, the therapeutic peptide or a therapeutic protein includes a vaccine.

In some embodiments, the mRNA and/or the cis-regulatory element is the same as or similar to those detailed elsewhere herein, such as the “mRNA Molecule or DNA Molecule Encoding the Same” section.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Nap-Trap, a Novel Massive Parallel Reporter Assay to Quantify Translation Control

The cis-regulatory elements encoded in the sequence and structure of an mRNA determine its stability and translational output. While there has been a considerable effort to understand the factors driving transcription, the regulatory framework governing post-transcriptional control remains elusive. This is particularly true in non-steady state conditions (e.g., embryogenesis), where correlations between mRNA abundance and protein production break down.

According to some non-limiting embodiments, the study described herein (“the present study”) developed a novel massive parallel reporter assay (MPRA) to measure translation through immunocapture, Nascent Peptide Translating Ribosome Affinity Purification (NaP-TRAP). In contrast to other TRAP and polysome profiling based MPRAs, NaP-TRAP only captures actively translating ribosomes in a frame specific manner, resulting in a more accurate quantification of translation.

The present study employed NaP-TRAP to quantify the regulatory landscapes of the Kozak sequence and maternally derived 5′ UTRs during early zebrafish embryogenesis, identifying general and developmentally dynamic cis-regulatory elements. The present study identified U-rich motifs as general enhancers of translation, and upstream ORFs and GC-rich motifs as general repressors of translation. Strikingly, the present study reports on a previously uncharacterized translational switch. During the maternal-to-zygotic transition, that pyrimidine-rich motifs shift from a repressor to a prominent activator of translation was observed. The present study also characterized 5′ UTR microRNA mediated translation repression driven by the zygotic expression of miR430. Lastly, the present study also adapted the method to human cell lines.

Life requires spatial and temporal control of protein expression. The protein output of a given transcript reflects the integration of translation and mRNA stability. While there has been a considerable effort to understand the factors driving mRNA synthesis, maturation, and decay, the regulatory frameworks governing translational control remain elusive. Cis-regulatory elements encoded in the sequence and structure of mRNAs modulate these processes. Although these elements are distributed throughout the transcript, they are often concentrated in the regions up- and downstream of the main coding sequence, the 5′ and 3′ untranslated regions (UTRs) respectively. Given that initiation is the rate limiting step of translation, there has been a particular interest in understanding the regulatory elements found in 5′ UTRs. These elements include internal ribosome entry sites (IRESs), G-quadruplexes, iron response elements (IREs), and upstream Open Reading Frames (uORFs).

To investigate translation control several studies have utilized ribosome profiling. While this method quantifies accurately the translation efficiency of individual genes, its capacity to characterize the function of cis-regulatory elements is limited. The generation of ribosome protected fragments decouples the translation measurements of a given mRNA from their cognate untranslated regions. Thus, measurements of translation efficiency often reflect the amalgamation of several isoforms of a given gene each with a unique set of regulatory elements. This process is complicated further by the fact that endogenous transcripts contain multiple regulatory elements that exert differential and often competing effects on translation.

Massive parallel reporter assays (MPRAs) are particularly suitable to address these challenges. MPRAs measure the abundance and/or translation efficiency of thousands of reporters simultaneously. These assays have improved the understanding of translation control and mRNA stability. Previous works have characterized Kozak strength, uORFs, IRESs, codon optimality, RNA structure, and microRNA binding sites, as well as identified novel motifs driving translation and decay. Despite these insights, conclusions based on MPRAs have been limited by the methods used to measure translation: growth-selection, fluorescent-cell sorting (FACS), and polysome profiling. In MPRAs where reporters are encoded in DNA, measurements of translation can be confounded by additional layers of transcriptional regulation. In polysome fractionation, which quantifies the number of ribosomes on each transcript, inactive ribosomes, ribosomes translating out of frame, or ribosomes translating ORFs outside of the coding sequence can skew the measurement of translation.

Here, the present study developed NaP-TRAP (Nascent Peptide Translating Ribosome Affinity Purification), a novel method to measure in-frame translation through immunocapture of nascent peptides. More specifically, by including an N-terminal tag in the coding sequences of reporter mRNAs, the present study enriched for reporters in a manner proportional to the number of active ribosomes translating the main ORF in frame. Using this method, the present study quantified the Kozak strength and regulatory potential of 5′ UTRs in the developing zebrafish embryo and human cell lines. The present study demonstrate that the non-limiting NaP-TRAP assay has the capacity to measure the regulatory activity of 5′UTRs across multiple systems. Using NaP-TRAP the present study identified developmentally controlled translational switches as well as common cis-regulatory elements conserved across vertebrates.

### Example 1-1

The present study developed NaP-TRAP to measure translation in a frame specific manner. Given the prevalence of upstream open reading frames in 5′ UTRs and the repressed translational state of early embryogenesis, it was concerned that inactive ribosomes or ribosomes translating outside of the main open reading frame would confound the quantification of translation. In addition, the present study wanted to develop a translation based MPRA method that did not require a large amount of source material or specialized equipment.

It was hypothesized that, contrary to other TRAP methods that tag ribosomes, methods that quantify mRNA molecules associated with ribosomes and tagged partial translation products would produce broader readout of translation.

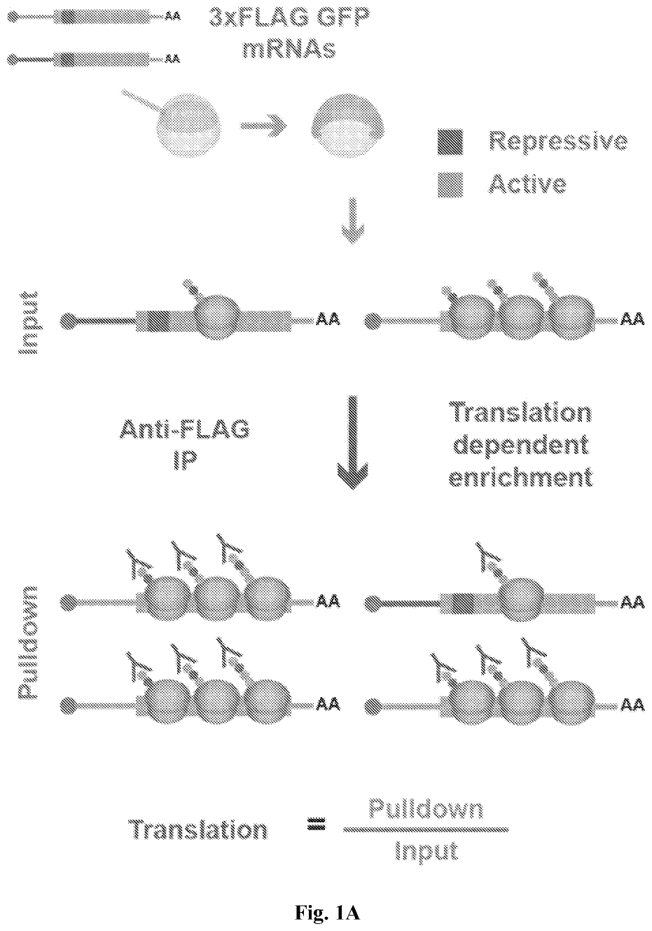

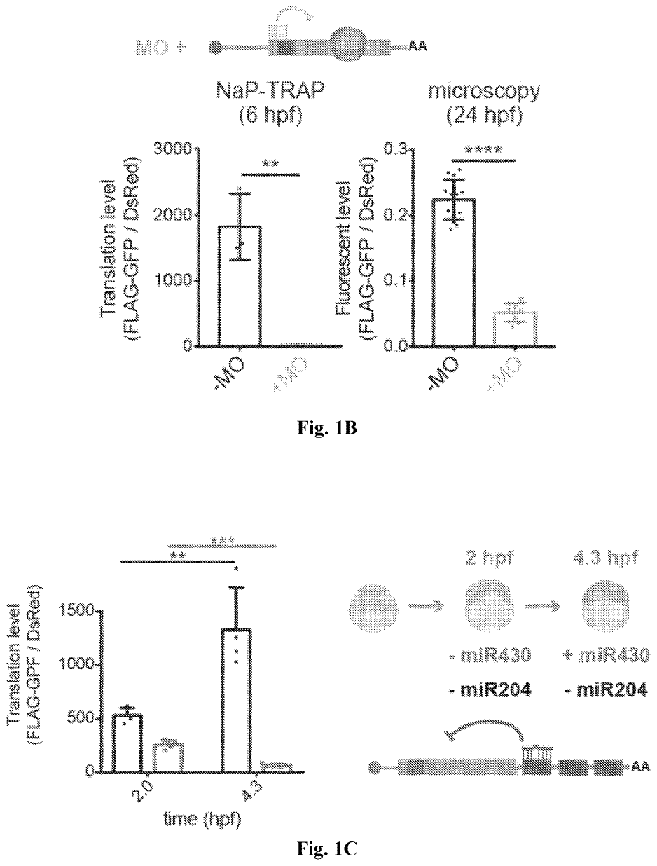

In conventional TRAP-based assays, the immunocapture of epitope tagged ribosomes immobilized by cycloheximide treatment enriches reporters in a manner proportional to their mean ribosome load. Like polysome profiling these assays capture all bound ribosomes. To measure frame specific translation, the present study elected to tag the nascent chain complex instead of the ribosome with an epitope. By incorporating a 3×FLAG-tag into the N-terminus of the coding sequence of GFP and performing an anti-FLAG immunoprecipitation, it was reasoned that translation could be measured as a ratio of reporter mRNA levels in the pulldown relative to the input (FIG. 1A).

Here the present study validated NaP-TRAP using reporters assays of increasing complexity. First, the present study measured the translation of individual reporters containing known cis-regulatory elements. Next, the present study quantified Kozak strength in zebrafish for the first time experimentally, measuring the translation of thousands of reporters simultaneously. Lastly, the present study assessed the regulatory potential of endogenous 5′ UTR sequences in the developing zebrafish embryo and HEK293T cells.

### Example 1-2: NaP-TRAP Measures Translation Via the Immunocapture of Nascent Chains

Cis-regulatory elements encoded in mRNAs modulate translation efficiency. To measure the regulatory potential of these elements the present study developed a novel massive parallel reporter assay (MPRA), NaP-TRAP (Nascent Peptide Translating Ribosome Affinity Purification). It was hypothesized that the present study could enrich reporters in a manner proportional to their translation efficiency through the immunocapture of FLAG-tagged nascent chain complexes immobilized by cycloheximide treatment. To evaluate the capacity of NaP-TRAP to measure translation the present study performed two reporter assays.

First, the present study measured the effect of a translation blocking morpholino. To this end, the present study co-injected 3×FLAG-GFP and dsRED mRNAs into single cell zebrafish embryos in the presence or absence of a translation blocking morpholino, targeting the start codon of 3×FLAG-GFP. Using NaP-TRAP the present study observed a 48-fold decrease in translation in the presence of the morpholino at 6hpf (FIG. 1B). These results are consistent with the translational repression in morpholino injected embryos observed by the decrease in fluorescence (GFP/dsRED) at 24 hpf (FIG. 1B). Next, the present study tested the capacity of NaP-TRAP to capture dynamic translation control. In the developing zebrafish embryo, the microRNA miR-430 is one of the first zygotically transcribed genes. At 4.3 hpf, the expression of miR-430 results in the translation repression, deadenylation, and eventual decay of targeted mRNAs. To quantify this effect, the present study measured the translation of 3×FLAG-GFP reporters with partial complementarity to two different microRNAs in their 3′ UTRs, miR-430 (3×FLAG-GFP-3×miR430) and miR-204 (3×FLAG-GFP-3×miR-204) respectively. The present study selected miR-204 target sites as the microRNA is not expressed in the early embryo. Using NaP-TRAP the present study measured the translation of mRNA reporters at 2 and 4.3 hpf, before and after miR-430 expression. Between the two timepoints, the translation of the 3×miR430 reporter decreased, whereas the translation of the 3×miR204 reporter increased. These results are consistent with the role of miR-430 in translational repression and the translation ramp observed during the early stages of development. Together these results demonstrate that NaP-TRAP measures the translation of individual reporters targeting general and developmentally dynamic cis-regulatory elements. Further, these experiments highlight the versatility of the method as they demonstrate the capacity of NaP-TRAP to quantify cis-regulation in both the 5′ and 3′ UTRs.

### Example 1-3: Using NaP-TRAP to Investigate Kozak Strength in the Developing Zebrafish Embryo

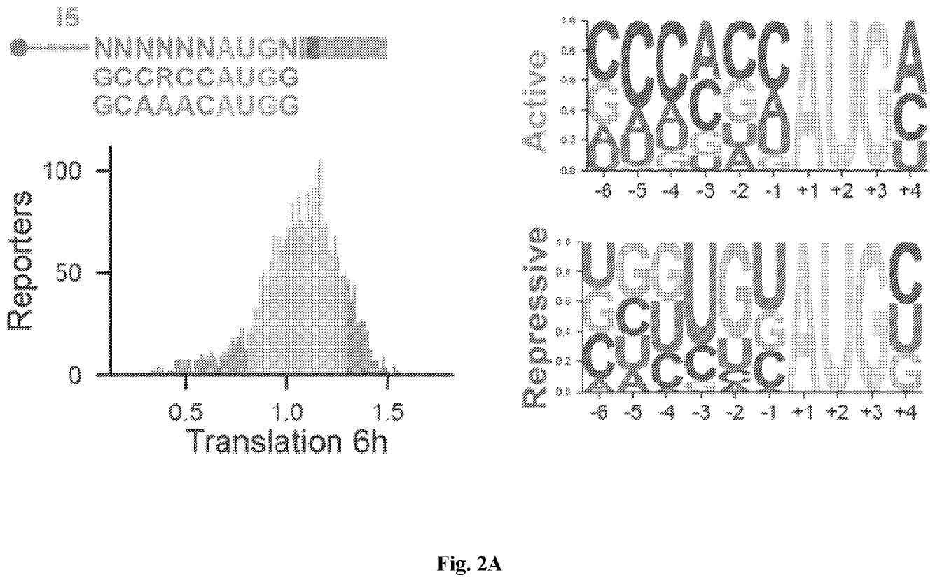

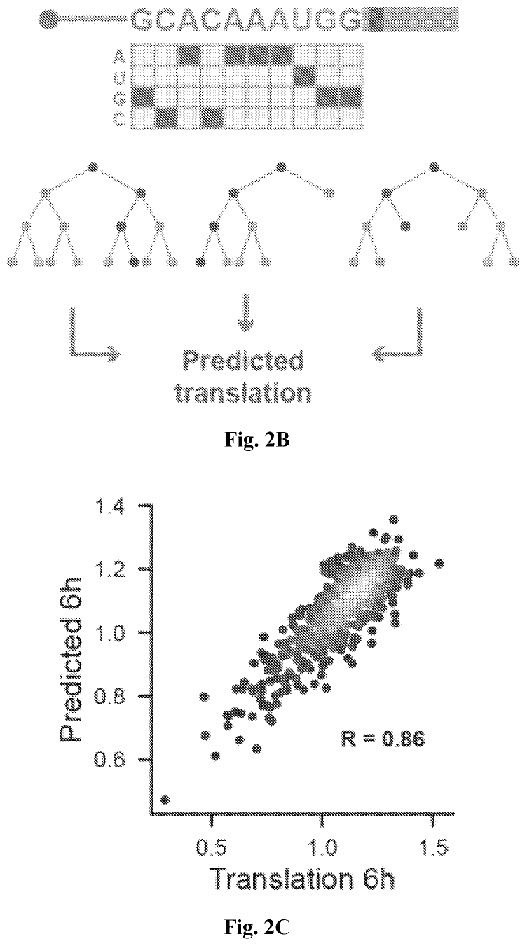

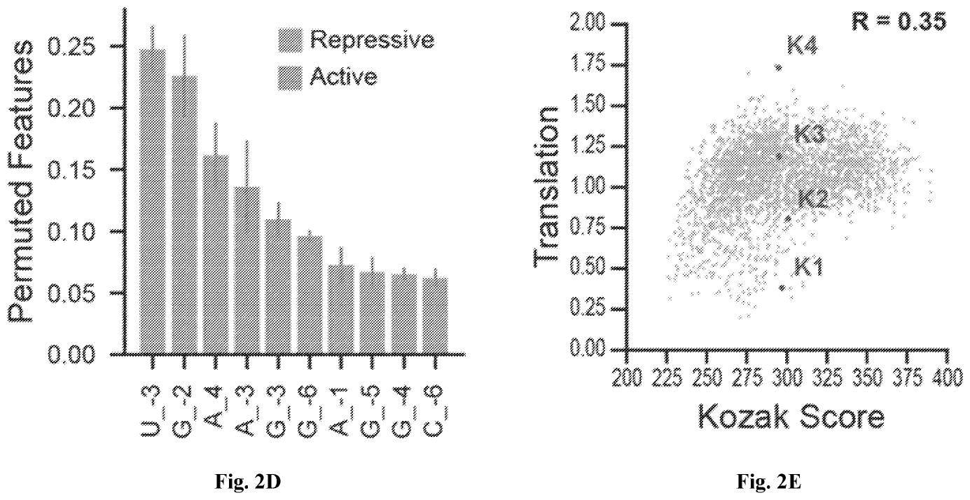

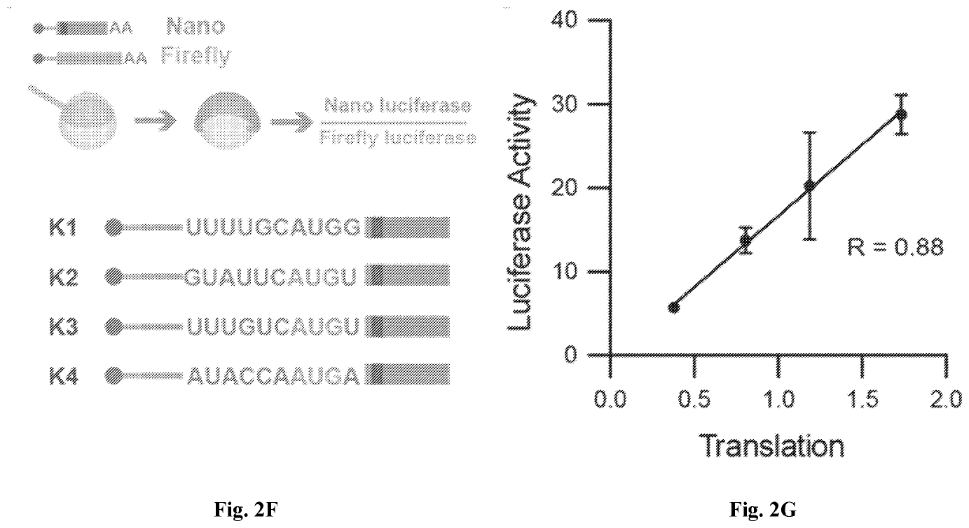

Next, to evaluate the capacity of NaP-TRAP to function as a MPRA, the present study designed a library to quantify the regulatory potential of the Kozak sequence, the present study elected to measure Kozak strength for three reasons: (1) the Kozak sequence is a strong determinant of translation initiation and (2) the Kozak strength is often inferred based on the frequency of these sequences in the genome, (3) Kozak strength has not been measured during development in vivo. To generate a library of diverse Kozak sequences, the present study incorporated six random nucleotides upstream and one random nucleotide downstream of the AUG of the 3×FLAG GFP reporter (FIG. 2A). The present study injected the in vitro transcribed mRNA library into single cell zebrafish embryos and measured the translation using NaP-TRAP at 6 hpf. The present study observed a strong correlation in translation values between replicates (Pearson's r=0.8). To identify active and repressive Kozak sequences, the present study generated position weight matrices for the top and bottom 10% of reporters based on their translation (FIG. 2A). The present study observed that active Kozak sequences were enriched in Cs and As, whereas repressive Kozak sequences were enriched in Us and Gs.

To examine the effect of nucleotide identity and position on translation the present study employed a random forest regression model (RFM). Model features were generated based on nucleotide identities and positions within the Kozak sequence. Prior to training the model, the present study divided the data into a test and a training set, 20% and 80% of reporters respectively. Using the training set, the present study employed a 5-fold cross validation to optimize model parameters. The model's prediction explained 86% of the variance observed in the test set data. Given this strong correlation the present study employed the model to predict the translation of all possible Kozaks. To identify the features informing the predictive power of the model, the present study performed a permutated feature importance analysis. Briefly, the present study shuffled the identities of the features of the model one at a time and measured the effect of the change on the predictive power of the model. At 6 hpf bases G, U, G, and C in positions −4, −3, −2 and +4 were the most repressive features, whereas C, A/G, and A in positions −6, −3, +4 were the most activating features.

Lastly, the present study compared NaP-TRAP translation values to an in silico derived metric (Kozak score). In zebrafish Kozak strength has been previously inferred based on the frequency of a given Kozak sequence within the transcriptome. This metric associates Kozak sequence abundance with increased translation initiation. The experimentally derived translation values here challenge this hypothesis as the present study observed a weak correlation between NaP-TRAP and Kozak score (R=0.35, FIG. 2E). To validate this conclusion, the present study identified sequences that had similar Kozak scores (300±5) yet exhibited different NaP-TRAP derived translation values and measured their translation using a dual luciferase reporter assay (FIG. 2F). The luciferase translation values correlated strongly with NaP-TRAP (Pearson R 0.88). This result not only highlights the importance of measuring Kozak strength experimentally, but also demonstrates that NaP-TRAP derived translation values correlate well with protein production. Taken together, these results demonstrate that NaP-TRAP can be employed as a MPRA. By measuring the translation of thousands of reporters simultaneously, the present study quantified and modeled Kozak strength for the first time in a vertebrate model.

### Example 1-4: Investigating the Regulatory Potential of Maternally Supplied 5′ UTRS

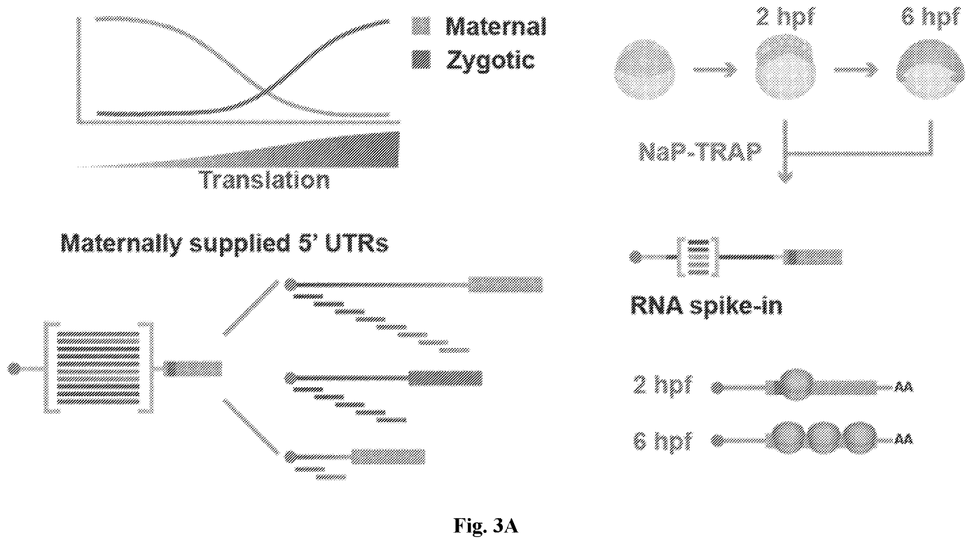

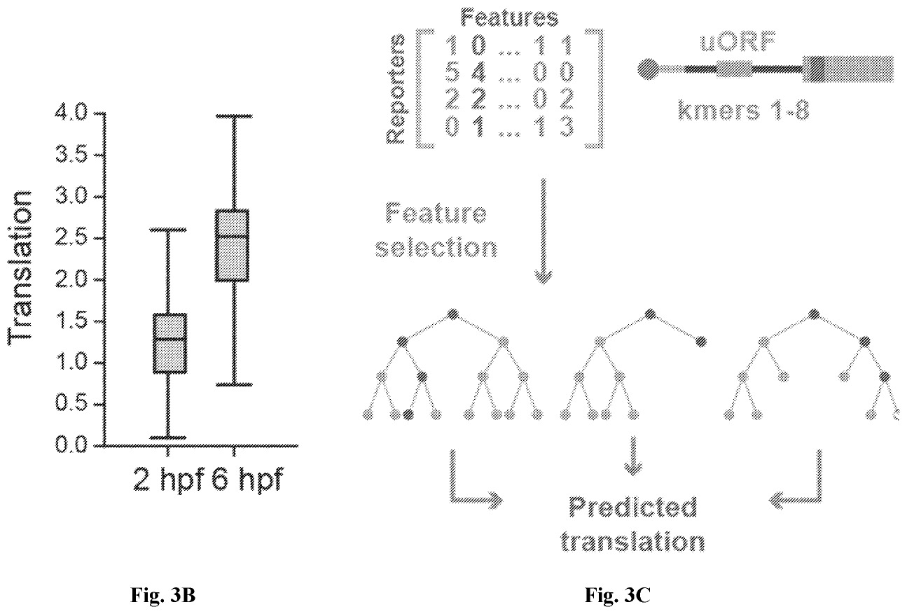

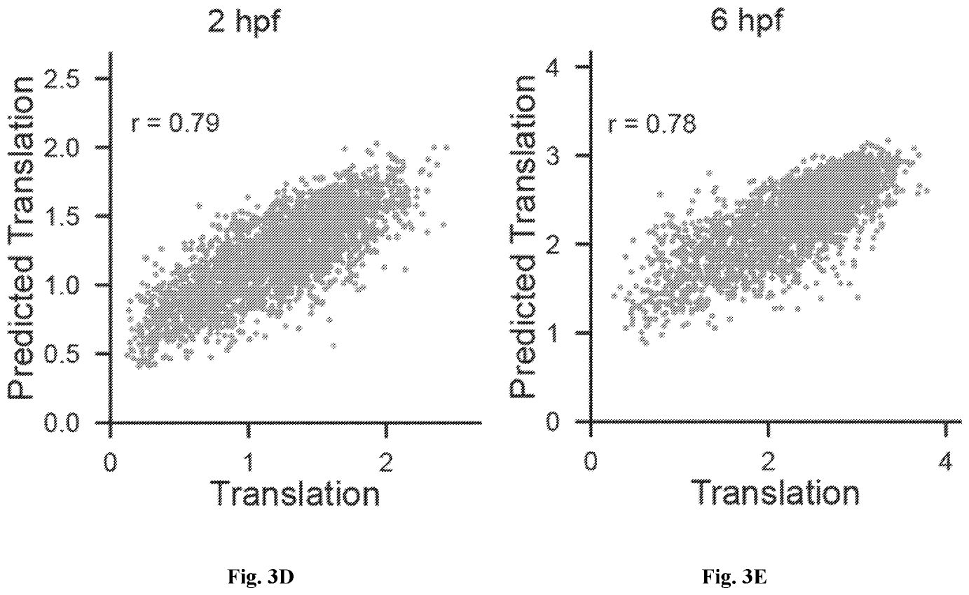

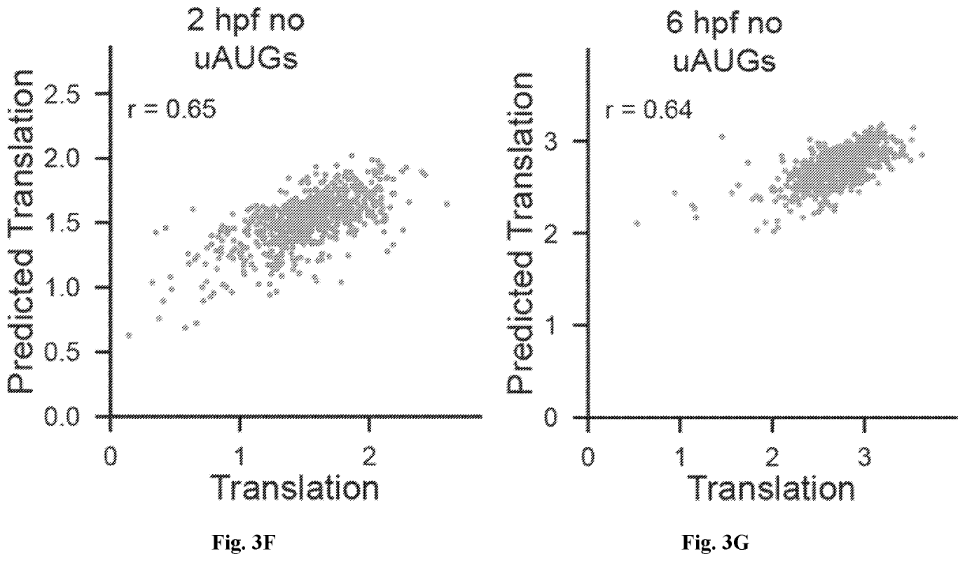

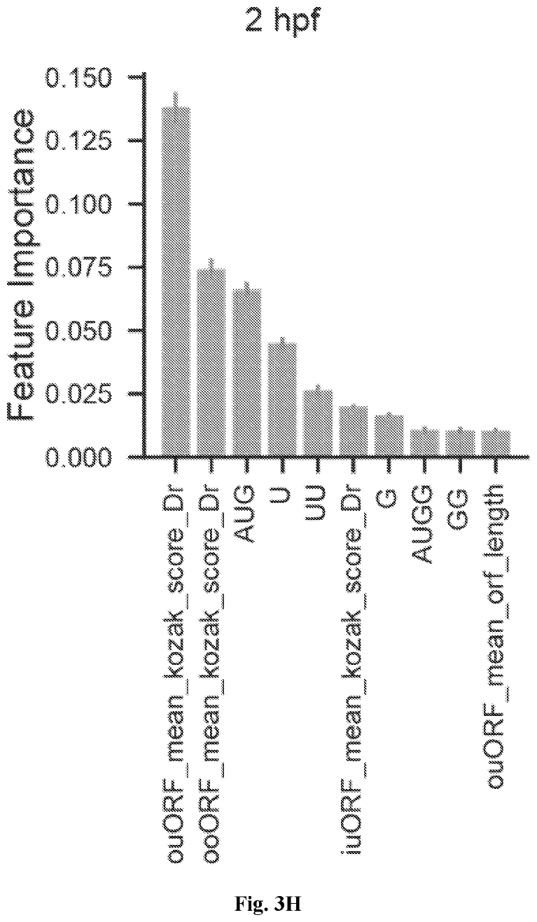

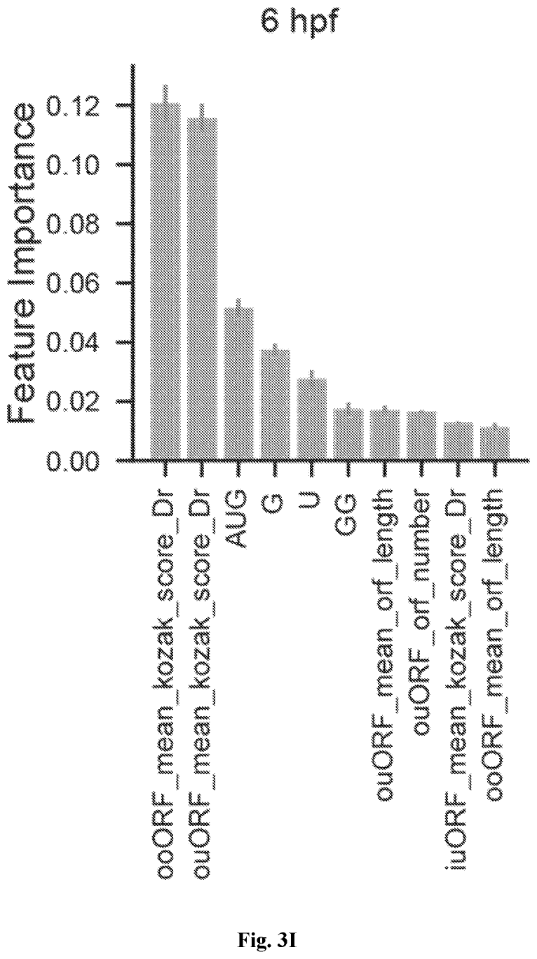

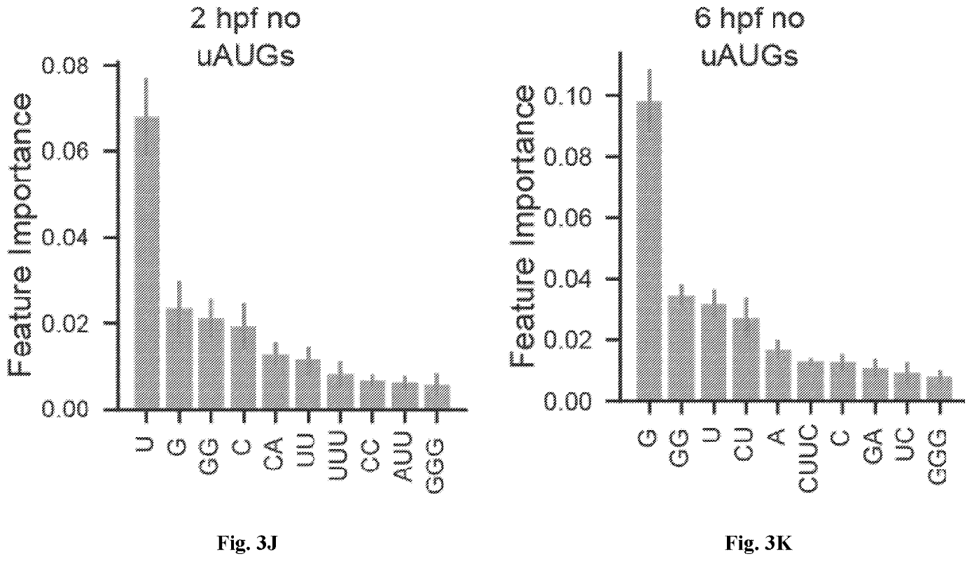

Upon validating NaP-TRAP's capacity to function as an MPRA, the present study investigated the regulatory complexity of endogenous 5′ UTRs during the early stages of embryogenesis. Prior to zygotic genome activation, the translation of maternally supplied mRNAs drives development. Given that initiation is the rate limiting step of translation, 5′ UTRs provide a mechanism for maternally supplied mRNAs to modulate their protein output. To study the cis-regulatory elements encoded in these mRNAs, the present study designed an 11,088-sequence synthetic oligo library (124-nt long) by tiling the 5′-UTRs of 1,725 maternal mRNAs every 25 nucleotides (FIG. 3A). The present study injected the in vitro transcribed library into single cell zebrafish embryos and measured translation at 2 hpf and 6 hpf using NaP-TRAP. Using an internal spike in at the RNA extraction step, allowed the present study to quantify relative changes in translation across developmental stages (FIG. 3A). The present study observed a mean increase in translation between 2 hpf and 6 hpf of 1.8-fold, consistent with gradual de-repression of translation during the first 24 hours of development (FIG. 3B).

To model translation at each time point, the present study utilized random forest regression models. For each reporter, the present study generated kmer counts (1-6 nucleotides) and characterized upstream open reading frames (uORFs) for a number of features (FIG. 3C). The present study filtered the features based on the strength of their correlation to translation at each timepoint (abs SPR>=0.05). Model parameters were optimized using a 5-fold cross validation on the training data (80% of reporters). The models predicted translation well at both timepoints, explaining >78% of variance observed in the test set data at 2 hpf and 6 hpf (FIG. 3D). The present study identified Kozak strength and the number of uORFs and out of frame oORFs as the features contributing most significantly to the predictive power of the model at both timepoints (FIG. 3F). Interestingly, features associated with upstream open reading frames exhibited larger feature importance at 6 hpf than at 2 hpf.

Given the prominent repressive effect of upstream open reading frames on translation, the present study repeated the analysis modeling reporters containing no uORFs or oORFs. Given that this subpopulation of reporters consisted of only about 20% of the original library, the present study trained the models on kmer counts ranging from 1 to 4 nucleotides. At 2 and 6 hpf the models explained 64% and 57% of the observed translation values respectively (FIGS. 3G-3H). At 2 hpf the most predictive features were U-rich and UA-rich kmers, whereas at 6 hpf G-rich and UC rich kmers were the most predictive features (FIGS. 31-3J). Interesting the present study observed that the effect of C and UC-rich kmers on translation changed during development. To explore this observation further the present study ranked kmers based on the mean difference in translation between reporters that were enriched or depleted in said kmer, the 80th and 20th quantile respectively. The present study compared the rank of features with a significant difference at both timepoints. The present study observed that C, CC, and UCC rich were some of the most repressive features at 2 hpf yet activated translation at 6 hpf (FIG. 3K), suggesting that there is a developmental switch on the regulatory role of these motifs during development.

All together these results demonstrate the following: (1) uAUGs are the most prominent repressive elements during early embryogenesis and (2) that 5′UTR sequences can confer dynamic regulation during development.

### Example 1-5: Identifying Sequences Driving Differential Translation

To identify systematically the sequence elements modulating differential translation during development the present study first divided the reporters into four groups: (1) repressed, (2) active, (3) early activated, and (4) late activated based on their relative translation at 2 and 6 hpf. Next, the present study performed a differential k-mer enrichment analysis in each group relative to the reporter library.

This analysis revealed that repressed reporters were significantly enriched in upstream ORFs and GC-rich kmers and depleted in U-rich tracks (p>10-5 hypergeometric test after Bonferroni correction). Conversely, active reporters were enriched in U-rich pentamers (UUUUU, CUUUU, UUUUA, GUUUU) and depleted in upstream start codons. Early activated reporters were enriched in AG/UG rich sequences (UAGUG, UAUUG, AAGAA, AGACU), as well as overlapping pentamers that were complementary to the seed site of the microRNA miR-430 (GCACU and AGCAC) and depleted in uORFs and pyrimidine repeats. In contrast, late activated reporters were enriched in pyrimidine repeats (CCUCC, UCUCU, CCCUC) and depleted in U-repeats.

To determine whether the observed regulation is the product of individual elements driving differential translation or the result of a combination of elements, the present study designed a library of repeats for all possible tetramers (FIG. 3H). The present study injected this in vitro transcribed library into single-cell zebrafish embryos and measured translation using NaP-TRAP at 2 and 6 hpf. Interestingly, the present study identified GUCU as the most active tetramer translating over 2-folds more than the nearest reporter at both timepoints. The present study compared the results of the validation library to the maternal 5′ UTR library by identifying tetramers enriched in the groups of reporters described above. The present study then plotted the distribution of the validation library, labeling each tetramer repeat based on their group enrichment in the maternal 5′UTR library (FIGS. 31-3M). While the distributions of repressed, early activated, and late activated tetramer reporters largely recapitulated the distributions of their respective groups in the maternal 5′ UTR library, the active tetramers exhibited a more heterogenous distribution. More specifically, U-rich kmers were not the principal driver of translation activation. The present study proposes that this discrepancy is the results of a difference in U repeat length between the respective libraries (FIG. 3J). In the tetramer library the longest U-repeat is five nucleotides, whereas U-repeats of 6 to 8 nucleotides are significantly enriched in the active reporters of the maternal 5′ UTR library. Interestingly, the present study once again observed translation repression of reporters with miR430 seeds at 6hpf (CACU and UUGC repeats).

Using this differential enrichment analysis, the present study identified general and developmentally dynamic regulators of translation, repressed and active reporters, and early and late activated reporters respectively.

### Example 1-6: Investigating the Mechanisms Driving Developmentally Dynamic Translation Control

In the developing zebrafish embryo, the pool of factors that regulate mRNA translation and decay changes overtime. Prior to zygotic genome activation at 4.3 hpf maternally supplied mRNAs and proteins drive translation control. Following genome activation zygotically expressed factors begin to reshape the post-transcriptional regulatory landscape. Given that the microRNA miR430 is one of the first zygotically transcribed factors, the enrichment of miR430 seeds in early activated reporters suggested that ZGA alters the translation of maternal 5′ UTRs. To determine the effect of ZGA on maternally supplied mRNAs, the present study injected the maternal library into single cell zebrafish embryos. The present study soaked the embryos in flavopiridol, a transcription inhibitor or DMSO and measured translation at 2 and 6 hpf.

### Example 1-7: 5′ UTR miR430 Recruitment Suppresses Translation

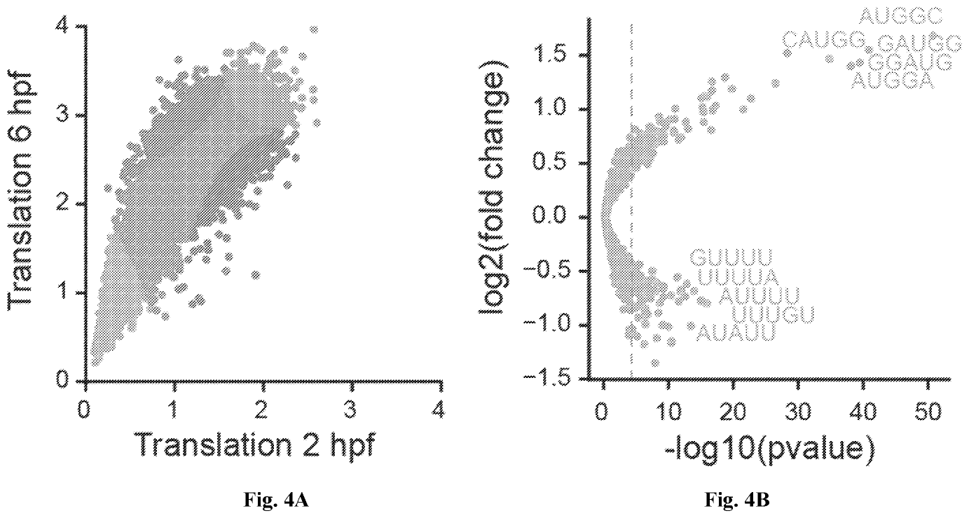

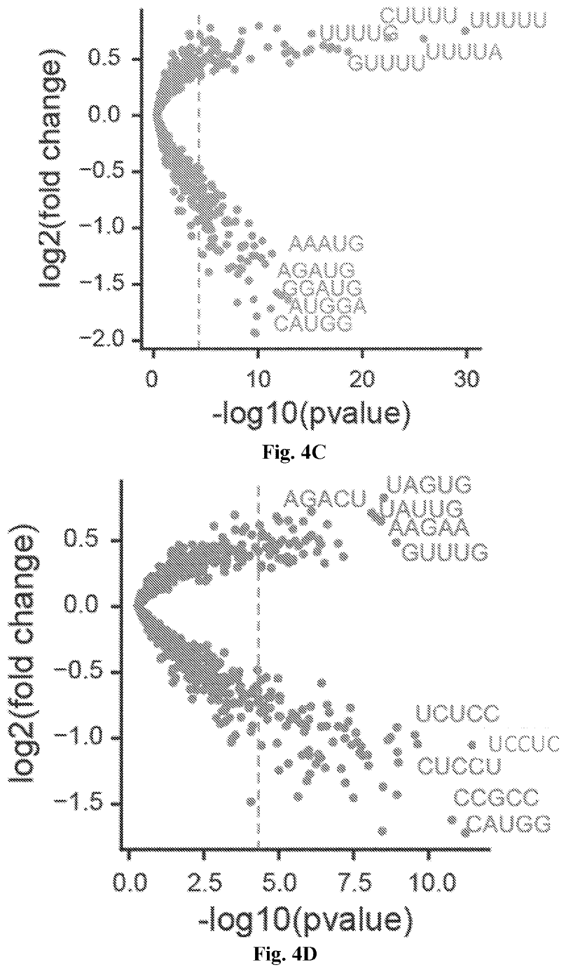

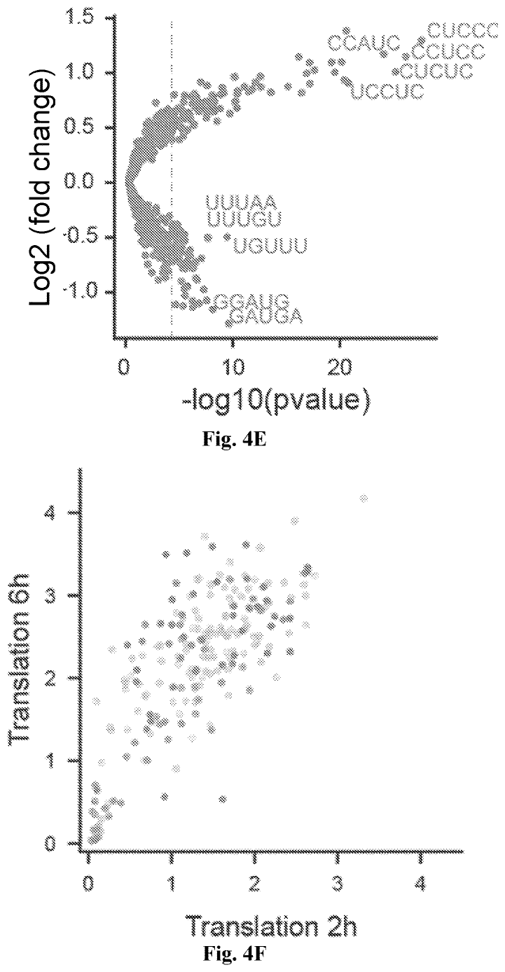

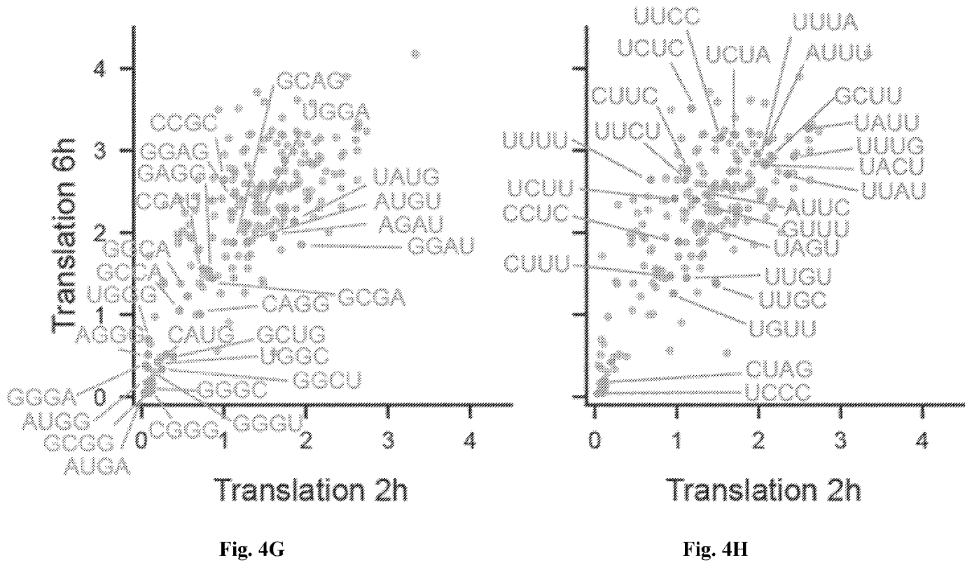

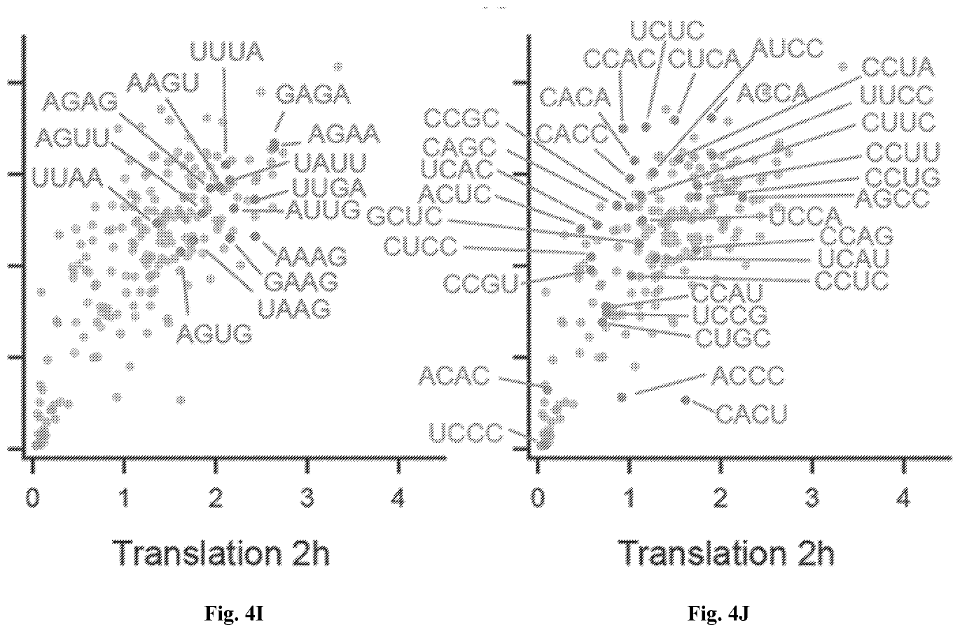

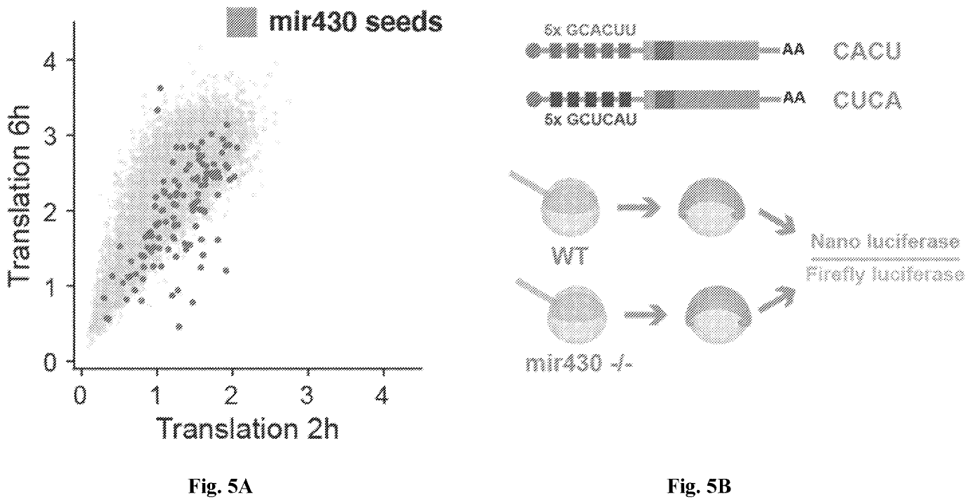

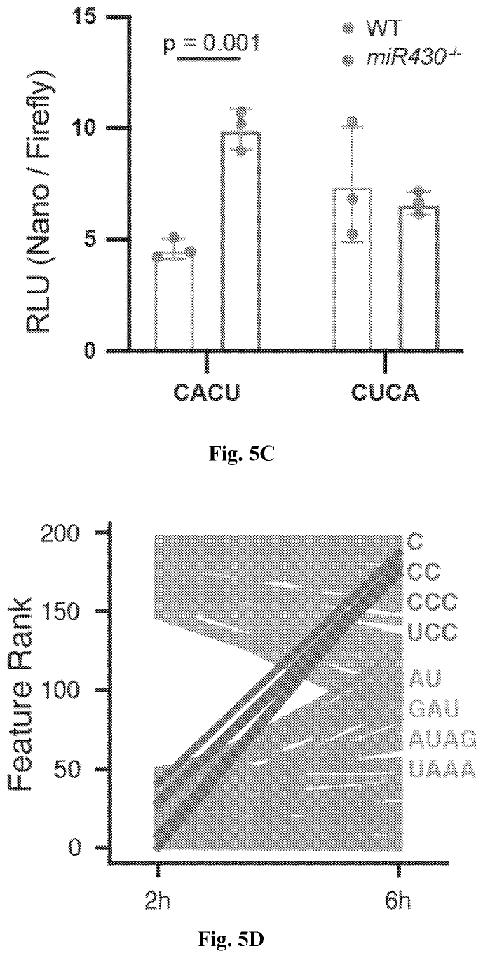

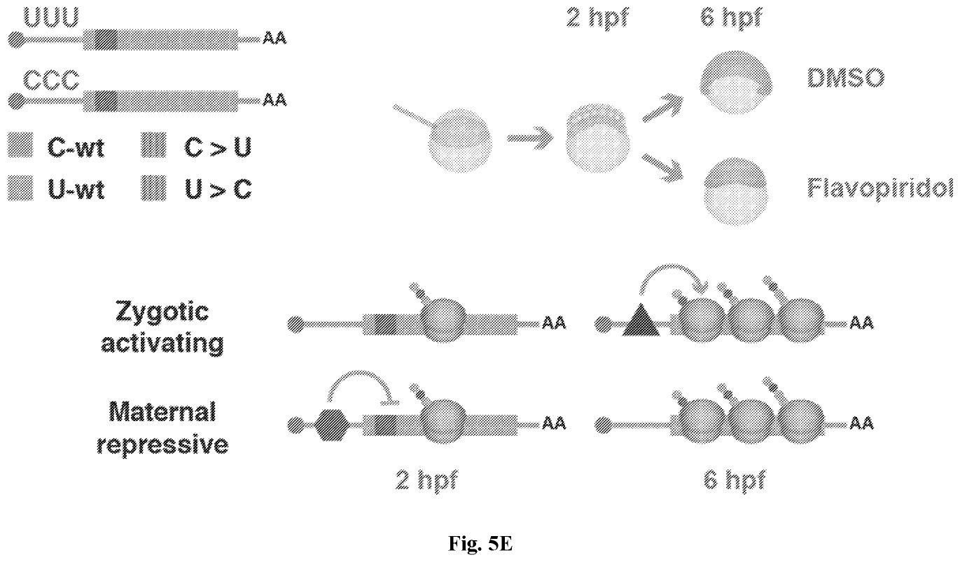

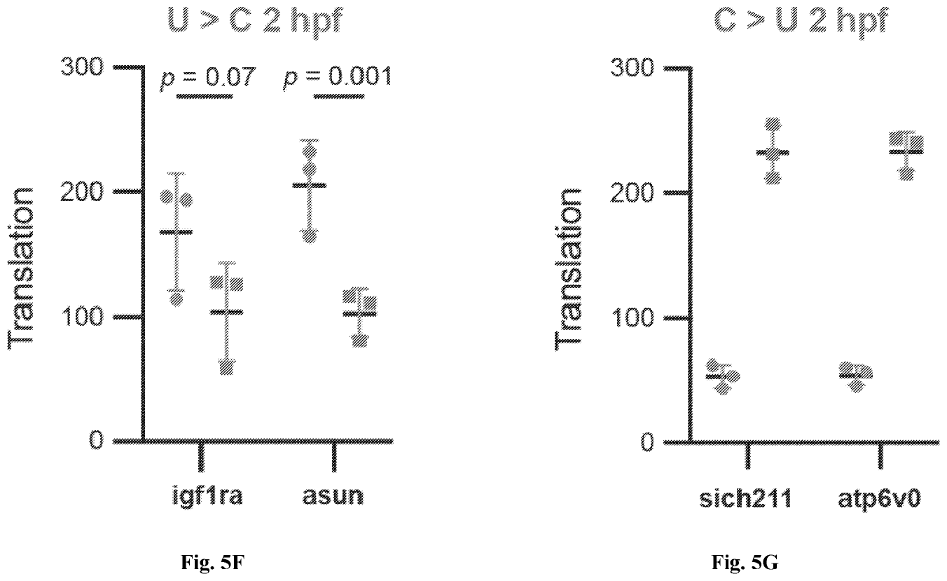

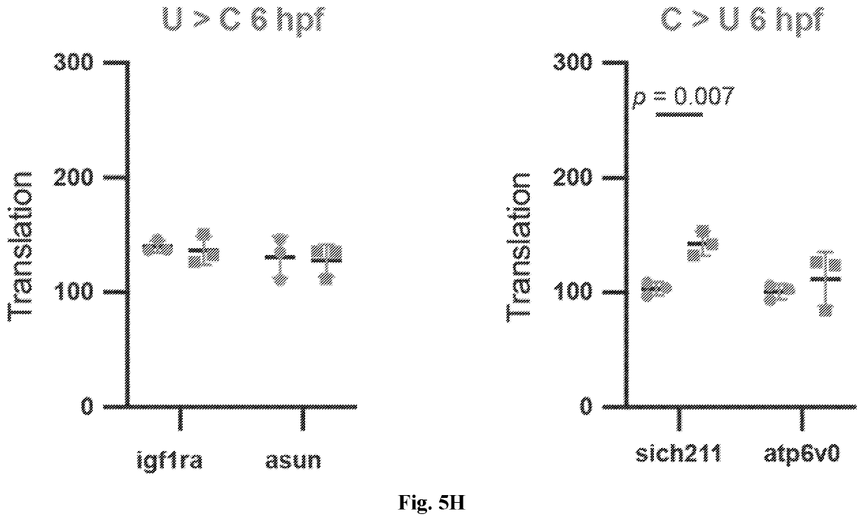

First, given that the microRNA miR430 is one of the first zygotically transcribed genes in the developing zebrafish embryo, the enrichment of miR430 seeds in early activated reporters suggests that genome activation shapes 5′ UTR mediated translation control (FIG. 4A). To validate this hypothesis, the present study constructed two nano-luciferase reporters: 5×miR430-nanoluc and 5×miR430-MUT-nanoluc. These reporters were identical with the exception of the fact that the 5′ UTR microRNA binding sites of the 3×miR430-shuffled-nanoluc reporter were rearranged to prevent miR430 recruitment. The present study injected these reporters into single-cell wild-type and miR430−/− mutant embryos in the presence of firefly luciferase and measured dual-luciferase activity at 6 hpf. The present study observed a 35% decrease in the ratio of wildtype to mutant luciferase activity for the miR430 reporter relative to the 3×miR430-shuffled-nanoluc reporter, confirming the hypothesis that the zygotic transcription of miR430 suppresses the translation of mRNAs with seed sites in their 5′ UTRs (FIG. 4B).

### Example 1-8: Zygotic Genome Activation Modulates the Effect of C-Richness on Translation?

Second, the present study observed an enrichment of pyrimidine pentamers in the late activated reporters. To investigate this differential regulation further the present study selected two active (U-rich reporters) and two late activated reporters (C-rich reporters) from the maternal 5′ UTR library. While the active reporters were translated ~2 fold more than the late activated reporters at 2 hpf, the translation of all reporters were similar at 6 hpf. To determine the role of C-richness in this translation regulation the present study mutated Us in the active reporters to Cs and mutated Cs in the late activated reporters to Us for a total of eight reporters: four wildtype and four mutants respectively. The present study injected the reporters into single cell zebrafish embryos in the presence and absence of a flavopiridol, a transcription inhibitor, and measured translation at 2 hpf and 6 hpf using NaP-TRAP. The present study observed that the C to U mutants enhanced translation at 2 hpf, whereas the U to C repressed translation at 2 hpf in both conditions. Strikingly, while these mutations did not significantly affect translation at 6 hpf in wild type conditions, the differences in the wild type and mutant reporters were maintained in the absence of zygotic genome activation. This observation suggests that a zygotic factor is driving the switch in the regulatory potential of pyrimidine rich elements. Given that the mutations were distributed randomly throughout each wild-type reporter, the present study proposes that the observed translational switch is driven by C-richness rather than a single motif. That being said, C-richness is not considered a universal activator at 6 hpf. The enrichment of GC rich pentamers in repressed reporters and the fact that the tetramer reporter CCCC was lowly translated at both timepoints suggests that the local context and abundance of Cs within a 5′ UTR are also important.

All together these results, validate novel developmentally dynamic mechanisms of 5′ UTR mediated translation control. Demonstrating the importance of quantifying cis regulation in non-study state systems.

### Example 1-9: NaP-TRAP can be Readily Adapted to Other Model Systems

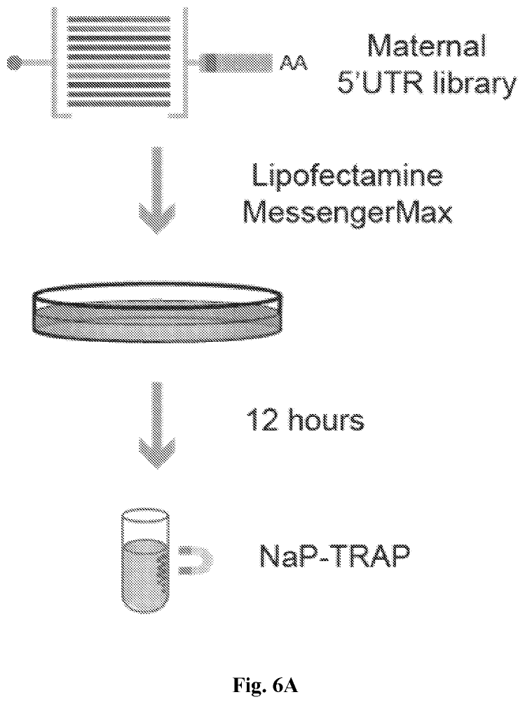

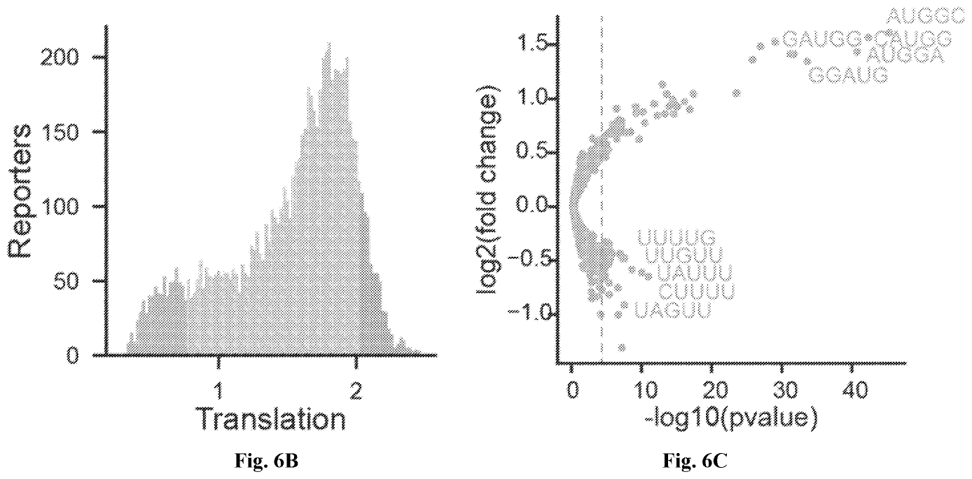

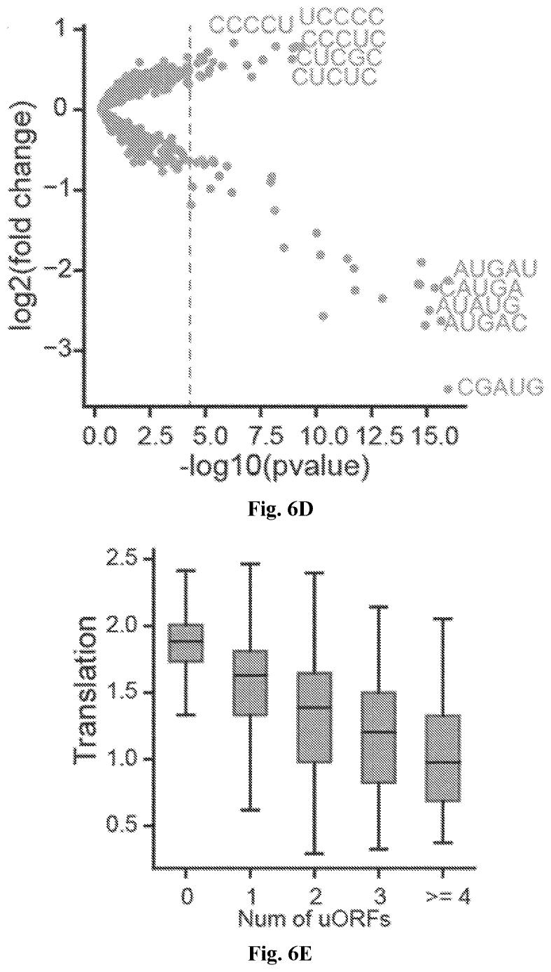

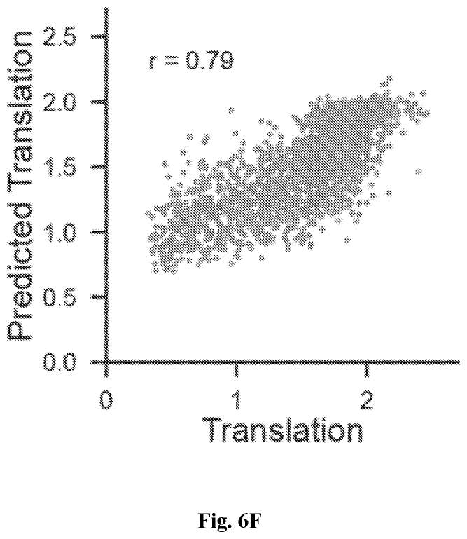

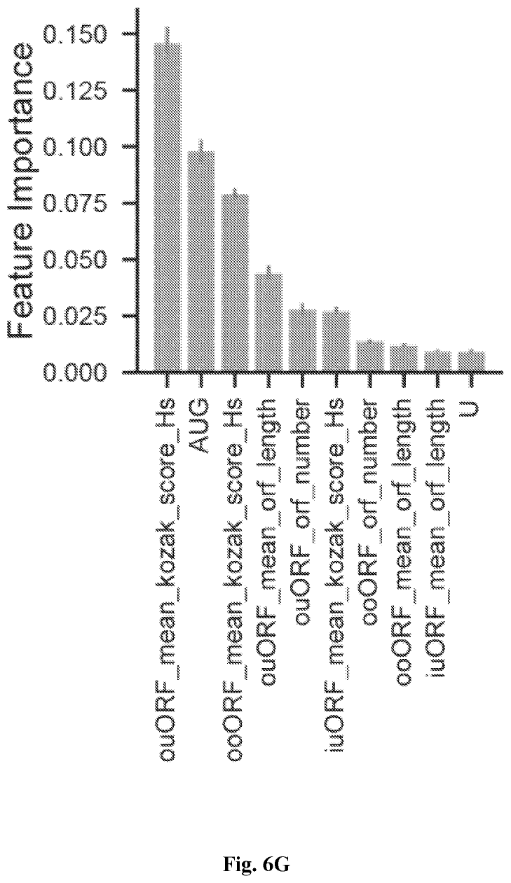

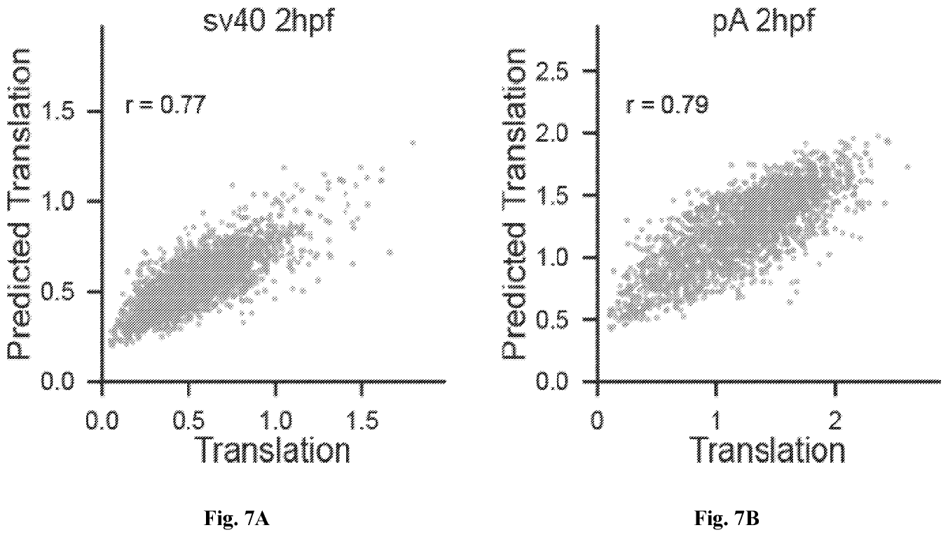

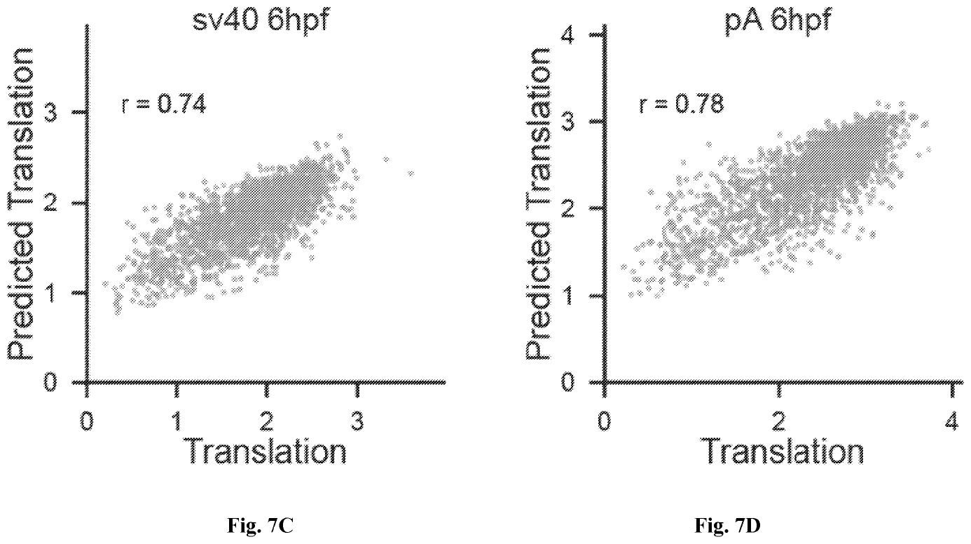

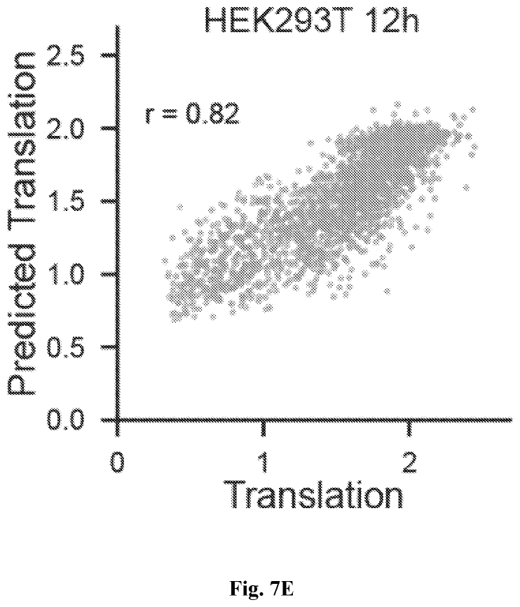

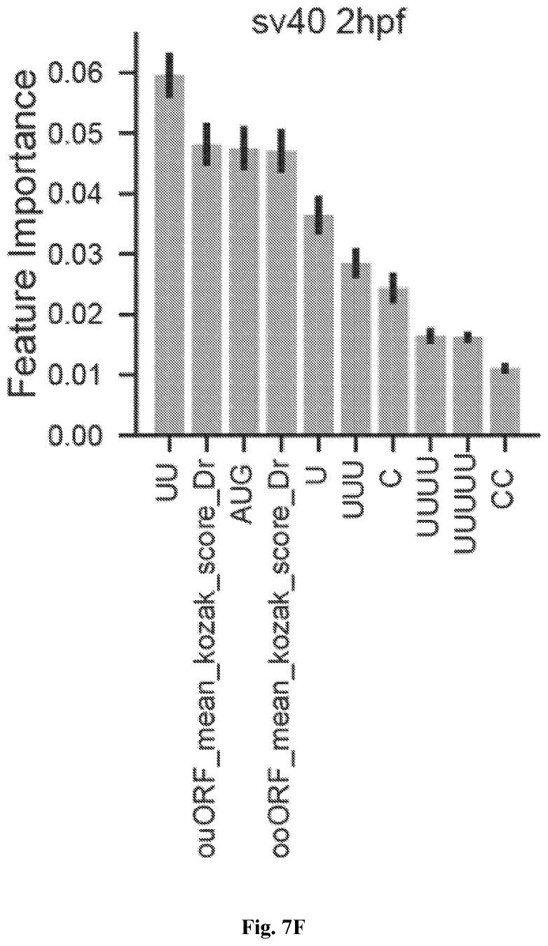

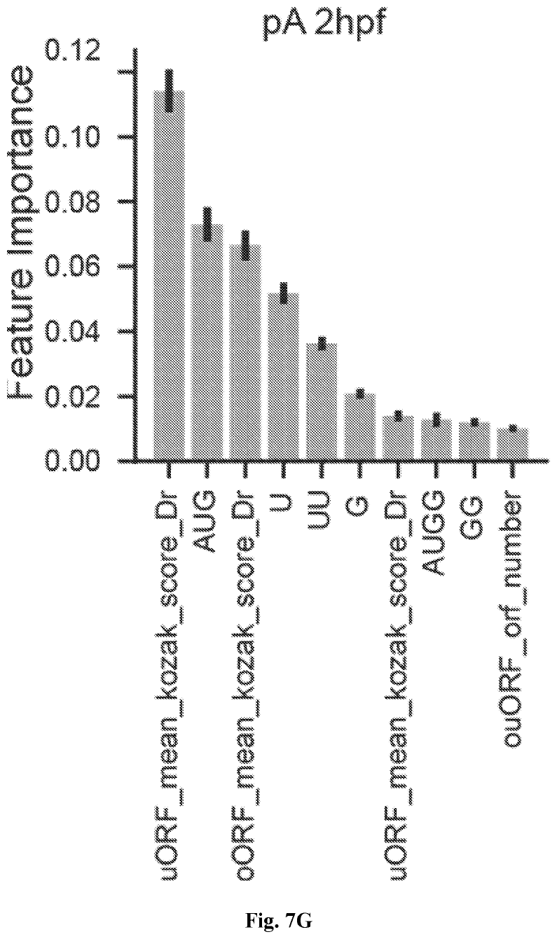

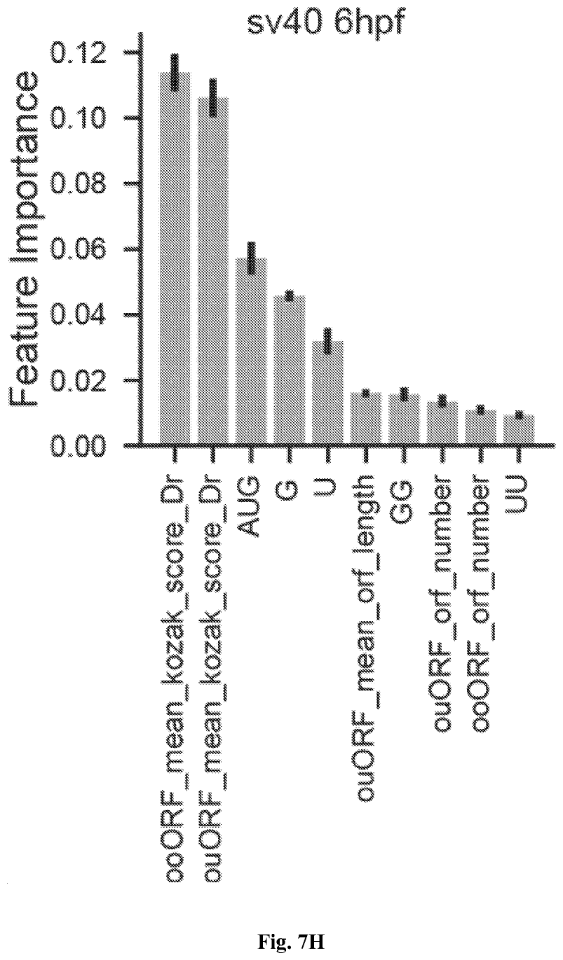

Lastly, to demonstrate the broad applicability of the method the present study performed NaP-TRAP in HEK293T cells. The present study transfected the in vitro transcribed maternal library into HEK293T cells using lipid nanoparticles and measured translation at 12-hour post transfection using NaP-TRAP. Replicates were strongly correlated (Pearson Correlation Coefficient of 0.92). To identify motifs modulating translation the present study performed a differential kmer enrichment analysis on active and repressed reporters, the top and bottom 10% of translation values respectively. Repressed reporters were enriched in AUG containing motifs and depleted in U-rich pentamers, whereas active reporters were enriched in pyrimidine and poly U rich pentamers, and depleted in AUG containing motifs (FIGS. 6C-6D). Interestingly the human translation values correlated equally well with the zebrafish translation values at 2 hpf and 6 hpf (SPR 0.78 and 0.79 respectively), despite the fact that pyrimidine rich pentamers were only enriched in the active reporters of the human and 6 hpf data.

The magnitude of the fold-change enrichment and depletion of AUG pentamers in repressed and active reporters respectively suggests that the presence or absence of upstream ORFs are a dominant driver of translation in HEK293T cells. In agreement with this observation, the present study observe a strong negative association between uORF number and translation (FIG. 6E). Lastly, the present study employed a RFM to model translation; the model explained 75% of the experimental data (FIG. 6F). In agreement with the zebrafish data, Kozak strength and number of upstream AUGs were the most predictive features. Interestingly, these features had a greater contribution to the predictive power of the model when compared to the zebrafish feature importance analysis (FIG. 6G).

All together these results demonstrate that (1) NaP-TRAP is a robust method that can measure translation across multiple model systems, and (2) that upstream AUGs are a dominant driver of 5′ UTR mediated translation control in Hek293T cells.

### Example 1-10

Here, the present study developed a novel method (NaP-TRAP) and demonstrate its capacity to measure translation quantitatively. The present study use NaP-TRAP to characterize cis-regulatory elements in the 5′ and 3′ UTRs, the strength of the Kozak sequence, and the regulatory landscape of the 5′ UTRs of maternally supplied mRNAs in the developing zebrafish embryo and HEK293T cells. The present study identified conserved and developmentally dynamic cis-regulatory elements as well as characterized global changes to translation associated with early embryogenesis.

NaP-TRAP marks a significant improvement to translation based MPRAs. In contrast to existing methods NaP-TRAP measures translation an open reading frame specific manner without the potential for transcriptional bias. By enriching for reporters through the immunocapture of FLAG-tagged nascent chain complexes, NaP-TRAP measures active ribosomes translating in frame the tagged ORF. This specificity results in more accurate quantification of translation, as NaP-TRAP derived translation measurements, correlate well with protein output (FIG. 2G).

To demonstrate the capacity of NaP-TRAP to quantify the translation of thousands of reporters simultaneously the present study measure Kozak strength for the first time in the developing zebrafish embryo. Interestingly, the present study observe a weak correlation between Kozak strength and the frequency of Kozak sequences in the genome, an in silico derived Kozak score. This observation highlights the importance of measuring Kozak strength experimentally. Novel Kozak sequences that differ from the consensus zebrafish or vertebrate Kozaks have been identified. Further, the present study has utilized the random forest regression model to predict the Kozak strength of all zebrafish Kozak sequences (position-6 to +4). This prediction will improve the annotation of ORFs in the zebrafish transcriptome as well as assist with the development of more efficiently expressed transgenes.

Development requires dynamic spatial and temporal control of translation. Using NaP-TRAP the present study has identified cis-regulatory elements that differentially regulate translation in development in the 5′ UTR. For example, the present study has shown for the first time that the zygotically expressed microRNA miR430 represses the translation of reporters containing miR430 seeds in the 5′ UTR. This work adds to a growing body of literature demonstrating the capacity of microRNAs to regulate translation outside of canonical 3′ UTR binding sites. The present study also identified a translation switch driven by pyrimidine rich motifs. At 2 hpf pyrimidine rich kmers suppress translation, whereas at 6 hpf these kmers activate translation. The present study proposes two potential mechanisms to explain this novel 5′ UTR mediated translation control.

First, the expression or loss of a trans-acting factor (TAFs) that binds pyrimidine rich tracts may drive differential translation. During the maternal-to-zygotic transition the pool of regulatory TAFs is dynamic. The present study proposes two potential candidates for activation of pyrimidine tracts: (1) a member of the poly-pyrimidine tract-binding protein family (PTBP) or (2) a component of the EIF3 complex. Previous work has demonstrated that both of these factors are recruited to pyrimidine tracts in the 5′ UTR. (1) While PTBPs have been primarily associated with the translation activation of viral IRESs, recent work from Pan et al has shown how the loss of a PTBP2 binding site in the 5′ UTR of FGF13 represses cap-dependent translation in humans. Further, in the developing zebrafish the translation efficiency of PTBP1a increases dramatically following zygotic genome activation. (2) The EIF3 complex is a potent regulator of 5′ UTR mediated translation control, as it is a component of the scanning 40S ribosome. Several works have suggested that differential expression of EIF3 components during development contributes to the translation of mRNAs in a lineage specific manner.

Second, recruitment of trans-factors depends not only on the presence of a given cis-regulatory element, but rather the composition of the transcriptome and the pool of trans-factors shapes the regulatory potential of a given element. In fact, the present study observed that the relative importance of U-richness depends on the global rate of translation. When translation is low U-richness is the prominent feature predicting translation (sv40 supplement). In contrast, as translation increases the relative importance of U-richness declines, whereas the relative importance of uORFs increases. It is proposed that this change, reflects a change in the availability of the translation initiation machinery. It is speculated that when the supply of ribosomes is limited, the capacity of the 5′ UTR to recruit ribosomes drives translation. In contrast, as the ribosome pool increases the effect of ribosome recruitment diminishes.

This competition model can also be employed to explain the differential effect of pyrimidine tracts on translation. In the early embryo reporters enriched in U-repeats may recruit the limited supply of ribosomes more efficiently than reporters enriched in pyrimidine tracks. As the supply of translation machinery increases the effect of competition diminishes, resulting in the efficient initiation of pyrimidine rich 5′ UTRs. Repressive TAFs may amplify the effect of competition, as in the absence of scanning 40S ribosomes, these factors can be more readily recruited to the 5′ UTR. Work from Xiang and Bartel investigating the strong correlation between translation efficiency and poly-A tail length exemplifies this model. More specifically, they report that prior to gastrulation the pool of PAPBC1 is limited, driving competition between transcripts for PAPBC1 occupancy. This competition is eliminated following gastrulation as the relative abundance of PAPBC1 increases following zygotic expression resulting in the decoupling of poly-A tail length and translation efficiency.

NaP-TRAP also identifies general regulators of translation. In both the developing zebrafish embryo and HEK293T cells, the present study observed poly-U repeats greater than five nucleotides as a general activator of translation initiation. The fact that U repeats are activators of translation across timepoints and experimental systems, suggests that this observation may be driven by a component of the canonical translation initiation machinery.

### Example 1-11: Methods

### Zebrafish Maintenance and Mating.

Wild-type zebrafish embryos were obtained through natural mating of TU-AB strain of mixed ages (5-18 months). Mating pairs were randomly chosen from a pool of 60 males and 60 females allocated for each day of the month. Fish lines were maintained following the International Association for Assessment and Accreditation of Laboratory Animal Care research guidelines and approved by the Yale University Institutional Animal Care and Use Committee (IACUC).

### NaP-TRAP Reporter Controls

To enable nascent chain immunocapture a 3×FLAG tag was incorporated after the first 18 nucleotides of GFP-3×AID* (auxin inducible domain) using infusion cloning. While AID* domains were included in the initial NaP-TRAP vector to enable the future use of an auxin-inducible degron system, this system was not utilized in this study. The vector also included an SP6 promoter sequence and SV40 poly-adenylation signal. NaP-TRAP reporter mRNAs were generated using the mMESSAGE mMACHINE™ SP6 transcription kit from linearized reporter plasmids (NOT1 restriction enzyme).

To validate NaP-TRAP, two reporter experiments were performed. First, to quantify morpholino mediated translation repression using NaP-TRAP, 100 μg of 3×FLAG-GFP-3×AID and 75 μg of dsRED were injected into single cell zebrafish embryos in the presence or absence of 250 μM of a morpholino targeting GFP (Primer X). Embryos were collected at 6 and 24 hpf (25 embryos per NaP-TRAP replicate). NaP-TRAP was performed at 6 hpf whereas immunofluorescence was performed at 24 hpf using Microscope and images were quantified using ImageJ. Second, to assess the capacity of NaP-TRAP to measure microRNA mediated repression, two additional reporters were generated: (1) 3×FLAG-GFP-3×miR430 and (2) 3×FLAG-GFP-3×miR204. Three binding sites of either miR430 or miR204 were cloned into the 3′ UTR of 3×-FLAG-GFP-3×AID*. Single cell embryos were injected with 20 μg of the 3×FLAG-GFP-3×miR430 and 3×FLAG-GFP-3×miR204, and 160 ng of dsRED mRNAs. Twenty-five embryos per replicate were collected and flash frozen in liquid nitrogen at 2 and 4.3 hpf.

Following NaP-TRAP, cDNA was synthesized using random hexamer and SuperScript III reverse transcriptase following the manufacturer's protocol. Translation values were determined by qPCR using the Applied Biosystems power SYBR Green PCR master mix. Levels of reporters in the input and pulldown were normalized using the 2^{-ΔΔCt} method (ref, PMID: 11846609), where the NaP-TRAP reporter is the target and dsRED the control. Translation was calculated as a ratio of fold enrichment in the pulldown relative to the input.

### NaP-TRAP Reporter Library Assembly

To eliminate excess cytoplasmic 3×FLAG-GFP, a C-terminal PEST domain was incorporated into the NaP-TRAP reporter plasmid using infusion cloning. The 3×FLAG-GFP vector was amplified from the 3×FLAG-GFP-3×AID* plasmid, whereas the PEST domain insert was generated using PCR overlap extension. For the sake of brevity, 3×-FLAG-GFP-PEST is referred to as 3×FLAG-GFP in the text and figures of this section.

NaP-TRAP reporter libraries were constructed using three different PCR reactions. First, the common coding sequence and 3′ UTR of the reporters were amplified from the 3×FLAG-GFP plasmid, using a forward primer targeting the N-terminus of GFP and reverse primer targeting the 3′ end of the 3′ UTR. For the Kozak library a forward primer targeting downstream of the Kozak sequence was used. All PCR amplicons were gel purified (Monarch).

Second, reporter libraries were amplified from single stranded oligo pools (KAPA, 20 cycles). PCR overlap extension was used to generate double stranded DNA (0.1-1 ng of template DNA). For the Kozak library primer XX was utilized as a reverse primer, whereas the maternal 5′ UTR and the validation libraries utilized primer XX. After 10 cycles a forward primer, containing an SP6 promoter sequence was added to the PCR reaction. Amplicons were purified using the Zymo DNA Clean & Concentrator (DCC) kit in accordance with the manufacturer's instructions.

Third, to generate a template for in vitro transcription, a PCR overlap extension was performed between the reporter library and the purified 3×FLAG-GFP-PEST amplicons (KAPA HiFi, 20 cycles). To amplify the reporter library primers targeting the SP6 promoter sequence and the 3′ end of the 3×FLAG-GFP-PEST amplicons were added after 10 cycles of overlap extension. To generate reporters with a hard encoded poly-A tail, the reverse primer had a 10 or 60 T 3′ overhang. Unless stated explicitly reporter libraries contained a 60A tail. For libraries with an SV40 polyadenylation signal, a reverse primer targeting the 3′ end of the SV40 pA signal (located directly downstream of the 3′ UTR) was used for the amplification of 3×FLAG-GFP-PEST and the reporter library.

Lastly, templates for in vitro transcription were gel purified. Reporter mRNAs were generated using the mMESSAGE mMACHINE™ SP6 transcription kit. In vitro transcribed mRNAs were purified using Monarch kit.

### Random Kozak Library

The variable region of the random Kozak library consisted of an 15 Illumina adaptor followed the 5′ UTR of xenopus beta-globin, seven random nucleotides, six upstream and one downstream of a start codon, and the N-terminus of 3×FLAG GFP.

### Maternal 5′UTR Library

A custom single stranded DNA oligo pool, consisting of 11,088 oligos, was ordered from Genscript. Each 170 nt oligo contained an Illumina 15 adaptor sequence, a 124-nucleotide variable region, and 22 nt region with homology to the Kozak sequence and N-terminus of 3×FLAG-GFP. The variable region of the maternal 5′ UTR library was generated using a custom script, tiling the 5′ UTRs of 1,775 maternally supplied transcripts and six IRES sequences (human AQP4, human MYT2, human NRF, human XIAP, EMCV and crTMV) in 124 nucleotide segments every 25 nucleotides.

### Kmer Validation Library

A custom singled stranded DNA oligo pool, consisting of 256 oligos was ordered from Twist Biosciences. The design of the common regions of library were identical to that of the maternal 5′ UTR library. The variable region (124 nucleotides) consisted of repeats of all possible tetramers. Each repeat occurred 21 times and was separated by two nucleotides. The nucleotides were repeated in a pattern across the variable region (TC, AC, AG, CG, TC). These dinucleotides were selected to prevent the creation of unintended upstream ORFs.

### NaP-TRAP (Nascent Peptide Translating Ribosome Affinity Purification)

To capture tagged nascent chains of reporter mRNAs, an immunoprecipitation using anti-FLAG magnetic beads was performed. Magnetic beads were purchased from three suppliers during the course study do to supply chain issues. For both the zebrafish and human cell experiments 10 μL of beads (binding capacity of 15 μg of FLAG peptide) were utilized. The magnetic beads were washed with 800 μL of wash buffer three times prior to being added to the lysis solution.

Frozen embryos and HEK293T cells were lysed in 500 μL of lysis buffer. After 10 minutes at 4 C the lysate was passed through a 25 G needle (5-10 times). Samples were then centrifuged at 16,000 g for 5 minutes at 4 C. The supernatants were transferred to a new Eppendorf tube and 2 μL of DNASEI were added. After a 15-minute incubation at 4 C, the samples were diluted to 1 mL using additional lysate buffer and 75 μL of lysate was collected from each sample to serve as an input. Samples were placed on a rotator at 4 C for 2 hrs. Following incubation, the beads were washed with 800 μL of wash buffer three times. The beads (pulldown) and the inputs were then resuspended in 1 mL of Trizol. For the maternal 5′ UTR and tetramer validation libraries, spike in reporters were added. Trizol extractions were performed in accordance with the manufacture's protocol. RNA pellets were resuspended in 11 μL of nuclease free H₂O.

### Reporter Library Preparation

Reverse primers (4 μM total) targeting the N-terminus of 3×FLAG GFP were added to purified input and pulldown RNAs. These primers contained a 6 nt sample barcode and 10 nt Unique Molecular Identifier to allow for demultiplexing and read deduplication as well as a 3′ 17 Illumina adaptor sequence. To increase library complexity the primer pairs were staggered by 1 nt. Reverse transcription was performed using the Superscript III kit in accordance with manufacturer's instructions. Reverse transcription reactions were performed at 55 C. cDNA from replicates were pooled and purified using AMPURE XP beads. Illumina I5 and I7 forward and reverse primers containing a 10 nucleotides indexes were utilized to amplify cDNA libraries via PCR (Kappa Polymerase Master Mix). To reduce the number of PCR duplicates 12-18 cycles were utilized. Amplicons were purified by using an Agarose Gel Electrophoresis followed by size selection and gel extraction (Monarch Kit). Libraries were sequenced using NovaSeq 6000.

### Read Trimming and Translation Calculation

Paired-end reads were trimmed and demultiplexed using ReadKnead (github). Barcodes identifying replicates and UMIs were extracted from read two. Common regions were trimmed from read one to facilitate accurate mapping. For the Kozak library reporters were counted using a custom python script. Reporter with indels in the Kozak sequence or reporters without an AUG in the appropriate position were eliminated. For the maternal 5′ UTR and validation libraries reads were mapped to a library specific index using Bowtie2 (command here). PCR duplicates were eliminated using UMIs. UMIs were considered identical if they had a Hamming Distance less than 1. Reads for each experiment were normalized by dividing the read counts of each reporter by the sum the total number of reads mapped to the spike-ins. In the absence of spike-ins read counts were normalized reads based on the total number of mapped reads per replicate (reads per million, RPM).

### Differential Motif Enrichment Analysis

Reporters were ranked based on their translation at 2 and 6 hpf. Using the sum and difference of these rankings across timepoints four groups of reporters were generated: (1) repressed, (2) active, (3) early activating, and (4) late activating. Repressed and active reporters constituted the top and bottom 10% of reporters based on the sum of their ranks at 2 and 6 hpf respectively, whereas the early activated and late activated groups were the top and bottom 10% of groups based on the difference between their ranks at 2 and 6 hpf. A differential motif enrichment analysis was performed on each group. Fold enrichment values were determined by dividing the count of each kmer in the reporter group by the count of the kmer in the library, whereas the significance of the fold-change was determined using a hypergeometric test (Bonferroni corrected p-value).

### Random Forest Regression Model

Random forest regression models were employed to predict translation. For the Kozak library features were generated by one-hot encoding positions −6 to −1 and position +4 of each reporter sequence. In contrast, for the maternal 5′ UTR library Kmer counts (1-8 nucleotides) and uORF features were generated using a custom python script. For the maternal 5′ UTR models features were filtered by calculating a spearman rank correlation coefficient (SPR) between each feature and translation. Features that had a correlation greater than 0.05 or less than-0.05 were included in the Random Forest Model.

To prevent model overfitting the data were divided randomly into two groups: a test and training set, 20% and 80% of the reporters respectively. The training data were divided into five different groups of equal size and then trained the model on four of the groups, using the fifth group as test set to measure the accuracy of the model. The present study trained each model five times using each of the groups as a test set. To quantify model performance, the present study performed a linear regression between the model prediction and the experiment data of the test set, taking the mean prediction across each of the test sets.

### Dual Luciferase Assay

Single cell zebrafish embryos were co-injected mRNAs encoding nano and firefly luciferase (0.5 μg of nano/19.5 μg of firefly). Embryos were collected at 6 hpf and frozen in liquid nitrogen (5 embryos per replicate). Nano and firefly luciferase activity were measured using the Nano-Glo® Dual-Luciferase® Reporter Assay System. Firefly luciferase activity was utilized to normalized nano luciferase measurement across reporters.

### Data Analysis

All custom scripts were written in Python. Plots were generated using the Matplotlib package. Statistical analysis performed using the scipy package, whereas the random forest analysis was performed using the scikit-learn package. Feature and experimental data were stored using an SQLite database in conjunction with the HDF5 file format. All scripts can be accessed NaP-TRAP repository on Github.

### Example 2: NaP-TRAP Reveals the Regulatory Grammar in 5′UTR-Mediated Translation Regulation During Zebrafish Development

Example 2 section is directed to a further study based on that of Example 1. In one non-limiting aspect, the study of Example 2 overlaps with that described in Example 1, albeit includes more extensive data.

The cis-regulatory elements encoded in a mRNA determine its stability and translational output. While there has been a considerable effort to understand the factors driving mRNA stability, the regulatory frameworks governing translational control remain elusive. The present study has developed a novel massively parallel reporter assay (MPRA) to measure mRNA translation, Nascent Peptide Translating Ribosome Affinity Purification (NaP-TRAP). NaP-TRAP measures translation in a frame specific manner through the immunocapture of epitope tagged nascent peptides of reporter mRNAs. In contrast to existing MPRA methods, NaP-TRAP does not require specialized equipment and is readily adaptable to steady-state and dynamic model systems. The present study has employed NaP-TRAP to quantify Kozak strength and the regulatory landscapes of 5′ UTRs in the developing zebrafish embryo and in human cells, characterizing general and developmentally dynamic cis-regulatory elements. To this end, the present study identifies U-rich motifs as general enhancers, and upstream ORFs and GC-rich motifs as global repressors of translation. The present study also observes a translational switch during the maternal-to-zygotic transition, where C-rich motifs shift from repressors to prominent activators of translation. Conversely, the present study shows that microRNA sites in the 5′ UTR repress translation following the zygotic expression of miR-430.

Together these results demonstrate that NaP-TRAP is a versatile, accessible, and powerful method to decode the regulatory functions of UTRs across different systems.

### Example 2-1

Life requires spatial and temporal control of protein expression. The protein output of a given transcript reflects the integration of translation and mRNA stability. While there has been a considerable effort to understand the factors driving mRNA synthesis, maturation, and decay, the regulatory frameworks governing translational control remain elusive. Cis-regulatory elements encoded in the sequence and structure of mRNAs modulate these processes. Although these elements are distributed throughout the transcript, they are often concentrated in the regions up- and downstream of the main coding sequence, the 5′ and 3′ untranslated regions (UTRs) respectively. Given that initiation is the rate limiting step of translation, there has been a particular interest in understanding the regulatory elements found in 5′ UTRs. These elements include internal ribosome entry sites (IRESs), G-quadruplexes, iron response elements (IREs), and upstream Open Reading Frames (uORFs).

To investigate translation control, several studies have utilized ribosome profiling. While this method accurately quantifies the translation efficiency of individual genes, its capacity to characterize the function of cis-regulatory elements is limited. The generation of ribosome protected fragments decouples the translation measurement of a given mRNA from its cognate untranslated regions. Thus, measurements of translation efficiency often reflect the amalgamation of several isoforms of a given gene, each with a unique set of regulatory elements. This process is complicated further by the fact that endogenous transcripts contain multiple regulatory elements that exert differential and often competing effects on translation.

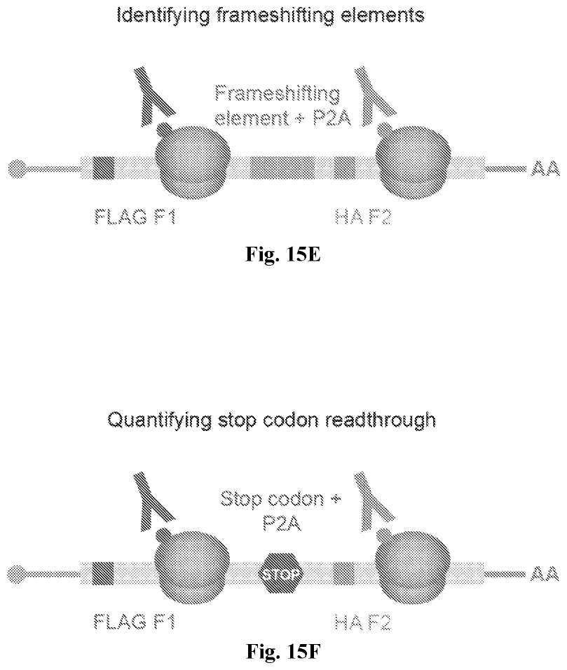

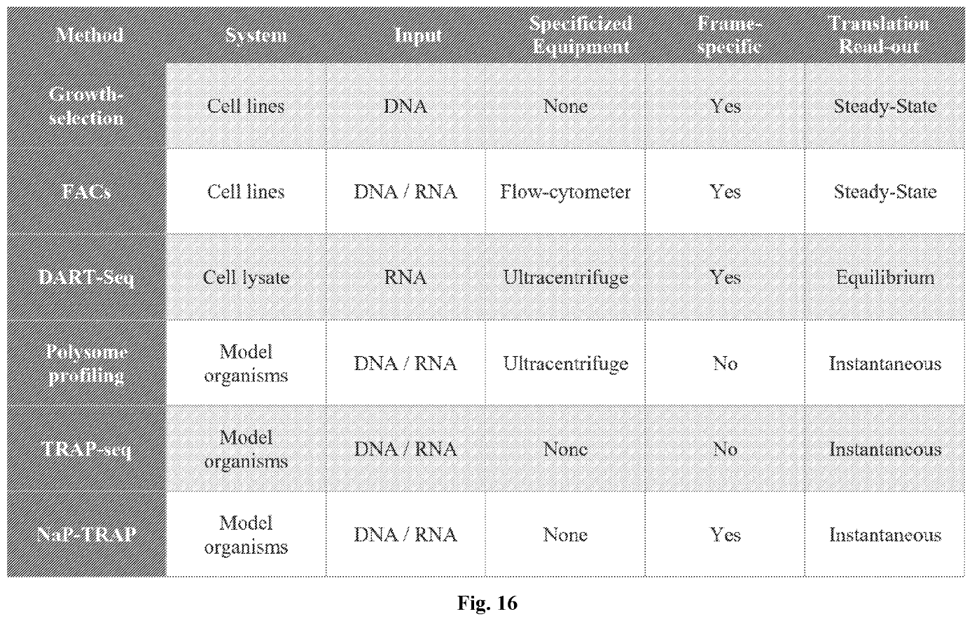

Massively parallel reporter assays (MPRAs) are particularly suited to address these challenges. MPRAs measure the abundance and/or translation efficiency of thousands of reporters simultaneously. These assays have improved the understanding of translation control and mRNA stability. Previous works have characterized Kozak strength, uORFs, IRESs, codon optimality, RNA structure, microRNA binding sites, and the effects of variation in human UTRs. These assays have also identified novel sequence motifs driving translation and decay. Despite these insights, conclusions based on MPRAs have been limited by the methods used to measure translation: growth-selection, fluorescent-cell sorting (FACS), polysome profiling, and direct analysis of ribosome targeting (DART) (FIG. 16). In MPRAs where reporters are encoded in DNA, measurements of translation can be confounded by additional layers of transcriptional regulation. In polysome fractionation, which quantifies the number of ribosomes on each transcript, inactive ribosomes, ribosomes translating out of frame, or ribosomes translating ORFs outside of the coding sequence may skew translation measurements. Additionally, the methodological complexity of these assays has reduced the use of translation-based MPRAs across diverse systems.

Here, the present study develops NaP-TRAP (Nascent Peptide Translating Ribosome Affinity Purification), a novel method to measure in-frame translation through the immunocapture of nascent peptides. More specifically, by including an N-terminal FLAG tag in the coding sequences of reporter mRNAs, the present study enriches for reporters in a manner proportional to the number of active ribosomes translating the main ORF in frame. The present study validates NaP-TRAP using reporter assays of increasing complexity. First, the present study measures the translation of individual reporters containing known cis-regulatory elements. Next, the present study quantifies Kozak strength in zebrafish by measuring the translation of thousands of reporters simultaneously. Lastly, the present study assesses the regulatory potential of endogenous 5′ UTR sequences in the developing zebrafish embryo and HEK293T cells to identify common and developmentally modulated motifs that regulate translation in vertebrates. In doing so the present study demonstrates that NaP-TRAP is an accessible, versatile, and quantitative method, which has the capacity to measure translation control across multiple systems.

### Example 2-2: NaP-TRAP Measures Translation Via the Immunocapture of Nascent Chains

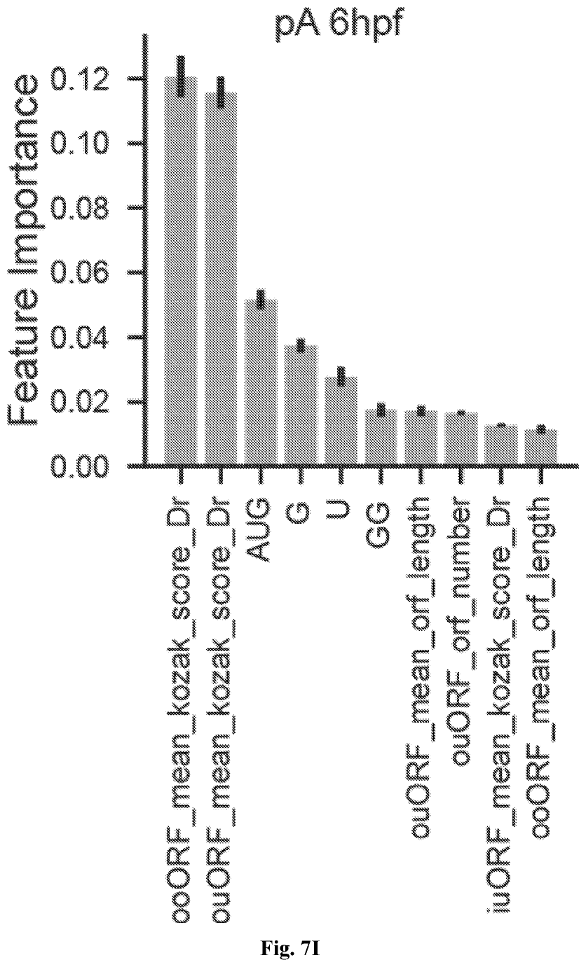

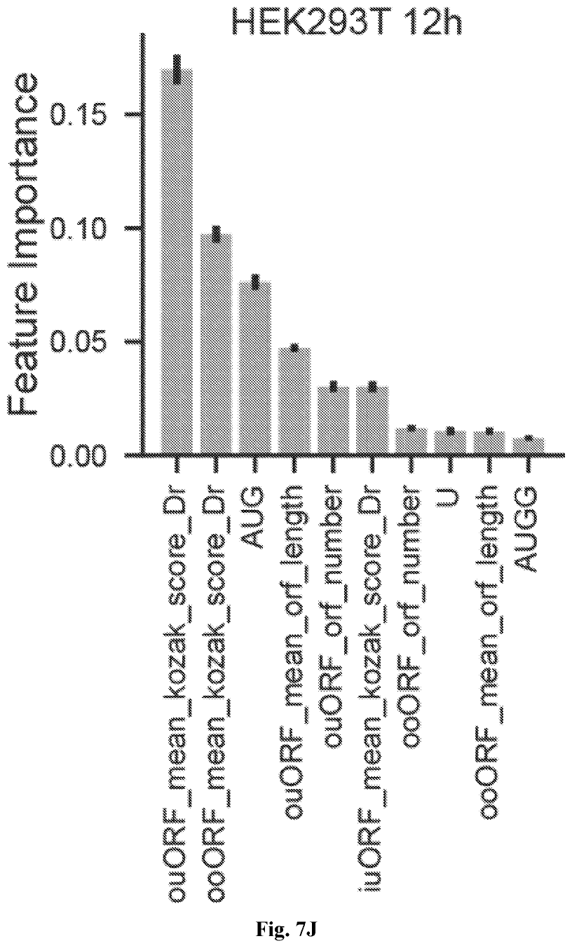

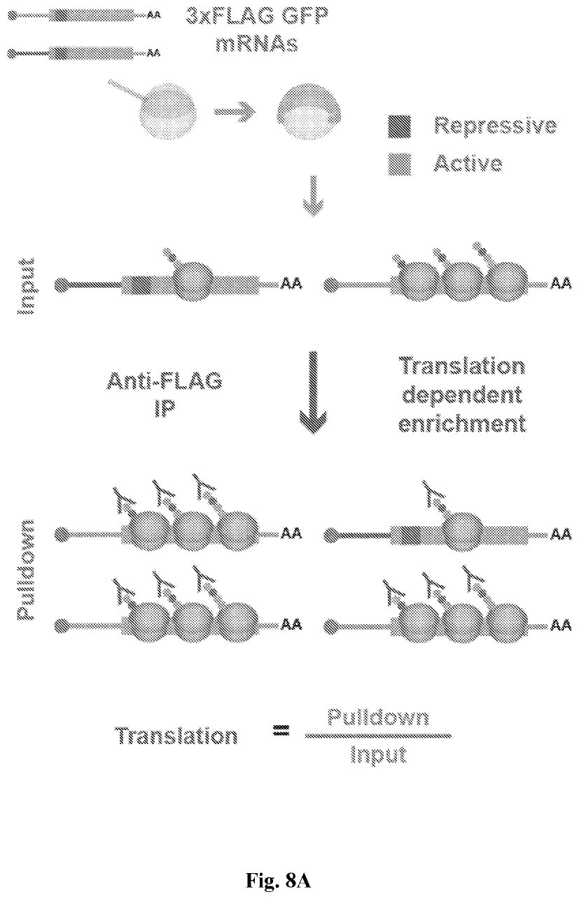

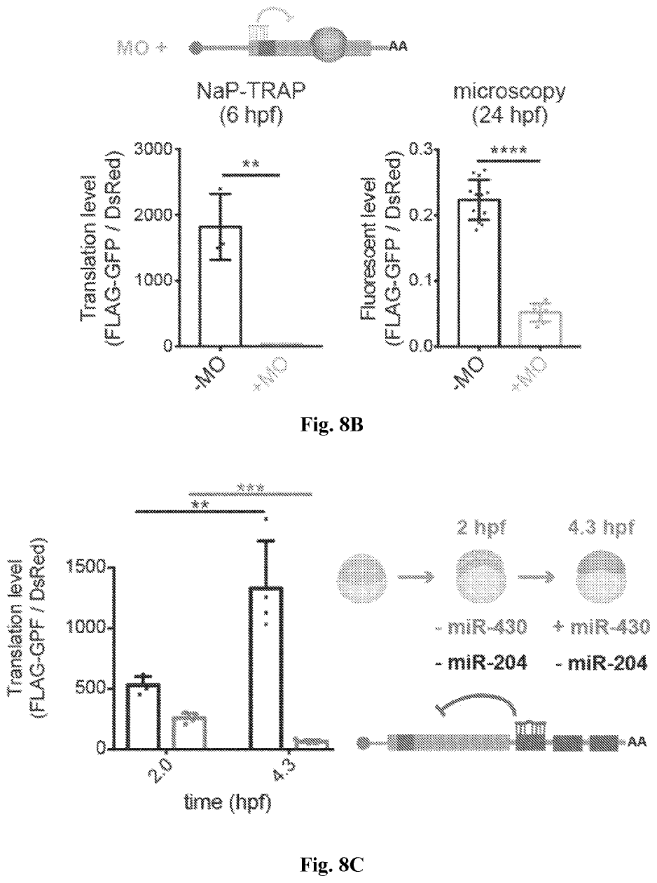

Cis-regulatory elements encoded in mRNAs modulate translation efficiency. To measure the regulatory potential of these elements the present study developed a novel massively parallel reporter assay (MPRA), NaP-TRAP (Nascent Peptide Translating Ribosome Affinity Purification). It was reasoned that the present study could enrich for reporters in a manner proportional to their translation efficiency through the immunocapture of FLAG-tagged nascent chain complexes immobilized by cycloheximide treatment (FIG. 8A). To this end, the present study measured translation as a ratio of the amount of a reporter mRNA in the pulldown relative to its input. This approach decouples translation measurements from differences in mRNA abundance.

To evaluate the capacity of NaP-TRAP to measure translation the present study performed two reporter assays. First, the present study measured the effect of a translation blocking morpholino. The present study co-injected 3×FLAG-GFP and dsRED mRNAs into single cell zebrafish embryos in the presence or absence of a translation blocking morpholino, targeting the start codon of 3×FLAG-GFP and performed NaP-TRAP at 6 hpf. To measure the amount of 3×FLAG-GFP reporter mRNA present in the input and pulldown fractions of each anti-FLAG immunoprecipitation, the present study employed reverse transcription-quantitative polymerase chain reaction (RT-qPCR). Given that the dsRED reporter mRNA was neither enriched by the anti-FLAG immunoprecipitation nor targeted by the translation blocking morpholino, the present study utilized the relative abundance of the dsRED reporter mRNA in each fraction to normalize NaP-TRAP derived translation values across experimental conditions. Using NaP-TRAP the present study observed a 48-fold decrease in translation in the presence of the morpholino at 6 hours post fertilization (hpf) (FIG. 8B). These results are consistent with the translational repression in morpholino injected embryos observed by the decrease in fluorescence (GFP/dsRED) at 24 hpf (FIG. 8B).

Next, the present study tested the capacity of NaP-TRAP to capture dynamic translation control. In the developing zebrafish embryo, the microRNA miR-430 is one of the first zygotically transcribed genes. At 4.3 hpf (hours post fertilization), the expression of miR-430 results in the translation repression, deadenylation, and eventual decay of targeted mRNAs. To quantify this effect, the present study measured the translation of 3×FLAG-GFP reporters with partial complementarity to two different microRNAs in their 3′ UTRs, miR-430 (3×FLAG-GFP-3×miR-430) and miR-204 (3×FLAG-GFP-3×miR-204). The present study selected miR-204 target sites as a control given that this microRNA is not expressed in the early embryo. Using NaP-TRAP the present study measured the translation of mRNA reporters at 2 and 4.3 hpf, before and after miR-430 expression. As described elsewhere herein, the present study utilized the abundance of the dsRED control RNA to normalize translation values across experimental conditions. Between the two timepoints, the translation of the 3×miR-430 reporter decreased by ~4.3 fold, whereas the translation of the 3×miR-204 reporter increased by ~2.5 fold (FIG. 8C). These results are consistent with the role of miR-430 in translational repression and the global increase in translation observed during the early stages of development.

Together these results demonstrate that NaP-TRAP measures the translation of individual reporters targeting general and developmentally dynamic cis-regulatory elements. Further, these experiments highlight the versatility of the method as they demonstrate the capacity of NaP-TRAP to quantify cis-regulation mediated by the 5′ and 3′ UTRs.

### Example 2-3: Using NaP-TRAP to Investigate Kozak Strength in the Developing Zebrafish Embryo

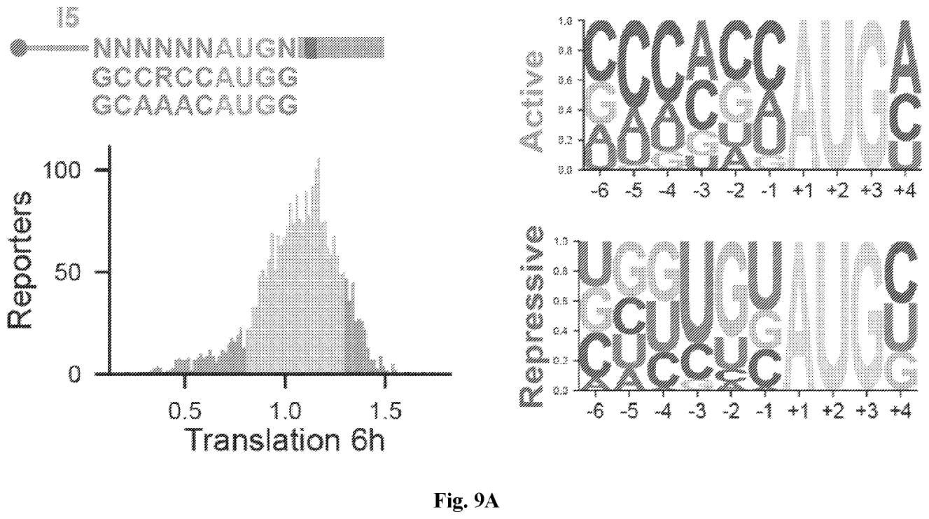

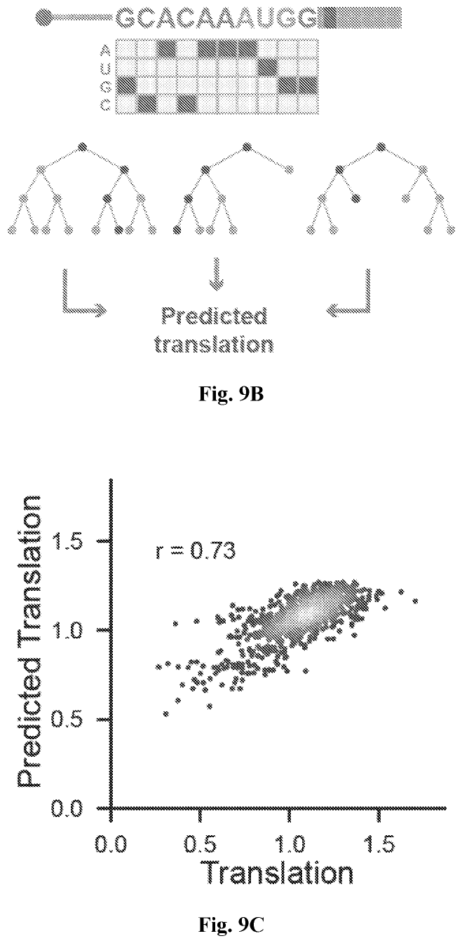

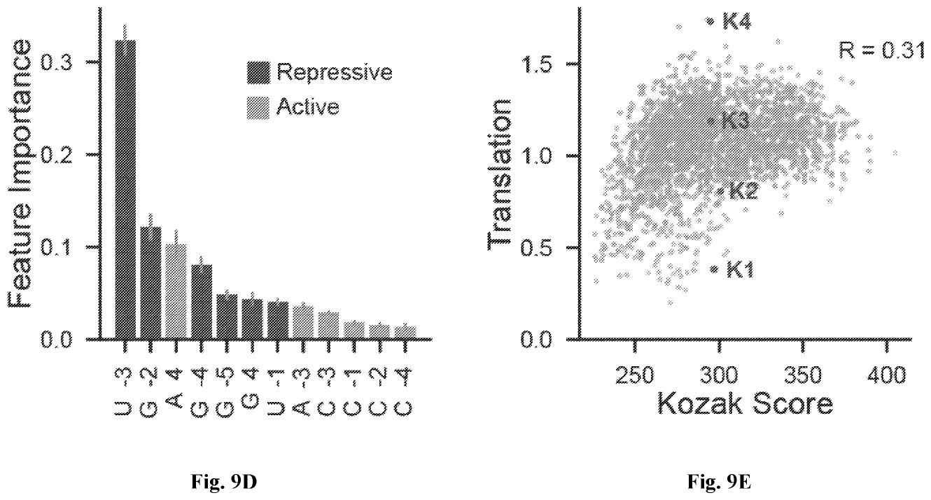

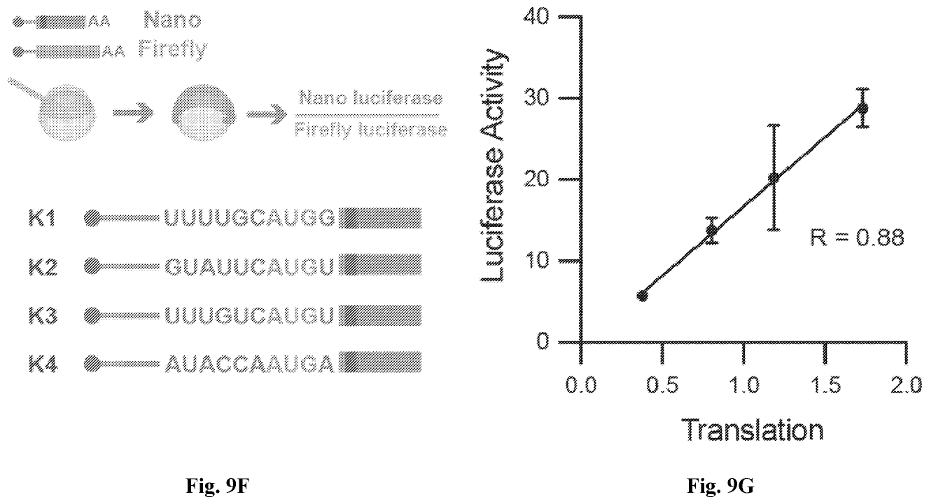

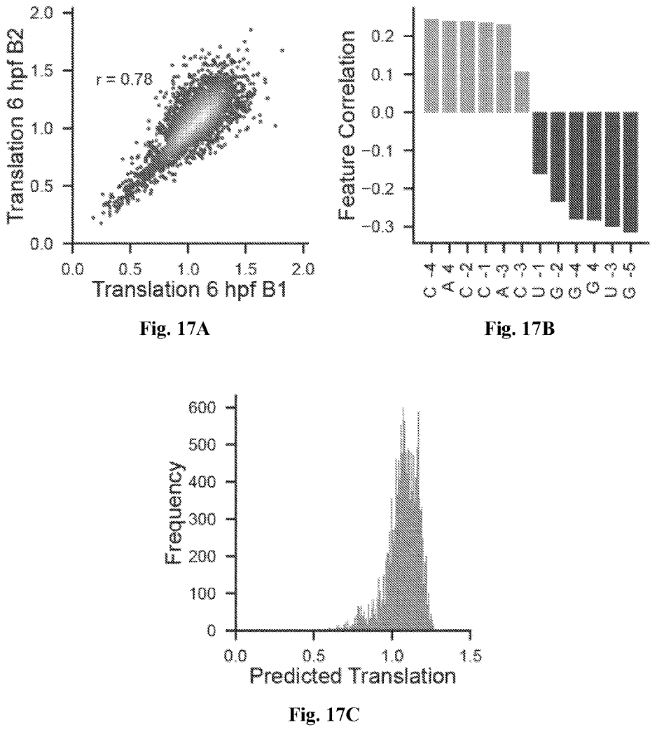

Next, to evaluate the capacity of NaP-TRAP to function as a MPRA, the present study designed a library to quantify the regulatory potential of the Kozak sequence. The present study elected to measure Kozak strength for two reasons: (1) the Kozak sequence is a strong determinant of translation initiation and (2) Kozak strength is often inferred based on the frequency of these sequences in the genome. To generate a library of diverse Kozak sequences, the present study incorporated six random nucleotides upstream and one random nucleotide downstream of the AUG of the 3×FLAG-GFP reporter (FIG. 9A). The present study injected the in vitro transcribed mRNA library into single cell zebrafish embryos and measured the translation using NaP-TRAP at 6 hpf. The present study observed a strong correlation in translation values between replicates (Pearson's r=0.78, FIG. 17A). To identify active and repressive Kozak sequences, the present study generated position weight matrices for the top and bottom 10% of reporters based on their translation (FIG. 9A). The present study observed that active Kozak sequences were enriched in C's and A's, whereas repressive Kozak sequences were enriched in U's and G's.

To examine the effect of nucleotide identity and position on translation the present study employed a random forest regression model (RFM). Model features were generated based on nucleotide identities and positions within the Kozak sequence (FIG. 9B). Prior to training the model, the present study divided the data into a test and a training set, 30% and 70% of reporters respectively. Using the training set, the present study employed a 5-fold cross validation to optimize model parameters. The model's prediction explained 73% of the variance observed in the test set data (FIG. 9C). Given this strong correlation the present study employed the model to predict the translation of all possible Kozak sequences (FIG. 17C). To identify the features informing the predictive power of the model, the present study performed a permutated feature importance analysis. Briefly, the present study shuffled the identities of the features of the model one at a time and measured the effect of the change on the predictive power of the model. At 6 hpf bases G, G, U, and G in positions −5, −4, −3, and −2 were the most repressive features, whereas C, A/C, and A in positions −4, −3, +4 were the most activating features (FIGS. 9D and 17B).

The present study compared NaP-TRAP translation values to an in silico derived metric, Kozak score. In zebrafish Kozak strength has been previously inferred based on the frequency of a given Kozak sequence within the transcriptome. This metric associates Kozak sequence abundance with increased translation initiation. The experimentally derived translation values challenge this hypothesis as the present study observed a weak correlation between NaP-TRAP and Kozak score (R=0.31, FIG. 9E). To validate this conclusion, the present study identified sequences that had similar Kozak scores (300±5) yet exhibited different NaP-TRAP derived translation values and measured their translation using a dual luciferase reporter assay (FIG. 9F). The luciferase translation values correlated strongly with NaP-TRAP (Pearson's R 0.88; FIG. 9G). This result not only highlights the importance of measuring Kozak strength experimentally, but also demonstrates that NaP-TRAP derived translation values correlate well with protein production.

Taken together, these results demonstrate that NaP-TRAP can be employed as a MPRA. By measuring the translation of thousands of reporters simultaneously, the present study quantifies and model Kozak strength in a vertebrate system.

### Example 2-4: Investigating the Regulatory Potential of Endogenous 5′ UTRS

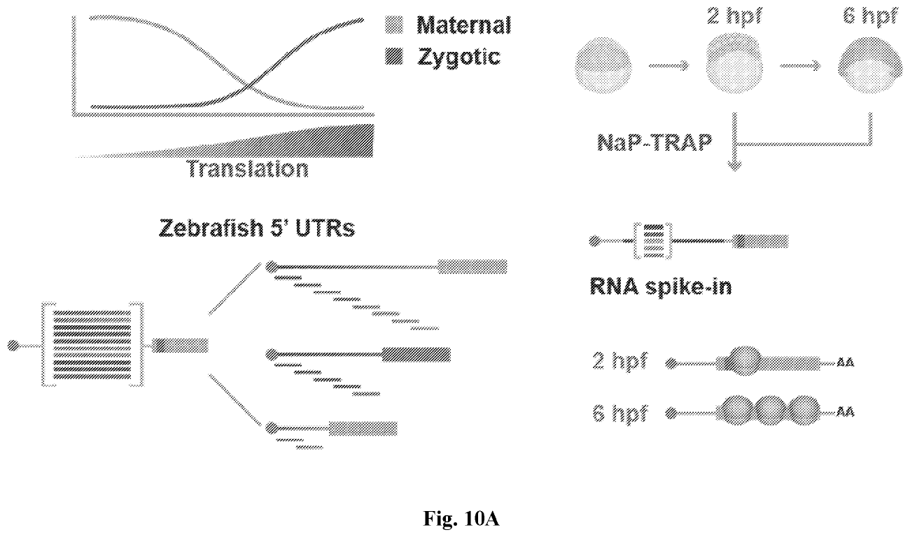

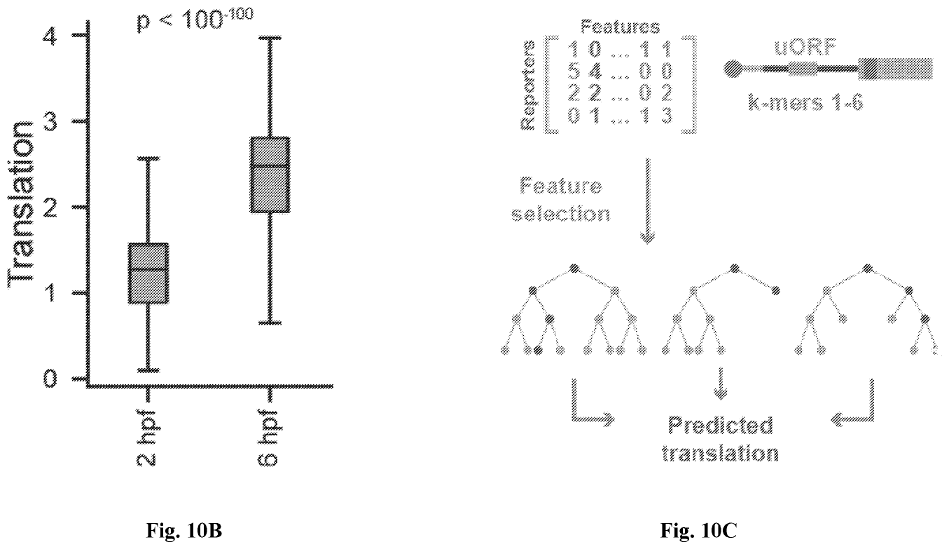

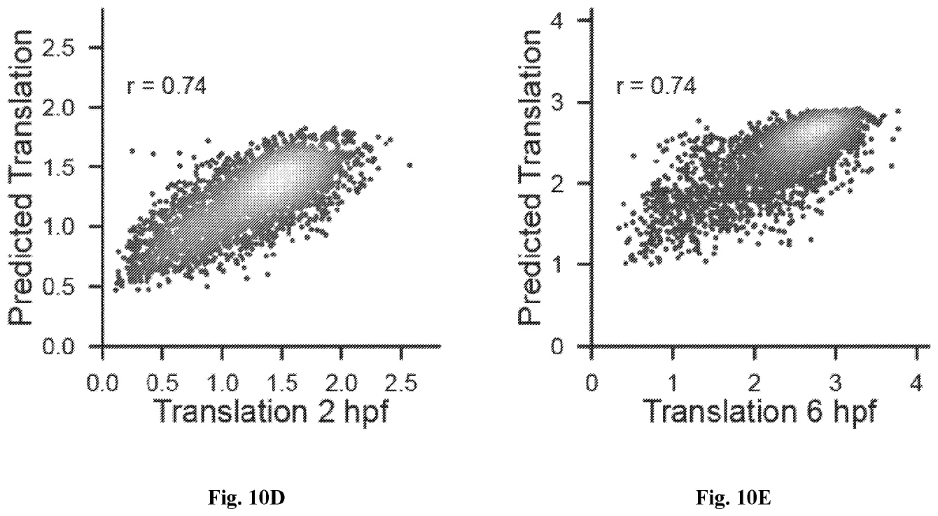

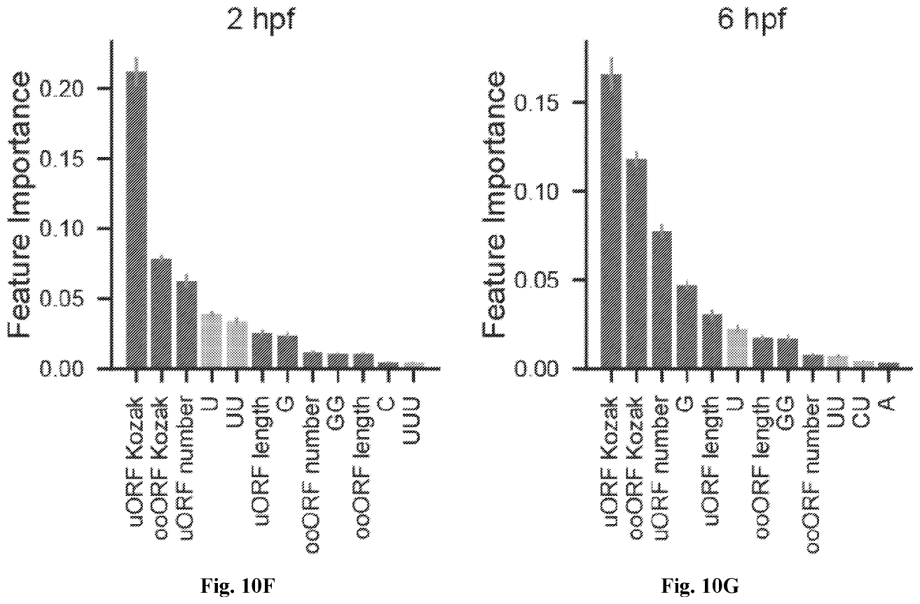

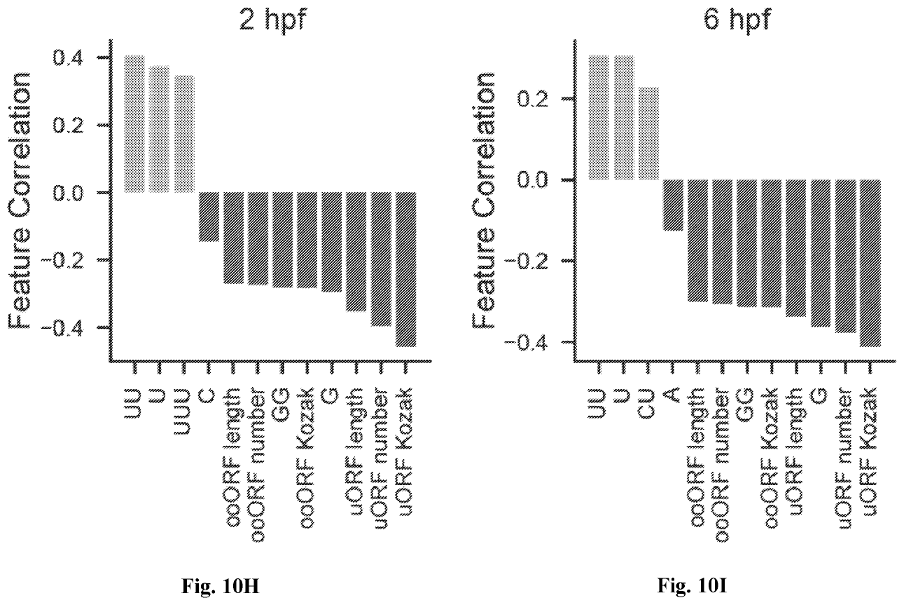

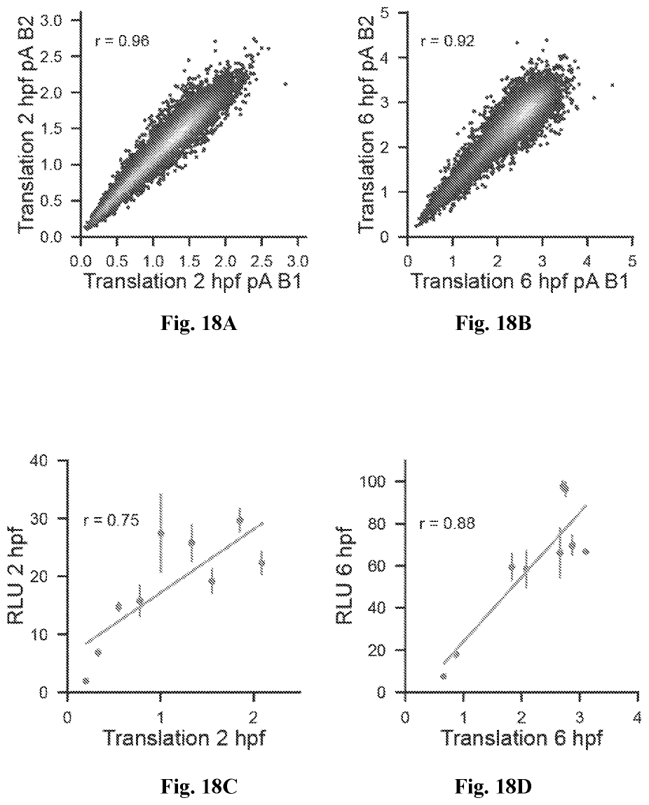

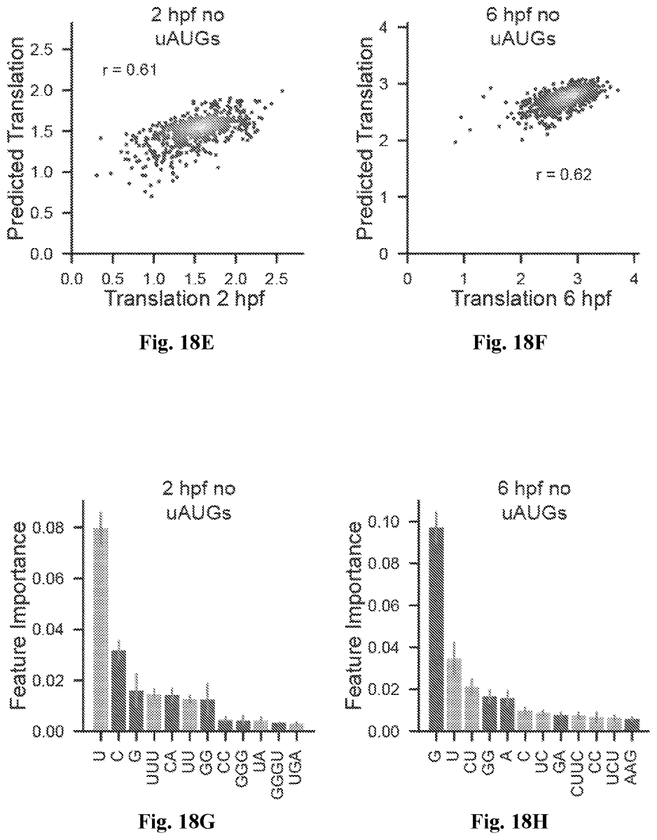

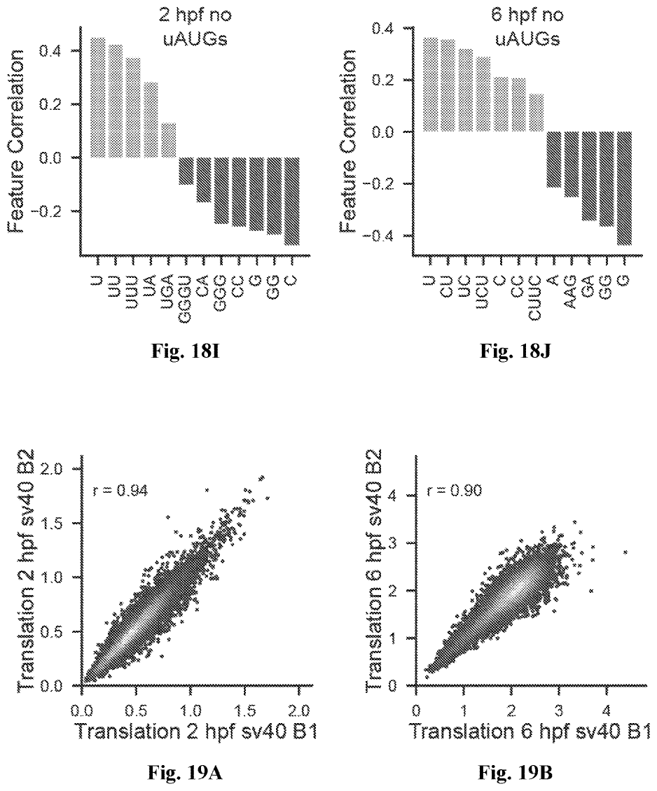

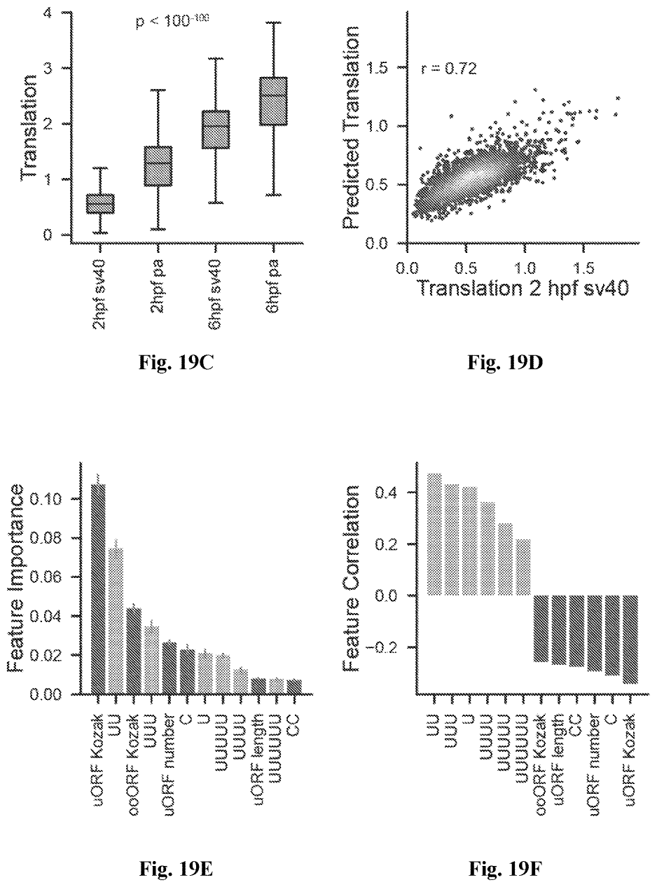

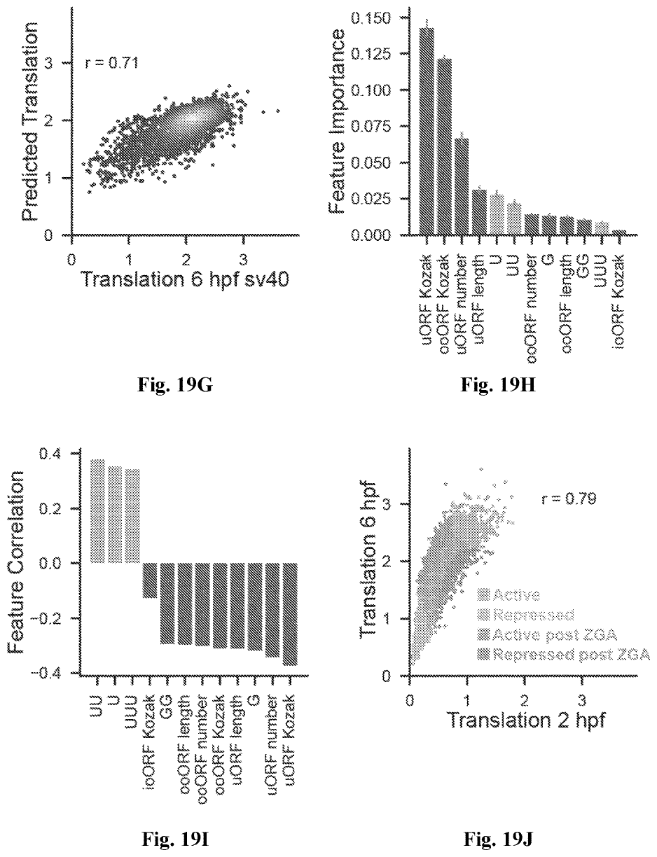

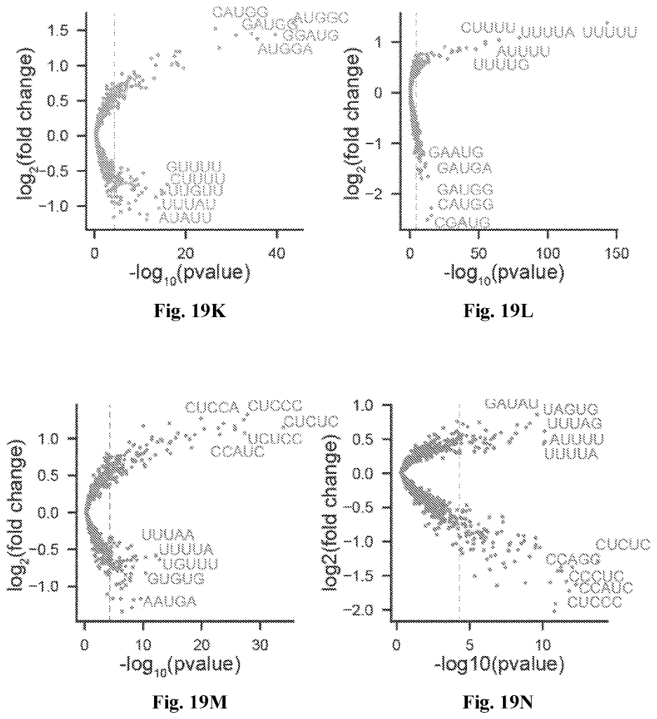

Upon validating NaP-TRAP's capacity to function as a MPRA, the present study investigated the regulatory complexity of endogenous 5′ UTRs during the early stages of embryogenesis. Prior to zygotic genome activation, the translation of maternally supplied mRNAs drives development. Given that initiation is the rate limiting step of translation, 5′ UTRs provide a mechanism for maternally supplied mRNAs to modulate their protein output. To study the cis-regulatory elements encoded in these mRNAs, the present study designed an 11,088-sequence synthetic oligo library (124-nt long) by tiling the 5′-UTRs of 1,725 zebrafish genes every 25 nucleotides (FIG. 10A). As the present study were interested in identifying specific sequence elements, which modulated differential translation regulation the present study elected to keep the 5′ UTR length and Kozak sequence of reporter mRNAs constant as both of these features correlate with translation efficiency in the developing embryo. The present study injected the in vitro transcribed library into single cell zebrafish embryos and measured translation at 2 hpf and 6 hpf using NaP-TRAP. The addition of an internal spike-in at the RNA extraction step allowed us to quantify relative changes in translation across developmental stages (FIG. 10A). Translation values were strongly correlated across replicates (Pearson's R>=0.90; FIGS. 18A-18B and 19A-19B) and with protein abundance at both time points (dual luciferase assay; 2 hpf Pearson's R 0.75, 6 hpf Pearson's R 0.88, FIGS. 18C-18D). The present study observed a mean increase in translation between 2 hpf and 6 hpf of ~1.9 fold, consistent with the gradual de-repression of translation during the first 24 hours of development (FIG. 19B).

Next, the present study utilized a random forest regression model trained on sequence elements and features characterizing upstream AUGs (uAUGs) to predict translation (FIGS. 10C and 19D-19I). These models explained 74% of the variance observed in the test set data at both timepoints (FIGS. 10D-10E). Using a permuted feature importance analysis, the present study identified the number of upstream uORFs (uORFs) as well as the Kozak strength of uORFs and out of frame overlapping ORFs (ooORFs) as the features contributing most significantly to the predictive power of the model at both timepoints (FIGS. 10F and 10G). When the present study examined the correlation of selected features with translation, the present study observed that U-repeats activated translation and G-repeats and uORFs suppressed translation at both timepoints. Interestingly, the present study also observed that C-rich k-mers suppressed translation at 2 hpf, whereas at 6 hpf C- and CU-rich k-mers activated translation (FIGS. 10H and 10I). Given the prominent role of upstream open reading frames in the feature importance analysis, the present study repeated the random forest analysis on reporters that contained no upstream start codons (FIGS. 18E-18J). In the absence of upstream start codons, the importance and prominence of C- and CU-rich k-mers increase at 6 hpf (FIG. 18J).

Altogether these results demonstrate the following: (1) uAUGs are the most prominent repressive elements during early embryogenesis, and (2) 5′ UTR regulatory landscapes are dynamic during development.

### Example 2-5: Identifying Sequences Driving Differential Translation

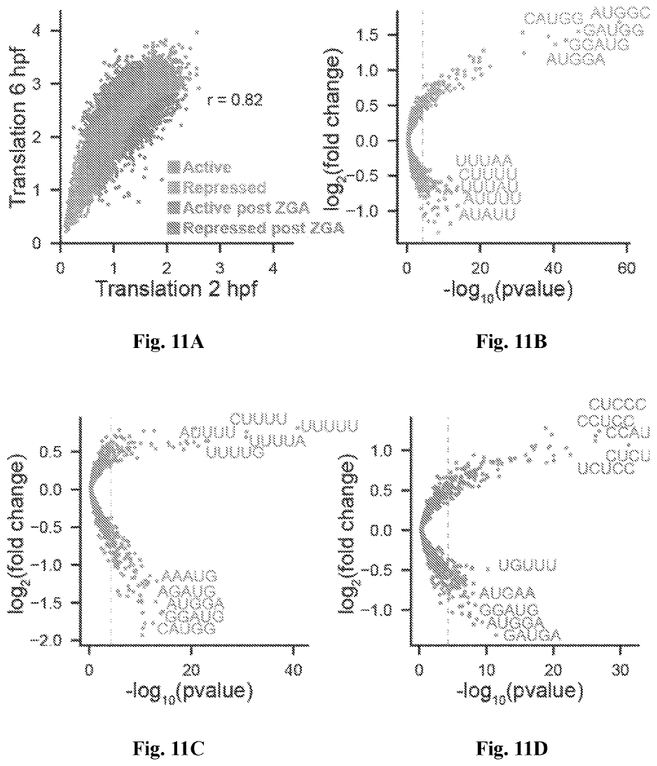

To identify the sequence elements modulating differential translation during development the present study divided the 5′ UTR reporters into four groups based on their relative translation at 2 and 6 hpf: (1) repressed, (2) active, (3) repressed post ZGA, and (4) active post ZGA (FIGS. 11A and 19J). Next, the present study performed a differential pentamer enrichment analysis on each group relative to the reporter library. This analysis revealed that repressed reporters were significantly enriched in upstream ORFs and GC-rich pentamers and depleted in U-rich tracks (p>10-5 hypergeometric test, FIGS. 11B and 19K). Conversely, active reporters were enriched in U-rich pentamers (UUUUU, CUUUU, UUUUA, GUUUU) and depleted in upstream start codons (FIGS. 11C and 19L). Reporters that were more highly translated after genome activation were enriched in C-rich pentamers (CUCUC, CUCCC, CCAUC, CCUCC) and depleted in U-repeats (FIGS. 11D and 19M). In contrast, reporters that were repressed post ZGA were enriched in AG/UG-rich sequences (UAGUG, UAUUG, AAGAA, AGACU), as well as sequence motifs complementary to the seed site of the microRNA miR-430 (GCACU and GCACUU) and depleted in uORFs and pyrimidine repeats (FIGS. 11E and 19N).

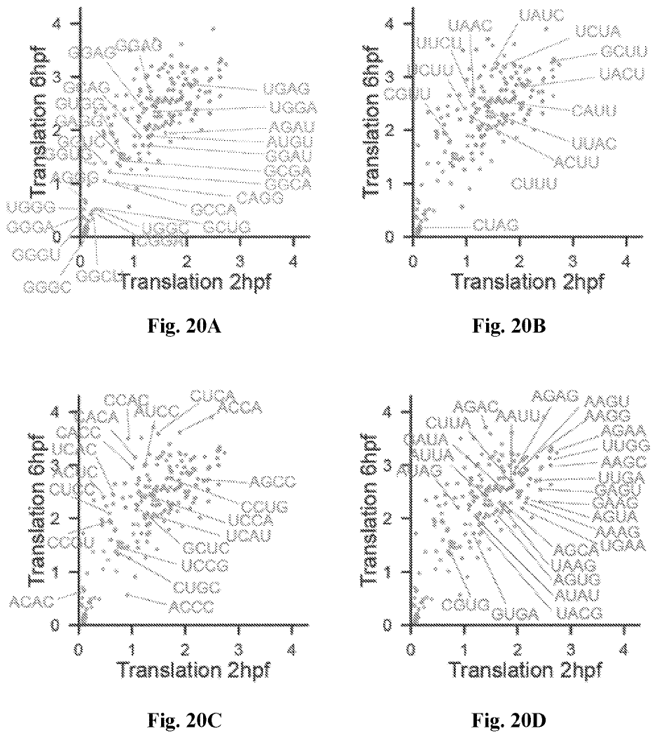

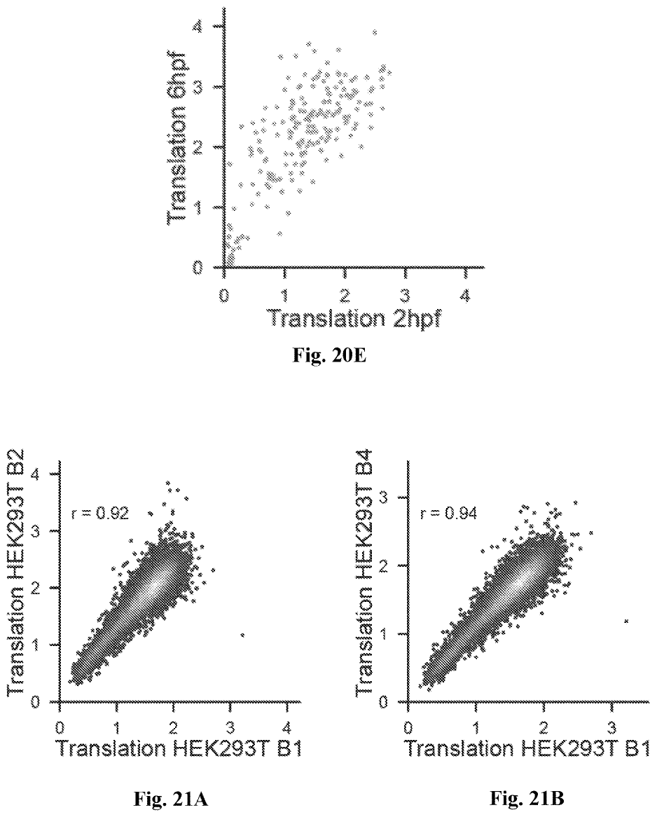

To validate these results, the present study generated a library of 5′ UTR sequences containing all possible tetramer repeats separated by dinucleotide spacers and measured translation using NaP-TRAP at 2 and 6 hpf (FIG. 11F). Next, the present study plotted the translation values of the validation library labeling each reporter on the basis of whether its encoded tetramer repeat was enriched in the reporter groups described above (FIG. 11G). The distributions of reporters encoding repressed, repressed post ZGA, and active post ZGA tetramers largely recapitulated the distributions of their respective groups in the 5′ UTR library (FIGS. 20B-20E). Consistent with this, a cumulative analysis of the translation (2 hpf/6 hpf) of reporters enriched in “active motifs post ZGA” revealed a larger increase in translation compared to those enriched in “repressed motifs post ZGA” (FIG. 11H).

Altogether these results suggest that there are general and dynamic sequence motifs in zebrafish 5′ UTRs that modulate translation during the early stages of development.

### Example 2-6: Complementary Sequences to miR-430 in the 5′ UTR Suppress Translation

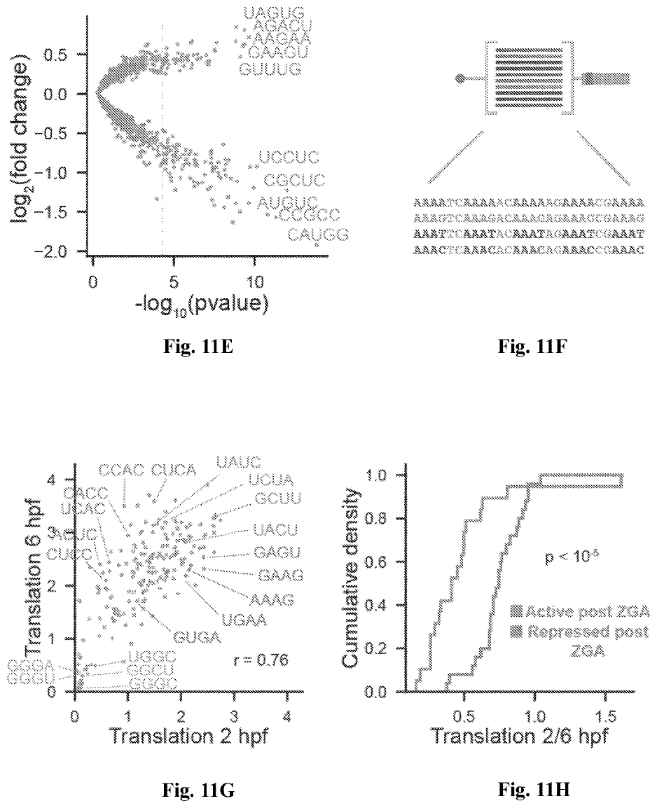

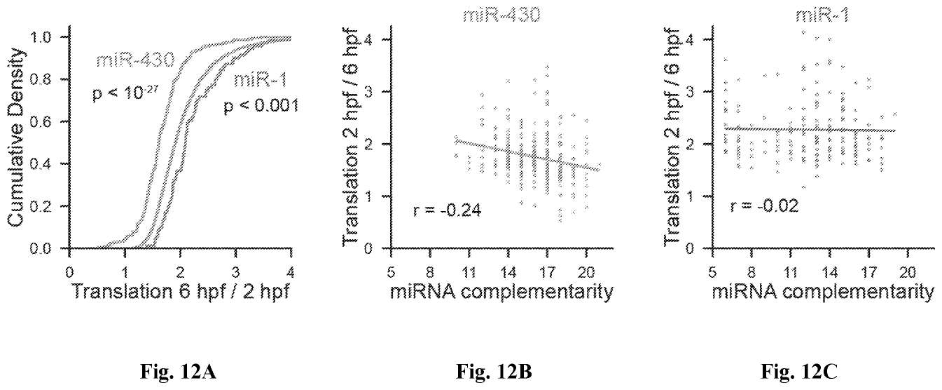

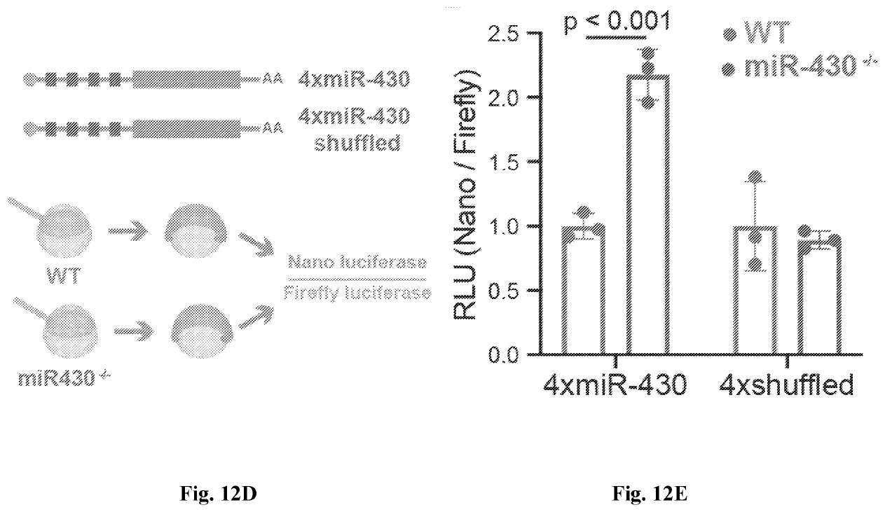

miR-430 is one of the first zygotically transcribed genes in the developing zebrafish embryo. The enrichment of miR-430 seeds in reporters that were repressed after ZGA suggests that the maternal-to-zygotic transition is shaped by 5′ UTR mediated translation control. To determine whether this repressive effect is specific for miR-430, the present study compared the translation between 2 hpf and 6 hpf for reporters containing seeds for miR-430 or miR-1, a microRNA that is not expressed in the early stages of development. The present study observed a significant decrease in the translation of miR-430 containing reporters (6 vs 2 hpf) relative to the translation of reporters lacking either seed. In contrast, there was a slight increase in translation of miR-1 control reporters (FIG. 12A). To investigate the mechanism driving miR-430 mediated translation repression, the present study measured the degree of complementarity between the microRNA and the targeted reporter. Translation of the miR-430 reporters was negatively correlated to miRNA-5′ UTR complementarity (FIG. 12B). Whereas translation of the miR-1 control reporters was not significantly correlated with miRNA-5′ UTR complementarity (FIG. 12C). This observation suggests that 5′ UTR microRNA mediated translation repression is driven by sequences with homology beyond the seed site of the microRNA.

To demonstrate further that miR-430 expression drives the translation repression of 5′ UTRs with seed sites, the present study constructed two nano-luciferase reporters: 4×miR-430-nanoluc and 4×miR-430-MUT-nanoluc. The present study shuffled the miR-430 sites in the 4×miR-430-MUT reporter to prevent miR-430 targeting (GCACUU to GCUCUA). The present study injected these reporter mRNAs together with a firefly luciferase control mRNA into wild-type and miR-430−/− mutant embryos and measured relative luciferase activity at 6 hpf (FIG. 12D). The present study observed a ~2-fold decrease in the relative luciferase activity of the 4×miR-430 reporter in the wild-type condition when compared to the mutant. In contrast, the present study observed no significant difference in luciferase activity between the shuffled reporters when comparing the mutant and wild-type embryos (FIG. 12E). This finding suggests that the zygotic expression of microRNA miR-430 represses the translation of mRNAs with 5′ UTR seed sites.

Taken together these results demonstrate that miRNAs that target sites in the 5′ UTR can provide significant translational repression in vivo.

### Example 2-7: The Developmentally Dynamic Role of C-Rich Motifs

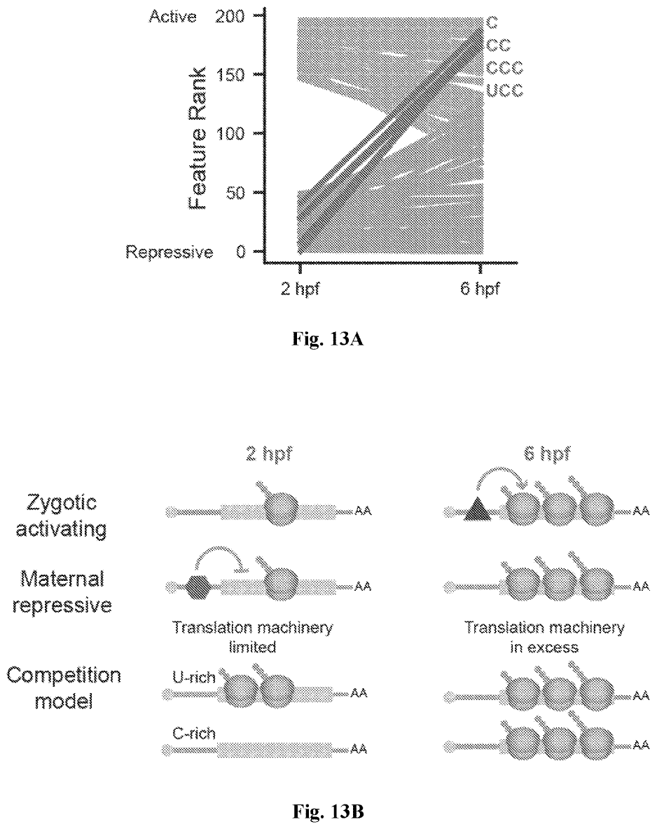

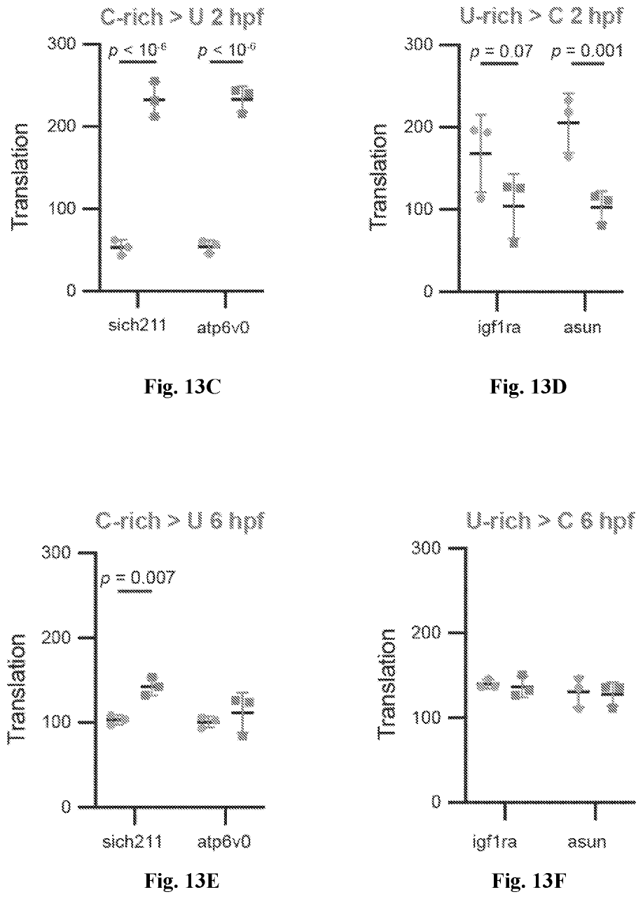

The present study also observed an enrichment of C-rich pentamers in reporters that were active following ZGA. To explore this observation in greater detail, the present study measured the mean difference in translation between reporters enriched and depleted in each feature (k-mers≤4) that lacked upstream open reading frames. Next, the present study compared the rank of features with a significant difference in translation at 2 hpf and 6 hpf. The present study observed that the k-mers C, CC, CCC, and UCC were some of the most repressive features at 2 hpf yet activated translation at 6 hpf (FIG. 13A). To investigate this differential regulation further, the present study selected two active (U-rich) and two active post ZGA (C-rich) reporters from the 5′ UTR library. To determine the role of C-rich sequences in this translation regulation the present study mutated U's in the active reporters to C's and mutated C's in the active post ZGA reporters to U's for a total of eight reporters: four wild-type and four mutants, respectively. The present study injected the reporters into single cell zebrafish embryos and measured translation at 2 hpf and 6 hpf using NaP-TRAP. At 2 hpf the present study observed that mutating C's to U's in the C-rich reporters enhanced translation (FIG. 13C), whereas mutating U's to C's in U-rich reporters repressed translation (FIG. 13D). In contrast, this effect was largely reduced at 6 hpf (FIGS. 13E and 13F). These results support the findings of the differential enrichment analysis and indicate that there is a translational switch driven by C-rich sequences following ZGA (FIG. 13B).

Altogether, these results validate novel developmentally dynamic mechanisms of 5′ UTR mediated translation control, demonstrating the importance of quantifying cis-regulation in non-steady state systems.

### Example 2-8: NaP-TRAP can be Readily Adapted to Human Cells

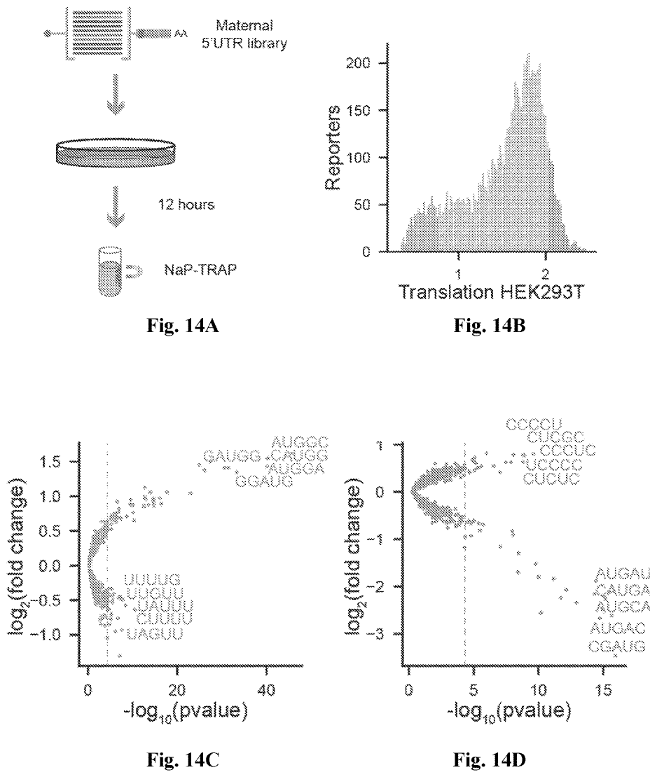

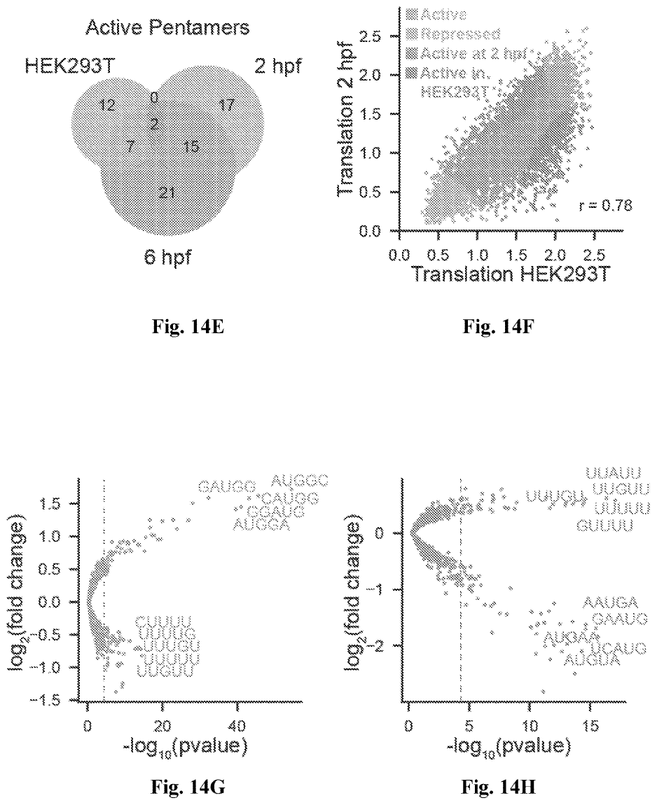

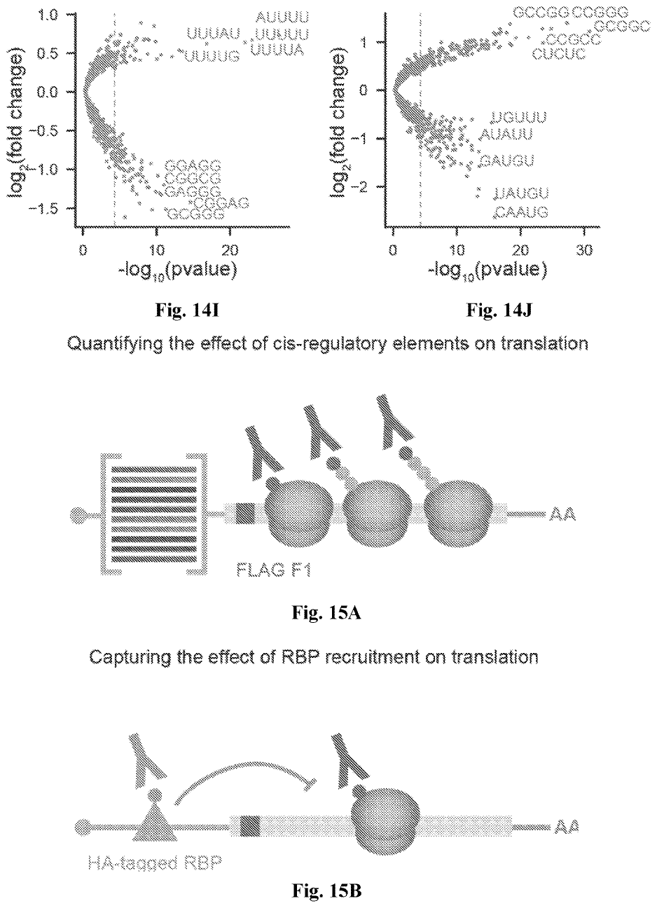

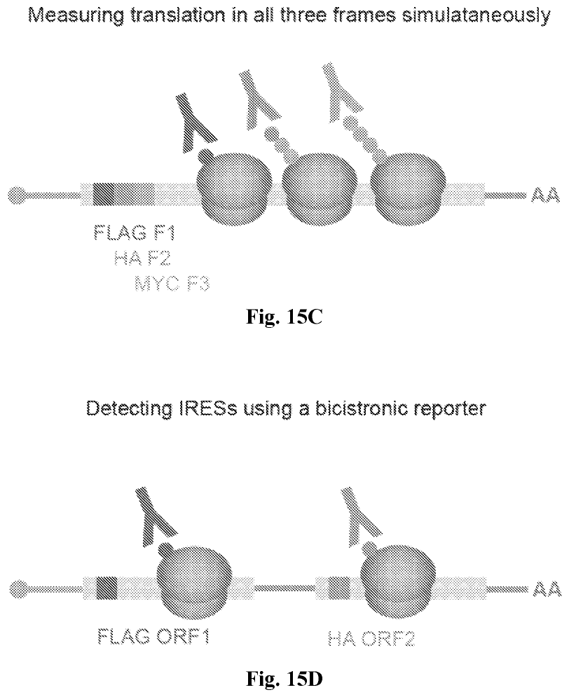

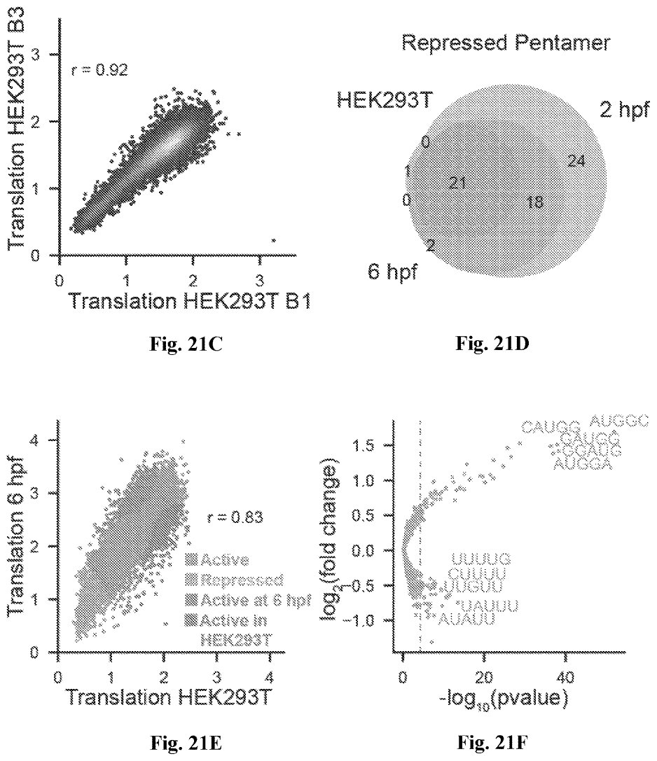

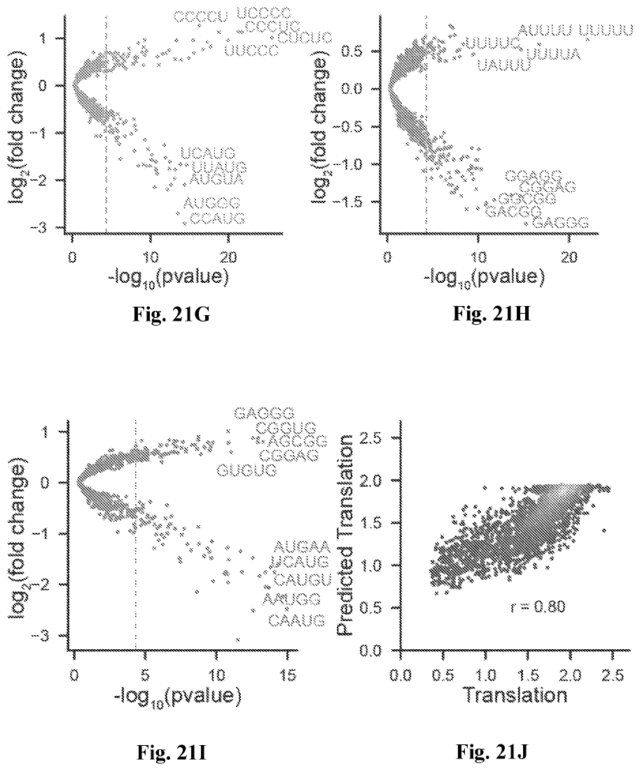

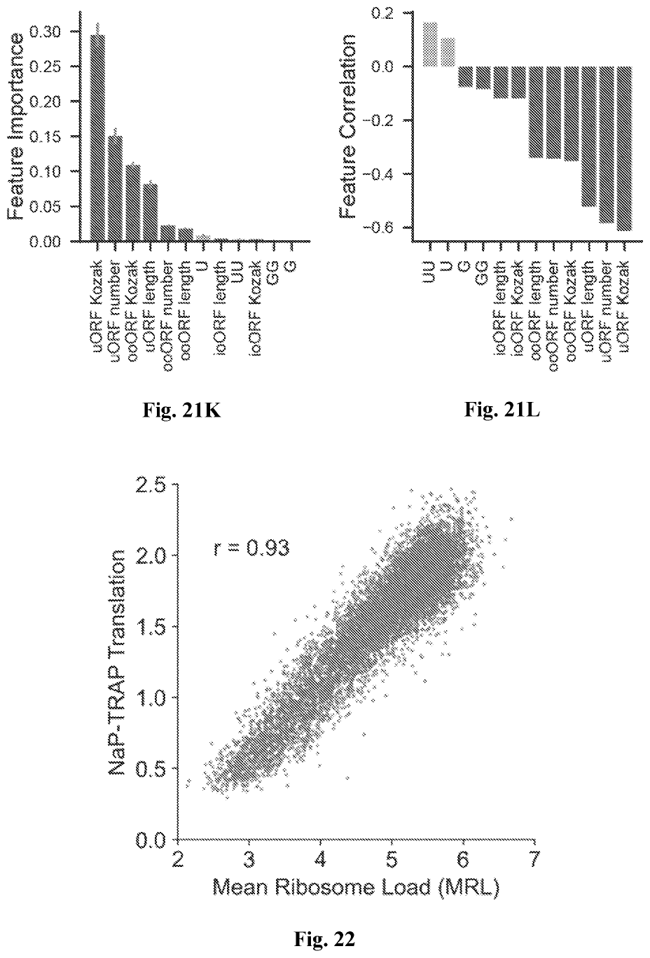

To demonstrate the broad applicability of the method herein, the present study adapted NaP-TRAP to human cells. To this end, the present study transfected the in vitro transcribed mRNA 5′ UTR library into HEK293T cells and measured translation at 12 hours post transfection (hpt) using NaP-TRAP (FIGS. 14A and 14B). Replicates were strongly correlated (Pearson's R≥0.92, FIGS. 21A-21C). To identify motifs modulating translation in HEK293T cells the present study performed a differential enrichment analysis on active and repressed reporters. Repressed reporters were enriched in AUG-containing motifs and depleted in U-rich motifs (FIG. 14C), whereas active reporters were enriched in C-rich pentamers and depleted in AUG containing motifs (FIG. 14D), consistent with those observed in zebrafish at 6 hpf (FIGS. 14E and 21D).

Next, the present study performed a differential enrichment analysis comparing the human data to each of the zebrafish timepoints (FIGS. 14F-14J and 21E-21I, 2 hpf and 6 hpf, respectively). The present study divided the reporters into four groups: (1) repressed, (2) active, (3) active in zebrafish, and (4) active in HEK293T cells. From these analyses, the present study conclude that uAUGs are general repressors of translation (FIGS. 14G and 21F) and U-rich motifs are general activators of translation (FIGS. 14H, 21G and 21L). The present study also observed that reporters that are active only in HEK293T cells are enriched in G-rich k-mers, whereas reporters active at 2 hpf or 6 hpf are depleted in these motifs (FIGS. 14J and 21I). Next, when the present study employed a random forest model to predict translation. The model explained 80% of the variation in the experimental data (FIG. 21J). Consistent with the results observed in zebrafish, Kozak strength and number of upstream AUGs were the most predictive features of translation in HEK293T cells, exhibiting a strong negative correlation with translation (FIGS. 21K and 21L).

Altogether these results demonstrate that (1) NaP-TRAP is a robust method that can measure translation across multiple model systems, (2) upstream AUGs are a dominant driver of 5′ UTR mediated translation control in HEK293T cells, and (3) U-rich motifs are general activators of translation.

### Example 2-9

Here, the present study develops a novel MPRA method, NaP-TRAP, and demonstrate its capacity to measure translation quantitatively. The present study uses NaP-TRAP to characterize cis-regulatory elements in the 5′ and 3′ UTRs, the strength of the Kozak sequence, and the regulatory landscape of the 5′ UTRs of mRNAs in the developing zebrafish embryo and HEK293T cells. Using NaP-TRAP the present study has identified general and developmentally dynamic cis-regulatory elements, as well as characterized global changes to translation associated with early embryogenesis.

NaP-TRAP is an accessible, versatile, and quantitative MPRA method to measure translation. In contrast to existing approaches NaP-TRAP does not require a large amount of input material or specialized equipment, making the method adaptable to a wide range of model systems, different cell types, and physiological states (FIG. 16). Further, NaP-TRAP measures frame-specific translation as only the nascent chains of ribosomes translating the main ORF are FLAG-tagged and thereby immunocaptured. This approach is particularly important in systems with a low basal level of translation (e.g., the early stages of vertebrate embryogenesis and neurons) and results in translation measurements that reflect protein output (FIGS. 9G and 17C-17D). In polysome profiling and other TRAP methods, inactive ribosomes or ribosomes translating outside of the main open reading frame may affect the measurements of translation. By enriching for reporters through the immunocapture of FLAG-tagged nascent chain complexes, NaP-TRAP measures translation in a manner that is proportional to the number of ribosomes actively translating the tagged ORF. Second, by injecting or transfecting in vitro transcribed mRNA reporter libraries, NaP-TRAP eliminates the confounding effects of transcriptional regulation.

Previous studies have assumed that the frequency of a given Kozak sequence within the genome correlates strongly with its effect on translation. While this hypothesis has been challenged by MPRAs performed in cell culture, to date there is a lack of measurements of the effect of Kozak strength associated with MPRAs that introduce reporters as DNA. When quantifying the regulatory potential of the 5′ UTR, transcriptional bias may be more pronounced given the region's proximity to the promoter sequence on translation in zebrafish. To demonstrate the capacity of NaP-TRAP to quantify the translation of thousands of mRNA reporters simultaneously, the present study measured the Kozak strength in zebrafish embryos. In support of this approach, the present study observed a weak correlation between Kozak strength and an in silico derived Kozak score (FIG. 9E). While the approach has identified conserved activators and repressors (−3 A and −3/−2 U/G) of translation, the present study has also characterized novel Kozak sequences that differ from the zebrafish or vertebrate consensus Kozaks, including the activating effect of adenosine in the +4 position. Further, the present study has utilized the random forest regression model to predict the Kozak strength of all zebrafish Kozak sequences (positions-6 to +4). This prediction will improve the annotation of Kozak strength in the zebrafish, as well as the capacity to modulate protein output.

Development requires dynamic spatial and temporal control of translation. Using NaP-TRAP the present study has identified 5′ UTR cis-regulatory elements that differentially regulate translation in development. For example, the present study shows that the zygotically expressed microRNA miR-430 represses the translation of reporters containing miR-430 seeds in the 5′ UTR.

The present study has also identified a translation switch driven by C-rich motifs. At 2 hpf C-rich k-mers suppress translation, whereas at 6 hpf these k-mers activate translation. The present study proposes two potential mechanisms to explain this novel 5′ UTR mediated translation control (FIG. 13B). First, the expression or loss of a trans-acting factor (TAFs) that binds pyrimidine rich tracts may drive differential translation. Members of the poly-pyrimidine tract-binding protein family (PTBP) and components of the EIF3 complex have been shown to activate translation by binding pyrimidine tracts in the 5′ UTR. Second, recruitment of trans-factors depends not only on the presence of a given cis-regulatory element, but rather the composition of the transcriptome and the pool of available trans-factors shapes the regulatory potential of a given element. The present study observes that the relative importance of U-rich sequences depends on the global rate of translation. When translation is low, the prevalence of U-rich sequences is a prominent predictor of translation (FIGS. 19D-19F). In contrast, as translation increases, the relative importance of U-rich sequences declines, whereas the relative importance of uORFs increases (FIGS. 19G-19I). The present study proposes that this change reflects a change in the availability of the translation initiation machinery. The present study speculates that when the supply of ribosomes is limited, the capacity of the 5′ UTR to recruit ribosomes drives translation. In contrast, as the ribosome pool increases, the effect of 5′ UTRs on ribosome recruitment diminishes (FIG. 13B).

This competition model can also be employed to explain the differential effect of C-rich tracts on translation. In the early embryo, reporters enriched in U-repeats may recruit the limited supply of ribosomes more efficiently than reporters enriched in C's. As the supply of translation machinery increases, the effect of competition diminishes, resulting in the efficient initiation of C-rich 5′ UTRs (FIG. 13B). Repressive trans-acting factors (TAFs) may amplify the effect of competition, as in the absence of scanning 40S ribosomes, these factors can be more readily recruited to the 5′ UTR.

NaP-TRAP also identifies conserved regulators of translation. In both the developing zebrafish embryo and HEK293T cells, poly-U repeats activate translation whereas uORFs repress translation.

### Example 2-10: Materials and Methods

### Zebrafish Maintenance and Mating

Wild-type zebrafish embryos were obtained through natural mating of TU-AB strain of mixed ages (5-18 months). Mating pairs were randomly chosen from a pool of 60 males and 60 females allocated for each day of the month.

### Hek293T Cells

HEK293T cells were grown in a media consisting of Dulbecco's Modified Eagle Medium (DMEM) (ThermoFisher Scientific #10569010), 10% heat inactivated Fetal Bovine Serum (FBS) (ThermoFisher Scientific #16140071), 20 mM HEPES (ThermoFisher Scientific #15630080), 2 mM L-Glutamine (ThermoFisher Scientific #25030081), 1× Penicillin/Streptomycin (ThermoFisher Scientific #15140122) at 37 °C and 5% CO₂. mRNA transfections were performed using Lipofectamine™ MessengerMAX™ Transfection Reagent (ThermoFisher Scientific #LMRNA008) in accordance with the manufacture's protocol.

### NaP-TRAP Reporter Controls

To enable nascent chain immunocapture, a 3×FLAG tag was incorporated after the first 18 nucleotides of GFP-3×AID* (auxin inducible domain) using an In-Fusion® HD Cloning kit (Takara #638946) (F: 3×FLAG_inf_fwd; R: AID_inf_rev). While AID* domains were included in the initial NaP-TRAP vector to enable future use of an auxin-inducible degron system, this system was not utilized in this study. The vector also included an SP6 promoter sequence and SV40 poly-adenylation signal. NaP-TRAP reporter and dsRED control (plasmid pCS2+-dsRED) mRNAs were generated using a mMESSAGE mMACHINE™ SP6 transcription kit (Invitrogen™ #AM1340) from linearized reporter plasmids via NotI-HF® (NEB #R3189L) restriction enzyme digest. In vitro transcribed mRNAs were purified using a Monarch® RNA Cleanup Kit (NEB #T2040L) prior to injection.

To validate NaP-TRAP, two reporter experiments were performed. First, to quantify morpholino mediated translation repression using NaP-TRAP, 100 μg of 3×FLAG-GFP-3×AID mRNA and 75 μg of dsRED mRNA were injected into single cell zebrafish embryos in the presence or absence of a morpholino targeting the start codon of 3×FLAG-GFP (250 μM, GFP-MO 5′-ACAGCTCCTCGCCCTTGCTCACCAT-3′, SEQ ID NO:2365, Gene Tools LLC). Embryos were collected at 6 and 24 hpf (25 embryos per NaP-TRAP replicate). Embryos collected for NaP-TRAP were flash frozen in liquid nitrogen prior to sample processing. NaP-TRAP was performed at 6 hpf whereas immunofluorescence was measured at 24 hpf. Images were quantified using ImageJ.58 Second, to assess the capacity of NaP-TRAP to measure microRNA mediated repression in the 3′ UTR, two additional reporters were generated: (1) 3×FLAG-GFP-3×miR-430 and (2) 3×FLAG-GFP-3×miR-204. Three binding sites of either miR-430 or miR-204 were cloned into the 3′ UTR of 3×-FLAG-GFP-3×AID*, using an In-Fusion® HD Cloning kit (Takara #638946) (F: FGFP_inf_fwd; R: 3×mir430_inf_rev and 3×mir204_inf_rev, respectively). Single cell embryos were injected with 20 μg of both the 3×FLAG-GFP-3×miR-430 and 3×FLAG-GFP-3×miR-204 mRNAs, as well as 160 μg of dsRED mRNA. Twenty-five embryos per replicate were collected and flash frozen in liquid nitrogen at 2 and 4.3 hpf.

For methods detailing NaP-TRAP and qPCR translation measurements see sections: (1) NaP-TRAP (Nascent Peptide Translating Ribosome Affinity Purification) and (2) NaP-TRAP qPCR analysis, respectively.

### NaP-TRAP Reporter Library Assembly

To eliminate excess cytoplasmic 3×FLAG-GFP, a C-terminal PEST domain was incorporated into the NaP-TRAP reporter plasmid using an In-Fusion® HD Cloning Kit (Takara #638946). The 3×FLAG-GFP vector was amplified from the 3×FLAG-GFP-3×AID* plasmid (F: GFP_dd1_inf_fwd, R: GFP_dd1_inf_rev), whereas the PEST domain insert was generated using PCR overlap extension (F: PEST_fwd, R: PEST_rev). For the sake of brevity, 3×-FLAG-GFP-PEST is referred to as 3×FLAG-GFP in the text and figures of this section unless stated otherwise.

NaP-TRAP Reporter Libraries were Constructed Using Three Different PCR Reactions:

First, the common coding sequence and 3′ UTR of the reporters were amplified from the 3×FLAG-GFP plasmid, using a forward primer targeting the N-terminus of GFP and a reverse primer targeting the 3′ end of the 3′ UTR (F: 3×FLAG_GFP_fwd, pA-R: pcr_II_pA_rev, sv40-R: sv40_rev). For the Kozak library, a forward primer targeting the sequence immediately downstream of the variable Kozak sequence was used (F: ntrapK_GPF_fwd). All PCR amplicons were gel purified using a Monarch® DNA Gel Extraction Kit (NEB #T1020L).

Second, reporter libraries were amplified from 1 ng of single stranded DNA oligo pools using KAPA HiFi HotStart ReadyMix (Roche #7958935001) for 10-20 cycles of 98 °C, 60 °C, and 72 °C for 15, 20, and 30 seconds, respectively (F: SP6_II_adapt, R: GFP-aug_rev). For initial Kozak library generation see Random Kozak Library section. Each product was PCR purified using a DNA Clean & Concentrator-5 (Zymo #D4014).

Third, to generate a template for in vitro transcription, a PCR overlap extension was performed between the reporter library and the purified 3×FLAG-GFP-PEST amplicon using KAPA HiFi HotStart Ready Mix (Roche #7958935001) (0.1-1 ng of template DNA). After 10 cycles of 98 °C, 60 °C, and 72 °C for 15, 20, and 30 seconds respectively, primers targeting the 5′ SP6 promoter sequence and the 3′ end of the 3×FLAG-GFP-PEST amplicons were added (F: SP6_II_adapt, pA-R: pCS2_3utr_60A, sv40-R: sv40_rev) followed by an additional 20 cycles at the same conditions. Unless stated explicitly, reporter libraries contained a 60A tail (pA-R: pCS2_3utr_60A). For libraries with an SV40 polyadenylation signal, a reverse primer targeting the 3′ end of the SV40 poly-adenylation signal was used for the amplification of 3×FLAG-GFP-PEST and the assembly of reporter library (R: SV40_rev).

Lastly, templates for in vitro transcription were gel purified using a Monarch® DNA Gel Extraction Kit (NEB #T1020L). Reporter mRNAs were generated using a mMESSAGE mMACHINE™ SP6 transcription kit (Invitrogen™ #AM1340). In vitro transcribed mRNAs were purified using a Monarch® RNA Cleanup Kit (NEB #T2040L). All libraries were injected into single cell zebrafish embryos at 20 μg per embryo.

### Random Kozak Library

The random Kozak library consisted of an 15 Illumina adaptor (5′-CCCTACACGACGCTCTTCCGATCT-3′, SEQ ID NO:2366) followed by the 5′ UTR of Xenopus beta-globin, seven random nucleotides, six upstream and one downstream of the start codon, and the N-terminus of 3×FLAG GFP. The Kozak library was generated by performing a PCR overlap extension using KAPA HiFi HotStart ReadyMix (Roche #7958935001) for 10 cycles of 98 °C, 60 °C, and 72 °C for 15, 20, and 30 seconds respectively (F: kozak_ntrap_fwd, R: kozak_7_nt_rev, 1 μL of each primer at 100 μM).

### Zebrafish 5′UTR Library

A custom single stranded DNA oligo pool consisting of 11,088 oligos was ordered from GenScript (12 K oligo pool). Each 170 nt oligo contained an Illumina 15 (5′-CCCTACACGACGCTCTTCCGATCT-3′, SEQ ID NO:2366) adaptor sequence, a 124-nucleotide variable region, and 22 nt region with homology to the Kozak sequence and N-terminus of 3×FLAG-GFP (5′-GTAAACATGGTGAGCAAGGGCG-3′, SEQ ID NO:2367). The variable region of the 5′ UTR library was generated using a custom script, tiling the 5′ UTRs of 1,775 maternally supplied genes and six IRES sequences (human AQP4, human MYT2, human NRF, human XIAP, EMCV and crTMV) in 124 nucleotide segments every 25 nucleotides.

### Validation Library (Tetramer Repeats)

A custom single stranded DNA oligo pool consisting of 256 oligos was ordered from Twist Bioscience as part of a 12 K oligo pool. The design of the common regions of library were identical to that of the zebrafish 5′ UTR library. The variable region (124 nucleotides) consisted of repeats of all possible tetramers. Each repeat occurred 21 times and was separated by a dinucleotide spacer. The dinucleotide spacers were repeated in a pattern across the variable region (TC, AC, AG, CG). These dinucleotide spacers were selected to prevent the creation of unintended upstream ORFs.

### NaP-TRAP Spike-Ins

The design of the spike-in reporters was identical to that of the zebrafish 5′ UTR and tetramer validation libraries. Each spike-in reporter contained a 20 nucleotide identifier (see below). NaP-TRAP spike-ins were generated by performing a PCR overlap extension using KAPA HiFi HotStart Ready Mix (Roche #7958935001) (F: ntrap_sp1_fwd, ntrap_sp2_fwd, ntrap_sp3_fwd, ntrap_sp4_fwd, ntrap_sp5_fwd; R: ntrap_sp_rev) (1 μL of each primer at 100 μM) for 10 cycles of 98 °C, 60 °C, and 72 °C for 15, 20, and 30 seconds, respectively. Amplicons were gel purified using a Monarch® DNA Gel Extraction Kit (NEB #T1020L). Next, the purified amplicons were cloned into the 3×FLAG-GFP-PEST vector using In-Fusion® HD Cloning (Takara #638946) (vector F: ntrap_spike_inf_fwd, R: ntrap_spike_inf_rev). To generate spike-in mRNAs, plasmids were amplified using KAPA HiFi HotStart ReadyMix (Roche #7958935001) (F: Sp6-Il-adapt, R: pCS2_3utr_60A) for 20 cycles at 98 °C, 60 °C, and 72 °C for 15, 20, and 30 seconds, respectively. Products were then gel purified with a Monarch® DNA Gel Extraction Kit (NEB #T1020L) and then in vitro transcribed with a mMESSAGE mMACHINE™ SP6 transcription kit (Invitrogen™ #AM1340). mRNAs were purified with a Monarch® RNA Cleanup Kit (NEB #T2040L) prior to use. Spike-ins were pooled and added at the RNA extraction step at concentrations of 1, 5, 25, 50, and 125 fg for the 5′ UTR library.

Spike-in #1
(SEQ ID NO: 2368)
TGACGTGGAAGTCGGTCAAG
Spike-in #2
(SEQ ID NO: 2369)
GTCCAGAGACAAAGTCCGGG
Spike-in #3
(SEQ ID NO: 2370)
CACGAGGAGGAACCAGTGAC
Spike-in #4
(SEQ ID NO: 2371)
CTGTTGTTGTGTGAAGGGCG
Spike-in #5
(SEQ ID NO: 2372)
GCTCTCGGTCTCGGAAGAAG

### NaP-TRAP (Nascent Peptide Translating Ribosome Affinity Purification)

To capture tagged nascent chains of reporter mRNAs, an immunoprecipitation using anti-FLAG magnetic beads was performed. Magnetic beads were purchased from three suppliers during the course of the study due to supply chain issues (ANTI-FLAG® M2 Magnetic Beads, Millipore® #M8823; Anti-Flag Magnetic Beads, BioTools LLC #B26102; Pierce™ Anti-DYKDDDDK (SEQ ID NO:2373) Magnetic Agarose, Thermo Fisher Scientific #88836). For the zebrafish and human cell experiments 10 μL and 20 μL of beads (binding capacity of >0.8 mg of FLAG peptide/mL) were utilized, respectively. The magnetic beads were washed with 800 μL of wash buffer three times prior to being added to the lysis solution.

Briefly, frozen embryos and HEK293T cells were lysed in 500 μL of lysis buffer. After 10 minutes at 4 °C the lysate was passed through a 25-gauge needle (5-10 times). Next, the sample was centrifuged at 16,000 g for 5 minutes at 4 °C. The supernatant was transferred to a new Eppendorf tube and 2 μL of DNase I (NEB #M0303L) was added. After a 15 minute incubation at 4 °C, the samples were diluted to 1 mL using additional lysis buffer and 75 μL of lysate was collected from each sample to serve as an input. Samples were placed on a rotator at 4 °C for 2 hrs. Following incubation and bead capture, the beads were washed with 800 μL of wash buffer three times. The beads (pulldown) and the inputs were then resuspended in 1 mL of Trizol (Invitrogen #15596-018). For the zebrafish 5′ UTR and tetramer validation libraries, spike in reporters were added. RNA extractions were performed in accordance with the manufacture's protocol. RNA pellets were resuspended in 11 μL of nuclease free H₂O.

### Library Preparation for Next-Generation Sequencing

Reverse transcription primers (4 μM total) targeting the N-terminus of 3×FLAG GFP were added to purified input and pulldown RNAs (5′-GTGACTGGAGTTCAGACGTGTGCTCTTCCGATCT (SEQ ID NO:2374)-CTAC-10N UMI-TAAC-6 nt sample barcode-GCGTCATGGTCTTTGTAGTCCTC (SEQ ID NO:2375)-3′). These primers contained a 6-nucleotide sample barcode and a 10-nucleotide Unique Molecular Identifier (UMI) to allow for demultiplexing and read deduplication, as well as a 3′ I7 Illumina adaptor sequence. To increase library complexity the primer pairs were staggered by 1 nt (e.g., ntrap_RT_b1.1, ntrap_RT_b1.2). Reverse transcription was performed using the Superscript III kit (Invitrogen #18080044) in accordance with manufacturer's instructions. Reverse transcription reactions were performed at 55 °C cDNA from replicates were pooled and purified by adding AMPure XP Reagent (Beckman Coulter #A63881) at 1.8× the original sample volume. Illumina 15 and 17 forward and reverse primers containing a 10-nucleotide index (see below) were utilized to amplify cDNA libraries via PCR (Kappa Polymerase Master Mix). To reduce the number of PCR duplicates 12-18 cycles were utilized. Amplicons were purified by adding AMPure XP Reagent (Beckman Coulter #A63881) at 0.9× the volume of the PCR reaction. Libraries were sequenced on Illumina NovaSeq 6000 platform.

Illumina 15 primer:
(SEQ ID NO: 2376)
5′-AATGATACGGCGACCACCGAGATCTACAC-10 nt
(SEQ ID NO: 2377)
index-ACACTCTTTCCCTACACGACGCTCTTCCGATCT-3′
812 Illumina I7 primer:
(SEQ ID NO: 2378)
5′-CAAGCAGAAGACGGCATACGAGAT-10 nt index-
(SEQ ID NO: 2379)
GTGACTGGAGTTCAGACGTGTGCTCTTCCGATCT-3′

### NaP-TRAP-qPCR Analysis

Following NaP-TRAP, cDNA was synthesized using random hexamers (ThermoFisher Scientific #N8080127) and SuperScript™ III Reverse Transcriptase (Invitrogen #18080044) following the manufacturer's protocol. Translation values were determined by qPCR using the Power SYBR™ Green PCR Master Mix (Applied Biosystems #4367659). Levels of reporters in the input and pulldown were normalized using the 2-ΔΔCt method,59 where the NaP-TRAP reporter was the target and dsRED the control. Translation was calculated as a ratio of fold enrichment in the pulldown relative to the input.

Primers Utilized in qPCR:
Translation blocking morpholino (GFP_qpcr_fwd, GFP_qpcr_rev)
MicroRNA seeds in 3′ UTR (3×mir-430_qpcr_fwd, 3×mir-204_qpcr_fwd, T7_qpcr_rev)
U/C rich reporters (igf1ra_wt_qPCR_fwd, igflra_mut_qPCR_fwd, asun_wt_qPCR_fwd, asun_mut_qPCR_fwd, sich211_wt_qPCR_fwd, sich211_mut_qPCR_fwd, atp6v0_wt_qPCR_fwd, atp6v0_mut_qPCR_fwd, GFP_qPCR_5p_rev)
dsRED (dsRED_qpcr_fwd, dsRED_qpcr_rev)

### NaP-TRAP NanoLuc Reporter Construction

The present study substituted NanoLuc luciferase (amplified from a gBlocks™ Gene Fragment, Integrated DNA Technologies IDT) for GFP in the 3×FLAG-GFP-PEST vector using In-Fusion® HD Cloning (Takara #638946) (3×FLAG-PEST vector F: PEST_fwd, R: FLAG_rev_inf; NanoLuc insert F: FLAG-Nluc_inf_fwd, R: Nluc_inf_rev). Note: The PEST domain was included in NanoLuc constructs to reduce the half-life of the NanoLuc protein and thereby improve the sensitivity of the assay. To generate NanoLuc reporters 3×FLAG-NanoLuc-PEST was amplified using KAPA HiFi HotStart ReadyMix (Roche #7958935001) (F: 3×FLAG_GFP_fwd R: pcr_II_pa_rev) and gel purified using a Monarch® DNA Gel Extraction Kit (NEB #T1020L).

### Kozak NanoLuc Reporters

To construct Kozak NanoLuc reporters, 3×FLAG-NanoLuc (amplicon from previous section) was amplified with four different forward primers (ntrap_k1_fwd, ntrap_k2_fwd, ntrap_k3_fwd, and ntrap_k4_fwd) and a common reverse primer (pcr_II_pa_rev) using KAPA HiFi HotStart Ready Mix (Roche #7958935001) at 20 cycles of 98 °C, 60 °C, and 72 °C for 15, 20, and 45 seconds, respectively. Products were gel purified using a Monarch® DNA Gel Extraction Kit (NEB #T1020L). To add an SP6 promoter sequence and a 60A hard-encoded tail the purified product (1 ng) was amplified using KAPA HiFi HotStart Ready Mix (Roche #7958935001) at 20 cycles of 98 °C, 60 °C, and 72 °C for 15, 20, and 45 seconds, respectively (F: Sp6-Il-adapt, R: pCS2_3utr_60A). Products were gel purified with a Monarch® DNA Gel Extraction Kit (NEB #T1020L) and then in vitro transcribed with a mMESSAGE mMACHINE™ SP6 transcription kit (Invitrogen™ #AM1340). mRNAs were purified with a Monarch® RNA Cleanup Kit (NEB #T2040L) prior to injection. Note, the input of one of the replicates of the Kozak reporter K4 was below the read filter cutoff in the NaP-TRAP Kozak Library experiment.

### Generating NanoLuc Reporters Using PCR Overlap

The 5′ UTRs of NanoLuc Reporters were generated using PCR overlap (see below for forward and reverse primers), using KAPA HiFi HotStart ReadyMix (Roche #7958935001) at 10 cycles of 98 °C, 60 °C, and 72 °C for 15, 20, and 30 seconds, respectively (1 μL of each primer at 100 μM). The extension products were then gel purified using a Monarch® DNA Gel Extraction Kit (NEB #T1020L).

Full-length reporters were constructed by performing a PCR overlap extension of the 5′ UTR products with the NanoLuc amplicon described in the Cloning NanoLuc section using KAPA HiFi HotStart Ready Mix (Roche #7958935001). After 10 cycles of 98 °C, 60 °C, and 72 °C for 15, 20, and 40 seconds, respectively, primers targeting the SP6 promoter sequence (F: Sp6-II-adapt), and the 3′ end of the nanoLuc amplicon were added (R: pCS2_3utr_60A). The PCR reaction was continued for an additional 20 cycles at the same cycle conditions. Products were gel purified with a Monarch® DNA Gel Extraction Kit (NEB #T1020L) and then in vitro transcribed with ta mMESSAGE mMACHINE™ SP6 transcription kit (Invitrogen™ #AM1340). mRNAs were purified with a Monarch® RNA Cleanup Kit (NEB #T2040L) prior to injection.

Zebrafish 5′ UTR library validation reporters (slc16al_fwd, slc16a1_rev, spred1_fwd, spredl_rev, eif4ebp2_fwd, eif4ebp2_rev, fth1a_fwd, fth1a_rev, wdr41_fwd, wdr41_rev, slc38a7_fwd, slc38a7_rev, atp2b4_fwd, atp2b4_rev, atp6v0_wt_fwd, atp6v0_wt_rev, Igf1ra_wt_fwd, Igf1ra_wt_rev).

4×miR-430 NanoLuc—(WT-F: 4×miR-430-fwd, WT-R: 4×miR-430-rev; MUT-F: 4×miR-430-MUT-fwd, MUT-R: 4×miR-430-MUT-rev).

### Dual Luciferase Assay

Firefly luciferase was amplified from a pCS2+-Fluc plasmid using KAPA HiFi HotStart Ready Mix (Roche #7958935001) at 20 cycles of 98 °C, 60 °C, and 72 °C for 15, 20, and 50 seconds (F: SP6_ext_fwd, R: SV40_60A_rev). Products were gel purified with a Monarch® DNA Gel Extraction Kit (NEB #T1020L) and then in vitro transcribed with a mMESSAGE mMACHINE™ SP6 transcription kit (Invitrogen™ #AM1340). mRNAs were purified with a Monarch® RNA Cleanup Kit (NEB #T2040L) prior to injection.

Single cell zebrafish embryos were co-injected with mRNAs encoding nano and firefly luciferase (0.5 μg of nano/19.5 μg of firefly). Embryos were collected at either 2 hpf or 6 hpf and frozen in liquid nitrogen (5 embryos per replicate). Nano and firefly luciferase activities were measured using the Nano-Glo® Dual-Luciferase® Reporter Assay System (Promega #N1610). Firefly luciferase activity was utilized to normalize nano luciferase measurement across reporters.

### C and U Rich Reporters

C- and U-rich wild-type and mutant reporters were generated through PCR overlap extension (Primers used: igf1ra_wt_fwd, igf1ra_wt_rev; igf1ra_mut_fwd, igf1ra_mut_rev; asun_wt_fwd, asun_wt_rev; asun_mut_fwd, asun_mut_rev; sich211_wt_fwd, sich211_wt_rev; sich211_mut_fwd, sich211_mut_R atp6v0_wt_fwd, atp6v0_wt_rev; atp6v0_mut_F atp6v0_mut_R) using KAPA HiFi HotStart ReadyMix (Roche #7958935001) at 10 cycles of 98 °C, 60 °C, and 72 °C for 15, 20, and 30 seconds, respectively (1 μL of each primer at 100 μM). The extension products were then gel purified using a using Monarch® DNA Gel Extraction Kit (NEB #T1020L).

Extension products were cloned into the 3×FLAG-GFP-3×AID* vector using In-Fusion® HD Cloning (Takara #638946) (vector F: 3×FLAG_GFP_F R: 15_inf_rev). Plasmids were digested with NotI-HF® (NEB #R3189L) and in vitro transcribed with a mMESSAGE mMACHINE™ SP6 transcription kit (Invitrogen™ #AM1340). mRNAs were purified with a Monarch® RNA Cleanup Kit (NEB #T2040L) prior to injection.

### Quantification and Statistical Analysis

### Read Trimming and Translation Calculation

Reporter libraries were sequenced on an Illumina NovaSeq 6000 platform. Sequencing data were stored using LabxDB.60 Paired-end reads were trimmed and demultiplexed using ReadKnead (github dot com/vejnar/ReadKnead). Barcodes identifying replicates and UMIs were extracted from read two. Common regions were trimmed from read one to facilitate accurate mapping. For the Kozak library, reporters were counted using a custom python script. Reporters with indels in the Kozak sequence or reporters without an AUG in the appropriate position were eliminated. For the zebrafish 5′ UTR and validation libraries, reads were mapped to a library specific index using Bowtie2.61 PCR duplicates were eliminated using UMIs. UMIs were considered identical if they had a Hamming Distance less than 2. Reads for each experiment were normalized by dividing the read counts of each reporter by the sum of the total number of reads mapped to the spike-ins. In the 5′ UTR reporter library (pA and sv40 in zebrafish) spike-in #3 was eliminated from the analysis, because in some of the samples the read counts for spike-in #3 did not correlate with amount of spike-in added. In the absence of spike-ins, read counts were normalized based on the total number of mapped reads per replicate (reads per million, RPM). Translation values were calculated as a ratio of reads in the pulldown relative to reads in the input. Translation values were only included in the downstream analyses if the input contained greater than or equal to 100 unique reads across all replicates.

### Random Forest Regression Models

Random forest regression models were employed to predict translation (scikit-learn 1.3; RandomForestRegressor).62 For the Kozak library, features were generated by one-hot encoding positions −6 to −1 and position +4 of each reporter sequence. In contrast, for the 5′ UTR library, k-mer counts (1-6 nucleotides) and uORF features were generated using a custom python script. For the 5′ UTR library, features were filtered by calculating the Spearman Rank Correlation Coefficient (SPR) between each feature and translation prior to model training. Features that had a correlation greater than 0.05 or less than-0.05 were included in the random forest model.

To prevent model overfitting, the data were divided randomly into two groups: a test and training set, comprised of 30% and 70% of the reporters, respectively. To optimize model parameters, the training data were divided into five different groups of equal size and a 5-fold cross-validation was performed. The following parameters were optimized using an exhaustive grid search (n_estimators: 20, 100, 200; max_features: 10, 20 and 30 percent of supplied features; max_depth: 3, 5, 7 and min_samples_split: 2, 4, 8). Bootstrapping was employed to select samples used to train each tree. The predictive power of the model was assessed using the test set. A permuted feature importance analysis was performed to identify the features with the greatest predictive power.

### Differential Motif Enrichment Analysis

Reporters were ranked based on their translation at 2 and 6 hpf. Using the sum and difference of these rankings across timepoints, four groups of reporters were generated: (1) repressed, (2) active, (3) repressed post ZGA (active in HEK293T cells), and (4) active post ZGA (active in zebrafish). Repressed and active reporters constituted the top and bottom 10% of reporters based on the sum of their ranks at 2 and 6 hpf, respectively, whereas the repress post ZGA and active post-ZGA. were the top and bottom 10% of groups based on the difference between their ranks at 2 and 6 hpf. A differential motif enrichment analysis was performed on each group. Fold enrichment values were determined by dividing the count of each k-mer in the reporter group by the count of the k-mer in the library, whereas the significance of the fold-change was determined using a hypergeometric test (Bonferroni corrected p-value threshold).

miRNA Complementarity Analysis

miR-430 (GCACUU) and miR-1 (ACAUUC) seeds were identified in the reporter library. The Vienna RNAcofold program63 was utilized to measure the complementarity between the section of the reporter mRNA (20 nt upstream and 7 nt downstream of the 5′ end of seed site) and the microRNA. For miR-430 the miRNA species with the highest complementary was selected for downstream analysis.

miR-430a:
(SEQ ID NO: 2380)
5′-UAAGUGCUAUUUGUUGGGGUAG-3′
miR-430b:
(SEQ ID NO: 2381)
5′-AAAGUGCUAUCAAGUUGGGGUAG-3′
miR-430c:
(SEQ ID NO: 2382)
5′-UAAGUGCUUCUCUUUGGGGUAG-3′
miR-1-1/mir-1-2:
(SEQ ID NO: 2383)
5′-UGGAAUGUAAAGAAGUAUGUAU-3′

### Feature Rank Analysis

To generate the feature rank plot (FIG. 13A), features (k-mer counts of four nucleotides or fewer) were ranked based on the mean difference in translation between reporters enriched and depleted in the feature, top and bottom 20% respectively, at 2 and 6 hpf. Given the prominent effect of upstream open reading frames on translation, reporters with uORFs were excluded from the analysis. Features were only included in the analysis if there was a significant mean difference in translation at either timepoint (Mann-Whitney U test with Bonferroni corrected p-value threshold).

### Statistical Analyses

The plots and statistical analyses in FIGS. 8B, 8C, 9G, 12E and 13C-13F were generated using GraphPad Prism. All other analyses unless otherwise stated were performed using custom scripts written in Python 3. Plots were generated using the Matplotlib package. Venn diagrams were generated using Matplot-venn (github dot com/konstantint/matplotlib-venn). Statistical analyses were performed using the SciPy and NumPy packages, whereas the random forest analysis was performed using the scikit-learn package. Feature and experimental data were stored using an SQLite database (https://www.sqlite.org/).

### Detailed Protocol for NaP-TRAP

### Buffers

10× salt buffer—15 mM Tris 7.4 (ThermoFisher Scientific #612021000, 100 mM NaCl (Sigma-Aldrich #S6546-1L), 10 mM MgCl₂ (Invitrogen™ #AM9530G).
Bead wash buffer—1× salt buffer, 2 mM DTT (ThermoFisher Scientific #P2325).
Lysis buffer—1× salt buffer, 10% triton-X (Sigma-Aldrich #X100-500ML), 2 mM DTT (ThermoFisher Scientific #P2325), 100 μg/mL Cycloheximide (Sigma-Aldrich #01810-1G), 40 U RNaseOUT™ Recombinant Ribonuclease Inhibitor (ThermoFisher Scientific #10777019), 1× cOmplete™ EDTA-free Protease Inhibitor (Roche #11873580001).
NaP-TRAP wash buffer—Lysis buffer+400 mM NaCl (Sigma-Aldrich #S6546-1L).
HEK293T media-Dulbecco's Modified Eagle Medium (DMEM) (ThermoFisher Scientific #10569010), 10% heat inactivated Fetal Bovine Serum (FBS) (ThermoFisher Scientific #16140071), 20 mM HEPES (ThermoFisher Scientific #15630080), 2 mM L-Glutamine (ThermoFisher Scientific #25030081), 1× Penicillin/Streptomycin (ThermoFisher Scientific #15140122).
DPBS—cycloheximide-Dulbecco's phosphate-buffered saline (DPBS) (ThermoFisher Scientific #14190144), 100 μg/mL cycloheximide (Sigma-Aldrich #01810-1G).

### Zebrafish Injections

1. After zebrafish mating, collect embryos and remove their chorion through treatment with Pronase (Sigma-Aldrich #10165921001).
2. Inject dechorionated embryos with 20 μg of an in vitro transcribed mRNA reporter library.
3. Incubate embryos at 28 °C.
4. Collect 25-50 embryos per replicate at 2 and 6 hpf. Transfer the embryos to a 1.5 mL Eppendorf tube and freeze them in liquid nitrogen (store at −80 °C).
5. Prior to NaP-TRAP, add 500 mL of lysis buffer to each set of frozen embryos. Vortex the samples to resuspend the frozen pellet (proceed to NaP-TRAP protocol).

### HEK293T RNA Transfection

1. Coat plates with poly-d-lysine (1 mL per well) (ThermoFisher Scientific #A3890401). Incubate for 1 hour at room temperature. Wash plates with sterile water three times. Allow plates to dry.
2. Seed a 6-well plate at 200,000 to 300,000 cells per well (1 mL HEK293T media). Incubate the cells for 24-36 hours prior to transfection.
3. For each well mix 3.75 μL of Lipofectamine™ MessengerMAX™ Transfection Reagent (ThermoFisher Scientific #LMRNA008) with 121.25 μL of Opti-MEM™ I Reduced Serum Medium (ThermoFisher Scientific #31985062). Incubate the solution for 10 minutes at room temperature.
4. Dilute 1-2 μg of mRNA in 125 μL of Opti-MEM™ I Reduced Serum Medium (ThermoFisher Scientific #31985062) and add to the Lipofectamine™ mixture. Gently mix and incubate for another 5 minutes at room temperature.
5. Add 250 μL of mRNA-Lipofectamine™ solution to each well drop-wise distributing the solution throughout the plate.
6. Incubate for 1 hour at 37 °C with 5% CO₂.
7. Aspirate cells and wash with prewarmed DPBS (ThermoFisher Scientific #14190144). Add new media (1 mL prewarmed)
8. Grow the cells for 12 hours at 37 °C with 5% CO₂.
9. Add 100 μg/mL cycloheximide (Sigma-Aldrich #01810-1G) to cells and incubate for 10 minutes at 37 °C with 5% CO₂.
10. Place cells on ice and aspirate media and wash cells with ice cold DPBS+cycloheximide.
11. Add 500 μL of lysis buffer to the cells.
12. Disrupt cells mechanically with a plastic cell scraper and transfer the lysate to a new tube.
13. Incubate for 10 minutes on ice.

### Nascent Peptide Ribosome Affinity Purification

1. Using a magnetic stand (DynaMag™-2 Magnet, Invitrogen™ #12321D), wash the anti-FLAG magnetic beads, 10 μL per zebrafish sample and 20 μL per HEK293T sample (binding capacity of >0.8 mg of FLAG peptide/mL) with 800 μL of the bead wash buffer three times. Until the samples are ready, leave the beads in the final wash on ice. When working with the anti-FLAG beads it is important to utilize pre-lubricated Eppendorf tubes.

Note: Magnetic beads were purchased from three suppliers during the course of the study due to supply chain issues (ANTI-FLAG® M2 Magnetic Beads, Millipore® #M8823; Anti-Flag magnetic beads, BioTools LLC #B26102; Pierce™ Anti-DYKDDDDK Magnetic Agarose, ThermoFisher Scientific #88836). Pierce™ Anti-DYKDDDDK Magnetic Agarose is recommend for this experiment.
2. Pass lysed cells through a 26 G needle 5-10 times.
3. Spin lysed cells at 16,000 g for 5 minutes at 4 °C.
4. Transfer the supernatant to a new tube and add 2 μL of DNASE 1 (0.006 U/mL) (NEB #M0303L).
5. Incubate samples on ice for 15 minutes.
6. Add 500 μL of lysis buffer to bring the total volume of the solution to 1 mL.
7. Collect 75 μL of lysate as input (set aside on ice).
8. Add supernatant to 10 μL of washed beads from step 1.
9. Rotate the beads on a rotor for 2 hours at 4 °C.
10. Using a magnetic stand (DynaMag™-2 Magnet, Invitrogen™ #12321D), remove the supernatant from the beads and wash the beads with 800 μL of the NaP-TRAP wash buffer three times. To limit experimental noise, when adding the NaP-TRAP wash buffer be sure to resuspend the magnetic beads.
11. Add 1 mL of Trizol (Invitrogen #15596-018) to the washed beads and the input. (Samples can be stored here at −20 °C.).

### RNA Extraction

12. Add in vitro transcribed NaP-TRAP spike-ins to Trizol (Invitrogen #15596-018). The present study utilized 1, 5, 25, 50 and 125 fg for spike-ins #1-5, respectively. This total will need to be adjusted for the experimental system.
13. Add 200 μL of chloroform to samples. Vortex and incubate samples for 2-3 minutes at RT.
14. Centrifuge samples 12,000 g for 15 minutes at 4 °C.
15. Transfer the aqueous phase to a fresh 1.5 ml Eppendorf tube and add 1.5 μL of GlycoBlue (Invitrogen #AM9516) and 500 μL of Isopropanol. Vortex and incubate samples at RT for 10 minutes.
16. Centrifuge samples at 12,000 g for 15 minutes at 4 °C.
17. Remove supernatant. Wash RNA pellet twice with 1 mL of 75% ethanol. Centrifuge samples 7500 g for 10 minutes at 4 °C.
18. Allow pellet to dry (5-10 minutes). Resuspend pellet in 11 μL of nuclease free H₂O.

### Library Preparation

19. Transfer resuspended RNA to PCR tubes. Add 1 μL of 10 mM dNTPs (NEB #N0446S) and 1 μL of 4 μM of barcoded reverse transcription primers to the samples.
20. For primer annealing, heat samples to 95 °C for 2 minutes and then 65 °C for 5 minutes. Place samples on ice for at least 1 minute.
21. Add 7 μL of SuperScript™ III Reverse Transcriptase master mix to each sample (per sample: 4 μL 5×First Strand Synthesis Buffer, 1 μL 0.1 M DTT, 1 μL RNaseOUT, and 1 μL of SuperScript™ III)
22. For the reverse transcription reaction, heat samples for 1 hour at 55 °C and then 15 minutes at 70 °C.
23. Add 2 μL of RNase H (NEB #M0297L) to each sample. Heat samples for 15 minutes at 37 °C.
24. Combine RT barcoded replicates (Note: keep the input and pulldown samples separate).
25. Add 1.8×AMPure XP Reagent (Beckman Coulter #A63881) to each sample. Mix the beads by pipetting the samples.
26. Incubate the mixture at RT for 15 minutes.
27. Using a magnetic stand (DynaMag™-2 Magnet, Invitrogen™ #12321D) remove the supernatant and wash the beads twice with 75% ethanol.
28. After washing the beads, allow them to dry at RT (~10 minutes).
29. Resuspend the beads in 30 μL of nuclease free H₂O and incubate for 5 minutes at RT. Using a magnetic stand (DynaMag™-2 Magnet, Invitrogen™ #12321D) immobilize the beads and transfer the purified cDNA to a new tube.
30. Amplify library for Next-Generation sequencing. Mix 12.5 μL of 2×KAPA HiFi HotStart Ready Mix (Roche #7958935001), 1 μL of an 15 forward, 1 μL 17 reverse primer, 1-10.5 μL of cDNA, 0-9.5 μL of nuclease-free H₂O. PCR specifications: 95 °C. 3 minutes, 10-20 cycles of 98 °C, 60 °C, and 72 °C for 15, 20, and 30 seconds, respectively, 72 °C. 5 minutes (F: 15_10 nt_[id] _fwd; R: 17_10 nt_[id]_rev). Cycle conditions were optimized by running amplicons of a 20 cycle test reaction on an agarose gel.
31. Purify library with AMPure XP Reagent (Beckman Coulter #A63881) at a concentration of 0.9× the sample volume.
32. Note: Gel extract for precise library selection and noise reduction. Check sample purity and concentration with a Bioanalyzer prior to sequencing.

### Methods for 5′Utr 3′Utr Hexamer Library Half-Life Calculation

5UTR and 3UTR hexamer decay library transfected into HEK293T cells and collected at 6 h, 18 h, 24 h, 36 h, 48 h, 72 h. RNA was extracted from the collected samples, five spike-ins designed from random 25mer were added, and reverse transcribed using Superscript III kit (Invitrogen). cDNA was size purified using AmPure XP beads (Beckman Coulter) and purified again after amplifying the inserted codon repeats directly using Illumina 5′ and 3′ adaptors fused with reverse transcription barcode region. Libraries were sequenced on Illumina NovaSeq 6000 with paired-end 150 bp.

Sequencing data were stored using LabxDB (Vejnar, C. E. and A. J. Giraldez, Bioinformatics, 2020. 36 (16): p. 4530-4531). Barcodes were extracted from the read two. Trimmed reads were mapped using Bowtie2 (Langmead, B. and Salzberg, S. L., Nature Methods, 2012. 9 (4): 357-9). Counts of constructs were normalized using five spike-ins. Normalized counts of 6 h, 18 h, 24 h, 36 h, 48 h, 72 h were plotted, which follows an exponential decay graph, where the present study can calculate half-lives from graphs.

### Example 3: Translational Efficiencies as Determined by NaP-TRAP Matches Those Determined by Polysome Profiling

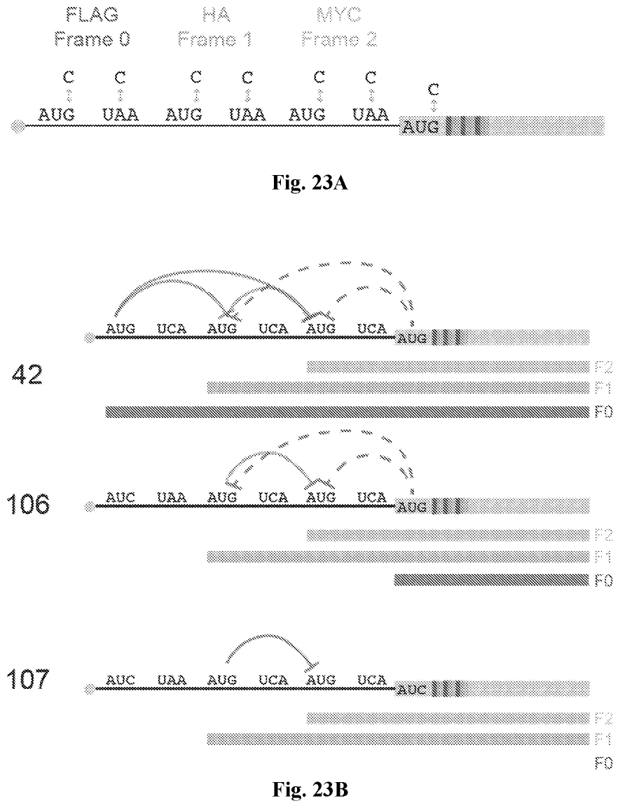

Referring to FIG. 22, the present study benchmarked NaP-TRAP against polysome profiling, a well-established translation based MPRA method. To minimize the effect of ribosomes translation out-of-frame, the present study incorporated stop codons early in the coding sequence of 3×FLAG-GFP.

The present study observed a strong correlation between NaP-TRAP derived translation values and Mean Ribosome load, demonstrating that NaP-TRAP captures ribosomes in a manner proportional to the number of ribosomes translating the main coding sequence.

It should be noted that in the absence of these out-of-frame stop codons, these translation metrics are likely to diverge. The reason is that the polysome profiling method cannot distinguish translation products produced by frameshifts. NaP-TRAP results, due to the specificity to the sequences of the translation products, filter the translations that involve frameshift (See e.g., Example 4).

### Example 4: NaP-TRAP is Able to Capture Translation in Multiple Frames Simultaneously

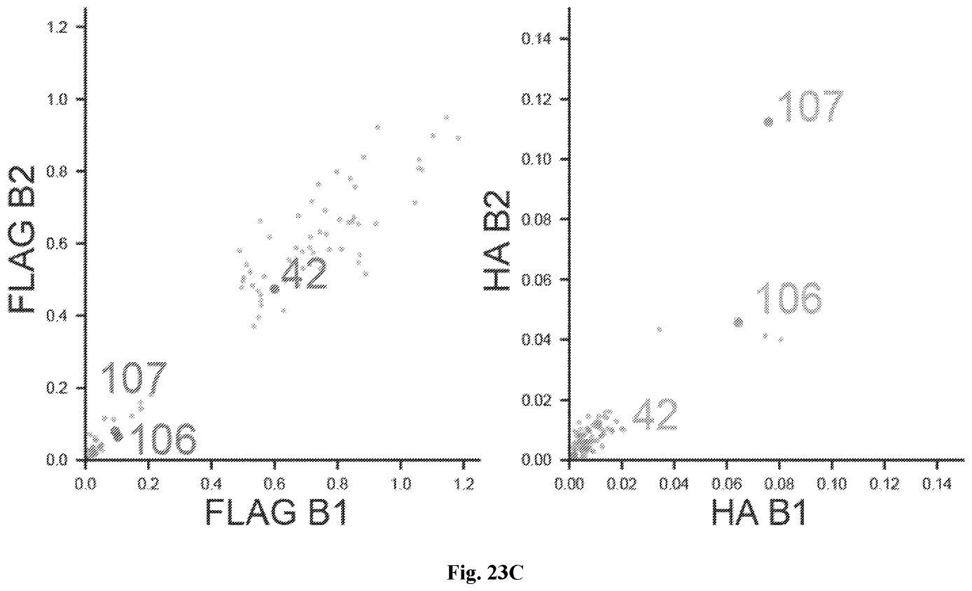

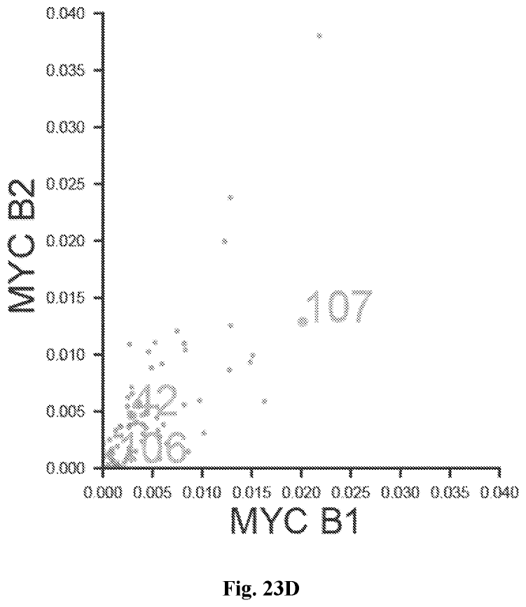

Referring to FIGS. 23A-23D, the present study demonstrated the capacity of NaP-TRAP to measure translation in multi-frames simultaneously.

Specifically, the present study incorporated Ha-tags in frame +1 and MYC-tags frame +2, in addition to the FLAG tag in frame 0. Translations in all the three frames were detected by detecting HA, MYC and FLAG tags, respectively. This experiment demonstrated the capacity of NaP-TRAP to measure translation in multiple frames simultaneously.

Using a library with upstream start codons in each frame, the present study further demonstrated the frame specificity of the method. The present study only observed translation in frames+1 and 2+ when an overlapping open reading frame is present.

### Example 5: Identification of a 5′UTR with High Translation Potential

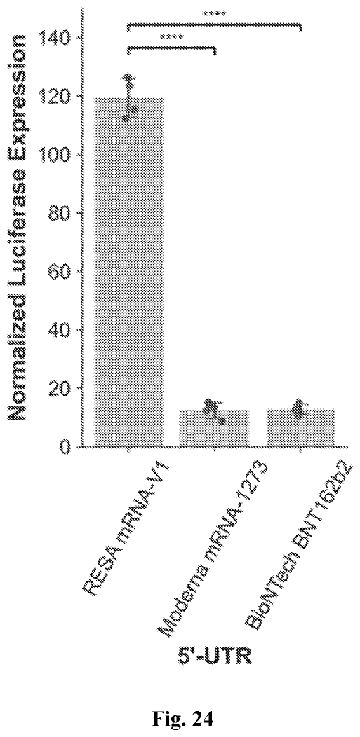

The present study performed NaP-TRAP to test the translation efficiencies of several 5′UTRs in HEK293T cells. The present study further selected a 5′UTR that showed high translation potential. The present study further engineered this 5′UTR by introducing base changes based on the understanding of how sequences variations in 5′UTR affect translation. The present study tested the translation efficiency of this engineered 5′UTR (RESA mRNA-V1) against the 5′UTRs used in commercially available CoVID vaccines (BNT 162b2 and mRNA-1273). The present study used a luciferase reporter to measure the protein output from these 5′UTRs and observed a 10-fold increased protein output from RESA mRNA-V1 as compared to BNT 162b2 and mRNA-1273 at the time-point tested (FIG. 24).

RESA mRNA-V1 (derived from Vax1) 5′UTR sequence
(SEQ ID NO: 2384)
5′GUACGGGCCGCGUUGUCGGGGGUUUUGUCCCCUUUUUCCUCUUUUUU
UUUUCCUUCUCCGCCCCACCCCCUUUUCUUUUUCUUUGCUUUUUUUUUC
CUUUCUGUUUUUGUUGUUGUUCUUGCCU3′

### Example 6: Identification of cis-Regulatory Elements in Human and Zebrafish Cells

Using the methods developed herein, the present study further identified cis-regulatory elements that modulates translation in human cells and zebrafish cells.

### Example 6-1: cis-Regulatory Elements Identified in Human Cells

In HEK293T cells, the following sequences were identified:

| SEQ ID NOs | Samples | Effects | Sequence | Fold change | p-value |
|---|---|---|---|---|---|
| SEQ ID NO: 1 | 12h_hek293t_utr5 | active | CCCUC | 1.743013 | 2.38E−10 |
| SEQ ID NO: 2 | 12h_hek293t_utr5 | active | CUCGC | 1.703225 | 1.25E−09 |
| SEQ ID NO: 3 | 12h_hek293t_utr5 | active | CUCUC | 1.551436 | 1.52E−09 |
| SEQ ID NO: 4 | 12h_hek293t_utr5 | active | UCCCC | 1.720921 | 1.53E−09 |
| SEQ ID NO: 5 | 12h_hek293t_utr5 | active | CCCCU | 1.726596 | 8.43E−09 |
| SEQ ID NO: 6 | 12h_hek293t_utr5 | active | CGGAG | 1.550163 | 1.26E−08 |
| SEQ ID NO: 7 | 12h_hek293t_utr5 | active | CUCGCG | 2.336449 | 2.03E−08 |
| SEQ ID NO: 8 | 12h_hek293t_utr5 | active | UUCCC | 1.539268 | 3.28E−08 |
| SEQ ID NO: 9 | 12h_hek2931_utr5 | active | UUUCU | 1.332462 | 9.33E−08 |
| SEQ ID NO: 10 | 12h_hek293t_utr5 | active | CUGAG | 1.460175 | 2.99E−07 |
| SEQ ID NO: 11 | 12h_hek293t_utr5 | active | CCCCUC | 2.135435 | 6.38E−07 |
| SEQ ID NO: 12 | 12h_hek293t_utr5 | active | UUUCCC | 1.719551 | 8.41E−07 |
| SEQ ID NO: 13 | 12h_hek293t_utr5 | active | UUCCCC | 1.874533 | 8.9E−07 |
| SEQ ID NO: 14 | 12h_hek293t_utr5 | active | CCCCC | 1.767378 | 9.56E−07 |
| SEQ ID NO: 15 | 12h_hek293t_utr5 | active | CUCUCU | 1.776675 | 1.08E−06 |
| SEQ ID NO: 16 | 12h_hek293t_utr5 | active | UCUCUC | 1.795162 | 1.2E−06 |
| SEQ ID NO: 17 | 12h_hek293t_utr5 | active | UCUCGC | 2.089013 | 1.58E−06 |
| SEQ ID NO: 18 | 12h_hek293t_utr5 | active | CCUCU | 1.45348 | 2.69E−06 |
| SEQ ID NO: 19 | 12h_hek293t_utr5 | active | UUUUCC | 1.567235 | 3.79E−06 |
| SEQ ID NO: 20 | 12h_hek293t_utr5 | active | GAGCG | 1.425081 | 4.61E−06 |
| SEQ ID NO: 21 | 12h_hek293t_utr5 | active | CGCCG | 1.506455 | 6.62E−06 |
| SEQ ID NO: 22 | 12h_hek293t_utr5 | active | CGAUUU | 1.925234 | 7.83E−06 |
| SEQ ID NO: 23 | 12h_hek293t_utr5 | active | CAUAC | 1.651939 | 8.02E−06 |
| SEQ ID NO: 24 | 12h_hek293t_utr5 | active | GCCGA | 1.549183 | 8.5E−06 |
| SEQ ID NO: 25 | 12h_hek293t_utr5 | active | GAGCU | 1.369919 | 8.53E−06 |
| SEQ ID NO: 26 | 12h_hek293t_utr5 | active | UCCCUC | 1.817238 | 8.98E−06 |
| SEQ ID NO: 27 | 12h_hek293t_utr5 | repressed | AUGGC | 3.077536 | 2.15E−46 |
| SEQ ID NO: 28 | 12h_hek293t_utr5 | repressed | CAUGG | 2.920373 | 5E−41 |
| SEQ ID NO: 29 | 12h_hek293t_utr5 | repressed | AUGGA | 2.689591 | 8.26E−41 |
| SEQ ID NO: 30 | 12h_hek293t_utr5 | repressed | GGAUG | 2.540254 | 5.12E−34 |
| SEQ ID NO: 31 | 12h_hek293t_utr5 | repressed | GAUGG | 2.660789 | 3.25E−32 |
| SEQ ID NO: 32 | 12h_hek293t_utr5 | repressed | AAUGG | 2.652044 | 2.63E−31 |
| SEQ ID NO: 33 | 12h_hek293t_utr5 | repressed | AGGAUG | 4.398545 | 3.13E−31 |
| SEQ ID NO: 34 | 12h_hek293t_utr5 | repressed | AUGGG | 2.823235 | 4.23E−28 |
| SEQ ID NO: 35 | 12h_hek293t_utr5 | repressed | CAAUG | 2.586673 | 7.57E−27 |
| SEQ ID NO: 36 | 12h_hek293t_utr5 | repressed | CCAUG | 2.73247 | 1.13E−25 |
| SEQ ID NO: 37 | 12h_hek293t_utr5 | repressed | AAAAUG | 3.397314 | 2.24E−25 |
| SEQ ID NO: 38 | 12h_hek293t_utr5 | repressed | AAAUG | 2.059367 | 1.15E−23 |
| SEQ ID NO: 39 | 12h_hek293t_utr5 | repressed | AUGGAG | 3.191252 | 2.88E−22 |
| SEQ ID NO: 40 | 12h_hek293t_utr5 | repressed | AAAUGG | 3.298909 | 6.92E−19 |
| SEQ ID NO: 41 | 12h_hek293t_utr5 | repressed | AGAUG | 2.078437 | 1.86E−18 |
| SEQ ID NO: 42 | 12h_hek293t_utr5 | repressed | AAUGGC | 3.407963 | 4.39E−18 |
| SEQ ID NO: 43 | 12h_hek293t_utr5 | repressed | AUGGCG | 3.451239 | 7.93E−18 |
| SEQ ID NO: 44 | 12h_hek293t_utr5 | repressed | AUGUG | 1.855269 | 3.52E−17 |
| SEQ ID NO: 45 | 12h_hek293t_utr5 | repressed | ACAAUG | 3.667851 | 3.89E−17 |
| SEQ ID NO: 46 | 12h_hek293t_utr5 | repressed | UCAUG | 1.961655 | 9.05E−17 |
| SEQ ID NO: 47 | 12h_hek293t_utr5 | repressed | AUCAUG | 3.275752 | 1.24E−16 |
| SEQ ID NO: 48 | 12h_hek293t_utr5 | repressed | GGAUGC | 3.416591 | 1.73E−16 |
| SEQ ID NO: 49 | 12h_hek293t_utr5 | repressed | GAUGGC | 3.544926 | 3.96E−16 |
| SEQ ID NO: 50 | 12h_hek293t_utr5 | repressed | AUGGCA | 3.254737 | 5.64E−16 |
| SEQ ID NO: 51 | 12h_hek293t_utr5 | repressed | CAUGGG | 4.018278 | 1.1E−15 |
| SEQ ID NO: 52 | 12h_hek293t_utr5 | repressed | AACAUG | 3.134098 | 1.19E−15 |
| SEQ ID NO: 53 | 12h_hek293t_utr5 | repressed | AAUGU | 1.819944 | 1.76E−15 |
| SEQ ID NO: 54 | 12h_hek293t_utr5 | repressed | AUGAG | 1.951154 | 1.89E−15 |
| SEQ ID NO: 55 | 12h_hek293t_utr5 | repressed | GAUGU | 1.889671 | 1.94E−15 |
| SEQ ID NO: 56 | 12h_hek293t_utr5 | repressed | UCAUGG | 3.100578 | 2.03E−15 |
| SEQ ID NO: 57 | 12h_hek293t_utr5 | repressed | GAUGA | 1.939293 | 2.12E−15 |
| SEQ ID NO: 58 | 12h_hek293t_utr5 | repressed | GAUGC | 2.081231 | 1.03E−14 |
| SEQ ID NO: 59 | 12h_hek293t_utr5 | repressed | AUGAA | 1.826349 | 2.89E−14 |
| SEQ ID NO: 60 | 12h_hek293t_utr5 | repressed | GUCAUG | 3.18366 | 5.22E−14 |
| SEQ ID NO: 61 | 12h_hek293t_utr5 | repressed | CAUGGC | 2.946394 | 6.85E−14 |
| SEQ ID NO: 62 | 12h_hek293t_utr5 | repressed | GCCAUG | 3.38234 | 8.03E−14 |
| SEQ ID NO: 63 | 12h_hek293t_utr5 | repressed | GAUGGA | 2.8356 | 9.61E−14 |
| SEQ ID NO: 64 | 12h_hek293t_utr5 | repressed | CAUGGA | 2.726371 | 1.04E−13 |
| SEQ ID NO: 65 | 12h_hek293t_utr5 | repressed | ACAUG | 1.939218 | 1.36E−13 |
| SEQ ID NO: 66 | 12h_hek293t_utr5 | repressed | CGAUG | 2.18248 | 1.75E−13 |
| SEQ ID NO: 67 | 12h_hek293t_utr5 | repressed | AUGUC | 1.981869 | 1.87E−13 |
| SEQ ID NO: 68 | 12h_hek293t_utr5 | repressed | AUGGCU | 3.052882 | 1.87E−13 |
| SEQ ID NO: 69 | 12h_hek293t_utr5 | repressed | GCAAUG | 3.283585 | 2.68E−13 |
| SEQ ID NO: 70 | 12h_hek293t_utr5 | repressed | AGGAU | 1.809015 | 3.09E−13 |
| SEQ ID NO: 71 | 12h_hek293t_utr5 | repressed | ACCAUG | 3.887603 | 4.17E−13 |
| SEQ ID NO: 72 | 12h_hek293t_utr5 | repressed | GGGAUG | 3.042472 | 6.33E−13 |
| SEQ ID NO: 73 | 12h_hek293t_utr5 | repressed | AAGAUG | 2.856198 | 1.97E−12 |
| SEQ ID NO: 74 | 12h_hek293t_utr5 | repressed | GAAAUG | 2.700528 | 2.28E−12 |
| SEQ ID NO: 75 | 12h_hek293t_utr5 | repressed | CAAUGU | 2.906015 | 2.54E−12 |
| SEQ ID NO: 76 | 12h_hek293t_utr5 | repressed | CCAUGG | 3.205046 | 4.03E−12 |
| SEQ ID NO: 77 | 12h_hek293t_utr5 | repressed | AUGGGA | 2.773951 | 4.24E−12 |
| SEQ ID NO: 78 | 12h_hek293t_utr5 | repressed | AUGAGG | 2.773951 | 4.24E−12 |
| SEQ ID NO: 79 | 12h_hek293t_utr5 | repressed | AAAUGA | 2.33897 | 5.01E−12 |
| SEQ ID NO: 80 | 12h_hek293t_utr5 | repressed | ACAUGG | 2.986025 | 6.52E−12 |
| SEQ ID NO: 81 | 12h_hek293t_utr5 | repressed | AUGUGG | 2.555208 | 2.55E−11 |
| SEQ ID NO: 82 | 12h_hek293t_utr5 | repressed | AGAUGG | 2.789775 | 3.37E−11 |
| SEQ ID NO: 83 | 12h_hek293t_utr5 | repressed | UGGAG | 1.569211 | 4.3E−11 |
| SEQ ID NO: 84 | 12h_hek293t_utr5 | repressed | AAUGGA | 2.757708 | 5.25E−11 |
| SEQ ID NO: 85 | 12h_hek293t_utr5 | repressed | AAUGA | 1.698285 | 8.13E−11 |
| SEQ ID NO: 86 | 12h_hek293t_utr5 | repressed | CAUGA | 1.830449 | 1.12E−10 |
| SEQ ID NO: 87 | 12h_hek293t_utr5 | repressed | UAUGG | 1.927187 | 2.34E−10 |
| SEQ ID NO: 88 | 12h_hek293t_utr5 | repressed | AUGGAA | 2.669571 | 2.75E−10 |
| SEQ ID NO: 89 | 12h_hek293t_utr5 | repressed | AUGGAC | 2.726371 | 3.09E−10 |
| SEQ ID NO: 90 | 12h_hek293t_utr5 | repressed | UGGCA | 1.840687 | 3.32E−10 |
| SEQ ID NO: 91 | 12h_hek293t_utr5 | repressed | GGAUGG | 2.856198 | 6.45E−10 |
| SEQ ID NO: 92 | 12h_hek293t_utr5 | repressed | GAUGUG | 2.245634 | 1.06E−09 |
| SEQ ID NO: 93 | 12h_hek293t_utr5 | repressed | GGAUGA | 2.369906 | 1.15E−09 |
| SEQ ID NO: 94 | 12h_hek293t_utr5 | repressed | AGCAUG | 2.686611 | 1.24E−09 |
| SEQ ID NO: 95 | 12h_hek293t_utr5 | repressed | UGAUG | 1.634118 | 1.87E−09 |
| SEQ ID NO: 96 | 12h_hek293t_utr5 | repressed | AGAAUG | 2.856198 | 4.48E−09 |
| SEQ ID NO: 97 | 12h_hek293t_utr5 | repressed | ACGAUG | 3.332231 | 5.34E−09 |
| SEQ ID NO: 98 | 12h_hek293t_utr5 | repressed | CGAUGG | 2.905022 | 7.37E−09 |
| SEQ ID NO: 99 | 12h_hek293t_utr5 | repressed | AUGAUG | 2.433057 | 1.04E−08 |
| SEQ ID NO: 100 | 12h_hek293t_utr5 | repressed | AUAUGA | 2.40758 | 1.47E−08 |
| SEQ ID NO: 101 | 12h_hek293t_utr5 | repressed | CAGGAU | 2.349223 | 2.34E−08 |
| SEQ ID NO: 102 | 12h_hek293t_utr5 | repressed | AAUAUG | 2.499173 | 2.82E−08 |
| SEQ ID NO: 103 | 12h_hek293t_utr5 | repressed | GAUGCG | 2.763313 | 2.94E−08 |
| SEQ ID NO: 104 | 12h_hek293t_utr5 | repressed | GAUGAG | 2.236102 | 3.46E−08 |
| SEQ ID NO: 105 | 12h_hek293t_utr5 | repressed | GGGAU | 1.595125 | 3.96E−08 |
| SEQ ID NO: 106 | 12h_hek293t_utr5 | repressed | CCAUGU | 2.7191 | 4.54E−08 |
| SEQ ID NO: 107 | 12h_hek293t_utr5 | repressed | CAAUGG | 2.74909 | 5.33E−08 |
| SEQ ID NO: 108 | 12h_hek293t_utr5 | repressed | AUGAAU | 2.252776 | 6.72E−08 |
| SEQ ID NO: 109 | 12h_hek293t_utr5 | repressed | GCGAUG | 2.589071 | 7.31E−08 |
| SEQ ID NO: 110 | 12h_hek293t_utr5 | repressed | CAUGU | 1.609627 | 8.04E−08 |
| SEQ ID NO: 111 | 12h_hek293t_utr5 | repressed | CAUGAC | 2.856198 | 8.31E−08 |
| SEQ ID NO: 112 | 12h_hek293t_utr5 | repressed | AUGGGG | 2.977738 | 8.37E−08 |
| SEQ ID NO: 113 | 12h_hek293t_utr5 | repressed | AUGCG | 1.756039 | 1.15E−07 |
| SEQ ID NO: 114 | 12h_hek293t_utr5 | repressed | UAUGA | 1.708571 | 1.27E−07 |
| SEQ ID NO: 115 | 12h_hek293t_utr5 | repressed | AAUGGG | 2.574906 | 1.91E−07 |
| SEQ ID NO: 116 | 12h_hek293t_utr5 | repressed | GAUGUC | 2.535393 | 1.91E−07 |
| SEQ ID NO: 117 | 12h_hek293t_utr5 | repressed | AUGAAG | 2.04312 | 2E−07 |
| SEQ ID NO: 118 | 12h_hek293t_utr5 | repressed | UAUGGA | 2.377262 | 2.49E−07 |
| SEQ ID NO: 119 | 12h_hek293t_utr5 | repressed | AUGCC | 1.890745 | 2.96E−07 |
| SEQ ID NO: 120 | 12h_hek293t_utr5 | repressed | AAUGUG | 1.942931 | 3.01E−07 |
| SEQ ID NO: 121 | 12h_hek293t_utr5 | repressed | AGCCAU | 2.205153 | 3.23E−07 |
| SEQ ID NO: 122 | 12h_hek293t_utr5 | repressed | AUGAC | 1.691985 | 3.53E−07 |
| SEQ ID NO: 123 | 12h_hek293t_utr5 | repressed | GCCAU | 1.636678 | 3.56E−07 |
| SEQ ID NO: 124 | 12h_hek293t_utr5 | repressed | AUGAU | 1.612204 | 3.69E−07 |
| SEQ ID NO: 125 | 12h_hek293t_utr5 | repressed | AUGGGC | 2.841165 | 4.05E−07 |
| SEQ ID NO: 126 | 12h_hek293t_utr5 | repressed | UGGCG | 1.607572 | 4.26E−07 |
| SEQ ID NO: 127 | 12h_hek293t_utr5 | repressed | UGUGGC | 2.113838 | 5.03E−07 |
| SEQ ID NO: 128 | 12h_hek293t_utr5 | repressed | GAAUG | 1.541435 | 5.24E−07 |
| SEQ ID NO: 129 | 12h_hek293t_utr5 | repressed | GCAUGG | 2.399206 | 5.56E−07 |
| SEQ ID NO: 130 | 12h_hek293t_utr5 | repressed | AUGUCG | 2.429752 | 5.75E−07 |
| SEQ ID NO: 131 | 12h_hek293t_utr5 | repressed | CAUGC | 1.683281 | 5.85E−07 |
| SEQ ID NO: 132 | 12h_hek293t_utr5 | repressed | CAGGGG | 2.499173 | 6E−07 |
| SEQ ID NO: 133 | 12h_hek2931_utr5 | repressed | CAAUGC | 2.717548 | 6.78E−07 |
| SEQ ID NO: 134 | 12h_hek293t_utr5 | repressed | AUGCU | 1.565986 | 1.02E−06 |
| SEQ ID NO: 135 | 12h_hek293t_utr5 | repressed | GGACC | 1.677993 | 1.51E−06 |
| SEQ ID NO: 136 | 12h_hek293t_utr5 | repressed | AAUGUC | 2.145887 | 2.18E−06 |
| SEQ ID NO: 137 | 12h_hek293t_utr5 | repressed | GCAUG | 1.593967 | 2.2E−06 |
| SEQ ID NO: 138 | 12h_hek293t_utr5 | repressed | GAAGAU | 1.870349 | 2.5E−06 |
| SEQ ID NO: 139 | 12h_hek293t_utr5 | repressed | GAACGG | 2.31216 | 2.75E−06 |
| SEQ ID NO: 140 | 12h_hek293t_utr5 | repressed | AAUGAG | 1.991043 | 3E−06 |
| SEQ ID NO: 141 | 12h_hek293t_utr5 | repressed | CAGAUG | 1.975153 | 3.02E−06 |
| SEQ ID NO: 142 | 12h_hek293t_utr5 | repressed | UGGAGG | 1.90078 | 3.3E−06 |
| SEQ ID NO: 143 | 12h_hek293t_utr5 | repressed | AUAUG | 1.620467 | 3.41E−06 |
| SEQ ID NO: 144 | 12h_hek293t_utr5 | repressed | AUGUCA | 2.153983 | 3.45E−06 |
| SEQ ID NO: 145 | 12h_hek293t_utr5 | repressed | AGAUGU | 2.008069 | 3.74E−06 |
| SEQ ID NO: 146 | 12h_hek293t_utr5 | repressed | GACAUG | 2.242848 | 4.12E−06 |
| SEQ ID NO: 147 | 12h_hek293t_utr5 | repressed | AUGCUG | 1.81758 | 4.38E−06 |
| SEQ ID NO: 148 | 12h_hek293t_utr5 | repressed | AAGAU | 1.51878 | 4.5E−06 |
| SEQ ID NO: 149 | 12h_hek293t_utr5 | repressed | AUGUU | 1.441039 | 4.93E−06 |
| SEQ ID NO: 150 | 12h_hek293t_utr5 | repressed | GGCAA | 1.648234 | 5.21E−06 |
| SEQ ID NO: 151 | 12h_hek293t_utr5 | repressed | AUGACG | 2.347708 | 5.28E−06 |
| SEQ ID NO: 152 | 12h_hek293t_utr5 | repressed | GUGAUG | 2.093548 | 5.43E−06 |
| SEQ ID NO: 153 | 12h_hek293t_utr5 | repressed | CCAUGC | 2.73719 | 5.67E−06 |
| SEQ ID NO: 154 | 12h_hek293t_utr5 | repressed | AUGUGU | 1.774061 | 5.81E−06 |
| SEQ ID NO: 155 | 12h_hek293t_utr5 | repressed | UCCUGG | 2.107411 | 5.99E−06 |
| SEQ ID NO: 156 | 12h_hek293t_utr5 | repressed | AUGGUG | 2.330056 | 6.23E−06 |
| SEQ ID NO: 157 | 12h_hek293t_utr5 | repressed | UGAUGG | 2.05962 | 6.39E−06 |
| SEQ ID NO: 158 | 12h_hek293t_utr5 | repressed | AGAUGA | 1.999339 | 6.73E−06 |
| SEQ ID NO: 159 | 12h_hek293t_utr5 | repressed | GGAUGU | 1.981723 | 6.78E−06 |
| SEQ ID NO: 160 | 12h_hek293t_utr5 | repressed | UCAUGA | 2.096081 | 6.85E−06 |
| SEQ ID NO: 161 | 12h_hek293t_utr5 | repressed | CCUGGU | 2.244155 | 7.52E−06 |
| SEQ ID NO: 162 | 12h_hek293t_utr5 | repressed | AUGGGU | 2.836899 | 7.74E−06 |
| SEQ ID NO: 163 | 12h_hek293t_utr5 | repressed | AGGGG | 1.593373 | 8.5E−06 |
| SEQ ID NO: 164 | 12h_hek293t_utr5-2hpf_pa | active | UUUGUU | 1.850889 | 6.27E−18 |
| SEQ ID NO: 165 | 12h_hek293t_utr5-2hpf_pa | active | UUUUU | 1.499769 | 8.57E−18 |
| SEQ ID NO: 166 | 12h_hek293t_utr5-2hpf_pa | active | UUGUU | 1.542633 | 4.35E−17 |
| SEQ ID NO: 167 | 12h_hek293t_utr5-2hpf_pa | active | UUAUU | 1.526709 | 2.4E−15 |
| SEQ ID NO: 168 | 12h_hek293t_utr5-2hpf_pa | active | UUUGU | 1.468372 | 3.34E−15 |
| SEQ ID NO: 169 | 12h_hek293t_utr5-2hpf_pa | active | GUUUU | 1.418091 | 3.86E−15 |
| SEQ ID NO: 170 | 12h_hek293t_utr5-2hpf_pa | active | UUUCU | 1.492045 | 1.62E−14 |
| SEQ ID NO: 171 | 12h_hek293t_utr5-2hpf_pa | active | UUUUG | 1.438608 | 2.57E−14 |
| SEQ ID NO: 172 | 12h_hek293t_utr5-2hpf_pa | active | CUUUU | 1.453174 | 1.03E−13 |
| SEQ ID NO: 173 | 12h_hek293t_utr5-2hpf_pa | active | UUUUA | 1.410931 | 1.18E−13 |
| SEQ ID NO: 174 | 12h_hek293t_utr5-2hpf_pa | active | UUGUUU | 1.66178 | 4.61E−13 |
| SEQ ID NO: 175 | 12h_hek293t_utr5-2hpf_pa | active | UAUUU | 1.431185 | 1.17E−12 |
| SEQ ID NO: 176 | 12h_hek293t_utr5-2hpf_pa | active | UUUUUU | 1.729172 | 2.03E−12 |
| SEQ ID NO: 177 | 12h_hek293t_utr5-2hpf_pa | active | UGUUU | 1.361424 | 2.22E−12 |
| SEQ ID NO: 178 | 12h_hek293t_utr5-2hpf_pa | active | UUCUU | 1.55481 | 4.3E−12 |
| SEQ ID NO: 179 | 12h_hek293t_utr5-2hpf_pa | active | UUUAUU | 1.647317 | 1.61E−11 |
| SEQ ID NO: 180 | 12h_hek293t_utr5-2hpf_pa | active | UUGUGU | 1.800126 | 1.96E−10 |
| SEQ ID NO: 181 | 12h_hek293t_utr5-2hpf_pa | active | GUUUUU | 1.603589 | 7.16E−10 |
| SEQ ID NO: 182 | 12h_hek293t_utr5-2hpf_pa | active | UUUGC | 1.46567 | 1.19E−09 |
| SEQ ID NO: 183 | 12h_hek293t_utr5-2hpf_pa | active | UUUUC | 1.354186 | 1.73E−09 |
| SEQ ID NO: 184 | 12h_hek293t_utr5-2hpf_pa | active | CUUUUU | 1.634861 | 1.83E−09 |
| SEQ ID NO: 185 | 12h_hek293t_utr5-2hpf_pa | active | UAUUGU | 2.123012 | 4.74E−09 |
| SEQ ID NO: 186 | 12h_hek293t_utr5-2hpf_pa | active | UUUUCU | 1.60562 | 5.53E−09 |
| SEQ ID NO: 187 | 12h_hek293t_utr5-2hpf_pa | active | UUUAU | 1.356702 | 6.08E−09 |
| SEQ ID NO: 188 | 12h_hek293t_utr5-2hpf_pa | active | GUGUU | 1.374056 | 1.05E−08 |
| SEQ ID NO: 189 | 12h_hek293t_utr5-2hpf_pa | active | UUUUUA | 1.54831 | 1.12E−08 |
| SEQ ID NO: 190 | 12h_hek293t_utr5-2hpf_pa | active | UUAGU | 1.564012 | 1.37E−08 |
| SEQ ID NO: 191 | 12h_hek293t_utr5-2hpf_pa | active | UUUUAU | 1.591783 | 1.52E−08 |
| SEQ ID NO: 192 | 12h_hek293t_utr5-2hpf_pa | active | UUCUA | 1.719992 | 2.16E−08 |
| SEQ ID NO: 193 | 12h_hek293t_utr5-2hpf_pa | active | UUUCUA | 2.056851 | 2.21E−08 |
| SEQ ID NO: 194 | 12h_hek293t_utr5-2hpf_pa | active | UGUUUU | 1.448513 | 2.33E−08 |
| SEQ ID NO: 195 | 12h_hek293t_utr5-2hpf_pa | active | UUUUGU | 1.558003 | 2.4E−08 |
| SEQ ID NO: 196 | 12h_hek293t_utr5-2hpf_pa | active | UAGUGU | 2.159694 | 2.81E−08 |
| SEQ ID NO: 197 | 12h_hek293t_utr5-2hpf_pa | active | UUUUUG | 1.564064 | 5.13E−08 |
| SEQ ID NO: 198 | 12h_hek293t_utr5-2hpf_pa | active | UAGCU | 1.528314 | 1E−07 |
| SEQ ID NO: 199 | 12h_hek293t_utr5-2hpf_pa | active | UACUU | 1.591997 | 1.08E−07 |
| SEQ ID NO: 200 | 12h_hek293t_utr5-2hpf_pa | active | AUUUU | 1.305287 | 1.19E−07 |
| SEQ ID NO: 201 | 12h_hek2931_utr5-2hpf_pa | active | AUUGU | 1.478677 | 1.2E−07 |
| SEQ ID NO: 202 | 12h_hek293t_utr5-2hpf_pa | active | CUAGCU | 2.130769 | 1.76E−07 |
| SEQ ID NO: 203 | 12h_hek293t_utr5-2hpf_pa | active | GUUUG | 1.341397 | 1.78E−07 |
| SEQ ID NO: 204 | 12h_hek293t_utr5-2hpf_pa | active | AUAUUU | 1.597013 | 1.96E−07 |
| SEQ ID NO: 205 | 12h_hek293t_utr5-2hpf_pa | active | UUUUGC | 1.633036 | 3.54E−07 |
| SEQ ID NO: 206 | 12h_hek293t_utr5-2hpf_pa | active | UAUUUU | 1.482434 | 3.64E−07 |
| SEQ ID NO: 207 | 12h_hek293t_utr5-2hpf_pa | active | UACUUU | 1.852843 | 5.27E−07 |
| SEQ ID NO: 208 | 12h_hek293t_utr5-2hpf_pa | active | UUAUUU | 1.455832 | 6.06E−07 |
| SEQ ID NO: 209 | 12h_hek293t_utr5-2hpf_pa | active | UAGUG | 1.629199 | 8.2E−07 |
| SEQ ID NO: 210 | 12h_hek293t_utr5-2hpf_pa | active | UAUUG | 1.500205 | 9.47E−07 |
| SEQ ID NO: 211 | 12h_hek293t_utr5-2hpf_pa | active | ACUUU | 1.354634 | 1.09E−06 |
| SEQ ID NO: 212 | 12h_hek293t_utr5-2hpf_pa | active | UUCUUG | 1.885984 | 1.29E−06 |
| SEQ ID NO: 213 | 12h_hek293t_utr5-2hpf_pa | active | UUCUAC | 2.234622 | 1.34E−06 |
| SEQ ID NO: 214 | 12h_hek293t_utr5-2hpf_pa | active | UUUCUU | 1.54764 | 1.37E−06 |
| SEQ ID NO: 215 | 12h_hek2931_utr5-2hpf_pa | active | GAGUU | 1.422932 | 1.54E−06 |
| SEQ ID NO: 216 | 12h_hek293t_utr5-2hpf_pa | active | UUGUG | 1.350744 | 1.72E−06 |
| SEQ ID NO: 217 | 12h_hek293t_utr5-2hpf_pa | active | GUGUUU | 1.442627 | 1.74E−06 |
| SEQ ID NO: 218 | 12h_hek293t_utr5-2hpf_pa | active | GUUGU | 1.392316 | 2E−06 |
| SEQ ID NO: 219 | 12h_hek293t_utr5-2hpf_pa | active | GUUUGU | 1.533407 | 3.15E−06 |
| SEQ ID NO: 220 | 12h_hek293t_utr5-2hpf_pa | active | CUUCUU | 1.823118 | 3.99E−06 |
| SEQ ID NO: 221 | 12h_hek293t_utr5-2hpf_pa | active | GUCGCA | 2.257769 | 4.83E−06 |
| SEQ ID NO: 222 | 12h_hek293t_utr5-2hpf_pa | active | UGUUUG | 1.516492 | 4.85E−06 |
| SEQ ID NO: 223 | 12h_hek293t_utr5-2hpf_pa | active | AUUUCU | 1.618482 | 5.78E−06 |
| SEQ ID NO: 224 | 12h_hek293t_utr5-2hpf_pa | active | AUUUG | 1.331542 | 6.73E−06 |
| SEQ ID NO: 225 | 12h_hek293t_utr5-2hpf_pa | active | AGCUU | 1.402778 | 7.59E−06 |
| SEQ ID NO: 226 | 12h_hek293t_utr5-2hpf_pa | active | UUAUUG | 1.713181 | 8.67E−06 |
| SEQ ID NO: 227 | 12h_hek293t_utr5-2hpf_pa | active | UGUGU | 1.292487 | 8.7E−06 |
| SEQ ID NO: 228 | 12h_hek293t_utr5-2hpf_pa | active | AGUUU | 1.292487 | 8.7E−06 |
| SEQ ID NO: 230 | 12h_hek293t_utr5-2hpf_pa | repressed | CAUGG | 3.065508 | 2.41E−46 |
| SEQ ID NO: 231 | 12h_hek293t_utr5-2hpf_pa | repressed | GAUGG | 2.996856 | 7.5E−44 |
| SEQ ID NO: 232 | 12h_hek293t_utr5-2hpf_pa | repressed | GGAUG | 2.739032 | 1.02E−41 |
| SEQ ID NO: 233 | 12h_hek293t_utr5-2hpf_pa | repressed | AUGGA | 2.678103 | 2.55E−40 |
| SEQ ID NO: 234 | 12h_hek293t_utr5-2hpf_pa | repressed | AGGAUG | 4.79933 | 1.92E−37 |
| SEQ ID NO: 235 | 12h_hek293t_utr5-2hpf_pa | repressed | CCAUG | 3.018134 | 5.11E−33 |
| SEQ ID NO: 236 | 12h_hek293t_utr5-2hpf_pa | repressed | AUGGG | 2.846355 | 1.13E−28 |
| SEQ ID NO: 237 | 12h_hek293t_utr5-2hpf_pa | repressed | AGAUG | 2.374612 | 1.34E−27 |
| SEQ ID NO: 238 | 12h_hek293t_utr5-2hpf_pa | repressed | AUGGCG | 4.10657 | 5.49E−26 |
| SEQ ID NO: 239 | 12h_hek293t_utr5-2hpf_pa | repressed | GAUGGC | 4.325638 | 1.32E−24 |
| SEQ ID NO: 240 | 12h_hek293t_utr5-2hpf_pa | repressed | GAUGU | 2.178805 | 1.4E−24 |
| SEQ ID NO: 241 | 12h_hek293t_utr5-2hpf_pa | repressed | AAUGG | 2.3929 | 1.46E−23 |
| SEQ ID NO: 242 | 12h_hek293t_utr5-2hpf_pa | repressed | AUGGAG | 3.153406 | 1.2E−21 |
| SEQ ID NO: 243 | 12h_hek293t_utr5-2hpf_pa | repressed | AAAAUG | 3.124564 | 6.98E−21 |
| SEQ ID NO: 244 | 12h_hek293t_utr5-2hpf_pa | repressed | CAAUG | 2.355925 | 1.05E−20 |
| SEQ ID NO: 245 | 12h_hek293t_utr5-2hpf_pa | repressed | AUGUG | 1.941854 | 3.75E−20 |
| SEQ ID NO: 246 | 12h_hek293t_utr5-2hpf_pa | repressed | GGAUGC | 3.733656 | 5.14E−20 |
| SEQ ID NO: 247 | 12h_hek293t_utr5-2hpf_pa | repressed | AAAUG | 1.941514 | 2.02E−19 |
| SEQ ID NO: 248 | 12h_hek293t_utr5-2hpf_pa | repressed | AUCAUG | 3.502336 | 2.59E−19 |
| SEQ ID NO: 249 | 12h_hek293t_utr5-2hpf_pa | repressed | UCAUG | 2.023197 | 1.47E−18 |
| SEQ ID NO: 250 | 12h_hek293t_utr5-2hpf_pa | repressed | AAUGGC | 3.408615 | 4.35E−18 |
| SEQ ID NO: 251 | 12h_hek293t_utr5-2hpf_pa | repressed | GAUGA | 2.029602 | 6.95E−18 |
| SEQ ID NO: 252 | 12h_hek293t_utr5-2hpf_pa | repressed | GAUGC | 2.203722 | 1.72E−17 |
| SEQ ID NO: 253 | 12h_hek293t_utr5-2hpf_pa | repressed | CGAUG | 2.386154 | 2.26E−17 |
| SEQ ID NO: 254 | 12h_hek293t_utr5-2hpf_pa | repressed | GCCAUG | 3.758874 | 2.4E−17 |
| SEQ ID NO: 255 | 12h_hek293t_utr5-2hpf_pa | repressed | CAUGGG | 4.215098 | 2.69E−17 |
| SEQ ID NO: 256 | 12h_hek293t_utr5-2hpf_pa | repressed | ACCAUG | 4.443824 | 3.56E−17 |
| SEQ ID NO: 257 | 12h_hek293t_utr5-2hpf_pa | repressed | AAAUGG | 3.149561 | 5.57E−17 |
| SEQ ID NO: 258 | 12h_hek293t_utr5-2hpf_pa | repressed | AAGAUG | 3.241306 | 1.19E−16 |
| SEQ ID NO: 259 | 12h_hek293t_utr5-2hpf_pa | repressed | GUCAUG | 3.439011 | 1.25E−16 |
| SEQ ID NO: 260 | 12h_hek293t_utr5-2hpf_pa | repressed | ACAAUG | 3.59662 | 2E−16 |
| SEQ ID NO: 261 | 12h_hek293t_utr5-2hpf_pa | repressed | CAUGGC | 3.157454 | 2.64E−16 |
| SEQ ID NO: 262 | 12h_hek293t_utr5-2hpf_pa | repressed | CCAUGG | 3.663611 | 3.38E−16 |
| SEQ ID NO: 263 | 12h_hek293t_utr5-2hpf_pa | repressed | AUGAG | 1.978144 | 3.68E−16 |
| SEQ ID NO: 264 | 12h_hek293t_utr5-2hpf_pa | repressed | UCAUGG | 3.15464 | 4.86E−16 |
| SEQ ID NO: 265 | 12h_hek293t_utr5-2hpf_pa | repressed | CAUGGA | 2.900028 | 6.35E−16 |
| SEQ ID NO: 266 | 12h_hek293t_utr5-2hpf_pa | repressed | UGGAG | 1.710031 | 7.02E−16 |
| SEQ ID NO: 267 | 12h_hek293t_utr5-2hpf_pa | repressed | GAUGGA | 2.932283 | 6.97E−15 |
| SEQ ID NO: 268 | 12h_hek293t_utr5-2hpf_pa | repressed | GGGAUG | 3.229363 | 8.65E−15 |
| SEQ ID NO: 269 | 12h_hek293t_utr5-2hpf_pa | repressed | AUGGCU | 3.17321 | 1.09E−14 |
| SEQ ID NO: 270 | 12h_hek293t_utr5-2hpf_pa | repressed | UGAUG | 1.831115 | 1.62E−14 |
| SEQ ID NO: 271 | 12h_hek293t_utr5-2hpf_pa | repressed | AGGAU | 1.844491 | 3.53E−14 |
| SEQ ID NO: 272 | 12h_hek293t_utr5-2hpf_pa | repressed | AGAUGG | 3.080965 | 4.23E−14 |
| SEQ ID NO: 273 | 12h_hek293t_utr5-2hpf_pa | repressed | GGAUGG | 3.285256 | 1.44E−13 |
| SEQ ID NO: 274 | 12h_hek293t_utr5-2hpf_pa | repressed | AUGGCA | 3.022834 | 1.7E−13 |
| SEQ ID NO: 275 | 12h_hek293t_utr5-2hpf_pa | repressed | AACAUG | 2.918512 | 2.89E−13 |
| SEQ ID NO: 276 | 12h_hek293t_utr5-2hpf_pa | repressed | UGGCG | 1.909932 | 3.52E−13 |
| SEQ ID NO: 277 | 12h_hek293t_utr5-2hpf_pa | repressed | GAUGUG | 2.499651 | 7E−13 |
| SEQ ID NO: 278 | 12h_hek293t_utr5-2hpf_pa | repressed | AUGAGG | 2.826831 | 1.18E−12 |
| SEQ ID NO: 279 | 12h_hek293t_utr5-2hpf_pa | repressed | AAUGU | 1.711986 | 2.61E−12 |
| SEQ ID NO: 280 | 12h_hek293t_utr5-2hpf_pa | repressed | AUGAUG | 2.803842 | 2.69E−12 |
| SEQ ID NO: 281 | 12h_hek293t_utr5-2hpf_pa | repressed | AUGAA | 1.739189 | 6.4E−12 |
| SEQ ID NO: 282 | 12h_hek293t_utr5-2hpf_pa | repressed | AUGGAC | 2.908685 | 6.6E−12 |
| SEQ ID NO: 283 | 12h_hek293t_utr5-2hpf_pa | repressed | ACGAUG | 3.808992 | 7.89E−12 |
| SEQ ID NO: 284 | 12h_hek293t_utr5-2hpf_pa | repressed | AUGUGG | 2.600534 | 7.96E−12 |
| SEQ ID NO: 285 | 12h_hek293t_utr5-2hpf_pa | repressed | ACAUG | 1.851984 | 1.23E−11 |
| SEQ ID NO: 286 | 12h_hek293t_utr5-2hpf_pa | repressed | AUGGGA | 2.722133 | 1.46E−11 |
| SEQ ID NO: 287 | 12h_hek293t_utr5-2hpf_pa | repressed | AUGUC | 1.886122 | 1.96E−11 |
| SEQ ID NO: 288 | 12h_hek293t_utr5-2hpf_pa | repressed | ACAUGG | 2.92167 | 2.49E−11 |
| SEQ ID NO: 289 | 12h_hek293t_utr5-2hpf_pa | repressed | CAUGA | 1.862391 | 2.55E−11 |
| SEQ ID NO: 290 | 12h_hek293t_utr5-2hpf_pa | repressed | AAAUGA | 2.275324 | 3.73E−11 |
| SEQ ID NO: 291 | 12h_hek293t_utr5-2hpf_pa | repressed | AAUGA | 1.699122 | 7.85E−11 |
| SEQ ID NO: 292 | 12h_hek293t_utr5-2hpf_pa | repressed | GCGAUG | 2.949229 | 1.34E−10 |
| SEQ ID NO: 293 | 12h_hek293t_utr5-2hpf_pa | repressed | CCAUGU | 3.039576 | 2.36E−10 |
| SEQ ID NO: 294 | 12h_hek293t_utr5-2hpf_pa | repressed | GGGGC | 1.897516 | 3.08E−10 |
| SEQ ID NO: 295 | 12h_hek293t_utr5-2hpf_pa | repressed | GCAAUG | 2.919301 | 3.14E−10 |
| SEQ ID NO: 296 | 12h_hek293t_utr5-2hpf_pa | repressed | CAAUGU | 2.674045 | 3.98E−10 |
| SEQ ID NO: 297 | 12h_hek293t_utr5-2hpf_pa | repressed | GAUGCG | 3.00771 | 5.63E−10 |
| SEQ ID NO: 298 | 12h_hek293t_utr5-2hpf_pa | repressed | AGCCAU | 2.499651 | 6.28E−10 |
| SEQ ID NO: 299 | 12h_hek293t_utr5-2hpf_pa | repressed | GAAAUG | 2.464111 | 7.42E−10 |
| SEQ ID NO: 300 | 12h_hek293t_utr5-2hpf_pa | repressed | AUGAU | 1.757017 | 7.59E−10 |
| SEQ ID NO: 301 | 12h_hek293t_utr5-2hpf_pa | repressed | UGAUGG | 2.512212 | 7.62E−10 |
| SEQ ID NO: 302 | 12h_hek293t_utr5-2hpf_pa | repressed | AUGGAA | 2.613272 | 8.99E−10 |
| SEQ ID NO: 303 | 12h_hek293t_utr5-2hpf_pa | repressed | AGAAUG | 2.936098 | 1.19E−09 |
| SEQ ID NO: 304 | 12h_hek293t_utr5-2hpf_pa | repressed | UGUGGC | 2.378523 | 1.4E−09 |
| SEQ ID NO: 305 | 12h_hek293t_utr5-2hpf_pa | repressed | CGAUGG | 2.991036 | 1.91E−09 |
| SEQ ID NO: 306 | 12h_hek293t_utr5-2hpf_pa | repressed | AGAUGU | 2.35775 | 1.96E−09 |
| SEQ ID NO: 307 | 12h_hek293t_utr5-2hpf_pa | repressed | UAUGG | 1.869113 | 2.24E−09 |
| SEQ ID NO: 308 | 12h_hek293t_utr5-2hpf_pa | repressed | CAUGU | 1.687108 | 2.28E−09 |
| SEQ ID NO: 309 | 12h_hek293t_utr5-2hpf_pa | repressed | AGGGG | 1.791189 | 8.66E−09 |
| SEQ ID NO: 310 | 12h_hek293t_utr5-2hpf_pa | repressed | GGAUGA | 2.284164 | 8.82E−09 |
| SEQ ID NO: 311 | 12h_hek293t_utr5-2hpf_pa | repressed | GAGAUG | 2.594575 | 8.9E−09 |
| SEQ ID NO: 312 | 12h_hek293t_utr5-2hpf_pa | repressed | AGCAUG | 2.562142 | 1.32E−08 |
| SEQ ID NO: 313 | 12h_hek293t_utr5-2hpf_pa | repressed | GCAUGG | 2.599637 | 1.9E−08 |
| SEQ ID NO: 314 | 12h_hek293t_utr5-2hpf_pa | repressed | AUGGGG | 3.084676 | 2.08E−08 |
| SEQ ID NO: 315 | 12h_hek293t_utr5-2hpf_pa | repressed | UGGAGG | 2.112381 | 2.16E−08 |
| SEQ ID NO: 316 | 12h_hek293t_utr5-2hpf_pa | repressed | AUGGGC | 3.052206 | 2.7E−08 |
| SEQ ID NO: 317 | 12h_hek293t_utr5-2hpf_pa | repressed | AGAUGA | 2.27241 | 2.71E−08 |
| SEQ ID NO: 318 | 12h_hek293t_utr5-2hpf_pa | repressed | UAUGGA | 2.499651 | 2.8E−08 |
| SEQ ID NO: 319 | 12h_hek293t_utr5-2hpf_pa | repressed | GCCAU | 1.699276 | 3.09E−08 |
| SEQ ID NO: 320 | 12h_hek293t_utr5-2hpf_pa | repressed | AUGAAG | 2.116493 | 3.4E−08 |
| SEQ ID NO: 321 | 12h_hek293t_utr5-2hpf_pa | repressed | GAUGAG | 2.23653 | 3.44E−08 |
| SEQ ID NO: 322 | 12h_hek293t_utr5-2hpf_pa | repressed | GAAGAU | 2.031975 | 4.5E−08 |
| SEQ ID NO: 323 | 12h_hek293t_utr5-2hpf_pa | repressed | GAUGUC | 2.608332 | 5.96E−08 |
| SEQ ID NO: 324 | 12h_hek293t_utr5-2hpf_pa | repressed | AUGGCC | 2.792403 | 9.02E−08 |
| SEQ ID NO: 325 | 12h_hek293t_utr5-2hpf_pa | repressed | CAGAUG | 2.136799 | 9.26E−08 |
| SEQ ID NO: 326 | 12h_hek293t_utr5-2hpf_pa | repressed | UGGCGG | 2.187195 | 1.3E−07 |
| SEQ ID NO: 327 | 12h_hek293t_utr5-2hpf_pa | repressed | AUGUGU | 1.915226 | 1.48E−07 |
| SEQ ID NO: 328 | 12h_hek293t_utr5-2hpf_pa | repressed | CAGGAU | 2.249686 | 1.76E−07 |
| SEQ ID NO: 329 | 12h_hek293t_utr5-2hpf_pa | repressed | GGAUGU | 2.158289 | 2E−07 |
| SEQ ID NO: 330 | 12h_hek293t_utr5-2hpf_pa | repressed | UGAUGU | 1.961752 | 2.12E−07 |
| SEQ ID NO: 331 | 12h_hek293t_utr5-2hpf_pa | repressed | GCAUG | 1.657911 | 2.15E−07 |
| SEQ ID NO: 332 | 12h_hek293t_utr5-2hpf_pa | repressed | CGAUGA | 2.556034 | 2.31E−07 |
| SEQ ID NO: 333 | 12h_hek293t_utr5-2hpf_pa | repressed | AAUAUG | 2.377717 | 2.48E−07 |
| SEQ ID NO: 334 | 12h_hek293t_utr5-2hpf_pa | repressed | GGGGCG | 2.465409 | 2.73E−07 |
| SEQ ID NO: 335 | 12h_hek293t_utr5-2hpf_pa | repressed | AUGCC | 1.891678 | 2.91E−07 |
| SEQ ID NO: 336 | 12h_hek293t_utr5-2hpf_pa | repressed | CAUGAC | 2.761519 | 2.99E−07 |
| SEQ ID NO: 337 | 12h_hek293t_utr5-2hpf_pa | repressed | AAUGUG | 1.943302 | 2.99E−07 |
| SEQ ID NO: 338 | 12h_hek293t_utr5-2hpf_pa | repressed | GUGAUG | 2.250995 | 3.2E−07 |
| SEQ ID NO: 339 | 12h_hek293t_utr5-2hpf_pa | repressed | GGGAU | 1.549316 | 3.26E−07 |
| SEQ ID NO: 340 | 12h_hek293t_utr5-2hpf_pa | repressed | GAACGG | 2.448638 | 3.27E−07 |
| SEQ ID NO: 341 | 12h_hek293t_utr5-2hpf_pa | repressed | AUGAC | 1.692819 | 3.45E−07 |
| SEQ ID NO: 342 | 12h_hek293t_utr5-2hpf_pa | repressed | UGGCA | 1.657435 | 4.7E−07 |
| SEQ ID NO: 343 | 12h_hek293t_utr5-2hpf_pa | repressed | CCAUGA | 2.654497 | 4.8E−07 |
| SEQ ID NO: 344 | 12h_hek293t_utr5-2hpf_pa | repressed | GAUGGG | 2.604679 | 4.92E−07 |
| SEQ ID NO: 345 | 12h_hek293t_utr5-2hpf_pa | repressed | CAUGC | 1.684111 | 5.73E−07 |
| SEQ ID NO: 346 | 12h_hek293t_utr5-2hpf_pa | repressed | AUGACG | 2.499651 | 5.98E−07 |
| SEQ ID NO: 347 | 12h_hek293t_utr5-2hpf_pa | repressed | GAAGGG | 2.240125 | 6.78E−07 |
| SEQ ID NO: 348 | 12h_hek293t_utr5-2hpf_pa | repressed | CAGGG | 1.638497 | 7.07E−07 |
| SEQ ID NO: 349 | 12h_hek293t_utr5-2hpf_pa | repressed | AUGCG | 1.698341 | 7.93E−07 |
| SEQ ID NO: 350 | 12h_hek293t_utr5-2hpf_pa | repressed | UCCAUG | 2.352613 | 9.2E−07 |
| SEQ ID NO: 351 | 12h_hek293t_utr5-2hpf_pa | repressed | AAGAU | 1.558827 | 9.48E−07 |
| SEQ ID NO: 352 | 12h_hek293t_utr5-2hpf_pa | repressed | GAUGCU | 2.187195 | 9.78E−07 |
| SEQ ID NO: 353 | 12h_hek293t_utr5-2hpf_pa | repressed | AAUGGA | 2.241067 | 1.23E−06 |
| SEQ ID NO: 354 | 12h_hek293t_utr5-2hpf_pa | repressed | AAUGAG | 2.032911 | 1.28E−06 |
| SEQ ID NO: 355 | 12h_hek293t_utr5-2hpf_pa | repressed | AUGUCA | 2.209636 | 1.33E−06 |
| SEQ ID NO: 356 | 12h_hek293t_utr5-2hpf_pa | repressed | GGACC | 1.67882 | 1.48E−06 |
| SEQ ID NO: 357 | 12h_hek293t_utr5-2hpf_pa | repressed | UUAUGG | 2.248207 | 1.54E−06 |
| SEQ ID NO: 358 | 12h_hek293t_utr5-2hpf_pa | repressed | GAUGAU | 2.073443 | 1.94E−06 |
| SEQ ID NO: 359 | 12h_hek293t_utr5-2hpf_pa | repressed | GGAGG | 1.46459 | 2.04E−06 |
| SEQ ID NO: 360 | 12h_hek293t_utr5-2hpf_pa | repressed | AUAUGA | 2.146297 | 2.17E−06 |
| SEQ ID NO: 361 | 12h_hek293t_utr5-2hpf_pa | repressed | GAGGAU | 1.823275 | 2.77E−06 |
| SEQ ID NO: 362 | 12h_hek293t_utr5-2hpf_pa | repressed | UGGAGA | 1.831576 | 2.85E−06 |
| SEQ ID NO: 363 | 12h_hek293t_utr5-2hpf_pa | repressed | GCGCUG | 1.759958 | 2.9E−06 |
| SEQ ID NO: 364 | 12h_hek293t_utr5-2hpf_pa | repressed | GGCAUG | 2.687797 | 3.26E−06 |
| SEQ ID NO: 365 | 12h_hek293t_utr5-2hpf_pa | repressed | GAAUG | 1.495105 | 3.69E−06 |
| SEQ ID NO: 366 | 12h_hek293t_utr5-2hpf_pa | repressed | CGCUGU | 1.727253 | 4.17E−06 |
| SEQ ID NO: 367 | 12h_hek293t_utr5-2hpf_pa | repressed | CGCUG | 1.381875 | 4.26E−06 |
| SEQ ID NO: 368 | 12h_hek293t_utr5-2hpf_pa | repressed | AUGUA | 1.569951 | 4.31E−06 |
| SEQ ID NO: 369 | 12h_hek293t_utr5-2hpf_pa | repressed | UGGCCG | 2.13085 | 4.54E−06 |
| SEQ ID NO: 370 | 12h_hek293t_utr5-2hpf_pa | repressed | UAUGA | 1.604755 | 4.54E−06 |
| SEQ ID NO: 371 | 12h_hek293t_utr5-2hpf_pa | repressed | GGAGC | 1.413151 | 4.68E−06 |
| SEQ ID NO: 372 | 12h_hek293t_utr5-2hpf_pa | repressed | CAGGGG | 2.348157 | 5.26E−06 |
| SEQ ID NO: 373 | 12h_hek293t_utr5-2hpf_pa | repressed | UCAGGG | 2.214881 | 5.54E−06 |
| SEQ ID NO: 374 | 12h_hek293t_utr5-2hpf_pa | repressed | GAUGUU | 1.851593 | 6.07E−06 |
| SEQ ID NO: 375 | 12h_hek293t_utr5-2hpf_pa | repressed | CGUCAU | 2.122609 | 6.56E−06 |
| SEQ ID NO: 376 | 12h_hek293t_utr5-2hpf_pa | repressed | GGAGCG | 1.849057 | 7.63E−06 |
| SEQ ID NO: 377 | 12h_hek293t_utr5-2hpf_pa | repressed | AUGGGU | 2.837442 | 7.72E−06 |
| SEQ ID NO: 378 | 12h_hek293t_utr5-2hpf_pa | repressed | AUGCU | 1.512732 | 7.75E−06 |
| SEQ ID NO: 379 | 12h_hek293t_utr5-2hpf_pa | repressed | UGGCAG | 1.999721 | 8.45E−06 |
| SEQ ID NO: 380 | 12h_hek293t_utr5-2hpf_pa | repressed | GGCGG | 1.486221 | 8.8E−06 |
| SEQ ID NO: 381 | 12h_hek293t_utr5-2hpf_pa | s1_activated | GCGGC | 2.325827 | 1.71E−31 |
| SEQ ID NO: 382 | 12h_hek293t_utr5-2hpf_pa | s1_activated | CCGGG | 2.632329 | 5.13E−28 |
| SEQ ID NO: 383 | 12h_hek293t_utr5-2hpf_pa | s1_activated | CCGCC | 2.425927 | 1.54E−25 |
| SEQ ID NO: 384 | 12h_hek293t_utr5-2hpf_pa | s1_activated | CUCUC | 1.975956 | 3.01E−24 |
| SEQ ID NO: 385 | 12h_hek293t_utr5-2hpf_pa | s1_activated | GCCGG | 2.432323 | 3.93E−24 |
| SEQ ID NO: 386 | 12h_hek293t_utr5-2hpf_pa | s1_activated | GCCGC | 2.144753 | 5.5E−22 |
| SEQ ID NO: 387 | 12h_hek2931_utr5-2hpf_pa | s1_activated | CUCCG | 2.126101 | 5.71E−20 |
| SEQ ID NO: 388 | 12h_hek293t_utr5-2hpf_pa | s1_activated | AGCGG | 1.906847 | 2.61E−19 |
| SEQ ID NO: 389 | 12h_hek293t_utr5-2hpf_pa | s1_activated | CGCGC | 1.957115 | 4.6E−19 |
| SEQ ID NO: 390 | 12h_hek293t_utr5-2hpf_pa | s1_activated | GCCCC | 2.589131 | 4.64E−19 |
| SEQ ID NO: 391 | 12h_hek293t_utr5-2hpf_pa | s1_activated | CCCGG | 2.287301 | 1.4E−18 |
| SEQ ID NO: 392 | 12h_hek293t_utr5-2hpf_pa | s1_activated | CGGCC | 2.32297 | 1.29E−17 |
| SEQ ID NO: 393 | 12h_hek293t_utr5-2hpf_pa | s1_activated | CCCCC | 2.428921 | 1.76E−17 |
| SEQ ID NO: 394 | 12h_hek293t_utr5-2hpf_pa | s1_activated | GCGCG | 1.864566 | 1.77E−17 |
| SEQ ID NO: 395 | 12h_hek293t_utr5-2hpf_pa | s1_activated | CGGGG | 2.259836 | 2.4E−17 |
| SEQ ID NO: 396 | 12h_hek293t_utr5-2hpf_pa | s1_activated | CGGAG | 1.850301 | 2.79E−17 |
| SEQ ID NO: 397 | 12h_hek293t_utr5-2hpf_pa | s1_activated | GCCGCC | 3.404498 | 5.95E−17 |
| SEQ ID NO: 398 | 12h_hek293t_utr5-2hpf_pa | s1_activated | CCGCG | 2.019867 | 7.21E−17 |
| SEQ ID NO: 399 | 12h_hek293t_utr5-2hpf_pa | s1_activated | CCGGC | 2.232406 | 8.52E−17 |
| SEQ ID NO: 400 | 12h_hek293t_utr5-2hpf_pa | s1_activated | CGGCG | 1.947051 | 1.52E−16 |
| SEQ ID NO: 401 | 12h_hek293t_utr5-2hpf_pa | s1_activated | CCCCG | 2.275687 | 1.61E−16 |
| SEQ ID NO: 402 | 12h_hek293t_utr5-2hpf_pa | s1_activated | CUCUCC | 2.551774 | 2.17E−16 |
| SEQ ID NO: 403 | 12h_hek293t_utr5-2hpf_pa | s1_activated | GGAGG | 1.830131 | 1.05E−15 |
| SEQ ID NO: 404 | 12h_hek293t_utr5-2hpf_pa | s1_activated | CGGUG | 1.911795 | 1.24E−15 |
| SEQ ID NO: 405 | 12h_hek293t_utr5-2hpf_pa | s1_activated | GGCGG | 1.929496 | 1.83E−15 |
| SEQ ID NO: 406 | 12h_hek293t_utr5-2hpf_pa | s1_activated | GGUGG | 2.042959 | 1.87E−15 |
| SEQ ID NO: 407 | 12h_hek293t_utr5-2hpf_pa | s1_activated | GCUCG | 1.813967 | 2.49E−15 |
| SEQ ID NO: 408 | 12h_hek2931_utr5-2hpf_pa | s1_activated | CCCUC | 1.940969 | 4.63E−15 |
| SEQ ID NO: 409 | 12h_hek293t_utr5-2hpf_pa | s1_activated | GGGAGG | 3.07405 | 5.47E−15 |
| SEQ ID NO: 410 | 12h_hek293t_utr5-2hpf_pa | s1_activated | CGCGG | 1.849673 | 7.94E−15 |
| SEQ ID NO: 411 | 12h_hek293t_utr5-2hpf_pa | s1_activated | CGCCG | 1.923393 | 1E−14 |
| SEQ ID NO: 412 | 12h_hek293t_utr5-2hpf_pa | s1_activated | CCUCC | 1.84266 | 1.34E−14 |
| SEQ ID NO: 413 | 12h_hek293t_utr5-2hpf_pa | s1_activated | GAGGC | 1.929331 | 1.43E−14 |
| SEQ ID NO: 414 | 12h_hek293t_utr5-2hpf_pa | s1_activated | AGCCC | 2.052553 | 1.43E−14 |
| SEQ ID NO: 415 | 12h_hek293t_utr5-2hpf_pa | s1_activated | GAGAGG | 2.406307 | 3.62E−14 |
| SEQ ID NO: 416 | 12h_hek293t_utr5-2hpf_pa | s1_activated | CGCUC | 1.784123 | 4.5E−14 |
| SEQ ID NO: 417 | 12h_hek293t_utr5-2hpf_pa | s1_activated | CUCCC | 1.939684 | 9.02E−14 |
| SEQ ID NO: 418 | 12h_hek293t_utr5-2hpf_pa | s1_activated | GCGGCG | 2.525314 | 1.33E−13 |
| SEQ ID NO: 419 | 12h_hek293t_utr5-2hpf_pa | s1_activated | CGGGC | 2.045043 | 1.63E−13 |
| SEQ ID NO: 420 | 12h_hek293t_utr5-2hpf_pa | s1_activated | GGGAG | 1.872294 | 1.64E−13 |
| SEQ ID NO: 421 | 12h_hek293t_utr5-2hpf_pa | s1_activated | AGCGGC | 2.458605 | 1.84E−13 |
| SEQ ID NO: 422 | 12h_hek2931_utr5-2hpf_pa | s1_activated | GCGGAG | 2.348324 | 2.92E−13 |
| SEQ ID NO: 423 | 12h_hek293t_utr5-2hpf_pa | s1_activated | GGUGC | 2.05398 | 3.17E−13 |
| SEQ ID NO: 424 | 12h_hek293t_utr5-2hpf_pa | s1_activated | GCGGG | 1.879367 | 3.8E−13 |
| SEQ ID NO: 425 | 12h_hek293t_utr5-2hpf_pa | s1_activated | GGAGC | 1.687217 | 4.06E−13 |
| SEQ ID NO: 426 | 12h_hek293t_utr5-2hpf_pa | s1_activated | GGCCG | 2.002201 | 5.57E−13 |
| SEQ ID NO: 427 | 12h_hek293t_utr5-2hpf_pa | s1_activated | GCGCGC | 2.417922 | 6.45E−13 |
| SEQ ID NO: 428 | 12h_hek293t_utr5-2hpf_pa | s1_activated | CGCCGC | 2.646506 | 6.91E−13 |
| SEQ ID NO: 429 | 12h_hek293t_utr5-2hpf_pa | s1_activated | UCCGC | 1.806124 | 7.07E−13 |
| SEQ ID NO: 430 | 12h_hek293t_utr5-2hpf_pa | s1_activated | CUCGG | 1.860838 | 9.76E−13 |
| SEQ ID NO: 431 | 12h_hek293t_utr5-2hpf_pa | s1_activated | GGCGC | 1.991694 | 9.99E−13 |
| SEQ ID NO: 432 | 12h_hek293t_utr5-2hpf_pa | s1_activated | CCCGC | 2.039665 | 1.04E−12 |
| SEQ ID NO: 433 | 12h_hek293t_utr5-2hpf_pa | s1_activated | GGCAG | 1.775437 | 1.24E−12 |
| SEQ ID NO: 434 | 12h_hek293t_utr5-2hpf_pa | s1_activated | CCCCUC | 2.693539 | 1.68E−12 |
| SEQ ID NO: 435 | 12h_hek293t_utr5-2hpf_pa | s1_activated | AGCCG | 1.774801 | 1.76E−12 |
| SEQ ID NO: 436 | 12h_hek293t_utr5-2hpf_pa | s1_activated | CCGCCG | 2.681126 | 2.07E−12 |
| SEQ ID NO: 437 | 12h_hek293t_utr5-2hpf_pa | s1_activated | CACCG | 1.883593 | 2.17E−12 |
| SEQ ID NO: 438 | 12h_hek293t_utr5-2hpf_pa | s1_activated | UCUCC | 1.65871 | 2.23E−12 |
| SEQ ID NO: 439 | 12h_hek293t_utr5-2hpf_pa | s1_activated | GCCGGG | 3.113103 | 2.25E−12 |
| SEQ ID NO: 440 | 12h_hek293t_utr5-2hpf_pa | s1_activated | AGAGG | 1.717836 | 2.65E−12 |
| SEQ ID NO: 441 | 12h_hek293t_utr5-2hpf_pa | s1_activated | GAGGG | 1.786998 | 3.75E−12 |
| SEQ ID NO: 442 | 12h_hek293t_utr5-2hpf_pa | s1_activated | GCCCG | 2.024893 | 4.24E−12 |
| SEQ ID NO: 443 | 12h_hek2931_utr5-2hpf_pa | s1_activated | CCGGU | 1.929987 | 4.37E−12 |
| SEQ ID NO: 444 | 12h_hek293t_utr5-2hpf_pa | s1_activated | GGCCGG | 3.23839 | 5.02E−12 |
| SEQ ID NO: 445 | 12h_hek293t_utr5-2hpf_pa | s1_activated | CGGAC | 1.831484 | 6.5E−12 |
| SEQ ID NO: 446 | 12h_hek293t_utr5-2hpf_pa | s1_activated | GCGGA | 1.674305 | 1E−11 |
| SEQ ID NO: 447 | 12h_hek293t_utr5-2hpf_pa | s1_activated | CCUCCG | 2.765333 | 1.22E−11 |
| SEQ ID NO: 448 | 12h_hek293t_utr5-2hpf_pa | s1_activated | CGGGGG | 3.260111 | 1.3E−11 |
| SEQ ID NO: 449 | 12h_hek293t_utr5-2hpf_pa | s1_activated | CCUCGG | 2.824853 | 1.31E−11 |
| SEQ ID NO: 450 | 12h_hek293t_utr5-2hpf_pa | s1_activated | GCUCC | 1.761996 | 2.05E−11 |
| SEQ ID NO: 451 | 12h_hek293t_utr5-2hpf_pa | s1_activated | CCGCU | 1.712481 | 2.52E−11 |
| SEQ ID NO: 452 | 12h_hek293t_utr5-2hpf_pa | s1_activated | UCCGCC | 2.529584 | 2.69E−11 |
| SEQ ID NO: 453 | 12h_hek293t_utr5-2hpf_pa | s1_activated | CGGCU | 1.708996 | 3.39E−11 |
| SEQ ID NO: 454 | 12h_hek293t_utr5-2hpf_pa | s1_activated | GGGUG | 1.877417 | 4.78E−11 |
| SEQ ID NO: 455 | 12h_hek293t_utr5-2hpf_pa | s1_activated | GCUCGA | 2.554218 | 5.77E−11 |
| SEQ ID NO: 456 | 12h_hek293t_utr5-2hpf_pa | s1_activated | CGGGA | 1.767442 | 5.79E−11 |
| SEQ ID NO: 457 | 12h_hek2931_utr5-2hpf_pa | s1_activated | CUCCGC | 2.496827 | 6.87E−11 |
| SEQ ID NO: 458 | 12h_hek293t_utr5-2hpf_pa | s1_activated | CCCAG | 1.837841 | 8.83E−11 |
| SEQ ID NO: 459 | 12h_hek293t_utr5-2hpf_pa | s1_activated | CAGCGG | 2.157529 | 8.86E−11 |
| SEQ ID NO: 460 | 12h_hek293t_utr5-2hpf_pa | s1_activated | CCGGGG | 3.08042 | 8.97E−11 |
| SEQ ID NO: 461 | 12h_hek293t_utr5-2hpf_pa | s1_activated | CGCGGC | 2.543098 | 1.02E−10 |
| SEQ ID NO: 462 | 12h_hek293t_utr5-2hpf_pa | s1_activated | GCGCUC | 2.218719 | 1.26E−10 |
| SEQ ID NO: 463 | 12h_hek293t_utr5-2hpf_pa | s1_activated | CCACC | 1.906507 | 1.31E−10 |
| SEQ ID NO: 464 | 12h_hek293t_utr5-2hpf_pa | s1_activated | CCCGGG | 3.145348 | 1.37E−10 |
| SEQ ID NO: 465 | 12h_hek293t_utr5-2hpf_pa | s1_activated | CCGCGG | 2.434328 | 1.38E−10 |
| SEQ ID NO: 466 | 12h_hek293t_utr5-2hpf_pa | s1_activated | CAGCG | 1.578049 | 1.91E−10 |
| SEQ ID NO: 467 | 12h_hek293t_utr5-2hpf_pa | s1_activated | CGGCCU | 3.009333 | 1.95E−10 |
| SEQ ID NO: 468 | 12h_hek293t_utr5-2hpf_pa | s1__activated | GAGCG | 1.60661 | 2.54E−10 |
| SEQ ID NO: 469 | 12h_hek293t_utr5-2hpf_pa | s1_activated | GAGAG | 1.557373 | 2.56E−10 |
| SEQ ID NO: 470 | 12h_hek293t_utr5-2hpf_pa | s1_activated | CCCCU | 1.804273 | 2.82E−10 |
| SEQ ID NO: 471 | 12h_hek293t_utr5-2hpf_pa | s1_activated | CCCCCU | 2.452049 | 3.03E−10 |
| SEQ ID NO: 472 | 12h_hek293t_utr5-2hpf_pa | s1_activated | GGCGCG | 2.519892 | 3.18E−10 |
| SEQ ID NO: 473 | 12h_hek293t_utr5-2hpf_pa | s1_activated | GCGCU | 1.580338 | 4.37E−10 |
| SEQ ID NO: 474 | 12h_hek293t_utr5-2hpf_pa | s1_activated | ACCGG | 1.83724 | 4.51E−10 |
| SEQ ID NO: 475 | 12h_hek293t_utr5-2hpf_pa | s1_activated | GGGGG | 1.961224 | 4.96E−10 |
| SEQ ID NO: 476 | 12h_hek293t_utr5-2hpf_pa | s1_activated | CGGCAG | 2.339271 | 5.1E−10 |
| SEQ ID NO: 477 | 12h_hek293t_utr5-2hpf_pa | s1_activated | CGGUGC | 2.747374 | 5.86E−10 |
| SEQ ID NO: 478 | 12h_hek2931_utr5-2hpf_pa | s1_activated | GCCCCG | 2.993154 | 6.62E−10 |
| SEQ ID NO: 479 | 12h_hek293t_utr5-2hpf_pa | s1_activated | UCCCCC | 2.604763 | 6.76E−10 |
| SEQ ID NO: 480 | 12h_hek293t_utr5-2hpf_pa | s1_activated | GGCCU | 1.801871 | 7.12E−10 |
| SEQ ID NO: 481 | 12h_hek293t_utr5-2hpf_pa | s1_activated | CGCGCU | 2.110291 | 7.5E−10 |
| SEQ ID NO: 482 | 12h_hek293t_utr5-2hpf_pa | s1_activated | CGGCGC | 2.621767 | 8.1E−10 |
| SEQ ID NO: 483 | 12h_hek293t_utr5-2hpf_pa | s1_activated | GCUGG | 1.543685 | 1.05E−09 |
| SEQ ID NO: 484 | 12h_hek293t_utr5-2hpf_pa | s1_activated | GGGGC | 1.86943 | 1.06E−09 |
| SEQ ID NO: 485 | 12h_hek293t_utr5-2hpf_pa | s1_activated | CCGGCU | 2.805905 | 1.16E−09 |
| SEQ ID NO: 486 | 12h_hek293t_utr5-2hpf_pa | s1_activated | GUGCC | 1.871656 | 1.2E−09 |
| SEQ ID NO: 487 | 12h_hek293t_utr5-2hpf_pa | s1_activated | GGCGGC | 2.285079 | 1.33E−09 |
| SEQ ID NO: 488 | 12h_hek293t_utr5-2hpf_pa | s1_activated | CCUCU | 1.599963 | 1.48E−09 |
| SEQ ID NO: 489 | 12h_hek293t_utr5-2hpf_pa | s1_activated | GGCUGG | 2.314872 | 1.5E−09 |
| SEQ ID NO: 490 | 12h_hek293t_utr5-2hpf_pa | s1_activated | CGCGCG | 2.180676 | 1.54E−09 |
| SEQ ID NO: 491 | 12h_hek293t_utr5-2hpf_pa | s1_activated | CGCCGG | 2.521188 | 2.14E−09 |
| SEQ ID NO: 492 | 12h_hek2931_utr5-2hpf_pa | s1_activated | AGCGC | 1.584186 | 2.45E−09 |
| SEQ ID NO: 493 | 12h_hek293t_utr5-2hpf_pa | s1_activated | GGGGUG | 2.729554 | 2.8E−09 |
| SEQ ID NO: 494 | 12h_hek293t_utr5-2hpf_pa | s1_activated | GGCUC | 1.698828 | 2.97E−09 |
| SEQ ID NO: 495 | 12h_hek293t_utr5-2hpf_pa | s1_activated | CGGGAG | 2.521481 | 3.14E−09 |
| SEQ ID NO: 496 | 12h_hek293t_utr5-2hpf_pa | s1_activated | ACCGCG | 2.550282 | 3.15E−09 |
| SEQ ID NO: 497 | 12h_hek293t_utr5-2hpf_pa | s1_activated | GGAGCG | 2.164246 | 3.7E−09 |
| SEQ ID NO: 498 | 12h_hek293t_utr5-2hpf_pa | s1_activated | GCCCGG | 2.633166 | 3.71E−09 |
| SEQ ID NO: 499 | 12h_hek293t_utr5-2hpf_pa | s1_activated | AGCCCG | 2.48138 | 3.72E−09 |
| SEQ ID NO: 500 | 12h_hek293t_utr5-2hpf_pa | s1_activated | CAGCGC | 2.123851 | 4.67E−09 |
| SEQ ID NO: 501 | 12h_hek293t_utr5-2hpf_pa | s1_activated | GCGGCU | 2.073299 | 5.9E−09 |
| SEQ ID NO: 502 | 12h_hek293t_utr5-2hpf_pa | s1_activated | CCAGC | 1.603509 | 6.06E−09 |
| SEQ ID NO: 503 | 12h_hek293t_utr5-2hpf_pa | s1_activated | GGGCU | 1.713017 | 6.42E−09 |
| SEQ ID NO: 504 | 12h_hek293t_utr5-2hpf_pa | s1_activated | AGCGCG | 2.023081 | 6.42E−09 |
| SEQ ID NO: 505 | 12h_hek293t_utr5-2hpf_pa | s1_activated | GCGGCC | 2.522108 | 6.74E−09 |
| SEQ ID NO: 506 | 12h_hek293t_utr5-2hpf_pa | s1_activated | GCUCUC | 2.060772 | 7.63E−09 |
| SEQ ID NO: 507 | 12h_hek293t_utr5-2hpf_pa | s1_activated | CACCGG | 2.661315 | 9.5E−09 |
| SEQ ID NO: 508 | 12h_hek293t_utr5-2hpf_pa | s1_activated | GGCUG | 1.521843 | 9.56E−09 |
| SEQ ID NO: 509 | 12h_hek293t_utr5-2hpf_pa | s1_activated | UGCCCC | 2.903742 | 1.24E−08 |
| SEQ ID NO: 510 | 12h_hek293t_utr5-2hpf_pa | s1_activated | CUCCCC | 2.322016 | 1.3E−08 |
| SEQ ID NO: 511 | 12h_hek293t_utr5-2hpf_pa | s1_activated | UCUCUC | 1.949781 | 1.33E−08 |
| SEQ ID NO: 512 | 12h_hek293t_utr5-2hpf_pa | s1_activated | GGUGCC | 3.070748 | 1.36E−08 |
| SEQ ID NO: 513 | 12h_hek293t_utr5-2hpf_pa | s1_activated | CCGCUC | 2.215707 | 1.47E−08 |
| SEQ ID NO: 514 | 12h_hek293t_utr5-2hpf_pa | s1_activated | GGGCG | 1.814101 | 1.5E−08 |
| SEQ ID NO: 515 | 12h_hek293t_utr5-2hpf_pa | s1_activated | GCCGCU | 2.036619 | 1.59E−08 |
| SEQ ID NO: 516 | 12h_hek293t_utr5-2hpf_pa | s1_activated | CCCUCC | 2.348748 | 1.7E−08 |
| SEQ ID NO: 517 | 12h_hek293t_utr5-2hpf_pa | s1_activated | CCGCCU | 2.507778 | 1.75E−08 |
| SEQ ID NO: 518 | 12h_hek293t_utr5-2hpf_pa | s1_activated | CGGCUC | 2.185767 | 1.81E−08 |
| SEQ ID NO: 519 | 12h_hek293t_utr5-2hpf_pa | s1_activated | UCGGG | 1.773858 | 2.01E−08 |
| SEQ ID NO: 520 | 12h_hek293t_utr5-2hpf_pa | s1_activated | CCCCGC | 2.670152 | 2.04E−08 |
| SEQ ID NO: 521 | 12h_hek293t_utr5-2hpf_pa | s1_activated | GAGGCC | 2.523163 | 2.13E−08 |
| SEQ ID NO: 522 | 12h_hek293t_utr5-2hpf_pa | s1_activated | GGGGU | 1.830296 | 2.34E−08 |
| SEQ ID NO: 523 | 12h_hek293t_utr5-2hpf_pa | s1_activated | AGGCC | 1.779335 | 2.38E−08 |
| SEQ ID NO: 524 | 12h_hek293t_utr5-2hpf_pa | s1_activated | CCCCCC | 3.062128 | 2.52E−08 |
| SEQ ID NO: 525 | 12h_hek293t_utr5-2hpf_pa | s1_activated | CCGGGA | 2.478274 | 2.52E−08 |
| SEQ ID NO: 526 | 12h_hek293t_utr5-2hpf_pa | s1_activated | GGAGAG | 1.869434 | 2.63E−08 |
| SEQ ID NO: 527 | 12h_hek293t_utr5-2hpf_pa | s1_activated | GGGCC | 1.997416 | 2.79E−08 |
| SEQ ID NO: 528 | 12h_hek293t_utr5-2hpf_pa | s1_activated | GCGGGA | 2.193036 | 2.85E−08 |
| SEQ ID NO: 529 | 12h_hek293t_utr5-2hpf_pa | s1_activated | CCACCU | 2.721619 | 2.86E−08 |
| SEQ ID NO: 530 | 12h_hek293t_utr5-2hpf_pa | s1_activated | CUCCAC | 2.28616 | 3.05E−08 |
| SEQ ID NO: 531 | 12h_hek293t_utr5-2hpf_pa | s1_activated | AGCCCC | 2.55695 | 3.13E−08 |
| SEQ ID NO: 532 | 12h_hek293t_utr5-2hpf_pa | s1_activated | GCUGC | 1.451828 | 3.47E−08 |
| SEQ ID NO: 533 | 12h_hek293t_utr5-2hpf_pa | s1_activated | GCCGGC | 3.149302 | 3.9E−08 |
| SEQ ID NO: 534 | 12h_hek293t_utr5-2hpf_pa | s1_activated | CGGCA | 1.734408 | 3.94E−08 |
| SEQ ID NO: 535 | 12h_hek293t_utr5-2hpf_pa | s1_activated | UGCCC | 1.818823 | 4.47E−08 |
| SEQ ID NO: 536 | 12h_hek293t_utr5-2hpf_pa | s1_activated | UCGGU | 1.617441 | 4.89E−08 |
| SEQ ID NO: 537 | 12h_hek293t_utr5-2hpf_pa | s1_activated | UCCGG | 1.69811 | 4.91E−08 |
| SEQ ID NO: 538 | 12h_hek293t_utr5-2hpf_pa | s1_activated | CCGGA | 1.666993 | 5.1E−08 |
| SEQ ID NO: 539 | 12h_hek293t_utr5-2hpf_pa | s1_activated | UGCUC | 1.525233 | 5.48E−08 |
| SEQ ID NO: 540 | 12h_hek293t_utr5-2hpf_pa | s1_activated | UCCUC | 1.475261 | 5.51E−08 |
| SEQ ID NO: 541 | 12h_hek293t_utr5-2hpf_pa | s1_activated | ACCGC | 1.692761 | 5.79E−08 |
| SEQ ID NO: 542 | 12h_hek293t_utr5-2hpf_pa | s1_activated | UCCCC | 1.640135 | 5.8E−08 |
| SEQ ID NO: 543 | 12h_hek293t_utr5-2hpf_pa | s1_activated | GUCGG | 1.640135 | 5.8E−08 |
| SEQ ID NO: 544 | 12h_hek293t_utr5-2hpf_pa | s1_activated | CUCCCU | 2.130159 | 6.05E−08 |
| SEQ ID NO: 545 | 12h_hek293t_utr5-2hpf_pa | s1_activated | AGGGC | 1.79317 | 6.13E−08 |
| SEQ ID NO: 546 | 12h_hek293t_utr5-2hpf_pa | s1_activated | GGAGGG | 2.195651 | 6.49E−08 |
| SEQ ID NO: 547 | 12h_hek293t_utr5-2hpf_pa | s1_activated | CCGAG | 1.623701 | 7.07E−08 |
| SEQ ID NO: 548 | 12h_hek2931_utr5-2hpf_pa | s1_activated | CGUGG | 1.768221 | 7.21E−08 |
| SEQ ID NO: 549 | 12h_hek293t_utr5-2hpf_pa | s1_activated | CUCCGG | 2.314872 | 7.35E−08 |
| SEQ ID NO: 550 | 12h_hek293t_utr5-2hpf_pa | s1_activated | CGGCGG | 2.113836 | 7.93E−08 |
| SEQ ID NO: 551 | 12h_hek293t_utr5-2hpf_pa | s1_activated | AGCCGC | 2.096053 | 8.22E−08 |
| SEQ ID NO: 552 | 12h_hek293t_utr5-2hpf_pa | s1_activated | UCGGAC | 2.303061 | 8.62E−08 |
| SEQ ID NO: 553 | 12h_hek293t_utr5-2hpf_pa | s1_activated | CCGCCC | 3.009333 | 1.12E−07 |
| SEQ ID NO: 554 | 12h_hek293t_utr5-2hpf_pa | s1_activated | GGCGGG | 2.298796 | 1.26E−07 |
| SEQ ID NO: 555 | 12h_hek293t_utr5-2hpf_pa | s1_activated | GCCCCC | 2.925741 | 1.28E−07 |
| SEQ ID NO: 556 | 12h_hek293t_utr5-2hpf_pa | s1_activated | CCGGGU | 2.697441 | 1.37E−07 |
| SEQ ID NO: 557 | 12h_hek293t_utr5-2hpf_pa | s1_activated | GAGCGG | 2.049367 | 1.42E−07 |
| SEQ ID NO: 558 | 12h_hek293t_utr5-2hpf_pa | s1_activated | GUGGAG | 1.925743 | 1.48E−07 |
| SEQ ID NO: 559 | 12h_hek293t_utr5-2hpf_pa | s1_activated | CUCGA | 1.625266 | 1.54E−07 |
| SEQ ID NO: 560 | 12h_hek293t_utr5-2hpf_pa | s1_activated | CCGGUG | 2.111813 | 1.81E−07 |
| SEQ ID NO: 561 | 12h_hek293t_utr5-2hpf_pa | s1_activated | GCGCGG | 2.122766 | 1.99E−07 |
| SEQ ID NO: 562 | 12h_hek2931_utr5-2hpf_pa | s1_activated | CGGAGA | 1.950494 | 2.12E−07 |
| SEQ ID NO: 563 | 12h_hek293t_utr5-2hpf_pa | s1_activated | CCUCUC | 1.950494 | 2.12E−07 |
| SEQ ID NO: 564 | 12h_hek293t_utr5-2hpf_pa | s1_activated | GGGAUC | 2.134279 | 2.19E−07 |
| SEQ ID NO: 565 | 12h_hek293t_utr5-2hpf_pa | s1_activated | GGAGA | 1.447346 | 2.6E−07 |
| SEQ ID NO: 566 | 12h_hek293t_utr5-2hpf_pa | s1_activated | CACACU | 1.927901 | 2.64E−07 |
| SEQ ID NO: 567 | 12h_hek293t_utr5-2hpf_pa | s1_activated | GCUCU | 1.465192 | 2.78E−07 |
| SEQ ID NO: 568 | 12h_hek293t_utr5-2hpf_pa | s1_activated | GAGCCG | 2.116268 | 2.87E−07 |
| SEQ ID NO: 569 | 12h_hek293t_utr5-2hpf_pa | s1_activated | GCUCCG | 2.212745 | 2.93E−07 |
| SEQ ID NO: 570 | 12h_hek293t_utr5-2hpf_pa | s1_activated | CUCGGG | 2.526354 | 3.13E−07 |
| SEQ ID NO: 571 | 12h_hek293t_utr5-2hpf_pa | s1_activated | CUCGAG | 2.107376 | 3.29E−07 |
| SEQ ID NO: 572 | 12h_hek293t_utr5-2hpf_pa | s1_activated | GCGAG | 1.491859 | 4.4E−07 |
| SEQ ID NO: 573 | 12h_hek293t_utr5-2hpf_pa | s1_activated | GCACCG | 2.455167 | 4.44E−07 |
| SEQ ID NO: 574 | 12h_hek293t_utr5-2hpf_pa | s1_activated | GGCCUG | 2.121116 | 4.56E−07 |
| SEQ ID NO: 575 | 12h_hek293t_utr5-2hpf_pa | s1_activated | AGGCCG | 2.526921 | 4.61E−07 |
| SEQ ID NO: 576 | 12h_hek293t_utr5-2hpf_pa | s1_activated | GCACC | 1.736316 | 4.84E−07 |
| SEQ ID NO: 577 | 12h_hek293t_utr5-2hpf_pa | s1_activated | UCGGCC | 2.693539 | 5.34E−07 |
| SEQ ID NO: 578 | 12h_hek293t_utr5-2hpf_pa | s1_activated | CCCCGG | 2.693539 | 5.34E−07 |
| SEQ ID NO: 579 | 12h_hek293t_utr5-2hpf_pa | s1_activated | GAGCC | 1.557976 | 5.37E−07 |
| SEQ ID NO: 580 | 12h_hek293t_utr5-2hpf_pa | s1_activated | GGGUC | 1.703339 | 6.12E−07 |
| SEQ ID NO: 581 | 12h_hek293t_utr5-2hpf_pa | s1_activated | GUGGA | 1.430675 | 6.44E−07 |
| SEQ ID NO: 582 | 12h_hek293t_utr5-2hpf_pa | s1_activated | CCCUCU | 2.174231 | 6.57E−07 |
| SEQ ID NO: 583 | 12h_hek2931_utr5-2hpf_pa | s1_activated | GUGCG | 1.589886 | 6.68E−07 |
| SEQ ID NO: 584 | 12h_hek293t_utr5-2hpf_pa | s1_activated | UCCCG | 1.683555 | 6.72E−07 |
| SEQ ID NO: 585 | 12h_hek293t_utr5-2hpf_pa | s1_activated | CGGAGC | 1.984572 | 6.77E−07 |
| SEQ ID NO: 586 | 12h_hek293t_utr5-2hpf_pa | s1_activated | CGGCCG | 2.287797 | 7.16E−07 |
| SEQ ID NO: 587 | 12h_hek293t_utr5-2hpf_pa | s1_activated | CUCUCG | 2.104742 | 7.52E−07 |
| SEQ ID NO: 588 | 12h_hek293t_utr5-2hpf_pa | s1_activated | ACUCUC | 2.104742 | 7.52E−07 |
| SEQ ID NO: 589 | 12h_hek293t_utr5-2hpf_pa | s1_activated | CAGGGC | 2.436127 | 7.8E−07 |
| SEQ ID NO: 590 | 12h_hek293t_utr5-2hpf_pa | s1_activated | CCCCCG | 2.882503 | 8.01E−07 |
| SEQ ID NO: 591 | 12h_hek293t_utr5-2hpf_pa | s1_activated | GGGCGG | 2.360261 | 8.52E−07 |
| SEQ ID NO: 592 | 12h_hek293t_utr5-2hpf_pa | s1_activated | CGCUCG | 2.075402 | 8.85E−07 |
| SEQ ID NO: 593 | 12h_hek293t_utr5-2hpf_pa | s1_activated | UGCGG | 1.573938 | 8.9E−07 |
| SEQ ID NO: 594 | 12h_hek293t_utr5-2hpf_pa | s1_activated | CCGAAC | 2.38836 | 8.93E−07 |
| SEQ ID NO: 595 | 12h_hek293t_utr5-2hpf_pa | s1_activated | UCACC | 1.534446 | 8.96E−07 |
| SEQ ID NO: 596 | 12h_hek293t_utr5-2hpf_pa | s1_activated | GCCUC | 1.561802 | 9.61E−07 |
| SEQ ID NO: 597 | 12h_hek293t_utr5-2hpf_pa | s1_activated | CCCACC | 2.674963 | 9.67E−07 |
| SEQ ID NO: 598 | 12h_hek293t_utr5-2hpf_pa | s1_activated | CCCGCU | 2.344935 | 1.01E−06 |
| SEQ ID NO: 599 | 12h_hek293t_utr5-2hpf_pa | s1_activated | CGCUGC | 1.855465 | 1.06E−06 |
| SEQ ID NO: 600 | 12h_hek293t_utr5-2hpf_pa | s1_activated | GGUCGG | 2.507778 | 1.2E−06 |
| SEQ ID NO: 601 | 12h_hek293t_utr5-2hpf_pa | s1_activated | CCGGAC | 2.268942 | 1.22E−06 |
| SEQ ID NO: 602 | 12h_hek293t_utr5-2hpf_pa | s1_activated | CCAGCC | 2.268942 | 1.22E−06 |
| SEQ ID NO: 603 | 12h_hek293t_utr5-2hpf_pa | s1_activated | CCGCGC | 2.21682 | 1.23E−06 |
| SEQ ID NO: 604 | 12h_hek293t_utr5-2hpf_pa | s1_activated | AGCCAC | 2.356301 | 1.25E−06 |
| SEQ ID NO: 605 | 12h_hek293t_utr5-2hpf_pa | s1_activated | CAGCCC | 2.356301 | 1.25E−06 |
| SEQ ID NO: 606 | 12h_hek293t_utr5-2hpf_pa | s1_activated | CGAGG | 1.536862 | 1.33E−06 |
| SEQ ID NO: 607 | 12h_hek293t_utr5-2hpf_pa | s1_activated | CCAUC | 1.54257 | 1.33E−06 |
| SEQ ID NO: 608 | 12h_hek293t_utr5-2hpf_pa | s1_activated | CCCGGC | 2.314872 | 1.39E−06 |
| SEQ ID NO: 609 | 12h_hek293t_utr5-2hpf_pa | s1_activated | UGGGUC | 2.487715 | 1.45E−06 |
| SEQ ID NO: 610 | 12h_hek293t_utr5-2hpf_pa | s1_activated | CCCGU | 1.667986 | 1.5E−06 |
| SEQ ID NO: 611 | 12h_hek293t_utr5-2hpf_pa | s1_activated | CAGCC | 1.486319 | 1.58E−06 |
| SEQ ID NO: 612 | 12h_hek293t_utr5-2hpf_pa | s1_activated | CAGGAC | 2.149524 | 1.59E−06 |
| SEQ ID NO: 613 | 12h_hek293t_utr5-2hpf_pa | s1_activated | CCGGGC | 2.398744 | 1.62E−06 |
| SEQ ID NO: 614 | 12h_hek293t_utr5-2hpf_pa | s1_activated | GCGGCA | 2.300127 | 1.63E−06 |
| SEQ ID NO: 615 | 12h_hek293t_utr5-2hpf_pa | s1_activated | CCCAGA | 2.242248 | 1.66E−06 |
| SEQ ID NO: 616 | 12h_hek293t_utr5-2hpf_pa | s1_activated | CCACG | 1.749156 | 1.67E−06 |
| SEQ ID NO: 617 | 12h_hek293t_utr5-2hpf_pa | s1_activated | UCCUCC | 1.783883 | 1.79E−06 |
| SEQ ID NO: 618 | 12h_hek293t_utr5-2hpf_pa | s1_activated | GGUCC | 1.806607 | 1.9E−06 |
| SEQ ID NO: 619 | 12h_hek293t_utr5-2hpf_pa | s1_activated | GCUGGC | 1.975238 | 1.93E−06 |
| SEQ ID NO: 620 | 12h_hek293t_utr5-2hpf_pa | s1_activated | CGGUC | 1.633325 | 1.93E−06 |
| SEQ ID NO: 621 | 12h_hek293t_utr5-2hpf_pa | s1_activated | GCCGGA | 2.153275 | 2E−06 |
| SEQ ID NO: 622 | 12h_hek293t_utr5-2hpf_pa | s1_activated | CGGGGC | 2.487052 | 2.13E−06 |
| SEQ ID NO: 623 | 12h_hek293t_utr5-2hpf_pa | s1_activated | CUCUCA | 1.940324 | 2.16E−06 |
| SEQ ID NO: 624 | 12h_hek293t_utr5-2hpf_pa | s1_activated | GGUGGC | 2.271195 | 2.24E−06 |
| SEQ ID NO: 625 | 12h_hek293t_utr5-2hpf_pa | s1_activated | GAGGGU | 2.364476 | 2.28E−06 |
| SEQ ID NO: 626 | 12h_hek293t_utr5-2hpf_pa | s1_activated | CCGGAG | 1.997897 | 2.73E−06 |
| SEQ ID NO: 627 | 12h_hek293t_utr5-2hpf_pa | s1_activated | CUGCCC | 2.173407 | 2.76E−06 |
| SEQ ID NO: 628 | 12h_hek293t_utr5-2hpf_pa | s1_activated | GCCCAG | 2.279798 | 2.8E−06 |
| SEQ ID NO: 629 | 12h_hek293t_utr5-2hpf_pa | s1_activated | CGCGA | 1.531296 | 3.07E−06 |
| SEQ ID NO: 630 | 12h_hek293t_utr5-2hpf_pa | s1_activated | CCGUC | 1.534223 | 3.08E−06 |
| SEQ ID NO: 631 | 12h_hek293t_utr5-2hpf_pa | s1_activated | UCCCU | 1.510558 | 3.15E−06 |
| SEQ ID NO: 632 | 12h_hek2931_utr5-2hpf_pa | s1_activated | ACCCG | 1.637098 | 3.24E−06 |
| SEQ ID NO: 633 | 12h_hek293t_utr5-2hpf_pa | s1_activated | CACCCG | 2.360261 | 3.35E−06 |
| SEQ ID NO: 634 | 12h_hek293t_utr5-2hpf_pa | s1_activated | AGAGGG | 2.085676 | 3.58E−06 |
| SEQ ID NO: 635 | 12h_hek293t_utr5-2hpf_pa | s1_activated | GCCGAG | 2.122986 | 3.82E−06 |
| SEQ ID NO: 636 | 12h_hek293t_utr5-2hpf_pa | s1_activated | GCAGG | 1.493934 | 3.86E−06 |
| SEQ ID NO: 637 | 12h_hek293t_utr5-2hpf_pa | s1_activated | AAGCCC | 2.21546 | 4.1E−06 |
| SEQ ID NO: 638 | 12h_hek293t_utr5-2hpf_pa | s1_activated | GGCAGG | 2.033958 | 4.26E−06 |
| SEQ ID NO: 639 | 12h_hek293t_utr5-2hpf_pa | s1_activated | UCCCUC | 1.850399 | 4.28E−06 |
| SEQ ID NO: 640 | 12h_hek293t_utr5-2hpf_pa | s1_activated | CCUCG | 1.55577 | 4.3E−06 |
| SEQ ID NO: 641 | 12h_hek293t_utr5-2hpf_pa | s1_activated | GUGUGU | 1.503434 | 4.32E−06 |
| SEQ ID NO: 642 | 12h_hek293t_utr5-2hpf_pa | s1_activated | CGAGC | 1.452409 | 4.35E−06 |
| SEQ ID NO: 643 | 12h_hek293t_utr5-2hpf_pa | s1_activated | CCCGCC | 2.407466 | 4.35E−06 |
| SEQ ID NO: 644 | 12h_hek293t_utr5-2hpf_pa | s1_activated | GUGCGG | 2.183242 | 4.37E−06 |
| SEQ ID NO: 645 | 12h_hek293t_utr5-2hpf_pa | s1_activated | GGGCAG | 2.111813 | 4.38E−06 |
| SEQ ID NO: 646 | 12h_hek293t_utr5-2hpf_pa | s1_activated | GUGCCG | 2.326055 | 4.66E−06 |
| SEQ ID NO: 647 | 12h_hek293t_utr5-2hpf_pa | s1_activated | UGGUGG | 1.988927 | 4.91E−06 |
| SEQ ID NO: 648 | 12h_hek293t_utr5-2hpf_pa | s1_activated | GUGGG | 1.638543 | 5.13E−06 |
| SEQ ID NO: 649 | 12h_hek293t_utr5-2hpf_pa | s1_activated | GCGGGG | 2.222081 | 5.16E−06 |
| SEQ ID NO: 650 | 12h_hek293t_utr5-2hpf_pa | s1_activated | CUCGC | 1.510748 | 5.31E−06 |
| SEQ ID NO: 651 | 12h_hek293t_utr5-2hpf_pa | s1_activated | GCUGCG | 1.908905 | 5.37E−06 |
| SEQ ID NO: 652 | 12h_hek293t_utr5-2hpf_pa | s1_activated | GGGGCC | 2.612268 | 5.7E−06 |
| SEQ ID NO: 653 | 12h_hek2931_utr5-2hpf_pa | s1_activated | GUCAC | 1.591588 | 5.74E−06 |
| SEQ ID NO: 654 | 12h_hek293t_utr5-2hpf_pa | s1_activated | CGCCCC | 3.07405 | 5.75E−06 |
| SEQ ID NO: 655 | 12h_hek293t_utr5-2hpf_pa | s1_activated | AGAGGC | 2.066711 | 5.88E−06 |
| SEQ ID NO: 656 | 12h_hek293t_utr5-2hpf_pa | s1_activated | GGGCA | 1.632204 | 5.97E−06 |
| SEQ ID NO: 657 | 12h_hek293t_utr5-2hpf_pa | s1_activated | CACAC | 1.419617 | 6.1E−06 |
| SEQ ID NO: 658 | 12h_hek293t_utr5-2hpf_pa | s1_activated | CCCGAA | 2.369554 | 6.12E−06 |
| SEQ ID NO: 659 | 12h_hek293t_utr5-2hpf_pa | s1_activated | GGCUGC | 1.941505 | 6.19E−06 |
| SEQ ID NO: 660 | 12h_hek293t_utr5-2hpf_pa | s1_activated | UGCUCG | 1.894765 | 6.67E−06 |
| SEQ ID NO: 661 | 12h_hek293t_utr5-2hpf_pa | s1_activated | AGGCG | 1.577444 | 6.68E−06 |
| SEQ ID NO: 662 | 12h_hek293t_utr5-2hpf_pa | s1_activated | UCGGUG | 2.035298 | 6.81E−06 |
| SEQ ID NO: 663 | 12h_hek293t_utr5-2hpf_pa | s1_activated | CCUCCC | 2.015597 | 6.9E−06 |
| SEQ ID NO: 664 | 12h_hek293t_utr5-2hpf_pa | s1_activated | CUUCCC | 2.015597 | 6.9E−06 |
| SEQ ID NO: 665 | 12h_hek293t_utr5-2hpf_pa | s1_activated | CCUCUU | 1.855068 | 6.96E−06 |
| SEQ ID NO: 666 | 12h_hek293t_utr5-2hpf_pa | s1_activated | CGCAGG | 2.384444 | 7.61E−06 |
| SEQ ID NO: 667 | 12h_hek293t_utr5-2hpf_pa | s1_activated | GGGCUG | 1.925973 | 7.75E−06 |
| SEQ ID NO: 668 | 12h_hek293t_utr5-2hpf_pa | s1_activated | CGCCU | 1.582562 | 8.08E−06 |
| SEQ ID NO: 669 | 12h_hek293t_utr5-2hpf_pa | s1_activated | CGCCA | 1.579857 | 8.66E−06 |
| SEQ ID NO: 670 | 12h_hek293t_utr5-2hpf_pa | s1_activated | CGGUGG | 2.200373 | 8.72E−06 |
| SEQ ID NO: 671 | 12h_hek293t_utr5-2hpf_pa | s1_activated | CGGGCA | 2.44 | 9.81E−06 |
| SEQ ID NO: 672 | 12h_hek293t_utr5-2hpf_pa | s2_activated | AUUUU | 1.660548 | 1.98E−27 |
| SEQ ID NO: 673 | 12h_hek293t_utr5-2hpf_pa | s2_activated | AUUUUU | 2.111144 | 3.72E−24 |
| SEQ ID NO: 674 | 12h_hek293t_utr5-2hpf_pa | s2_activated | UUUUU | 1.590344 | 9.81E−24 |
| SEQ ID NO: 675 | 12h_hek293t_utr5-2hpf_pa | s2_activated | UUUUA | 1.554587 | 8.91E−23 |
| SEQ ID NO: 676 | 12h_hek293t_utr5-2hpf_pa | s2_activated | UUUUUU | 1.960521 | 1.58E−19 |
| SEQ ID NO: 677 | 12h_hek293t_utr5-2hpf_pa | s2_activated | UUUAU | 1.538397 | 1.47E−17 |
| SEQ ID NO: 678 | 12h_hek293t_utr5-2hpf_pa | s2_activated | AAUUUU | 2.38717 | 2.41E−16 |
| SEQ ID NO: 679 | 12h_hek293t_utr5-2hpf_pa | s2_activated | UUUUG | 1.435186 | 3.64E−14 |
| SEQ ID NO: 680 | 12h_hek293t_utr5-2hpf_pa | s2_activated | UAUUU | 1.453944 | 8.24E−14 |
| SEQ ID NO: 681 | 12h_hek293t_utr5-2hpf_pa | s2_activated | UGUUU | 1.369161 | 7.54E−13 |
| SEQ ID NO: 682 | 12h_hek293t_utr5-2hpf_pa | s2_activated | UUUUUA | 1.701528 | 8.31E−13 |
| SEQ ID NO: 683 | 12h_hek293t_utr5-2hpf_pa | s2_activated | UAUUUU | 1.699966 | 9.02E−13 |
| SEQ ID NO: 684 | 12h_hek293t_utr5-2hpf_pa | s2_activated | UUUUAA | 1.667804 | 2.24E−12 |
| SEQ ID NO: 685 | 12h_hek293t_utr5-2hpf_pa | s2_activated | UUUAA | 1.393173 | 2.4E−11 |
| SEQ ID NO: 686 | 12h_hek293t_utr5-2hpf_pa | s2_activated | UUUAAA | 1.661438 | 2.76E−11 |
| SEQ ID NO: 687 | 12h_hek293t_utr5-2hpf_pa | s2_activated | GUUUU | 1.339956 | 8.72E−11 |
| SEQ ID NO: 688 | 12h_hek293t_utr5-2hpf_pa | s2_activated | AAUUU | 1.56589 | 1.3E−10 |
| SEQ ID NO: 689 | 12h_hek293t_utr5-2hpf_pa | s2_activated | UAUGU | 1.800794 | 2.25E−10 |
| SEQ ID NO: 690 | 12h_hek293t_utr5-2hpf_pa | s2_activated | UUUUAU | 1.664202 | 3.15E−10 |
| SEQ ID NO: 691 | 12h_hek293t_utr5-2hpf_pa | s2_activated | UUUUUG | 1.657113 | 3.78E−10 |
| SEQ ID NO: 692 | 12h_hek293t_utr5-2hpf_pa | s2_activated | UUAUA | 1.593678 | 1.02E−09 |
| SEQ ID NO: 693 | 12h_hek293t_utr5-2hpf_pa | s2_activated | AAAUU | 1.599194 | 1.36E−09 |
| SEQ ID NO: 694 | 12h_hek293t_utr5-2hpf_pa | s2_activated | UUAAA | 1.400588 | 1.63E−09 |
| SEQ ID NO: 695 | 12h_hek293t_utr5-2hpf_pa | s2_activated | GUAUU | 1.604006 | 4.21E−09 |
| SEQ ID NO: 696 | 12h_hek293t_utr5-2hpf_pa | s2_activated | UGUAU | 1.586246 | 5.81E−09 |
| SEQ ID NO: 697 | 12h_hek293t_utr5-2hpf_pa | s2_activated | AUGUUU | 1.883122 | 8.63E−09 |
| SEQ ID NO: 698 | 12h_hek293t_utr5-2hpf_pa | s2_activated | UAUAUG | 2.696875 | 9.77E−09 |
| SEQ ID NO: 699 | 12h_hek293t_utr5-2hpf_pa | s2_activated | UAUGC | 1.85048 | 1.37E−08 |
| SEQ ID NO: 700 | 12h_hek2931_utr5-2hpf_pa | s2_activated | UUUGU | 1.326549 | 1.68E−08 |
| SEQ ID NO: 701 | 12h_hek293t_utr5-2hpf_pa | s2_activated | UUUAUG | 1.885344 | 3.86E−08 |
| SEQ ID NO: 702 | 12h_hek293t_utr5-2hpf_pa | s2_activated | AAUUA | 1.616038 | 4.55E−08 |
| SEQ ID NO: 703 | 12h_hek293t_utr5-2hpf_pa | s2_activated | UUUAG | 1.440595 | 5.82E−08 |
| SEQ ID NO: 704 | 12h_hek293t_utr5-2hpf_pa | s2_activated | UGAUUU | 1.709292 | 6.12E−08 |
| SEQ ID NO: 705 | 12h_hek293t_utr5-2hpf_pa | s2_activated | UUAUG | 1.581649 | 6.61E−08 |
| SEQ ID NO: 706 | 12h_hek293t_utr5-2hpf_pa | s2_activated | UGAUU | 1.451803 | 8.2E−08 |
| SEQ ID NO: 707 | 12h_hek293t_utr5-2hpf_pa | s2_activated | UUAUU | 1.343634 | 9.18E−08 |
| SEQ ID NO: 708 | 12h_hek293t_utr5-2hpf_pa | s2_activated | AUUUUA | 1.655376 | 1.08E−07 |
| SEQ ID NO: 709 | 12h_hek293t_utr5-2hpf_pa | s2_activated | UUGUU | 1.329664 | 1.13E−07 |
| SEQ ID NO: 710 | 12h_hek293t_utr5-2hpf_pa | s2_activated | AUGUU | 1.512144 | 1.77E−07 |
| SEQ ID NO: 711 | 12h_hek293t_utr5-2hpf_pa | s2_activated | GUAUUU | 1.783825 | 2.08E−07 |
| SEQ ID NO: 712 | 12h_hek293t_utr5-2hpf_pa | s2_activated | UGUUUU | 1.412573 | 2.21E−07 |
| SEQ ID NO: 713 | 12h_hek293t_utr5-2hpf_pa | s2_activated | UGCAUG | 2.12009 | 2.7E−07 |
| SEQ ID NO: 714 | 12h_hek293t_utr5-2hpf_pa | s2_activated | AUUUA | 1.378997 | 3.69E−07 |
| SEQ ID NO: 715 | 12h_hek293t_utr5-2hpf_pa | s2_activated | AAUGUU | 1.948524 | 5.18E−07 |
| SEQ ID NO: 716 | 12h_hek293t_utr5-2hpf_pa | s2_activated | UUUAUA | 1.70659 | 5.6E−07 |
| SEQ ID NO: 717 | 12h_hek293t_utr5-2hpf_pa | s2_activated | UAUAU | 1.552329 | 6.47E−07 |
| SEQ ID NO: 718 | 12h_hek293t_utr5-2hpf_pa | s2_activated | AAUUAG | 2.430229 | 8.24E−07 |
| SEQ ID NO: 719 | 12h_hek293t_utr5-2hpf_pa | s2_activated | UCUUG | 1.497224 | 8.9E−07 |
| SEQ ID NO: 720 | 12h_hek293t_utr5-2hpf_pa | s2_activated | UAUGUU | 2.12409 | 9.74E−07 |
| SEQ ID NO: 721 | 12h_hek2931_utr5-2hpf_pa | s2_activated | UUUCU | 1.299871 | 1.15E−06 |
| SEQ ID NO: 722 | 12h_hek293t_utr5-2hpf_pa | s2_activated | UUACGU | 2.299705 | 1.18E−06 |
| SEQ ID NO: 723 | 12h_hek293t_utr5-2hpf_pa | s2_activated | GUUUUU | 1.459132 | 1.49E−06 |
| SEQ ID NO: 724 | 12h_hek293t_utr5-2hpf_pa | s2_activated | UUUUGA | 1.589586 | 1.61E−06 |
| SEQ ID NO: 725 | 12h_hek293t_utr5-2hpf_pa | s2_activated | UUAUUU | 1.435687 | 1.63E−06 |
| SEQ ID NO: 726 | 12h_hek293t_utr5-2hpf_pa | s2_activated | UUAUAU | 1.912567 | 1.81E−06 |
| SEQ ID NO: 727 | 12h_hek293t_utr5-2hpf_pa | s2_activated | AUUAU | 1.442799 | 1.98E−06 |
| SEQ ID NO: 728 | 12h_hek293t_utr5-2hpf_pa | s2_activated | ACUUUU | 1.580025 | 2.12E−06 |
| SEQ ID NO: 729 | 12h_hek293t_utr5-2hpf_pa | s2_activated | UUUGA | 1.31271 | 2.13E−06 |
| SEQ ID NO: 730 | 12h_hek293t_utr5-2hpf_pa | s2_activated | UUGAU | 1.396147 | 2.24E−06 |
| SEQ ID NO: 731 | 12h_hek293t_utr5-2hpf_pa | s2_activated | UGCAU | 1.48408 | 2.36E−06 |
| SEQ ID NO: 732 | 12h_hek293t_utr5-2hpf_pa | s2_activated | GAUUU | 1.332804 | 2.48E−06 |
| SEQ ID NO: 733 | 12h_hek293t_utr5-2hpf_pa | s2_activated | UCUUU | 1.309425 | 2.49E−06 |
| SEQ ID NO: 734 | 12h_hek293t_utr5-2hpf_pa | s2_activated | AUAUU | 1.354429 | 2.66E−06 |
| SEQ ID NO: 735 | 12h_hek2931_utr5-2hpf_pa | s2_activated | UUUUAG | 1.658051 | 2.81E−06 |
| SEQ ID NO: 736 | 12h_hek293t_utr5-2hpf_pa | s2_activated | UGCUU | 1.389992 | 2.96E−06 |
| SEQ ID NO: 737 | 12h_hek293t_utr5-2hpf_pa | s2_activated | UUAGU | 1.452346 | 3.24E−06 |
| SEQ ID NO: 738 | 12h_hek293t_utr5-2hpf_pa | s2_activated | ACGCUU | 1.969214 | 3.3E−06 |
| SEQ ID NO: 739 | 12h_hek293t_utr5-2hpf_pa | s2_activated | GUUUAU | 1.571193 | 3.38E−06 |
| SEQ ID NO: 740 | 12h_hek293t_utr5-2hpf_pa | s2_activated | UUUUGU | 1.445209 | 5.24E−06 |
| SEQ ID NO: 741 | 12h_hek293t_utr5-2hpf_pa | s2_activated | GUAUGU | 2.277415 | 5.4E−06 |
| SEQ ID NO: 742 | 12h_hek293t_utr5-2hpf_pa | s2_activated | GAUUUU | 1.580025 | 5.58E−06 |
| SEQ ID NO: 743 | 12h_hek293t_utr5-2hpf_pa | s2_activated | UGUAUG | 1.892199 | 5.66E−06 |
| SEQ ID NO: 744 | 12h_hek293t_utr5-2hpf_pa | s2_activated | UAAAU | 1.381645 | 5.69E−06 |
| SEQ ID NO: 745 | 12h_hek293t_utr5-2hpf_pa | s2_activated | GUUUA | 1.296458 | 7.45E−06 |
| SEQ ID NO: 746 | 12h_hek293t_utr5-2hpf_pa | s2_activated | GUAUG | 1.664585 | 7.84E−06 |
| SEQ ID NO: 747 | 12h_hek293t_utr5-6hpf_pa | active | CUCUC | 2.02274 | 2.74E−26 |
| SEQ ID NO: 748 | 12h_hek293t_utr5-6hpf_pa | active | CCCUC | 2.199967 | 1.72E−22 |
| SEQ ID NO: 749 | 12h_hek293t_utr5-6hpf_pa | active | UCCCC | 2.210871 | 5.6E−22 |
| SEQ ID NO: 750 | 12h_hek293t_utr5-6hpf_pa | active | UUCCC | 1.958395 | 1.14E−20 |
| SEQ ID NO: 751 | 12h_hek293t_utr5-6hpf_pa | active | CCCCU | 2.198532 | 1.69E−19 |
| SEQ ID NO: 752 | 12h_hek293t_utr5-6hpf_pa | active | UCUCU | 1.777517 | 1.9E−18 |
| SEQ ID NO: 753 | 12h_hek293t_utr5-6hpf_pa | active | CCUCU | 1.888037 | 5.5E−18 |
| SEQ ID NO: 754 | 12h_hek2931_utr5-6hpf_pa | active | UUCUC | 1.704453 | 1.83E−17 |
| SEQ ID NO: 755 | 12h_hek293t_utr5-6hpf_pa | active | CCCCC | 2.401741 | 5.64E−17 |
| SEQ ID NO: 756 | 12h_hek293t_utr5-6hpf_pa | active | UUUCC | 1.611221 | 2.81E−16 |
| SEQ ID NO: 757 | 12h_hek293t_utr5-6hpf_pa | active | UCCCU | 1.949752 | 3.08E−16 |
| SEQ ID NO: 758 | 12h_hek293t_utr5-6hpf_pa | active | UUUUCC | 2.069382 | 3.1E−16 |
| SEQ ID NO: 759 | 12h_hek293t_utr5-6hpf_pa | active | UCUCUC | 2.410673 | 6.99E−16 |
| SEQ ID NO: 760 | 12h_hek293t_utr5-6hpf_pa | active | CUUUU | 1.490355 | 1.21E−15 |
| SEQ ID NO: 761 | 12h_hek293t_utr5-6hpf_pa | active | CUCUCC | 2.492184 | 2.09E−15 |
| SEQ ID NO: 762 | 12h_hek293t_utr5-6hpf_pa | active | UCCCUC | 2.563633 | 3.75E−15 |
| SEQ ID NO: 763 | 12h_hek293t_utr5-6hpf_pa | active | UUUUC | 1.462448 | 1.2E−14 |
| SEQ ID NO: 764 | 12h_hek293t_utr5-6hpf_pa | active | CUCCC | 1.951237 | 4.58E−14 |
| SEQ ID NO: 765 | 12h_hek293t_utr5-6hpf_pa | active | UUUCCC | 2.154643 | 5.9E−14 |
| SEQ ID NO: 766 | 12h_hek293t_utr5-6hpf_pa | active | UUUCU | 1.479348 | 6.66E−14 |
| SEQ ID NO: 767 | 12h_hek293t_utr5-6hpf_pa | active | UCUCC | 1.681709 | 4.21E−13 |
| SEQ ID NO: 768 | 12h_hek2931_utr5-6hpf_pa | active | UUCUCU | 2.04798 | 1E−12 |
| SEQ ID NO: 769 | 12h_hek293t_utr5-6hpf_pa | active | CUCUU | 1.612739 | 1.11E−12 |
| SEQ ID NO: 770 | 12h_hek293t_utr5-6hpf_pa | active | UUCCCC | 2.320876 | 1.49E−12 |
| SEQ ID NO: 771 | 12h_hek293t_utr5-6hpf_pa | active | CUCUCU | 2.166108 | 2.61E−12 |
| SEQ ID NO: 772 | 12h_hek293t_utr5-6hpf_pa | active | UUCCU | 1.598592 | 2.94E−12 |
| SEQ ID NO: 773 | 12h_hek293t_utr5-6hpf_pa | active | CUCCCC | 2.646113 | 5.67E−12 |
| SEQ ID NO: 774 | 12h_hek293t_utr5-6hpf_pa | active | GUUUU | 1.360109 | 8.05E−12 |
| SEQ ID NO: 775 | 12h_hek293t_utr5-6hpf_pa | active | UUUCUC | 1.85638 | 8.79E−12 |
| SEQ ID NO: 776 | 12h_hek293t_utr5-6hpf_pa | active | CCCCCU | 2.584834 | 1.05E−11 |
| SEQ ID NO: 777 | 12h_hek293t_utr5-6hpf_pa | active | UUCUU | 1.541736 | 1.22E−11 |
| SEQ ID NO: 778 | 12h_hek293t_utr5-6hpf_pa | active | CCCCUC | 2.553267 | 5.85E−11 |
| SEQ ID NO: 779 | 12h_hek293t_utr5-6hpf_pa | active | CCUCUC | 2.197357 | 1.98E−10 |
| SEQ ID NO: 780 | 12h_hek293t_utr5-6hpf_pa | active | UCCCCC | 2.659193 | 2.09E−10 |
| SEQ ID NO: 781 | 12h_hek293t_utr5-6hpf_pa | active | CCUCC | 1.673648 | 3.12E−10 |
| SEQ ID NO: 782 | 12h_hek293t_utr5-6hpf_pa | active | GCUCU | 1.572305 | 5.31E−10 |
| SEQ ID NO: 783 | 12h_hek293t_utr5-6hpf_pa | active | CCCCA | 1.958291 | 5.34E−10 |
| SEQ ID NO: 784 | 12h_hek293t_utr5-6hpf_pa | active | CCCUU | 1.758273 | 7.33E−10 |
| SEQ ID NO: 785 | 12h_hek293t_utr5-6hpf_pa | active | UUUUU | 1.347773 | 8.69E−10 |
| SEQ ID NO: 786 | 12h_hek293t_utr5-6hpf_pa | active | CUUCC | 1.639443 | 1.18E−09 |
| SEQ ID NO: 787 | 12h_hek293t_utr5-6hpf_pa | active | UUUAUU | 1.566777 | 2.38E−09 |
| SEQ ID NO: 788 | 12h_hek293t_utr5-6hpf_pa | active | UUUGUU | 1.563408 | 3.09E−09 |
| SEQ ID NO: 789 | 12h_hek293t_utr5-6hpf_pa | active | UUCCUC | 1.867446 | 4.17E−09 |
| SEQ ID NO: 790 | 12h_hek293t_utr5-6hpf_pa | active | ACCCC | 1.874339 | 4.18E−09 |
| SEQ ID NO: 791 | 12h_hek293t_utr5-6hpf_pa | active | UCUUC | 1.516739 | 4.6E−09 |
| SEQ ID NO: 792 | 12h_hek293t_utr5-6hpf_pa | active | CUUUUU | 1.613099 | 5.71E−09 |
| SEQ ID NO: 793 | 12h_hek293t_utr5-6hpf_pa | active | CUUCU | 1.532131 | 6.96E−09 |
| SEQ ID NO: 794 | 12h_hek293t_utr5-6hpf_pa | active | ACUUU | 1.427352 | 7.34E−09 |
| SEQ ID NO: 795 | 12h_hek293t_utr5-6hpf_pa | active | UCCUC | 1.504231 | 9.46E−09 |
| SEQ ID NO: 796 | 12h_hek293t_utr5-6hpf_pa | active | CUCUUU | 1.772604 | 1.09E−08 |
| SEQ ID NO: 797 | 12h_hek293t_utr5-6hpf_pa | active | CGCUCU | 2.148723 | 1.13E−08 |
| SEQ ID NO: 798 | 12h_hek293t_utr5-6hpf_pa | active | CUUUUC | 1.754791 | 1.16E−08 |
| SEQ ID NO: 799 | 12h_hek293t_utr5-6hpf_pa | active | CCCCUU | 2.293256 | 1.45E−08 |
| SEQ ID NO: 800 | 12h_hek293t_utr5-6hpf_pa | active | UCUUU | 1.377601 | 1.64E−08 |
| SEQ ID NO: 801 | 12h_hek293t_utr5-6hpf_pa | active | UCUUCU | 1.907647 | 2.31E−08 |
| SEQ ID NO: 802 | 12h_hek293t_utr5-6hpf_pa | active | UCUCGC | 2.274729 | 3.61E−08 |
| SEQ ID NO: 803 | 12h_hek2931_utr5-6hpf_pa | active | CCCUCC | 2.298959 | 4.79E−08 |
| SEQ ID NO: 804 | 12h_hek293t_utr5-6hpf_pa | active | CACCC | 1.722978 | 5.49E−08 |
| SEQ ID NO: 805 | 12h_hek293t_utr5-6hpf_pa | active | UCUCG | 1.601047 | 5.59E−08 |
| SEQ ID NO: 806 | 12h_hek293t_utr5-6hpf_pa | active | UCUCCC | 2.218446 | 6.15E−08 |
| SEQ ID NO: 807 | 12h_hek293t_utr5-6hpf_pa | active | UUCUUC | 1.862227 | 6.63E−08 |
| SEQ ID NO: 808 | 12h_hek293t_utr5-6hpf_pa | active | CGCUC | 1.538529 | 7.94E−08 |
| SEQ ID NO: 809 | 12h_hek293t_utr5-6hpf_pa | active | UCUCUU | 1.811112 | 1.08E−07 |
| SEQ ID NO: 810 | 12h_hek293t_utr5-6hpf_pa | active | UACUUU | 1.907381 | 1.16E−07 |
| SEQ ID NO: 811 | 12h_hek293t_utr5-6hpf_pa | active | UUUUA | 1.285196 | 1.26E−07 |
| SEQ ID NO: 812 | 12h_hek293t_utr5-6hpf_pa | active | CCUCCC | 2.202274 | 1.88E−07 |
| SEQ ID NO: 813 | 12h_hek293t_utr5-6hpf_pa | active | CUCUG | 1.440396 | 2.05E−07 |
| SEQ ID NO: 814 | 12h_hek293t_utr5-6hpf_pa | active | UUGUU | 1.320167 | 2.5E−07 |
| SEQ ID NO: 815 | 12h_hek293t_utr5-6hpf_pa | active | CUCGC | 1.585847 | 2.56E−07 |
| SEQ ID NO: 816 | 12h_hek293t_utr5-6hpf_pa | active | UUUUUU | 1.514338 | 3.01E−07 |
| SEQ ID NO: 817 | 12h_hek293t_utr5-6hpf_pa | active | CUCCU | 1.486391 | 3.64E−07 |
| SEQ ID NO: 818 | 12h_hek293t_utr5-6hpf_pa | active | UUUGC | 1.381916 | 3.93E−07 |
| SEQ ID NO: 819 | 12h_hek293t_utr5-6hpf_pa | active | CCCCAC | 2.779866 | 4.11E−07 |
| SEQ ID NO: 820 | 12h_hek293t_utr5-6hpf_pa | active | CUUCUU | 1.914317 | 4.2E−07 |
| SEQ ID NO: 821 | 12h_hek293t_utr5-6hpf_pa | active | UCCCCA | 2.422721 | 4.34E−07 |
| SEQ ID NO: 822 | 12h_hek293t_utr5-6hpf_pa | active | UCCUCC | 1.834906 | 4.41E−07 |
| SEQ ID NO: 823 | 12h_hek293t_utr5-6hpf_pa | active | GCCUCU | 2.080368 | 4.94E−07 |
| SEQ ID NO: 824 | 12h_hek293t_utr5-6hpf_pa | active | CCGUU | 1.525939 | 5.74E−07 |
| SEQ ID NO: 825 | 12h_hek293t_utr5-6hpf_pa | active | UAUUUC | 1.714422 | 6.16E−07 |
| SEQ ID NO: 826 | 12h_hek293t_utr5-6hpf_pa | active | CCCUCU | 2.173421 | 6.63E−07 |
| SEQ ID NO: 827 | 12h_hek293t_utr5-6hpf_pa | active | CCCUCA | 2.216578 | 6.77E−07 |
| SEQ ID NO: 828 | 12h_hek293t_utr5-6hpf_pa | active | CUUCUC | 1.832961 | 7.88E−07 |
| SEQ ID NO: 829 | 12h_hek293t_utr5-6hpf_pa | active | GUUUUU | 1.471562 | 8.43E−07 |
| SEQ ID NO: 830 | 12h_hek293t_utr5-6hpf_pa | active | CCUUUU | 1.741017 | 9.23E−07 |
| SEQ ID NO: 831 | 12h_hek293t_utr5-6hpf_pa | active | CCUGCC | 2.106207 | 9.58E−07 |
| SEQ ID NO: 832 | 12h_hek293t_utr5-6hpf_pa | active | UUUUCU | 1.499321 | 9.78E−07 |
| SEQ ID NO: 833 | 12h_hek293t_utr5-6hpf_pa | active | CCCUUC | 2.085339 | 9.9E−07 |
| SEQ ID NO: 834 | 12h_hek293t_utr5-6hpf_pa | active | UUUGCC | 1.943021 | 1.09E−06 |
| SEQ ID NO: 835 | 12h_hek293t_utr5-6hpf_pa | active | CUGCU | 1.355974 | 1.12E−06 |
| SEQ ID NO: 836 | 12h_hek293t_utr5-6hpf_pa | active | CUUCCC | 2.10856 | 1.21E−06 |
| SEQ ID NO: 837 | 12h_hek293t_utr5-6hpf_pa | active | UCUUUU | 1.555972 | 1.28E−06 |
| SEQ ID NO: 838 | 12h_hek2931_utr5-6hpf_pa | active | UUUUUC | 1.505779 | 1.29E−06 |
| SEQ ID NO: 839 | 12h_hek293t_utr5-6hpf_pa | active | CCCCCC | 2.744334 | 1.37E−06 |
| SEQ ID NO: 840 | 12h_hek293t_utr5-6hpf_pa | active | CCCAG | 1.601818 | 1.42E−06 |
| SEQ ID NO: 841 | 12h_hek293t_utr5-6hpf_pa | active | AGCUU | 1.436637 | 1.47E−06 |
| SEQ ID NO: 842 | 12h_hek293t_utr5-6hpf_pa | active | UUCCCU | 1.923058 | 1.52E−06 |
| SEQ ID NO: 843 | 12h_hek293t_utr5-6hpf_pa | active | CUCCCU | 1.974502 | 1.96E−06 |
| SEQ ID NO: 844 | 12h_hek293t_utr5-6hpf_pa | active | CACCCC | 2.248563 | 2.1E−06 |
| SEQ ID NO: 845 | 12h_hek293t_utr5-6hpf_pa | active | CUCUUC | 1.768051 | 2.11E−06 |
| SEQ ID NO: 846 | 12h_hek293t_utr5-6hpf_pa | active | UUUCUU | 1.53467 | 2.28E−06 |
| SEQ ID NO: 847 | 12h_hek293t_utr5-6hpf_pa | active | UCCCCU | 2.06042 | 2.33E−06 |
| SEQ ID NO: 848 | 12h_hek293t_utr5-6hpf_pa | active | UUUUAU | 1.483532 | 2.42E−06 |
| SEQ ID NO: 849 | 12h_hek293t_utr5-6hpf_pa | active | UACUU | 1.517575 | 2.5E−06 |
| SEQ ID NO: 850 | 12h_hek293t_utr5-6hpf_pa | active | UUUCCU | 1.60171 | 3.14E−06 |
| SEQ ID NO: 851 | 12h_hek293t_utr5-6hpf_pa | active | CUUUC | 1.368441 | 3.17E−06 |
| SEQ ID NO: 852 | 12h_hek293t_utr5-6hpf_pa | active | CCCACC | 2.578468 | 3.22E−06 |
| SEQ ID NO: 853 | 12h_hek293t_utr5-6hpf_pa | active | CUUCCU | 1.843038 | 3.36E−06 |
| SEQ ID NO: 854 | 12h_hek293t_utr5-6hpf_pa | active | CCACC | 1.62909 | 3.5E−06 |
| SEQ ID NO: 855 | 12h_hek293t_utr5-6hpf_pa | active | CGUUU | 1.315105 | 3.68E−06 |
| SEQ ID NO: 856 | 12h_hek293t_utr5-6hpf_pa | active | CUCGUU | 1.84631 | 3.81E−06 |
| SEQ ID NO: 857 | 12h_hek293t_utr5-6hpf_pa | active | ACUCUC | 2.014428 | 4.36E−06 |
| SEQ ID NO: 858 | 12h_hek293t_utr5-6hpf_pa | active | CCUCA | 1.486045 | 4.61E−06 |
| SEQ ID NO: 859 | 12h_hek2931_utr5-6hpf_pa | active | GCCUC | 1.519818 | 4.79E−06 |
| SEQ ID NO: 860 | 12h_hek293t_utr5-6hpf_pa | active | UUAUU | 1.289487 | 5.1E−06 |
| SEQ ID NO: 861 | 12h_hek293t_utr5-6hpf_pa | active | GCUCUU | 1.779506 | 5.28E−06 |
| SEQ ID NO: 862 | 12h_hek293t_utr5-6hpf_pa | active | CCGUUU | 1.694441 | 6.22E−06 |
| SEQ ID NO: 863 | 12h_hek293t_utr5-6hpf_pa | active | CUUUCU | 1.660584 | 6.6E−06 |
| SEQ ID NO: 864 | 12h_hek293t_utr5-6hpf_pa | active | CCUUCC | 2.045385 | 7.66E−06 |
| SEQ ID NO: 865 | 12h_hek293t_utr5-6hpf_pa | active | UUGUUU | 1.387537 | 8.75E−06 |
| SEQ ID NO: 866 | 12h_hek293t_utr5-6hpf_pa | active | CUCUA | 1.591339 | 8.98E−06 |
| SEQ ID NO: 867 | 12h_hek293t_utr5-6hpf_pa | repressed | AUGGC | 3.239571 | 1.27E−52 |
| SEQ ID NO: 868 | 12h_hek293t_utr5-6hpf_pa | repressed | GGAUG | 2.672621 | 4.48E−39 |
| SEQ ID NO: 869 | 12h_hek293t_utr5-6hpf_pa | repressed | GAUGG | 2.852782 | 1.11E−38 |
| SEQ ID NO: 870 | 12h_hek293t_utr5-6hpf_pa | repressed | AUGGA | 2.613477 | 9.66E−38 |
| SEQ ID NO: 871 | 12h_hek293t_utr5-6hpf_pa | repressed | AGGAUG | 4.798628 | 1.94E−37 |
| SEQ ID NO: 872 | 12h_hek293t_utr5-6hpf_pa | repressed | CAUGG | 2.809451 | 4.05E−37 |
| SEQ ID NO: 873 | 12h_hek293t_utr5-6hpf_pa | repressed | CCAUG | 2.886296 | 1.56E−29 |
| SEQ ID NO: 874 | 12h_hek293t_utr5-6hpf_pa | repressed | AAUGG | 2.522589 | 2.56E−27 |
| SEQ ID NO: 875 | 12h_hek293t_utr5-6hpf_pa | repressed | AUGGG | 2.758856 | 1.79E−26 |
| SEQ ID NO: 876 | 12h_hek293t_utr5-6hpf_pa | repressed | AGAUG | 2.307041 | 2.24E−25 |
| SEQ ID NO: 877 | 12h_hek293t_utr5-6hpf_pa | repressed | GAUGGC | 4.325005 | 1.33E−24 |
| SEQ ID NO: 878 | 12h_hek293t_utr5-6hpf_pa | repressed | CAAUG | 2.444644 | 5.51E−23 |
| SEQ ID NO: 879 | 12h_hek293t_utr5-6hpf_pa | repressed | AAAUG | 2.040136 | 6.08E−23 |
| SEQ ID NO: 880 | 12h_hek293t_utr5-6hpf_pa | repressed | GAUGU | 2.109187 | 3.29E−22 |
| SEQ ID NO: 881 | 12h_hek293t_utr5-6hpf_pa | repressed | AAAAUG | 3.20221 | 3.98E−22 |
| SEQ ID NO: 882 | 12h_hek293t_utr5-6hpf_pa | repressed | AUCAUG | 3.671267 | 1.99E−21 |
| SEQ ID NO: 883 | 12h_hek293t_utr5-6hpf_pa | repressed | AUGUG | 1.960474 | 7.94E−21 |
| SEQ ID NO: 884 | 12h_hek293t_utr5-6hpf_pa | repressed | UCAUG | 2.083328 | 2.11E−20 |
| SEQ ID NO: 885 | 12h_hek293t_utr5-6hpf_pa | repressed | AUGGCG | 3.629915 | 6.38E−20 |
| SEQ ID NO: 886 | 12h_hek293t_utr5-6hpf_pa | repressed | GAUGA | 2.092981 | 9.73E−20 |
| SEQ ID NO: 887 | 12h_hek293t_utr5-6hpf_pa | repressed | AAUGGC | 3.521721 | 1.88E−19 |
| SEQ ID NO: 888 | 12h_hek293t_utr5-6hpf_pa | repressed | ACAAUG | 3.883782 | 2.31E−19 |
| SEQ ID NO: 889 | 12h_hek293t_utr5-6hpf_pa | repressed | AUGGAG | 2.960692 | 1.24E−18 |
| SEQ ID NO: 890 | 12h_hek293t_utr5-6hpf_pa | repressed | GGAUGC | 3.543291 | 7.29E−18 |
| SEQ ID NO: 891 | 12h_hek293t_utr5-6hpf_pa | repressed | UCAUGG | 3.2611 | 2.64E−17 |
| SEQ ID NO: 892 | 12h_hek293t_utr5-6hpf_pa | repressed | AUGGCA | 3.371129 | 2.77E−17 |
| SEQ ID NO: 893 | 12h_hek293t_utr5-6hpf_pa | repressed | AUGAG | 2.016757 | 3.21E−17 |
| SEQ ID NO: 894 | 12h_hek293t_utr5-6hpf_pa | repressed | CGAUG | 2.340634 | 1.82E−16 |
| SEQ ID NO: 895 | 12h_hek293t_utr5-6hpf_pa | repressed | AAAUGG | 3.099114 | 2.32E−16 |
| SEQ ID NO: 896 | 12h_hek293t_utr5-6hpf_pa | repressed | AUGAGG | 3.140464 | 3.49E−16 |
| SEQ ID NO: 897 | 12h_hek293t_utr5-6hpf_pa | repressed | GAUGC | 2.133859 | 7.05E−16 |
| SEQ ID NO: 898 | 12h_hek293t_utr5-6hpf_pa | repressed | AAUGU | 1.830347 | 8.46E−16 |
| SEQ ID NO: 899 | 12h_hek293t_utr5-6hpf_pa | repressed | ACCAUG | 4.221016 | 1.73E−15 |
| SEQ ID NO: 900 | 12h_hek293t_utr5-6hpf_pa | repressed | GCCAUG | 3.532825 | 3.45E−15 |
| SEQ ID NO: 901 | 12h_hek293t_utr5-6hpf_pa | repressed | AUGAA | 1.848871 | 6.74E−15 |
| SEQ ID NO: 902 | 12h_hek293t_utr5-6hpf_pa | repressed | AGGAU | 1.867149 | 8.37E−15 |
| SEQ ID NO: 903 | 12h_hek293t_utr5-6hpf_pa | repressed | CAUGGG | 3.822437 | 3.78E−14 |
| SEQ ID NO: 904 | 12h_hek293t_utr5-6hpf_pa | repressed | AUGGCU | 3.112883 | 4.6E−14 |
| SEQ ID NO: 905 | 12h_hek293t_utr5-6hpf_pa | repressed | UGAUG | 1.808931 | 6.74E−14 |
| SEQ ID NO: 906 | 12h_hek293t_utr5-6hpf_pa | repressed | GGGAUG | 3.104703 | 1.56E−13 |
| SEQ ID NO: 907 | 12h_hek293t_utr5-6hpf_pa | repressed | UGGAG | 1.643343 | 1.65E−13 |
| SEQ ID NO: 908 | 12h_hek293t_utr5-6hpf_pa | repressed | CCAUGG | 3.357819 | 2.02E−13 |
| SEQ ID NO: 909 | 12h_hek293t_utr5-6hpf_pa | repressed | GUCAUG | 3.120127 | 2.19E−13 |
| SEQ ID NO: 910 | 12h_hek293t_utr5-6hpf_pa | repressed | CAUGGA | 2.683216 | 3.51E−13 |
| SEQ ID NO: 911 | 12h_hek293t_utr5-6hpf_pa | repressed | AUGUC | 1.966307 | 4.1E−13 |
| SEQ ID NO: 912 | 12h_hek293t_utr5-6hpf_pa | repressed | AAGAUG | 2.911256 | 5.3E−13 |
| SEQ ID NO: 913 | 12h_hek293t_utr5-6hpf_pa | repressed | AGAUGG | 2.964269 | 6.67E−13 |
| SEQ ID NO: 914 | 12h_hek293t_utr5-6hpf_pa | repressed | GAUGUG | 2.499286 | 7.05E−13 |
| SEQ ID NO: 915 | 12h_hek293t_utr5-6hpf_pa | repressed | UAUGG | 2.065422 | 7.3E−13 |
| SEQ ID NO: 916 | 12h_hek293t_utr5-6hpf_pa | repressed | CAAUGU | 2.906146 | 2.53E−12 |
| SEQ ID NO: 917 | 12h_hek293t_utr5-6hpf_pa | repressed | AUGAUG | 2.803431 | 2.71E−12 |
| SEQ ID NO: 918 | 12h_hek293t_utr5-6hpf_pa | repressed | GGAUGG | 3.141959 | 2.71E−12 |
| SEQ ID NO: 919 | 12h_hek293t_utr5-6hpf_pa | repressed | GAUGGA | 2.691538 | 4.07E−12 |
| SEQ ID NO: 920 | 12h_hek293t_utr5-6hpf_pa | repressed | AAAUGA | 2.339075 | 5E−12 |
| SEQ ID NO: 921 | 12h_hek293t_utr5-6hpf_pa | repressed | AUGUGG | 2.600154 | 8.01E−12 |
| SEQ ID NO: 922 | 12h_hek293t_utr5-6hpf_pa | repressed | AACAUG | 2.755969 | 1.38E−11 |
| SEQ ID NO: 923 | 12h_hek293t_utr5-6hpf_pa | repressed | AUGGGA | 2.721735 | 1.47E−11 |
| SEQ ID NO: 924 | 12h_hek293t_utr5-6hpf_pa | repressed | UAUGGA | 2.865035 | 1.95E−11 |
| SEQ ID NO: 925 | 12h_hek293t_utr5-6hpf_pa | repressed | ACAUG | 1.836895 | 2.57E−11 |
| SEQ ID NO: 926 | 12h_hek293t_utr5-6hpf_pa | repressed | CAUGA | 1.861995 | 2.59E−11 |
| SEQ ID NO: 927 | 12h_hek293t_utr5-6hpf_pa | repressed | CAUGGC | 2.683444 | 4.03E−11 |
| SEQ ID NO: 928 | 12h_hek293t_utr5-6hpf_pa | repressed | ACGAUG | 3.689422 | 4.35E−11 |
| SEQ ID NO: 929 | 12h_hek293t_utr5-6hpf_pa | repressed | CCAUGU | 3.119108 | 5.82E−11 |
| SEQ ID NO: 930 | 12h_hek293t_utr5-6hpf_pa | repressed | GGAUGA | 2.456194 | 1.35E−10 |
| SEQ ID NO: 931 | 12h_hek293t_utr5-6hpf_pa | repressed | GCGAUG | 2.948797 | 1.35E−10 |
| SEQ ID NO: 932 | 12h_hek293t_utr5-6hpf_pa | repressed | UGGCG | 1.78031 | 2.4E−10 |
| SEQ ID NO: 933 | 12h_hek293t_utr5-6hpf_pa | repressed | GAAAUG | 2.511131 | 2.46E−10 |
| SEQ ID NO: 934 | 12h_hek293t_utr5-6hpf_pa | repressed | AUGGAA | 2.669691 | 2.74E−10 |
| SEQ ID NO: 935 | 12h_hek293t_utr5-6hpf_pa | repressed | CGAUGG | 3.076044 | 4.79E−10 |
| SEQ ID NO: 936 | 12h_hek293t_utr5-6hpf_pa | repressed | AAUGA | 1.665451 | 4.99E−10 |
| SEQ ID NO: 937 | 12h_hek293t_utr5-6hpf_pa | repressed | GAGAUG | 2.720741 | 8.08E−10 |
| SEQ ID NO: 938 | 12h_hek293t_utr5-6hpf_pa | repressed | AUGGAC | 2.665905 | 1.04E−09 |
| SEQ ID NO: 939 | 12h_hek293t_utr5-6hpf_pa | repressed | AGAAUG | 2.935669 | 1.19E−09 |
| SEQ ID NO: 940 | 12h_hek293t_utr5-6hpf_pa | repressed | UGUGGC | 2.378175 | 1.41E−09 |
| SEQ ID NO: 941 | 12h_hek293t_utr5-6hpf_pa | repressed | AUGAU | 1.742244 | 1.49E−09 |
| SEQ ID NO: 942 | 12h_hek293t_utr5-6hpf_pa | repressed | AGCCAU | 2.45028 | 1.9E−09 |
| SEQ ID NO: 943 | 12h_hek293t_utr5-6hpf_pa | repressed | GAUGCG | 2.925993 | 2.19E−09 |
| SEQ ID NO: 944 | 12h_hek293t_utr5-6hpf_pa | repressed | GCAAUG | 2.77293 | 4.22E−09 |
| SEQ ID NO: 945 | 12h_hek293t_utr5-6hpf_pa | repressed | GAUGAG | 2.323897 | 4.69E−09 |
| SEQ ID NO: 946 | 12h_hek293t_utr5-6hpf_pa | repressed | AUAUGA | 2.46003 | 4.97E−09 |
| SEQ ID NO: 947 | 12h_hek293t_utr5-6hpf_pa | repressed | AGAUGU | 2.313749 | 5.51E−09 |
| SEQ ID NO: 948 | 12h_hek293t_utr5-6hpf_pa | repressed | CAUGAC | 3.046748 | 5.57E−09 |
| SEQ ID NO: 949 | 12h_hek293t_utr5-6hpf_pa | repressed | CAUGU | 1.661192 | 7.77E−09 |
| SEQ ID NO: 950 | 12h_hek293t_utr5-6hpf_pa | repressed | UGGCA | 1.757511 | 1.06E−08 |
| SEQ ID NO: 951 | 12h_hek293t_utr5-6hpf_pa | repressed | CAUGC | 1.793563 | 1.13E−08 |
| SEQ ID NO: 952 | 12h_hek293t_utr5-6hpf_pa | repressed | AUGAAG | 2.152669 | 1.37E−08 |
| SEQ ID NO: 953 | 12h_hek293t_utr5-6hpf_pa | repressed | CAGGAU | 2.349328 | 2.34E−08 |
| SEQ ID NO: 954 | 12h_hek293t_utr5-6hpf_pa | repressed | AAUGUG | 2.037035 | 2.53E−08 |
| SEQ ID NO: 955 | 12h_hek293t_utr5-6hpf_pa | repressed | AUGCC | 1.975959 | 2.74E−08 |
| SEQ ID NO: 956 | 12h_hek293t_utr5-6hpf_pa | repressed | AAUAUG | 2.499286 | 2.82E−08 |
| SEQ ID NO: 957 | 12h_hek293t_utr5-6hpf_pa | repressed | AGCAUG | 2.499286 | 4.12E−08 |
| SEQ ID NO: 958 | 12h_hek293t_utr5-6hpf_pa | repressed | GCCAU | 1.68347 | 5.81E−08 |
| SEQ ID NO: 959 | 12h_hek293t_utr5-6hpf_pa | repressed | GAAUG | 1.588946 | 6.22E−08 |
| SEQ ID NO: 960 | 12h_hek293t_utr5-6hpf_pa | repressed | GGGAU | 1.583926 | 6.73E−08 |
| SEQ ID NO: 961 | 12h_hek293t_utr5-6hpf_pa | repressed | UUAUGG | 2.425342 | 6.93E−08 |
| SEQ ID NO: 962 | 12h_hek293t_utr5-6hpf_pa | repressed | AGAUGA | 2.226636 | 7.28E−08 |
| SEQ ID NO: 963 | 12h_hek293t_utr5-6hpf_pa | repressed | UGAUGU | 1.993101 | 9.41E−08 |
| SEQ ID NO: 964 | 12h_hek293t_utr5-6hpf_pa | repressed | AUGCG | 1.756531 | 1.14E−07 |
| SEQ ID NO: 965 | 12h_hek293t_utr5-6hpf_pa | repressed | GUGAUG | 2.303007 | 1.18E−07 |
| SEQ ID NO: 966 | 12h_hek293t_utr5-6hpf_pa | repressed | UAUGA | 1.709049 | 1.25E−07 |
| SEQ ID NO: 967 | 12h_hek293t_utr5-6hpf_pa | repressed | UGAUGG | 2.26066 | 1.52E−07 |
| SEQ ID NO: 968 | 12h_hek293t_utr5-6hpf_pa | repressed | AAUGGG | 2.575022 | 1.91E−07 |
| SEQ ID NO: 969 | 12h_hek293t_utr5-6hpf_pa | repressed | GAUGUC | 2.535507 | 1.91E−07 |
| SEQ ID NO: 970 | 12h_hek293t_utr5-6hpf_pa | repressed | AAUGAG | 2.115578 | 2.21E−07 |
| SEQ ID NO: 971 | 12h_hek293t_utr5-6hpf_pa | repressed | AUGGCC | 2.70193 | 3.17E−07 |
| SEQ ID NO: 972 | 12h_hek293t_utr5-6hpf_pa | repressed | AUGGGU | 3.10722 | 5E−07 |
| SEQ ID NO: 973 | 12h_hek293t_utr5-6hpf_pa | repressed | CAGAUG | 2.055864 | 5.55E−07 |
| SEQ ID NO: 974 | 12h_hek293t_utr5-6hpf_pa | repressed | UGGCAG | 2.138924 | 6.04E−07 |
| SEQ ID NO: 975 | 12h_hek293t_utr5-6hpf_pa | repressed | UGGCCG | 2.239797 | 6.8E−07 |
| SEQ ID NO: 976 | 12h_hek293t_utr5-6hpf_pa | repressed | CGAUGA | 2.480494 | 7.21E−07 |
| SEQ ID NO: 977 | 12h_hek293t_utr5-6hpf_pa | repressed | GAUGAU | 2.11921 | 7.95E−07 |
| SEQ ID NO: 978 | 12h_hek293t_utr5-6hpf_pa | repressed | GAUGAA | 2.109489 | 9.1E−07 |
| SEQ ID NO: 979 | 12h_hek293t_utr5-6hpf_pa | repressed | ACAUGG | 2.336994 | 1.09E−06 |
| SEQ ID NO: 980 | 12h_hek293t_utr5-6hpf_pa | repressed | AUGUGU | 1.830463 | 1.41E−06 |
| SEQ ID NO: 981 | 12h_hek293t_utr5-6hpf_pa | repressed | AUGUA | 1.600698 | 1.46E−06 |
| SEQ ID NO: 982 | 12h_hek293t_utr5-6hpf_pa | repressed | AGGGG | 1.647543 | 1.5E−06 |
| SEQ ID NO: 983 | 12h_hek293t_utr5-6hpf_pa | repressed | AUGUCG | 2.360436 | 1.67E−06 |
| SEQ ID NO: 984 | 12h_hek293t_utr5-6hpf_pa | repressed | CAGGGG | 2.42355 | 1.81E−06 |
| SEQ ID NO: 985 | 12h_hek293t_utr5-6hpf_pa | repressed | GAGAU | 1.529624 | 1.83E−06 |
| SEQ ID NO: 986 | 12h_hek293t_utr5-6hpf_pa | repressed | CAAUGG | 2.499286 | 1.9E−06 |
| SEQ ID NO: 987 | 12h_hek293t_utr5-6hpf_pa | repressed | GGGGC | 1.652929 | 2.17E−06 |
| SEQ ID NO: 988 | 12h_hek293t_utr5-6hpf_pa | repressed | AAUGUC | 2.145983 | 2.18E−06 |
| SEQ ID NO: 989 | 12h_hek293t_utr5-6hpf_pa | repressed | GAGGAU | 1.823008 | 2.79E−06 |
| SEQ ID NO: 990 | 12h_hek293t_utr5-6hpf_pa | repressed | UGGAGA | 1.831308 | 2.86E−06 |
| SEQ ID NO: 991 | 12h_hek293t_utr5-6hpf_pa | repressed | GGAUGU | 2.025853 | 2.92E−06 |
| SEQ ID NO: 992 | 12h_hek293t_utr5-6hpf_pa | repressed | UGGCC | 1.618537 | 3.18E−06 |
| SEQ ID NO: 993 | 12h_hek293t_utr5-6hpf_pa | repressed | AAUGGA | 2.183284 | 3.26E−06 |
| SEQ ID NO: 994 | 12h_hek293t_utr5-6hpf_pa | repressed | UGGAGG | 1.900865 | 3.3E−06 |
| SEQ ID NO: 995 | 12h_hek293t_utr5-6hpf_pa | repressed | AUAUG | 1.62092 | 3.37E−06 |
| SEQ ID NO: 996 | 12h_hek293t_utr5-6hpf_pa | repressed | AUGUCA | 2.15408 | 3.45E−06 |
| SEQ ID NO: 997 | 12h_hek293t_utr5-6hpf_pa | repressed | AUGAC | 1.620439 | 3.84E−06 |
| SEQ ID NO: 998 | 12h_hek293t_utr5-6hpf_pa | repressed | AUGGGG | 2.658814 | 4.03E−06 |
| SEQ ID NO: 999 | 12h_hek293t_utr5-6hpf_pa | repressed | GACAUG | 2.242949 | 4.11E−06 |
| SEQ ID NO: 1000 | 12h_hek293t_utr5-6hpf_pa | repressed | GCAUGG | 2.266019 | 4.41E−06 |
| SEQ ID NO: 1001 | 12h_hek293t_utr5-6hpf_pa | repressed | GAUGGG | 2.436278 | 4.82E−06 |
| SEQ ID NO: 1002 | 12h_hek293t_utr5-6hpf_pa | repressed | CCAUGA | 2.477168 | 4.94E−06 |
| SEQ ID NO: 1003 | 12h_hek293t_utr5-6hpf_pa | repressed | AUGGGC | 2.630827 | 4.95E−06 |
| SEQ ID NO: 1004 | 12h_hek293t_utr5-6hpf_pa | repressed | GAAGAU | 1.838184 | 5.25E−06 |
| SEQ ID NO: 1005 | 12h_hek293t_utr5-6hpf_pa | repressed | GAGGA | 1.347824 | 5.44E−06 |
| SEQ ID NO: 1006 | 12h_hek293t_utr5-6hpf_pa | repressed | AUGAAU | 2.018202 | 6.64E−06 |
| SEQ ID NO: 1007 | 12h_hek293t_utr5-6hpf_pa | repressed | CAGGG | 1.572623 | 6.78E−06 |
| SEQ ID NO: 1008 | 12h_hek293t_utr5-6hpf_pa | repressed | UCAUGA | 2.096175 | 6.84E−06 |
| SEQ ID NO: 1009 | 12h_hek293t_utr5-6hpf_pa | repressed | UCCAUG | 2.221587 | 6.97E−06 |
| SEQ ID NO: 1010 | 12h_hek293t_utr5-6hpf_pa | repressed | CACGAU | 2.499286 | 8.9E−06 |
| SEQ ID NO: 1011 | 12h_hek293t_utr5-6hpf_pa | s1_activated | GUGUGU | 1.895916 | 1.02E−14 |
| SEQ ID NO: 1012 | 12h_hek293t_utr5-6hpf_pa | s1_activated | AGCGG | 1.746004 | 5.05E−14 |
| SEQ ID NO: 1013 | 12h_hek293t_utr5-6hpf_pa | s1_activated | CGGUG | 1.837691 | 1.2E−13 |
| SEQ ID NO: 1014 | 12h_hek293t_utr5-6hpf_pa | s1_activated | CGGAG | 1.735464 | 1.2E−13 |
| SEQ ID NO: 1015 | 12h_hek293t_utr5-6hpf_pa | s1_activated | GAGGG | 1.832447 | 2.59E−13 |
| SEQ ID NO: 1016 | 12h_hek293t_utr5-6hpf_pa | s1_activated | GUGUG | 1.518683 | 9.11E−12 |
| SEQ ID NO: 1017 | 12h_hek293t_utr5-6hpf_pa | s1_activated | UGUGUG | 1.787233 | 1.37E−11 |
| SEQ ID NO: 1018 | 12h_hek293t_utr5-6hpf_pa | s1_activated | GCGGC | 1.722584 | 1.38E−11 |
| SEQ ID NO: 1019 | 12h_hek293t_utr5-6hpf_pa | s1_activated | AGGGC | 2.014186 | 1.48E−11 |
| SEQ ID NO: 1020 | 12h_hek293t_utr5-6hpf_pa | s1_activated | GGCGG | 1.727637 | 1.77E−10 |
| SEQ ID NO: 1021 | 12h_hek293t_utr5-6hpf_pa | s1_activated | GGAGG | 1.638603 | 2.52E−10 |
| SEQ ID NO: 1022 | 12h_hek293t_utr5-6hpf_pa | s1_activated | GAGAG | 1.554956 | 2.91E−10 |
| SEQ ID NO: 1023 | 12h_hek293t_utr5-6hpf_pa | s1_activated | GGGAG | 1.727573 | 3.71E−10 |
| SEQ ID NO: 1024 | 12h_hek293t_utr5-6hpf_pa | s1_activated | GGGAGG | 2.586134 | 5.81E−10 |
| SEQ ID NO: 1025 | 12h_hek293t_utr5-6hpf_pa | s1_activated | AGAGG | 1.624899 | 6.75E−10 |
| SEQ ID NO: 1026 | 12h_hek293t_utr5-6hpf_pa | s1_activated | GAGGA | 1.478736 | 1.24E−09 |
| SEQ ID NO: 1027 | 12h_hek293t_utr5-6hpf_pa | s1_activated | AGGGCG | 2.86322 | 1.59E−09 |
| SEQ ID NO: 1028 | 12h_hek293t_utr5-6hpf_pa | s1_activated | GGGCG | 1.869714 | 1.84E−09 |
| SEQ ID NO: 1029 | 12h_hek293t_utr5-6hpf_pa | s1_activated | GGGUG | 1.792288 | 1.89E−09 |
| SEQ ID NO: 1030 | 12h_hek293t_utr5-6hpf_pa | s1_activated | GCGCG | 1.586221 | 2.22E−09 |
| SEQ ID NO: 1031 | 12h_hek293t_utr5-6hpf_pa | s1_activated | CGAGG | 1.671706 | 3.15E−09 |
| SEQ ID NO: 1032 | 12h_hek293t_utr5-6hpf_pa | s1_activated | GGACG | 1.673852 | 4.91E−09 |
| SEQ ID NO: 1033 | 12h_hek293t_utr5-6hpf_pa | s1_activated | GAGGGU | 2.720059 | 7.6E−09 |
| SEQ ID NO: 1034 | 12h_hek293t_utr5-6hpf_pa | s1_activated | GCGUG | 1.578834 | 1.36E−08 |
| SEQ ID NO: 1035 | 12h_hek293t_utr5-6hpf_pa | s1_activated | GGAAG | 1.530931 | 1.41E−08 |
| SEQ ID NO: 1036 | 12h_hek293t_utr5-6hpf_pa | s1_activated | GAAGAG | 1.916901 | 1.52E−08 |
| SEQ ID NO: 1037 | 12h_hek293t_utr5-6hpf_pa | s1_activated | AAGGG | 1.742183 | 2.31E−08 |
| SEQ ID NO: 1038 | 12h_hek293t_utr5-6hpf_pa | s1_activated | CGGCG | 1.610086 | 2.85E−08 |
| SEQ ID NO: 1039 | 12h_hek293t_utr5-6hpf_pa | s1_activated | GAGCG | 1.513998 | 5.21E−08 |
| SEQ ID NO: 1040 | 12h_hek293t_utr5-6hpf_pa | s1_activated | CGGGG | 1.76335 | 5.96E−08 |
| SEQ ID NO: 1041 | 12h_hek293t_utr5-6hpf_pa | s1_activated | GCGGG | 1.633457 | 6.41E−08 |
| SEQ ID NO: 1042 | 12h_hek293t_utr5-6hpf_pa | s1_activated | CGGAGG | 2.092353 | 6.75E−08 |
| SEQ ID NO: 1043 | 12h_hek293t_utr5-6hpf_pa | s1_activated | CGUGA | 1.639291 | 7.6E−08 |
| SEQ ID NO: 1044 | 12h_hek293t_utr5-6hpf_pa | s1_activated | AGCGCG | 1.936884 | 7.74E−08 |
| SEQ ID NO: 1045 | 12h_hek293t_utr5-6hpf_pa | s1_activated | AGGGU | 1.779558 | 8.68E−08 |
| SEQ ID NO: 1046 | 12h_hek293t_utr5-6hpf_pa | s1_activated | AAGAG | 1.514351 | 8.72E−08 |
| SEQ ID NO: 1047 | 12h_hek293t_utr5-6hpf_pa | s1_activated | ACCGG | 1.699755 | 1.08E−07 |
| SEQ ID NO: 1048 | 12h_hek293t_utr5-6hpf_pa | s1_activated | CGAGC | 1.530719 | 1.09E−07 |
| SEQ ID NO: 1049 | 12h_hek293t_utr5-6hpf_pa | s1_activated | GCGAG | 1.518561 | 1.14E−07 |
| SEQ ID NO: 1050 | 12h_hek293t_utr5-6hpf_pa | s1_activated | CGGUGU | 1.997704 | 1.37E−07 |
| SEQ ID NO: 1051 | 12h_hek293t_utr5-6hpf_pa | s1_activated | GAGUGU | 1.85122 | 1.49E−07 |
| SEQ ID NO: 1052 | 12h_hek293t_utr5-6hpf_pa | s1_activated | CGGGGG | 2.672338 | 1.73E−07 |
| SEQ ID NO: 1053 | 12h_hek293t_utr5-6hpf_pa | s1_activated | GCGGA | 1.501702 | 1.93E−07 |
| SEQ ID NO: 1054 | 12h_hek293t_utr5-6hpf_pa | s1_activated | GGGGG | 1.78412 | 2.08E−07 |
| SEQ ID NO: 1055 | 12h_hek293t_utr5-6hpf_pa | s1_activated | CCGGU | 1.663471 | 3.08E−07 |
| SEQ ID NO: 1056 | 12h_hek293t_utr5-6hpf_pa | s1_activated | GAGUG | 1.480611 | 3.1E−07 |
| SEQ ID NO: 1057 | 12h_hek293t_utr5-6hpf_pa | s1_activated | CGCGCG | 1.977441 | 3.11E−07 |
| SEQ ID NO: 1058 | 12h_hek293t_utr5-6hpf_pa | s1_activated | AGCGC | 1.487014 | 4.17E−07 |
| SEQ ID NO: 1059 | 12h_hek293t_utr5-6hpf_pa | s1_activated | CCGGUG | 2.069169 | 4.54E−07 |
| SEQ ID NO: 1060 | 12h_hek293t_utr5-6hpf_pa | s1_activated | GCCGAG | 2.226949 | 5.93E−07 |
| SEQ ID NO: 1061 | 12h_hek293t_utr5-6hpf_pa | s1_activated | CGUGAG | 2.226949 | 5.93E−07 |
| SEQ ID NO: 1062 | 12h_hek293t_utr5-6hpf_pa | s1_activated | GGAGA | 1.429561 | 6.76E−07 |
| SEQ ID NO: 1063 | 12h_hek293t_utr5-6hpf_pa | s1_activated | GUUUG | 1.322868 | 7E−07 |
| SEQ ID NO: 1064 | 12h_hek293t_utr5-6hpf_pa | s1_activated | GGUGU | 1.464069 | 7.79E−07 |
| SEQ ID NO: 1065 | 12h_hek293t_utr5-6hpf_pa | s1_activated | GCGGCG | 1.962206 | 7.87E−07 |
| SEQ ID NO: 1066 | 12h_hek293t_utr5-6hpf_pa | s1_activated | CGGGC | 1.665736 | 8.2E−07 |
| SEQ ID NO: 1067 | 12h_hek293t_utr5-6hpf_pa | s1_activated | AGGAG | 1.435208 | 8.42E−07 |
| SEQ ID NO: 1068 | 12h_hek293t_utr5-6hpf_pa | s1_activated | CCGGG | 1.662767 | 8.88E−07 |
| SEQ ID NO: 1069 | 12h_hek293t_utr5-6hpf_pa | s1_activated | GCGUGA | 2.140465 | 1.03E−06 |
| SEQ ID NO: 1070 | 12h_hek293t_utr5-6hpf_pa | s1_activated | GGAGGG | 2.064467 | 1.04E−06 |
| SEQ ID NO: 1071 | 12h_hek293t_utr5-6hpf_pa | s1_activated | GGGGAG | 2.180794 | 1.07E−06 |
| SEQ ID NO: 1072 | 12h_hek293t_utr5-6hpf_pa | s1_activated | AAAAGG | 2.028597 | 1.08E−06 |
| SEQ ID NO: 1073 | 12h_hek293t_utr5-6hpf_pa | s1_activated | AGGAAG | 1.840063 | 1.18E−06 |
| SEQ ID NO: 1074 | 12h_hek293t_utr5-6hpf_pa | s1_activated | GUGUGC | 1.777171 | 1.26E−06 |
| SEQ ID NO: 1075 | 12h_hek293t_utr5-6hpf_pa | s1_activated | AGUGG | 1.535493 | 1.26E−06 |
| SEQ ID NO: 1076 | 12h_hek293t_utr5-6hpf_pa | s1_activated | CGCGG | 1.503983 | 1.27E−06 |
| SEQ ID NO: 1077 | 12h_hek293t_utr5-6hpf_pa | s1_activated | CCGCG | 1.549278 | 1.74E−06 |
| SEQ ID NO: 1078 | 12h_hek293t_utr5-6hpf_pa | s1_activated | CCGAGC | 2.030171 | 2.17E−06 |
| SEQ ID NO: 1079 | 12h_hek293t_utr5-6hpf_pa | s1_activated | GGGGGA | 2.166761 | 2.24E−06 |
| SEQ ID NO: 1080 | 12h_hek293t_utr5-6hpf_pa | s1_activated | GAGGC | 1.54107 | 2.26E−06 |
| SEQ ID NO: 1081 | 12h_hek293t_utr5-6hpf_pa | s1_activated | UCGUG | 1.480046 | 2.33E−06 |
| SEQ ID NO: 1082 | 12h_hek293t_utr5-6hpf_pa | s1_activated | GUGAAG | 1.739633 | 3.23E−06 |
| SEQ ID NO: 1083 | 12h_hek293t_utr5-6hpf_pa | s1_activated | UCGAG | 1.496353 | 3.27E−06 |
| SEQ ID NO: 1084 | 12h_hek293t_utr5-6hpf_pa | s1_activated | GCGGCC | 2.176047 | 3.55E−06 |
| SEQ ID NO: 1085 | 12h_hek293t_utr5-6hpf_pa | s1_activated | AAGGGC | 2.4245 | 3.73E−06 |
| SEQ ID NO: 1086 | 12h_hek293t_utr5-6hpf_pa | s1_activated | GAGCGG | 1.903682 | 3.87E−06 |
| SEQ ID NO: 1087 | 12h_hek293t_utr5-6hpf_pa | s1_activated | GCGGAG | 1.795719 | 4.62E−06 |
| SEQ ID NO: 1088 | 12h_hek293t_utr5-6hpf_pa | s1_activated | GAGAGG | 1.795719 | 4.62E−06 |
| SEQ ID NO: 1089 | 12h_hek293t_utr5-6hpf_pa | s1_activated | AGUAGG | 2.991423 | 5.24E−06 |
| SEQ ID NO: 1090 | 12h_hek293t_utr5-6hpf_pa | s1_activated | AAGGCG | 2.112592 | 5.65E−06 |
| SEQ ID NO: 1091 | 12h_hek293t_utr5-6hpf_pa | s1_activated | GGCGGG | 2.087764 | 5.85E−06 |
| SEQ ID NO: 1092 | 12h_hek293t_utr5-6hpf_pa | s1_activated | AAAGG | 1.466214 | 6.55E−06 |
| SEQ ID NO: 1093 | 12h_hek293t_utr5-6hpf_pa | s1_activated | GACGUG | 2.143277 | 6.84E−06 |
| SEQ ID NO: 1094 | 12h_hek293t_utr5-6hpf_pa | s1_activated | CGGGA | 1.505184 | 6.88E−06 |
| SEQ ID NO: 1095 | 12h_hek293t_utr5-6hpf_pa | s1_activated | GAAGA | 1.375533 | 7.04E−06 |
| SEQ ID NO: 1096 | 12h_hek293t_utr5-6hpf_pa | s1_activated | ACGGC | 1.551384 | 7.24E−06 |
| SEQ ID NO: 1097 | 12h_hek293t_utr5-6hpf_pa | s1_activated | CGCGUG | 1.819147 | 7.28E−06 |
| SEQ ID NO: 1098 | 12h_hek293t_utr5-6hpf_pa | s1_activated | CGAGGG | 2.130959 | 7.85E−06 |
| SEQ ID NO: 1099 | 12h_hek293t_utr5-6hpf_pa | s1_activated | AGGAA | 1.404233 | 8.6E−06 |
| SEQ ID NO: 1100 | 12h_hek293t_utr5-6hpf_pa | s1_activated | GCCGA | 1.547876 | 8.8E−06 |
| SEQ ID NO: 1101 | 12h_hek293t_utr5-6hpf_pa | s1_activated | CCGAG | 1.502559 | 8.86E−06 |
| SEQ ID NO: 1102 | 12h_hek293t_utr5-6hpf_pa | s1_activated | GGACGU | 2.013843 | 8.94E−06 |
| SEQ ID NO: 1103 | 12h_hek293t_utr5-6hpf_pa | s1_activated | GUGAG | 1.43578 | 9.11E−06 |
| SEQ ID NO: 1104 | 12h_hek293t_utr5-6hpf_pa | s1_activated | GGUGC | 1.603347 | 9.39E−06 |
| SEQ ID NO: 1105 | 12h_hek293t_utr5-6hpf_pa | s2_activated | UUUUU | 1.57601 | 9.23E−23 |
| SEQ ID NO: 1106 | 12h_hek293t_utr5-6hpf_pa | s2_activated | AUUUUU | 2.026174 | 3.75E−21 |
| SEQ ID NO: 1107 | 12h_hek293t_utr5-6hpf_pa | s2_activated | AUUUU | 1.507131 | 1.99E−17 |
| SEQ ID NO: 1108 | 12h_hek293t_utr5-6hpf_pa | s2_activated | UUUUA | 1.441173 | 2.02E−15 |
| SEQ ID NO: 1109 | 12h_hek293t_utr5-6hpf_pa | s2_activated | UUUUUA | 1.765453 | 9.31E−15 |
| SEQ ID NO: 1110 | 12h_hek293t_utr5-6hpf_pa | s2_activated | UUUUUU | 1.715011 | 4.61E−12 |
| SEQ ID NO: 1111 | 12h_hek293t_utr5-6hpf_pa | s2_activated | UAUUUU | 1.653592 | 1.91E−11 |
| SEQ ID NO: 1112 | 12h_hek293t_utr5-6hpf_pa | s2_activated | UAUUU | 1.378418 | 2.84E−10 |
| SEQ ID NO: 1113 | 12h_hek293t_utr5-6hpf_pa | s2_activated | UUUUAA | 1.588805 | 3.9E−10 |
| SEQ ID NO: 1114 | 12h_hek293t_utr5-6hpf_pa | s2_activated | UUUUC | 1.368257 | 4.02E−10 |
| SEQ ID NO: 1115 | 12h_hek293t_utr5-6hpf_pa | s2_activated | AAUUUU | 2.000847 | 8.33E−10 |
| SEQ ID NO: 1116 | 12h_hek293t_utr5-6hpf_pa | s2_activated | UCAUC | 1.512921 | 4.66E−09 |
| SEQ ID NO: 1117 | 12h_hek293t_utr5-6hpf_pa | s2_activated | UUCAU | 1.435069 | 1.09E−08 |
| SEQ ID NO: 1118 | 12h_hek293t_utr5-6hpf_pa | s2_activated | UUUAU | 1.348783 | 1.17E−08 |
| SEQ ID NO: 1119 | 12h_hek293t_utr5-6hpf_pa | s2_activated | UUUAC | 1.420796 | 2.08E−08 |
| SEQ ID NO: 1120 | 12h_hek293t_utr5-6hpf_pa | s2_activated | UCAUA | 1.642814 | 3.62E−08 |
| SEQ ID NO: 1121 | 12h_hek293t_utr5-6hpf_pa | s2_activated | CUUUU | 1.325354 | 4.3E−08 |
| SEQ ID NO: 1122 | 12h_hek293t_utr5-6hpf_pa | s2_activated | AUACU | 1.758702 | 6.73E−08 |
| SEQ ID NO: 1123 | 12h_hek293t_utr5-6hpf_pa | s2_activated | CAAUUU | 2.289526 | 7.48E−08 |
| SEQ ID NO: 1124 | 12h_hek293t_utr5-6hpf_pa | s2_activated | UAUGCU | 2.535319 | 8.75E−08 |
| SEQ ID NO: 1125 | 12h_hek293t_utr5-6hpf_pa | s2_activated | UAUGC | 1.786393 | 1.19E−07 |
| SEQ ID NO: 1126 | 12h_hek293t_utr5-6hpf_pa | s2_activated | UCAAU | 1.617866 | 1.22E−07 |
| SEQ ID NO: 1127 | 12h_hek293t_utr5-6hpf_pa | s2_activated | UUUUUC | 1.546817 | 1.99E−07 |
| SEQ ID NO: 1128 | 12h_hek293t_utr5-6hpf_pa | s2_activated | UUUAA | 1.292426 | 3.77E−07 |
| SEQ ID NO: 1129 | 12h_hek293t_utr5-6hpf_pa | s2_activated | UUAUUU | 1.461099 | 4.47E−07 |
| SEQ ID NO: 1130 | 12h_hek293t_utr5-6hpf_pa | s2_activated | GAUAAU | 2.299824 | 4.48E−07 |
| SEQ ID NO: 1131 | 12h_hek293t_utr5-6hpf_pa | s2_activated | CCUUU | 1.443012 | 5.54E−07 |
| SEQ ID NO: 1132 | 12h_hek293t_utr5-6hpf_pa | s2_activated | UGCUU | 1.418614 | 6.2E−07 |
| SEQ ID NO: 1133 | 12h_hek293t_utr5-6hpf_pa | s2_activated | UCACC | 1.539965 | 6.69E−07 |
| SEQ ID NO: 1134 | 12h_hek293t_utr5-6hpf_pa | s2_activated | CUUUUU | 1.511509 | 7.39E−07 |
| SEQ ID NO: 1135 | 12h_hek293t_utr5-6hpf_pa | s2_activated | CUCAAU | 2.229204 | 7.77E−07 |
| SEQ ID NO: 1136 | 12h_hek293t_utr5-6hpf_pa | s2_activated | UACUU | 1.5381 | 1.05E−06 |
| SEQ ID NO: 1137 | 12h_hek293t_utr5-6hpf_pa | s2_activated | AUUCCG | 2.613719 | 1.05E−06 |
| SEQ ID NO: 1138 | 12h_hek293t_utr5-6hpf_pa | s2_activated | UUUUCU | 1.495861 | 1.11E−06 |
| SEQ ID NO: 1139 | 12h_hek293t_utr5-6hpf_pa | s2_activated | AAUUU | 1.411579 | 1.52E−06 |
| SEQ ID NO: 1140 | 12h_hek293t_utr5-6hpf_pa | s2_activated | UAAUCC | 2.48105 | 1.53E−06 |
| SEQ ID NO: 1141 | 12h_hek293t_utr5-6hpf_pa | s2_activated | CAUUU | 1.320178 | 1.54E−06 |
| SEQ ID NO: 1142 | 12h_hek293t_utr5-6hpf_pa | s2_activated | UUUCU | 1.295044 | 1.63E−06 |
| SEQ ID NO: 1143 | 12h_hek293t_utr5-6hpf_pa | s2_activated | AUCAU | 1.478549 | 2.09E−06 |
| SEQ ID NO: 1144 | 12h_hek293t_utr5-6hpf_pa | s2_activated | UUUCA | 1.318861 | 2.22E−06 |
| SEQ ID NO: 1145 | 12h_hek293t_utr5-6hpf_pa | s2_activated | CACCU | 1.557394 | 2.23E−06 |
| SEQ ID NO: 1146 | 12h_hek293t_utr5-6hpf_pa | s2_activated | AUACUU | 2.210238 | 2.39E−06 |
| SEQ ID NO: 1147 | 12h_hek293t_utr5-6hpf_pa | s2_activated | UCAUU | 1.370745 | 2.55E−06 |
| SEQ ID NO: 1148 | 12h_hek293t_utr5-6hpf_pa | s2_activated | UCAAUU | 2.056945 | 3.12E−06 |
| SEQ ID NO: 1149 | 12h_hek293t_utr5-6hpf_pa | s2_activated | UUUUAU | 1.469304 | 4.31E−06 |
| SEQ ID NO: 1150 | 12h_hek293t_utr5-6hpf_pa | s2_activated | UUUACU | 1.693719 | 4.76E−06 |
| SEQ ID NO: 1151 | 12h_hek293t_utr5-6hpf_pa | s2_activated | ACACCU | 2.000847 | 5.23E−06 |
| SEQ ID NO: 1152 | 12h_hek293t_utr5-6hpf_pa | s2_activated | UCCCA | 1.596351 | 7.07E−06 |
| SEQ ID NO: 1153 | 12h_hek293t_utr5-6hpf_pa | s2_activated | CAUCA | 1.427746 | 7.55E−06 |
| SEQ ID NO: 1154 | 12h_hek293t_utr5-6hpf_pa | s2_activated | UUACU | 1.458888 | 7.76E−06 |
| SEQ ID NO: 1155 | 12h_hek293t_utr5-6hpf_pa | s2_activated | AUAUU | 1.335459 | 7.89E−06 |
| SEQ ID NO: 1156 | 12h_hek293t_utr5-6hpf_pa | s2_activated | UUAAU | 1.372036 | 9.59E−06 |

### Example 6-2: cis-Regulatory Elements Identified in Zebrafish Cells

In zebrafish cells, the following cis-regulatory elements were identified:

| | | | | Fold | |
|---|---|---|---|---|---|
| SEQ ID NO | Sample | Effect | Sequence | change | p-value |
| SEQ ID NO: 1157 | 2hpf_pa | active | UUUUU | 1.80313 | 1.59E−40 |
| SEQ ID NO: 1158 | 2hpf_pa | active | UUUUG | 1.71091 | 8.44E−33 |
| SEQ ID NO: 1159 | 2hpf_pa | active | UUUUUU | 2.25031 | 2.42E−30 |
| SEQ ID NO: 1160 | 2hpf_pa | active | UUUGU | 1.65684 | 4.20E−27 |
| SEQ ID NO: 1161 | 2hpf_pa | active | AUUUU | 1.62542 | 6.92E−25 |
| SEQ ID NO: 1162 | 2hpf_pa | active | UUGUU | 1.66608 | 3.07E−24 |
| SEQ ID NO: 1163 | 2hpf_pa | active | UUUUA | 1.56774 | 1.17E−23 |
| SEQ ID NO: 1164 | 2hpf_pa | active | UGUUU | 1.51273 | 2.19E−22 |
| SEQ ID NO: 1165 | 2hpf_pa | active | UUUUUG | 2.04552 | 1.60E−21 |
| SEQ ID NO: 1166 | 2hpf_pa | active | UUUGUU | 1.94913 | 1.61E−21 |
| SEQ ID NO: 1167 | 2hpf_pa | active | CUUUU | 1.58839 | 2.63E−21 |
| SEQ ID NO: 1168 | 2hpf_pa | active | GUUUU | 1.50459 | 7.41E−21 |
| SEQ ID NO: 1169 | 2hpf_pa | active | UAUUU | 1.55486 | 2.59E−19 |
| SEQ ID NO: 1170 | 2hpf_pa | active | UUUGC | 1.6802 | 6.85E−18 |
| SEQ ID NO: 1171 | 2hpf_pa | active | UUAUU | 1.56623 | 2.52E−17 |
| SEQ ID NO: 1172 | 2hpf_pa | active | UUUCU | 1.54551 | 2.97E−17 |
| SEQ ID NO: 1173 | 2hpf_pa | active | UUGUUU | 1.78224 | 3.42E−17 |
| SEQ ID NO: 1174 | 2hpf_pa | active | AUUUUU | 1.90331 | 4.36E−17 |
| SEQ ID NO: 1175 | 2hpf_pa | active | UUCUU | 1.66908 | 1.92E−16 |
| SEQ ID NO: 1176 | 2hpf_pa | active | UUUUUA | 1.75974 | 1.50E−14 |
| SEQ ID NO: 1177 | 2hpf_pa | active | UUUAU | 1.47111 | 5.26E−14 |
| SEQ ID NO: 1178 | 2hpf_pa | active | CUUUUU | 1.8087 | 6.90E−14 |
| SEQ ID NO: 1179 | 2hpf_pa | active | UAUUUU | 1.73051 | 1.16E−13 |
| SEQ ID NO: 1180 | 2hpf_pa | active | UUUUGU | 1.75721 | 1.76E−13 |
| SEQ ID NO: 1181 | 2hpf_pa | active | UUUUC | 1.4375 | 2.35E−13 |
| SEQ ID NO: 1182 | 2hpf_pa | active | UUUUAA | 1.68818 | 5.73E−13 |
| SEQ ID NO: 1183 | 2hpf_pa | active | UUUAUU | 1.69157 | 8.20E−13 |
| SEQ ID NO: 1184 | 2hpf_pa | active | GUUUUU | 1.67854 | 7.05E−12 |
| SEQ ID NO: 1185 | 2hpf_pa | active | UUUAA | 1.40369 | 8.12E−12 |
| SEQ ID NO: 1186 | 2hpf_pa | active | UUGUGU | 1.8442 | 2.38E−11 |
| SEQ ID NO: 1187 | 2hpf_pa | active | AUUUG | 1.50678 | 3.17E−11 |
| SEQ ID NO: 1188 | 2hpf_pa | active | UUUUCU | 1.69017 | 5.17E−11 |
| SEQ ID NO: 1189 | 2hpf_pa | active | ACUUU | 1.48957 | 5.66E−11 |
| SEQ ID NO: 1190 | 2hpf_pa | active | UUAUUU | 1.6104 | 7.54E−11 |
| SEQ ID NO: 1191 | 2hpf_pa | active | UGUUUU | 1.52742 | 9.11E−11 |
| SEQ ID NO: 1192 | 2hpf_pa | active | UUUCUU | 1.75519 | 1.09E−10 |
| SEQ ID NO: 1193 | 2hpf_pa | active | UUUUGC | 1.81563 | 1.59E−10 |
| SEQ ID NO: 1194 | 2hpf_pa | active | UUUUAU | 1.65625 | 5.08E−10 |
| SEQ ID NO: 1195 | 2hpf_pa | active | UUCUA | 1.79966 | 7.64E−10 |
| SEQ ID NO: 1196 | 2hpf_pa | active | UUGCAU | 2.15953 | 1.33E−09 |
| SEQ ID NO: 1197 | 2hpf_pa | active | UUGUG | 1.4538 | 1.39E−09 |
| SEQ ID NO: 1198 | 2hpf_pa | active | GCUUU | 1.4253 | 1.74E−09 |
| SEQ ID NO: 1199 | 2hpf_pa | active | UUCUUG | 2.10537 | 3.97E−09 |
| SEQ ID NO: 1200 | 2hpf_pa | active | AUUGU | 1.53719 | 4.12E−09 |
| SEQ ID NO: 1201 | 2hpf_pa | active | UUUAUA | 1.84552 | 4.38E−09 |
| SEQ ID NO: 1202 | 2hpf_pa | active | GUUUG | 1.38675 | 4.73E−09 |
| SEQ ID NO: 1203 | 2hpf_pa | active | GAUUU | 1.42186 | 5.08E−09 |
| SEQ ID NO: 1204 | 2hpf_pa | active | UUGCA | 1.58529 | 6.09E−09 |
| SEQ ID NO: 1205 | 2hpf_pa | active | GAGUU | 1.51998 | 6.67E−09 |
| SEQ ID NO: 1206 | 2hpf_pa | active | UUUUGA | 1.72882 | 6.74E−09 |
| SEQ ID NO: 1207 | 2hpf_pa | active | UUUGCA | 1.89645 | 9.14E−09 |
| SEQ ID NO: 1208 | 2hpf_pa | active | ACUUUU | 1.71842 | 9.49E−09 |
| SEQ ID NO: 1209 | 2hpf_pa | active | UAGUGU | 2.19759 | 1.11E−08 |
| SEQ ID NO: 1210 | 2hpf_pa | active | UUAUA | 1.5508 | 1.22E−08 |
| SEQ ID NO: 1211 | 2hpf_pa | active | CAUUUG | 1.9385 | 1.71E−08 |
| SEQ ID NO: 1212 | 2hpf_pa | active | AGUUU | 1.37743 | 1.96E−08 |
| SEQ ID NO: 1213 | 2hpf_pa | active | AUUUCU | 1.78537 | 2.00E−08 |
| SEQ ID NO: 1214 | 2hpf_pa | active | UACUU | 1.62906 | 2.00E−08 |
| SEQ ID NO: 1215 | 2hpf_pa | active | GUUUGU | 1.65291 | 2.26E−08 |
| SEQ ID NO: 1216 | 2hpf_pa | active | GUGUU | 1.3607 | 3.13E−08 |
| SEQ ID NO: 1217 | 2hpf_pa | active | GUUUUG | 1.5863 | 3.62E−08 |
| SEQ ID NO: 1218 | 2hpf_pa | active | UUUUUC | 1.58334 | 3.64E−08 |
| SEQ ID NO: 1219 | 2hpf_pa | active | UGUGU | 1.36327 | 5.90E−08 |
| SEQ ID NO: 1220 | 2hpf_pa | active | UUCUAC | 2.40578 | 6.22E−08 |
| SEQ ID NO: 1221 | 2hpf_pa | active | AUAUUU | 1.6227 | 6.47E−08 |
| SEQ ID NO: 1222 | 2hpf_pa | active | UAUUGU | 2.02441 | 6.63E−08 |
| SEQ ID NO: 1223 | 2hpf_pa | active | AAUUUU | 1.85989 | 8.44E−08 |
| SEQ ID NO: 1224 | 2hpf_pa | active | UAUAU | 1.59341 | 1.15E−07 |
| SEQ ID NO: 1225 | 2hpf_pa | active | UUCCU | 1.44056 | 1.34E−07 |
| SEQ ID NO: 1226 | 2hpf_pa | active | CUUUUG | 1.72637 | 1.62E−07 |
| SEQ ID NO: 1227 | 2hpf_pa | active | UUAGU | 1.51486 | 1.73E−07 |
| SEQ ID NO: 1228 | 2hpf_pa | active | UCUUU | 1.34528 | 2.00E−07 |
| SEQ ID NO: 1229 | 2hpf_pa | active | UAUUG | 1.53216 | 2.17E−07 |
| SEQ ID NO: 1230 | 2hpf_pa | active | AUUGUU | 1.82256 | 2.36E−07 |
| SEQ ID NO: 1231 | 2hpf_pa | active | UUUGA | 1.34407 | 2.38E−07 |
| SEQ ID NO: 1232 | 2hpf_pa | active | UUUGUG | 1.62474 | 2.51E−07 |
| SEQ ID NO: 1233 | 2hpf_pa | active | GUUAUU | 1.77956 | 3.78E−07 |
| SEQ ID NO: 1234 | 2hpf_pa | active | UAGUG | 1.64537 | 4.56E−07 |
| SEQ ID NO: 1235 | 2hpf_pa | active | UACUUU | 1.85228 | 5.33E−07 |
| SEQ ID NO: 1236 | 2hpf_pa | active | UUGCGU | 2.07109 | 5.68E−07 |
| SEQ ID NO: 1237 | 2hpf_pa | active | UUACG | 1.65501 | 7.43E−07 |
| SEQ ID NO: 1238 | 2hpf_pa | active | GUUUUA | 1.54123 | 7.79E−07 |
| SEQ ID NO: 1239 | 2hpf_pa | active | AAUUU | 1.42347 | 8.35E−07 |
| SEQ ID NO: 1240 | 2hpf_pa | active | GAUUUU | 1.63221 | 9.57E−07 |
| SEQ ID NO: 1241 | 2hpf_pa | active | UUUAGU | 1.70796 | 9.62E−07 |
| SEQ ID NO: 1242 | 2hpf_pa | active | AACUUU | 1.69011 | 9.80E−07 |
| SEQ ID NO: 1243 | 2hpf_pa | active | UAUAUU | 1.8012 | 1.26E−06 |
| SEQ ID NO: 1244 | 2hpf_pa | active | AUUUUC | 1.62784 | 1.75E−06 |
| SEQ ID NO: 1245 | 2hpf_pa | active | UUGCG | 1.53561 | 2.22E−06 |
| SEQ ID NO: 1246 | 2hpf_pa | active | GCUUUU | 1.53712 | 2.32E−06 |
| SEQ ID NO: 1247 | 2hpf_pa | active | UGUUUC | 1.64031 | 2.33E−06 |
| SEQ ID NO: 1248 | 2hpf_pa | active | UUGAUU | 1.71971 | 2.34E−06 |
| SEQ ID NO: 1249 | 2hpf_pa | active | UUUGG | 1.35203 | 2.37E−06 |
| SEQ ID NO: 1250 | 2hpf_pa | active | CUAUA | 1.75586 | 2.71E−06 |
| SEQ ID NO: 1251 | 2hpf_pa | active | GUGUUU | 1.43329 | 2.72E−06 |
| SEQ ID NO: 1252 | 2hpf_pa | active | UGUGUA | 1.87336 | 2.89E−06 |
| SEQ ID NO: 1253 | 2hpf_pa | active | AUUUGU | 1.62601 | 2.97E−06 |
| SEQ ID NO: 1254 | 2hpf_pa | active | AUAUU | 1.3516 | 3.24E−06 |
| SEQ ID NO: 1255 | 2hpf_pa | active | UUAUAU | 1.88166 | 3.71E−06 |
| SEQ ID NO: 1256 | 2hpf_pa | active | CGCUUU | 1.63314 | 4.03E−06 |
| SEQ ID NO: 1257 | 2hpf_pa | active | UAAGA | 1.60748 | 4.78E−06 |
| SEQ ID NO: 1258 | 2hpf_pa | active | UGUUUG | 1.51603 | 4.92E−06 |
| SEQ ID NO: 1259 | 2hpf_pa | active | UUUCUA | 1.83133 | 5.92E−06 |
| SEQ ID NO: 1260 | 2hpf_pa | active | UCUUUU | 1.51558 | 5.97E−06 |
| SEQ ID NO: 1261 | 2hpf_pa | active | CUGUUU | 1.49558 | 6.04E−06 |
| SEQ ID NO: 1262 | 2hpf_pa | active | CUAUAU | 2.31901 | 6.94E−06 |
| SEQ ID NO: 1263 | 2hpf_pa | active | UUUACG | 1.8285 | 7.44E−06 |
| SEQ ID NO: 1264 | 2hpf_pa | active | UCUUG | 1.44835 | 7.76E−06 |
| SEQ ID NO: 1265 | 2hpf_pa | active | CUUCUU | 1.79219 | 8.19E−06 |
| SEQ ID NO: 1266 | 2hpf_pa | active | UGCAU | 1.45508 | 8.24E−06 |
| SEQ ID NO: 1267 | 2hpf_pa | active | AGUUUU | 1.51671 | 8.27E−06 |
| SEQ ID NO: 1268 | 2hpf_pa | active | CUUUG | 1.35965 | 8.41E−06 |
| SEQ ID NO: 1269 | 2hpf_pa | active | UUGCUA | 1.89645 | 9.70E−06 |
| SEQ ID NO: 1270 | 2hpf_pa | repressed | AUGGC | 3.31109 | 2.42E−55 |
| SEQ ID NO: 1271 | 2hpf_pa | repressed | CAUGG | 2.91143 | 1.35E−40 |
| SEQ ID NO: 1272 | 2hpf_pa | repressed | GGAUG | 2.63874 | 1.17E−37 |
| SEQ ID NO: 1273 | 2hpf_pa | repressed | GAUGG | 2.79498 | 1.37E−36 |
| SEQ ID NO: 1274 | 2hpf_pa | repressed | CCAUG | 3.11158 | 1.63E−35 |
| SEQ ID NO: 1275 | 2hpf_pa | repressed | AUGGA | 2.50351 | 1.99E−33 |
| SEQ ID NO: 1276 | 2hpf_pa | repressed | AGGAUG | 4.46305 | 3.89E−32 |
| SEQ ID NO: 1277 | 2hpf_pa | repressed | AUGGCG | 4.351 | 2.59E−29 |
| SEQ ID NO: 1278 | 2hpf_pa | repressed | AGAUG | 2.35229 | 8.48E−27 |
| SEQ ID NO: 1279 | 2hpf_pa | repressed | GAUGC | 2.36442 | 2.01E−21 |
| SEQ ID NO: 1280 | 2hpf_pa | repressed | AAUGGC | 3.64118 | 6.74E−21 |
| SEQ ID NO: 1281 | 2hpf_pa | repressed | GAUGGC | 3.97689 | 1.36E−20 |
| SEQ ID NO: 1282 | 2hpf_pa | repressed | CAUGGC | 3.42559 | 1.39E−19 |
| SEQ ID NO: 1283 | 2hpf_pa | repressed | AUGGAG | 3.00398 | 2.93E−19 |
| SEQ ID NO: 1284 | 2hpf_pa | repressed | UCAUG | 2.03922 | 5.28E−19 |
| SEQ ID NO: 1285 | 2hpf_pa | repressed | GCCAUG | 3.91496 | 7.04E−19 |
| SEQ ID NO: 1286 | 2hpf_pa | repressed | AUGGG | 2.41643 | 1.54E−18 |
| SEQ ID NO: 1287 | 2hpf_pa | repressed | GAUGU | 1.98663 | 3.01E−18 |
| SEQ ID NO: 1288 | 2hpf_pa | repressed | AUGUG | 1.87882 | 6.33E−18 |
| SEQ ID NO: 1289 | 2hpf_pa | repressed | UGGCG | 2.10064 | 6.72E−18 |
| SEQ ID NO: 1290 | 2hpf_pa | repressed | AUGAG | 2.0341 | 1.15E−17 |
| SEQ ID NO: 1291 | 2hpf_pa | repressed | UGGAG | 1.75762 | 1.19E−17 |
| SEQ ID NO: 1292 | 2hpf_pa | repressed | AAUGG | 2.16916 | 1.23E−17 |
| SEQ ID NO: 1293 | 2hpf_pa | repressed | GGGGC | 2.2588 | 1.81E−17 |
| SEQ ID NO: 1294 | 2hpf_pa | repressed | AAAAUG | 2.89446 | 2.31E−17 |
| SEQ ID NO: 1295 | 2hpf_pa | repressed | GUCAUG | 3.50783 | 2.42E−17 |
| SEQ ID NO: 1296 | 2hpf_pa | repressed | AUCAUG | 3.33775 | 2.56E−17 |
| SEQ ID NO: 1297 | 2hpf_pa | repressed | GGAUGC | 3.48563 | 3.35E−17 |
| SEQ ID NO: 1298 | 2hpf_pa | repressed | GGCGG | 1.98543 | 4.55E−17 |
| SEQ ID NO: 1299 | 2hpf_pa | repressed | GAUGA | 1.98234 | 1.58E−16 |
| SEQ ID NO: 1300 | 2hpf_pa | repressed | GGCUG | 1.75743 | 6.65E−16 |
| SEQ ID NO: 1301 | 2hpf_pa | repressed | AGGGG | 2.15355 | 7.81E−16 |
| SEQ ID NO: 1302 | 2hpf_pa | repressed | GGAGC | 1.76769 | 9.13E−16 |
| SEQ ID NO: 1303 | 2hpf_pa | repressed | UCAUGG | 3.10571 | 1.89E−15 |
| SEQ ID NO: 1304 | 2hpf_pa | repressed | GCGGC | 1.8613 | 2.12E−15 |
| SEQ ID NO: 1305 | 2hpf_pa | repressed | GGAGCG | 2.60619 | 4.72E−15 |
| SEQ ID NO: 1306 | 2hpf_pa | repressed | GGAUGG | 3.43312 | 6.40E−15 |
| SEQ ID NO: 1307 | 2hpf_pa | repressed | AAGAUG | 3.081 | 8.34E−15 |
| SEQ ID NO: 1308 | 2hpf_pa | repressed | CGAUG | 2.23286 | 2.27E−14 |
| SEQ ID NO: 1309 | 2hpf_pa | repressed | GGGGG | 2.19396 | 4.06E−14 |
| SEQ ID NO: 1310 | 2hpf_pa | repressed | ACCAUG | 4.0053 | 6.67E−14 |
| SEQ ID NO: 1311 | 2hpf_pa | repressed | AUGAGG | 2.93582 | 8.16E−14 |
| SEQ ID NO: 1312 | 2hpf_pa | repressed | GGAGG | 1.76485 | 8.67E−14 |
| SEQ ID NO: 1313 | 2hpf_pa | repressed | GCGGG | 1.90037 | 1.07E−13 |
| SEQ ID NO: 1314 | 2hpf_pa | repressed | GGGAUG | 3.10971 | 1.47E−13 |
| SEQ ID NO: 1315 | 2hpf_pa | repressed | CAUGGG | 3.73043 | 1.99E−13 |
| SEQ ID NO: 1316 | 2hpf_pa | repressed | CGGCC | 2.09218 | 5.28E−13 |
| SEQ ID NO: 1317 | 2hpf_pa | repressed | AUGGCA | 2.96905 | 6.27E−13 |
| SEQ ID NO: 1318 | 2hpf_pa | repressed | CGGGG | 2.04445 | 6.92E−13 |
| SEQ ID NO: 1319 | 2hpf_pa | repressed | AUGGCU | 2.99798 | 6.98E−13 |
| SEQ ID NO: 1320 | 2hpf_pa | repressed | GGGGA | 1.90814 | 8.43E−13 |
| SEQ ID NO: 1321 | 2hpf_pa | repressed | CCAUGG | 3.28679 | 8.67E−13 |
| SEQ ID NO: 1322 | 2hpf_pa | repressed | CAUGA | 1.92817 | 1.09E−12 |
| SEQ ID NO: 1323 | 2hpf_pa | repressed | GAUGGA | 2.74402 | 1.12E−12 |
| SEQ ID NO: 1324 | 2hpf_pa | repressed | GGGCUG | 2.56339 | 1.40E−12 |
| SEQ ID NO: 1325 | 2hpf_pa | repressed | GCAUGG | 3.07073 | 2.20E−12 |
| SEQ ID NO: 1326 | 2hpf_pa | repressed | AAAUGG | 2.75364 | 2.36E−12 |
| SEQ ID NO: 1327 | 2hpf_pa | repressed | CAAUG | 1.98492 | 2.42E−12 |
| SEQ ID NO: 1328 | 2hpf_pa | repressed | GGCGGC | 2.51298 | 2.46E−12 |
| SEQ ID NO: 1329 | 2hpf_pa | repressed | GGGGCG | 3.08628 | 3.12E−12 |
| SEQ ID NO: 1330 | 2hpf_pa | repressed | CAUGGA | 2.60084 | 3.53E−12 |
| SEQ ID NO: 1331 | 2hpf_pa | repressed | GCGGCG | 2.41578 | 3.90E−12 |
| SEQ ID NO: 1332 | 2hpf_pa | repressed | GAGAUG | 2.97863 | 4.31E−12 |
| SEQ ID NO: 1333 | 2hpf_pa | repressed | GAGCG | 1.66451 | 5.79E−12 |
| SEQ ID NO: 1334 | 2hpf_pa | repressed | UGAUG | 1.73635 | 6.32E−12 |
| SEQ ID NO: 1335 | 2hpf_pa | repressed | AAAUG | 1.69712 | 7.21E−12 |
| SEQ ID NO: 1336 | 2hpf_pa | repressed | GGGCU | 1.85231 | 8.49E−12 |
| SEQ ID NO: 1337 | 2hpf_pa | repressed | GAGGG | 1.7693 | 9.31E−12 |
| SEQ ID NO: 1338 | 2hpf_pa | repressed | CAGGG | 1.90431 | 1.28E−11 |
| SEQ ID NO: 1339 | 2hpf_pa | repressed | UAUGGA | 2.86965 | 1.84E−11 |
| SEQ ID NO: 1340 | 2hpf_pa | repressed | UAUGG | 1.99098 | 1.86E−11 |
| SEQ ID NO: 1341 | 2hpf_pa | repressed | CGGGCU | 2.54802 | 2.89E−11 |
| SEQ ID NO: 1342 | 2hpf_pa | repressed | ACAAUG | 3.02559 | 3.29E−11 |
| SEQ ID NO: 1343 | 2hpf_pa | repressed | GAUGUG | 2.35819 | 4.85E−11 |
| SEQ ID NO: 1344 | 2hpf_pa | repressed | UGGCGG | 2.50331 | 8.95E−11 |
| SEQ ID NO: 1345 | 2hpf_pa | repressed | CGGGC | 1.89551 | 1.23E−10 |
| SEQ ID NO: 1346 | 2hpf_pa | repressed | GAUGCG | 3.09353 | 1.34E−10 |
| SEQ ID NO: 1347 | 2hpf_pa | repressed | CCAUGA | 3.19006 | 1.54E−10 |
| SEQ ID NO: 1348 | 2hpf_pa | repressed | UGUGGC | 2.47023 | 1.55E−10 |
| SEQ ID NO: 1349 | 2hpf_pa | repressed | AAUGU | 1.64587 | 1.59E−10 |
| SEQ ID NO: 1350 | 2hpf_pa | repressed | CGGAGC | 2.30521 | 1.86E−10 |
| SEQ ID NO: 1351 | 2hpf_pa | repressed | ACGAUG | 3.57616 | 2.19E−10 |
| SEQ ID NO: 1352 | 2hpf_pa | repressed | AGAUGU | 2.44866 | 2.21E−10 |
| SEQ ID NO: 1353 | 2hpf_pa | repressed | GCAGC | 1.55587 | 2.26E−10 |
| SEQ ID NO: 1354 | 2hpf_pa | repressed | GCGCUG | 2.06907 | 2.52E−10 |
| SEQ ID NO: 1355 | 2hpf_pa | repressed | GGCAG | 1.68021 | 2.68E−10 |
| SEQ ID NO: 1356 | 2hpf_pa | repressed | AGGAU | 1.68633 | 3.55E−10 |
| SEQ ID NO: 1357 | 2hpf_pa | repressed | AGAUGG | 2.67796 | 3.79E−10 |
| SEQ ID NO: 1358 | 2hpf_pa | repressed | AGCCAU | 2.50331 | 5.97E−10 |
| SEQ ID NO: 1359 | 2hpf_pa | repressed | GAUGCU | 2.55547 | 6.01E−10 |
| SEQ ID NO: 1360 | 2hpf_pa | repressed | GGCCG | 1.83966 | 7.10E−10 |
| SEQ ID NO: 1361 | 2hpf_pa | repressed | CCAUGU | 2.96392 | 8.93E−10 |
| SEQ ID NO: 1362 | 2hpf_pa | repressed | UGGCC | 1.83355 | 1.23E−09 |
| SEQ ID NO: 1363 | 2hpf_pa | repressed | GGAGGA | 1.9359 | 1.81E−09 |
| SEQ ID NO: 1364 | 2hpf_pa | repressed | GGGCG | 1.86669 | 2.00E−09 |
| SEQ ID NO: 1365 | 2hpf_pa | repressed | GGGUG | 1.78939 | 2.07E−09 |
| SEQ ID NO: 1366 | 2hpf_pa | repressed | CUCGG | 1.70709 | 2.18E−09 |
| SEQ ID NO: 1367 | 2hpf_pa | repressed | AUGUGU | 2.05906 | 2.29E−09 |
| SEQ ID NO: 1368 | 2hpf_pa | repressed | CUCGGG | 2.81855 | 2.57E−09 |
| SEQ ID NO: 1369 | 2hpf_pa | repressed | GGGAG | 1.68617 | 2.71E−09 |
| SEQ ID NO: 1370 | 2hpf_pa | repressed | CGGAG | 1.5743 | 3.10E−09 |
| SEQ ID NO: 1371 | 2hpf_pa | repressed | AUGAUG | 2.49007 | 3.28E−09 |
| SEQ ID NO: 1372 | 2hpf_pa | repressed | GAAGGG | 2.51699 | 3.32E−09 |
| SEQ ID NO: 1373 | 2hpf_pa | repressed | AAAUGA | 2.11819 | 3.98E−09 |
| SEQ ID NO: 1374 | 2hpf_pa | repressed | GAGCGG | 2.18928 | 4.06E−09 |
| SEQ ID NO: 1375 | 2hpf_pa | repressed | GGCAGC | 2.27021 | 4.35E−09 |
| SEQ ID NO: 1376 | 2hpf_pa | repressed | AUGGGG | 3.19572 | 4.76E−09 |
| SEQ ID NO: 1377 | 2hpf_pa | repressed | AUGAU | 1.7171 | 4.80E−09 |
| SEQ ID NO: 1378 | 2hpf_pa | repressed | CCGGC | 1.8293 | 4.83E−09 |
| SEQ ID NO: 1379 | 2hpf_pa | repressed | AGCGC | 1.56972 | 5.21E−09 |
| SEQ ID NO: 1380 | 2hpf_pa | repressed | CGAUGA | 2.78564 | 5.87E−09 |
| SEQ ID NO: 1381 | 2hpf_pa | repressed | CGCUG | 1.49441 | 6.21E−09 |
| SEQ ID NO: 1382 | 2hpf_pa | repressed | GGGAU | 1.63399 | 6.31E−09 |
| SEQ ID NO: 1383 | 2hpf_pa | repressed | GCCGC | 1.64648 | 8.14E−09 |
| SEQ ID NO: 1384 | 2hpf_pa | repressed | UGGAGG | 2.15073 | 8.23E−09 |
| SEQ ID NO: 1385 | 2hpf_pa | repressed | GGGGGC | 2.65658 | 9.97E−09 |
| SEQ ID NO: 1386 | 2hpf_pa | repressed | AAUGUG | 2.07171 | 1.00E−08 |
| SEQ ID NO: 1387 | 2hpf_pa | repressed | AUGUGG | 2.29003 | 1.52E−08 |
| SEQ ID NO: 1388 | 2hpf_pa | repressed | GGCAUG | 3.12241 | 1.54E−08 |
| SEQ ID NO: 1389 | 2hpf_pa | repressed | AGAAUG | 2.78146 | 1.55E−08 |
| SEQ ID NO: 1390 | 2hpf_pa | repressed | GCAUG | 1.72437 | 1.62E−08 |
| SEQ ID NO: 1391 | 2hpf_pa | repressed | CAGGGG | 2.73089 | 1.65E−08 |
| SEQ ID NO: 1392 | 2hpf_pa | repressed | AUGUC | 1.72823 | 1.93E−08 |
| SEQ ID NO: 1393 | 2hpf_pa | repressed | GGCGA | 1.71624 | 2.09E−08 |
| SEQ ID NO: 1394 | 2hpf_pa | repressed | CCAGC | 1.57726 | 2.21E−08 |
| SEQ ID NO: 1395 | 2hpf_pa | repressed | UGGCCG | 2.40756 | 2.99E−08 |
| SEQ ID NO: 1396 | 2hpf_pa | repressed | GAAUG | 1.60413 | 3.17E−08 |
| SEQ ID NO: 1397 | 2hpf_pa | repressed | GCGCG | 1.53624 | 3.39E−08 |
| SEQ ID NO: 1398 | 2hpf_pa | repressed | UGGGG | 1.72482 | 3.86E−08 |
| SEQ ID NO: 1399 | 2hpf_pa | repressed | UGUGG | 1.5051 | 3.87E−08 |
| SEQ ID NO: 1400 | 2hpf_pa | repressed | AGCAUG | 2.50331 | 3.95E−08 |
| SEQ ID NO: 1401 | 2hpf_pa | repressed | GCCCGG | 2.50331 | 3.95E−08 |
| SEQ ID NO: 1402 | 2hpf_pa | repressed | CAUGC | 1.76071 | 3.98E−08 |
| SEQ ID NO: 1403 | 2hpf_pa | repressed | GCGCU | 1.4987 | 5.20E−08 |
| SEQ ID NO: 1404 | 2hpf_pa | repressed | UGAUGG | 2.31462 | 5.30E−08 |
| SEQ ID NO: 1405 | 2hpf_pa | repressed | UGAGGG | 2.24041 | 5.96E−08 |
| SEQ ID NO: 1406 | 2hpf_pa | repressed | AGGGA | 1.62678 | 7.12E−08 |
| SEQ ID NO: 1407 | 2hpf_pa | repressed | AUGGAA | 2.38953 | 7.61E−08 |
| SEQ ID NO: 1408 | 2hpf_pa | repressed | AUGGCC | 2.79649 | 8.72E−08 |
| SEQ ID NO: 1409 | 2hpf_pa | repressed | CGGCA | 1.7118 | 8.74E−08 |
| SEQ ID NO: 1410 | 2hpf_pa | repressed | GCCAU | 1.67158 | 9.63E−08 |
| SEQ ID NO: 1411 | 2hpf_pa | repressed | UGAGG | 1.52198 | 9.85E−08 |
| SEQ ID NO: 1412 | 2hpf_pa | repressed | CCAUGC | 3.09934 | 9.86E−08 |
| SEQ ID NO: 1413 | 2hpf_pa | repressed | CCCGG | 1.73285 | 1.01E−07 |
| SEQ ID NO: 1414 | 2hpf_pa | repressed | UCCAUG | 2.48695 | 1.02E−07 |
| SEQ ID NO: 1415 | 2hpf_pa | repressed | CAAUGU | 2.38688 | 1.11E−07 |
| SEQ ID NO: 1416 | 2hpf_pa | repressed | CGCGG | 1.55676 | 1.12E−07 |
| SEQ ID NO: 1417 | 2hpf_pa | repressed | GUAGGG | 3.56404 | 1.14E−07 |
| SEQ ID NO: 1418 | 2hpf_pa | repressed | AAUGA | 1.55774 | 1.20E−07 |
| SEQ ID NO: 1419 | 2hpf_pa | repressed | AUGAA | 1.55504 | 1.20E−07 |
| SEQ ID NO: 1420 | 2hpf_pa | repressed | GGGUUC | 2.92053 | 1.32E−07 |
| SEQ ID NO: 1421 | 2hpf_pa | repressed | AGCGG | 1.5021 | 1.46E−07 |
| SEQ ID NO: 1422 | 2hpf_pa | repressed | GAAUGU | 2.10596 | 1.52E−07 |
| SEQ ID NO: 1423 | 2hpf_pa | repressed | GGAUGA | 2.15803 | 1.52E−07 |
| SEQ ID NO: 1424 | 2hpf_pa | repressed | CGCGGG | 2.30251 | 1.65E−07 |
| SEQ ID NO: 1425 | 2hpf_pa | repressed | CAGCC | 1.53415 | 1.66E−07 |
| SEQ ID NO: 1426 | 2hpf_pa | repressed | AGAUGA | 2.18471 | 1.81E−07 |
| SEQ ID NO: 1427 | 2hpf_pa | repressed | CCGGCC | 2.75594 | 1.99E−07 |
| SEQ ID NO: 1428 | 2hpf_pa | repressed | GCCGCU | 1.94196 | 2.01E−07 |
| SEQ ID NO: 1429 | 2hpf_pa | repressed | GGCUGC | 2.09955 | 2.19E−07 |
| SEQ ID NO: 1430 | 2hpf_pa | repressed | CCGGGG | 2.60187 | 2.21E−07 |
| SEQ ID NO: 1431 | 2hpf_pa | repressed | GCGAUG | 2.52132 | 2.22E−07 |
| SEQ ID NO: 1432 | 2hpf_pa | repressed | GCAGCG | 1.90053 | 2.44E−07 |
| SEQ ID NO: 1433 | 2hpf_pa | repressed | GGCGC | 1.68932 | 2.52E−07 |
| SEQ ID NO: 1434 | 2hpf_pa | repressed | AUGGAC | 2.36677 | 2.83E−07 |
| SEQ ID NO: 1435 | 2hpf_pa | repressed | GGCCA | 1.78027 | 3.15E−07 |
| SEQ ID NO: 1436 | 2hpf_pa | repressed | CGAUGG | 2.65308 | 3.17E−07 |
| SEQ ID NO: 1437 | 2hpf_pa | repressed | GGCCGC | 2.43566 | 3.77E−07 |
| SEQ ID NO: 1438 | 2hpf_pa | repressed | CCCAGC | 2.18736 | 4.12E−07 |
| SEQ ID NO: 1439 | 2hpf_pa | repressed | UGGCA | 1.66062 | 4.31E−07 |
| SEQ ID NO: 1440 | 2hpf_pa | repressed | GACGG | 1.59664 | 4.82E−07 |
| SEQ ID NO: 1441 | 2hpf_pa | repressed | GAGGA | 1.38839 | 5.14E−07 |
| SEQ ID NO: 1442 | 2hpf_pa | repressed | UUAUGG | 2.31075 | 5.45E−07 |
| SEQ ID NO: 1443 | 2hpf_pa | repressed | GAUGAG | 2.10805 | 5.49E−07 |
| SEQ ID NO: 1444 | 2hpf_pa | repressed | GGGGGG | 2.63507 | 5.72E−07 |
| SEQ ID NO: 1445 | 2hpf_pa | repressed | AUGCC | 1.86702 | 5.78E−07 |
| SEQ ID NO: 1446 | 2hpf_pa | repressed | AGCGCU | 1.88485 | 6.01E−07 |
| SEQ ID NO: 1447 | 2hpf_pa | repressed | GGAAGC | 1.98819 | 6.36E−07 |
| SEQ ID NO: 1448 | 2hpf_pa | repressed | UCAGGG | 2.34488 | 7.16E−07 |
| SEQ ID NO: 1449 | 2hpf_pa | repressed | AUGAGC | 2.07025 | 7.39E−07 |
| SEQ ID NO: 1450 | 2hpf_pa | repressed | AGGGGG | 2.28372 | 7.48E−07 |
| SEQ ID NO: 1451 | 2hpf_pa | repressed | CGGCCG | 2.28372 | 7.48E−07 |
| SEQ ID NO: 1452 | 2hpf_pa | repressed | GAGGGG | 2.18563 | 7.50E−07 |
| SEQ ID NO: 1453 | 2hpf_pa | repressed | AACAUG | 2.21915 | 8.81E−07 |
| SEQ ID NO: 1454 | 2hpf_pa | repressed | GAACGG | 2.38411 | 9.30E−07 |
| SEQ ID NO: 1455 | 2hpf_pa | repressed | GCUGAA | 1.70611 | 1.00E−06 |
| SEQ ID NO: 1456 | 2hpf_pa | repressed | UGGAGC | 1.82059 | 1.01E−06 |
| SEQ ID NO: 1457 | 2hpf_pa | repressed | CAUGU | 1.54948 | 1.06E−06 |
| SEQ ID NO: 1458 | 2hpf_pa | repressed | GGAGGG | 2.06282 | 1.06E−06 |
| SEQ ID NO: 1459 | 2hpf_pa | repressed | GAAGAU | 1.90575 | 1.11E−06 |
| SEQ ID NO: 1460 | 2hpf_pa | repressed | UGGGUG | 2.19542 | 1.18E−06 |
| SEQ ID NO: 1461 | 2hpf_pa | repressed | GGUUC | 1.68189 | 1.20E−06 |
| SEQ ID NO: 1462 | 2hpf_pa | repressed | AGGGU | 1.69592 | 1.29E−06 |
| SEQ ID NO: 1463 | 2hpf_pa | repressed | AUGCG | 1.68204 | 1.37E−06 |
| SEQ ID NO: 1464 | 2hpf_pa | repressed | GGACGG | 2.23153 | 1.38E−06 |
| SEQ ID NO: 1465 | 2hpf_pa | repressed | AUGGGC | 2.74047 | 1.41E−06 |
| SEQ ID NO: 1466 | 2hpf_pa | repressed | UGGCAG | 2.0958 | 1.43E−06 |
| SEQ ID NO: 1467 | 2hpf_pa | repressed | AAGGGG | 2.31075 | 1.45E−06 |
| SEQ ID NO: 1468 | 2hpf_pa | repressed | AGGGGC | 2.44599 | 1.47E−06 |
| SEQ ID NO: 1469 | 2hpf_pa | repressed | GGGCGC | 2.56977 | 1.53E−06 |
| SEQ ID NO: 1470 | 2hpf_pa | repressed | GACCCG | 2.29603 | 1.70E−06 |
| SEQ ID NO: 1471 | 2hpf_pa | repressed | ACGGCC | 2.71192 | 1.75E−06 |
| SEQ ID NO: 1472 | 2hpf_pa | repressed | GCUCGG | 2.09802 | 1.81E−06 |
| SEQ ID NO: 1473 | 2hpf_pa | repressed | AAGGG | 1.62116 | 1.86E−06 |
| SEQ ID NO: 1474 | 2hpf_pa | repressed | GAUGAU | 2.07648 | 1.87E−06 |
| SEQ ID NO: 1475 | 2hpf_pa | repressed | GAAAUG | 2.08807 | 2.06E−06 |
| SEQ ID NO: 1476 | 2hpf_pa | repressed | GGGGAC | 2.44418 | 2.16E−06 |
| SEQ ID NO: 1477 | 2hpf_pa | repressed | GCCGUC | 2.2454 | 2.17E−06 |
| SEQ ID NO: 1478 | 2hpf_pa | repressed | AGGGUC | 2.48262 | 2.21E−06 |
| SEQ ID NO: 1479 | 2hpf_pa | repressed | CCUCCG | 2.16503 | 2.29E−06 |
| SEQ ID NO: 1480 | 2hpf_pa | repressed | AUGCU | 1.5427 | 2.60E−06 |
| SEQ ID NO: 1481 | 2hpf_pa | repressed | GGGAGG | 2.15339 | 2.63E−06 |
| SEQ ID NO: 1482 | 2hpf_pa | repressed | GUCAU | 1.50996 | 2.68E−06 |
| SEQ ID NO: 1483 | 2hpf_pa | repressed | GGGGAG | 2.12717 | 2.76E−06 |
| SEQ ID NO: 1484 | 2hpf_pa | repressed | GGCCU | 1.58953 | 2.79E−06 |
| SEQ ID NO: 1485 | 2hpf_pa | repressed | GGAUGU | 2.02912 | 2.80E−06 |
| SEQ ID NO: 1486 | 2hpf_pa | repressed | CAUGCC | 2.84327 | 2.81E−06 |
| SEQ ID NO: 1487 | 2hpf_pa | repressed | CCGCC | 1.58116 | 2.84E−06 |
| SEQ ID NO: 1488 | 2hpf_pa | repressed | GCGCGG | 1.99434 | 2.87E−06 |
| SEQ ID NO: 1489 | 2hpf_pa | repressed | CAGGAU | 2.10278 | 2.87E−06 |
| SEQ ID NO: 1490 | 2hpf_pa | repressed | CAGAUG | 1.97843 | 2.88E−06 |
| SEQ ID NO: 1491 | 2hpf_pa | repressed | CAUGAC | 2.57484 | 3.31E−06 |
| SEQ ID NO: 1492 | 2hpf_pa | repressed | CGGCG | 1.50032 | 3.38E−06 |
| SEQ ID NO: 1493 | 2hpf_pa | repressed | UGCAG | 1.40746 | 3.38E−06 |
| SEQ ID NO: 1494 | 2hpf_pa | repressed | GCGUC | 1.51345 | 3.72E−06 |
| SEQ ID NO: 1495 | 2hpf_pa | repressed | GGGCA | 1.64641 | 3.73E−06 |
| SEQ ID NO: 1496 | 2hpf_pa | repressed | GCUGA | 1.35905 | 4.26E−06 |
| SEQ ID NO: 1497 | 2hpf_pa | repressed | UGGCUG | 1.78013 | 4.41E−06 |
| SEQ ID NO: 1498 | 2hpf_pa | repressed | CCCGC | 1.63417 | 4.44E−06 |
| SEQ ID NO: 1499 | 2hpf_pa | repressed | GCUGG | 1.39676 | 4.44E−06 |
| SEQ ID NO: 1500 | 2hpf_pa | repressed | CCCAG | 1.56809 | 4.55E−06 |
| SEQ ID NO: 1501 | 2hpf_pa | repressed | GUUCCG | 2.32191 | 4.83E−06 |
| SEQ ID NO: 1502 | 2hpf_pa | repressed | GAUGUC | 2.32191 | 4.83E−06 |
| SEQ ID NO: 1503 | 2hpf_pa | repressed | AGCGGG | 2.06155 | 4.86E−06 |
| SEQ ID NO: 1504 | 2hpf_pa | repressed | GCAGCC | 1.93933 | 5.14E−06 |
| SEQ ID NO: 1505 | 2hpf_pa | repressed | GGCUC | 1.52011 | 5.19E−06 |
| SEQ ID NO: 1506 | 2hpf_pa | repressed | GUGAUG | 2.09702 | 5.22E−06 |
| SEQ ID NO: 1507 | 2hpf_pa | repressed | AUGGGA | 2.09702 | 5.22E−06 |
| SEQ ID NO: 1508 | 2hpf_pa | repressed | GUCGG | 1.52595 | 5.23E−06 |
| SEQ ID NO: 1509 | 2hpf_pa | repressed | GGGGU | 1.65864 | 5.31E−06 |
| SEQ ID NO: 1510 | 2hpf_pa | repressed | UGAUGC | 2.07345 | 5.39E−06 |
| SEQ ID NO: 1511 | 2hpf_pa | repressed | GAAGG | 1.46284 | 5.41E−06 |
| SEQ ID NO: 1512 | 2hpf_pa | repressed | GAGCGC | 1.777 | 5.52E−06 |
| SEQ ID NO: 1513 | 2hpf_pa | repressed | CAGCCA | 1.90551 | 5.64E−06 |
| SEQ ID NO: 1514 | 2hpf_pa | repressed | CGGCGG | 1.91825 | 5.71E−06 |
| SEQ ID NO: 1515 | 2hpf_pa | repressed | GCGGGA | 1.9459 | 5.79E−06 |
| SEQ ID NO: 1516 | 2hpf_pa | repressed | GGGGCC | 2.60762 | 5.89E−06 |
| SEQ ID NO: 1517 | 2hpf_pa | repressed | GGCUGA | 1.82059 | 5.94E−06 |
| SEQ ID NO: 1518 | 2hpf_pa | repressed | GGUCG | 1.62186 | 5.99E−06 |
| SEQ ID NO: 1519 | 2hpf_pa | repressed | AUGAC | 1.60585 | 6.25E−06 |
| SEQ ID NO: 1520 | 2hpf_pa | repressed | UGGCGA | 2.36534 | 6.34E−06 |
| SEQ ID NO: 1521 | 2hpf_pa | repressed | GGGCC | 1.78613 | 6.51E−06 |
| SEQ ID NO: 1522 | 2hpf_pa | repressed | AAUGAG | 1.95279 | 6.51E−06 |
| SEQ ID NO: 1523 | 2hpf_pa | repressed | CAGCG | 1.39467 | 6.52E−06 |
| SEQ ID NO: 1524 | 2hpf_pa | repressed | UCAUGA | 2.09955 | 6.59E−06 |
| SEQ ID NO: 1525 | 2hpf_pa | repressed | GAGGGU | 2.28874 | 6.66E−06 |
| SEQ ID NO: 1526 | 2hpf_pa | repressed | CGAGG | 1.49463 | 6.77E−06 |
| SEQ ID NO: 1527 | 2hpf_pa | repressed | CGCUGG | 1.87974 | 6.88E−06 |
| SEQ ID NO: 1528 | 2hpf_pa | repressed | ACAUGG | 2.21072 | 7.82E−06 |
| SEQ ID NO: 1529 | 2hpf_pa | repressed | UCAGG | 1.48587 | 9.68E−06 |
| SEQ ID NO: 1530 | 2hpf_pa-6hpf_pa | active | UUUUU | 1.68383 | 1.35E−30 |
| SEQ ID NO: 1531 | 2hpf_pa-6hpf_pa | active | UUUUA | 1.60721 | 1.30E−26 |
| SEQ ID NO: 1532 | 2hpf_pa-6hpf_pa | active | UUUUUU | 2.09655 | 2.53E−24 |
| SEQ ID NO: 1533 | 2hpf_pa-6hpf_pa | active | CUUUU | 1.61714 | 3.80E−23 |
| SEQ ID NO: 1534 | 2hpf_pa-6hpf_pa | active | GUUUU | 1.48323 | 2.23E−19 |
| SEQ ID NO: 1535 | 2hpf_pa-6hpf_pa | active | UUUUG | 1.50802 | 2.35E−18 |
| SEQ ID NO: 1536 | 2hpf_pa-6hpf_pa | active | UAUUU | 1.52741 | 9.89E−18 |
| SEQ ID NO: 1537 | 2hpf_pa-6hpf_pa | active | AUUUU | 1.5104 | 1.40E−17 |
| SEQ ID NO: 1538 | 2hpf_pa-6hpf_pa | active | UUUCU | 1.54017 | 5.53E−17 |
| SEQ ID NO: 1539 | 2hpf_pa-6hpf_pa | active | UUUUUA | 1.81474 | 2.56E−16 |
| SEQ ID NO: 1540 | 2hpf_pa-6hpf_pa | active | UUCUU | 1.65506 | 6.89E−16 |
| SEQ ID NO: 1541 | 2hpf_pa-6hpf_pa | active | UUUAU | 1.5054 | 9.28E−16 |
| SEQ ID NO: 1542 | 2hpf_pa-6hpf_pa | active | UUUUC | 1.47615 | 2.18E−15 |
| SEQ ID NO: 1543 | 2hpf_pa-6hpf_pa | active | UUUAUU | 1.74499 | 1.85E−14 |
| SEQ ID NO: 1544 | 2hpf_pa-6hpf_pa | active | UUUGU | 1.45302 | 2.17E−14 |
| SEQ ID NO: 1545 | 2hpf_pa-6hpf_pa | active | UUUGUU | 1.73328 | 4.79E−14 |
| SEQ ID NO: 1546 | 2hpf_pa-6hpf_pa | active | UUAUU | 1.49484 | 7.60E−14 |
| SEQ ID NO: 1547 | 2hpf_pa-6hpf_pa | active | UGUUU | 1.3851 | 9.21E−14 |
| SEQ ID NO: 1548 | 2hpf_pa-6hpf_pa | active | UUGUU | 1.46745 | 2.46E−13 |
| SEQ ID NO: 1549 | 2hpf_pa-6hpf_pa | active | ACUUU | 1.55016 | 2.89E−13 |
| SEQ ID NO: 1550 | 2hpf_pa-6hpf_pa | active | UACUUU | 2.28775 | 4.91E−13 |
| SEQ ID NO: 1551 | 2hpf_pa-6hpf_pa | active | UAUUUU | 1.70263 | 7.89E−13 |
| SEQ ID NO: 1552 | 2hpf_pa-6hpf_pa | active | CCUCU | 1.71285 | 1.43E−12 |
| SEQ ID NO: 1553 | 2hpf_pa-6hpf_pa | active | UUUUAU | 1.74258 | 3.52E−12 |
| SEQ ID NO: 1554 | 2hpf_pa-6hpf_pa | active | CUUUUU | 1.74305 | 4.01E−12 |
| SEQ ID NO: 1555 | 2hpf_pa-6hpf_pa | active | ACUUUU | 1.88393 | 4.59E−12 |
| SEQ ID NO: 1556 | 2hpf_pa-6hpf_pa | active | UUUGC | 1.52407 | 1.18E−11 |
| SEQ ID NO: 1557 | 2hpf_pa-6hpf_pa | active | GUUUUU | 1.64056 | 7.68E−11 |
| SEQ ID NO: 1558 | 2hpf_pa-6hpf_pa | active | UACUU | 1.73874 | 8.42E−11 |
| SEQ ID NO: 1559 | 2hpf_pa-6hpf_pa | active | UUUUUG | 1.68113 | 1.02E−10 |
| SEQ ID NO: 1560 | 2hpf_pa-6hpf_pa | active | UUGUUU | 1.57232 | 2.32E−10 |
| SEQ ID NO: 1561 | 2hpf_pa-6hpf_pa | active | AUUUUU | 1.65985 | 2.61E−10 |
| SEQ ID NO: 1562 | 2hpf_pa-6hpf_pa | active | UCUUU | 1.41888 | 5.15E−10 |
| SEQ ID NO: 1563 | 2hpf_pa-6hpf_pa | active | UUAUUU | 1.56709 | 1.15E−09 |
| SEQ ID NO: 1564 | 2hpf_pa-6hpf_pa | active | CCCUC | 1.68837 | 3.29E−09 |
| SEQ ID NO: 1565 | 2hpf_pa-6hpf_pa | active | UUUCUC | 1.72399 | 4.40E−09 |
| SEQ ID NO: 1566 | 2hpf_pa-6hpf_pa | active | UUCCC | 1.57544 | 4.48E−09 |
| SEQ ID NO: 1567 | 2hpf_pa-6hpf_pa | active | CUCUC | 1.52454 | 7.63E−09 |
| SEQ ID NO: 1568 | 2hpf_pa-6hpf_pa | active | UCUUUU | 1.67485 | 8.02E−09 |
| SEQ ID NO: 1569 | 2hpf_pa-6hpf_pa | active | UUUCUU | 1.66392 | 9.16E−09 |
| SEQ ID NO: 1570 | 2hpf_pa-6hpf_pa | active | UUUUGC | 1.72381 | 9.20E−09 |
| SEQ ID NO: 1571 | 2hpf_pa-6hpf_pa | active | UUCCU | 1.48259 | 9.89E−09 |
| SEQ ID NO: 1572 | 2hpf_pa-6hpf_pa | active | UUUUAA | 1.53049 | 1.31E−08 |
| SEQ ID NO: 1573 | 2hpf_pa-6hpf_pa | active | UUUUGU | 1.56727 | 1.46E−08 |
| SEQ ID NO: 1574 | 2hpf_pa-6hpf_pa | active | UUUAA | 1.32743 | 1.78E−08 |
| SEQ ID NO: 1575 | 2hpf_pa-6hpf_pa | active | UUCUC | 1.44247 | 2.82E−08 |
| SEQ ID NO: 1576 | 2hpf_pa-6hpf_pa | active | AUAUUU | 1.62244 | 6.53E−08 |
| SEQ ID NO: 1577 | 2hpf_pa-6hpf_pa | active | UUCUUU | 1.66054 | 8.09E−08 |
| SEQ ID NO: 1578 | 2hpf_pa-6hpf_pa | active | UUUUCU | 1.55174 | 8.34E−08 |
| SEQ ID NO: 1579 | 2hpf_pa-6hpf_pa | active | GCUUU | 1.37294 | 9.44E−08 |
| SEQ ID NO: 1580 | 2hpf_pa-6hpf_pa | active | UCUCUC | 1.87121 | 1.40E−07 |
| SEQ ID NO: 1581 | 2hpf_pa-6hpf_pa | active | UUAAU | 1.45056 | 1.45E−07 |
| SEQ ID NO: 1582 | 2hpf_pa-6hpf_pa | active | CUUCUU | 1.94376 | 1.95E−07 |
| SEQ ID NO: 1583 | 2hpf_pa-6hpf_pa | active | UCCCUC | 1.97855 | 1.95E−07 |
| SEQ ID NO: 1584 | 2hpf_pa-6hpf_pa | active | UGUUUU | 1.41479 | 1.98E−07 |
| SEQ ID NO: 1585 | 2hpf_pa-6hpf_pa | active | CUUUG | 1.42127 | 2.69E−07 |
| SEQ ID NO: 1586 | 2hpf_pa-6hpf_pa | active | UUUCC | 1.36638 | 3.23E−07 |
| SEQ ID NO: 1587 | 2hpf_pa-6hpf_pa | active | UUUACG | 1.9588 | 3.48E−07 |
| SEQ ID NO: 1588 | 2hpf_pa-6hpf_pa | active | AUAUU | 1.38094 | 5.46E−07 |
| SEQ ID NO: 1589 | 2hpf_pa-6hpf_pa | active | UUGCAU | 1.9336 | 5.48E−07 |
| SEQ ID NO: 1590 | 2hpf_pa-6hpf_pa | active | UUAGU | 1.48417 | 7.60E−07 |
| SEQ ID NO: 1591 | 2hpf_pa-6hpf_pa | active | CGUUUU | 1.59949 | 7.72E−07 |
| SEQ ID NO: 1592 | 2hpf_pa-6hpf_pa | active | UCCCU | 1.54427 | 7.73E−07 |
| SEQ ID NO: 1593 | 2hpf_pa-6hpf_pa | active | UUUUAC | 1.6532 | 7.79E−07 |
| SEQ ID NO: 1594 | 2hpf_pa-6hpf_pa | active | UUUAC | 1.36552 | 8.58E−07 |
| SEQ ID NO: 1595 | 2hpf_pa-6hpf_pa | active | AACUUU | 1.68984 | 9.86E−07 |
| SEQ ID NO: 1596 | 2hpf_pa-6hpf_pa | active | CGUUU | 1.3344 | 1.02E−06 |
| SEQ ID NO: 1597 | 2hpf_pa-6hpf_pa | active | UCCCC | 1.56862 | 1.05E−06 |
| SEQ ID NO: 1598 | 2hpf_pa-6hpf_pa | active | CUCUU | 1.40227 | 1.26E−06 |
| SEQ ID NO: 1599 | 2hpf_pa-6hpf_pa | active | UAUAUU | 1.80092 | 1.27E−06 |
| SEQ ID NO: 1600 | 2hpf_pa-6hpf_pa | active | UUUUCC | 1.59691 | 1.30E−06 |
| SEQ ID NO: 1601 | 2hpf_pa-6hpf_pa | active | UUUUUC | 1.50505 | 1.33E−06 |
| SEQ ID NO: 1602 | 2hpf_pa-6hpf_pa | active | ACCUCU | 2.12015 | 1.35E−06 |
| SEQ ID NO: 1603 | 2hpf_pa-6hpf_pa | active | UAAUUC | 2.01255 | 1.38E−06 |
| SEQ ID NO: 1604 | 2hpf_pa-6hpf_pa | active | UAUAU | 1.52995 | 1.66E−06 |
| SEQ ID NO: 1605 | 2hpf_pa-6hpf_pa | active | UCUCU | 1.40062 | 1.73E−06 |
| SEQ ID NO: 1606 | 2hpf_pa-6hpf_pa | active | AGUUU | 1.31505 | 1.93E−06 |
| SEQ ID NO: 1607 | 2hpf_pa-6hpf_pa | active | UAUUUC | 1.67374 | 2.20E−06 |
| SEQ ID NO: 1608 | 2hpf_pa-6hpf_pa | active | UUCUCU | 1.66076 | 2.32E−06 |
| SEQ ID NO: 1609 | 2hpf_pa-6hpf_pa | active | AUUAAU | 1.9074 | 2.43E−06 |
| SEQ ID NO: 1610 | 2hpf_pa-6hpf_pa | active | UUACU | 1.48404 | 2.79E−06 |
| SEQ ID NO: 1611 | 2hpf_pa-6hpf_pa | active | AUUUCU | 1.63633 | 3.30E−06 |
| SEQ ID NO: 1612 | 2hpf_pa-6hpf_pa | active | UUUAUA | 1.651 | 3.35E−06 |
| SEQ ID NO: 1613 | 2hpf_pa-6hpf_pa | active | CUUUUA | 1.60293 | 3.40E−06 |
| SEQ ID NO: 1614 | 2hpf_pa-6hpf_pa | active | AGCUU | 1.41878 | 3.51E−06 |
| SEQ ID NO: 1615 | 2hpf_pa-6hpf_pa | active | UCUAC | 1.60071 | 3.61E−06 |
| SEQ ID NO: 1616 | 2hpf_pa-6hpf_pa | active | CCCCC | 1.71799 | 3.78E−06 |
| SEQ ID NO: 1617 | 2hpf_pa-6hpf_pa | active | UAGCU | 1.4478 | 4.44E−06 |
| SEQ ID NO: 1618 | 2hpf_pa-6hpf_pa | active | UUCUA | 1.57544 | 4.59E−06 |
| SEQ ID NO: 1619 | 2hpf_pa-6hpf_pa | active | CUUUUC | 1.5869 | 5.19E−06 |
| SEQ ID NO: 1620 | 2hpf_pa-6hpf_pa | active | UUACUU | 1.80586 | 5.42E−06 |
| SEQ ID NO: 1621 | 2hpf_pa-6hpf_pa | active | UUAGCU | 1.72888 | 5.46E−06 |
| SEQ ID NO: 1622 | 2hpf_pa-6hpf_pa | active | CUUUAU | 1.72977 | 6.24E−06 |
| SEQ ID NO: 1623 | 2hpf_pa-6hpf_pa | active | CCUUUU | 1.67409 | 6.38E−06 |
| SEQ ID NO: 1624 | 2hpf_pa-6hpf_pa | active | UAAUU | 1.41802 | 6.47E−06 |
| SEQ ID NO: 1625 | 2hpf_pa-6hpf_pa | active | UUCCUC | 1.64337 | 6.64E−06 |
| SEQ ID NO: 1626 | 2hpf_pa-6hpf_pa | active | CCUCUA | 2.33082 | 8.68E−06 |
| SEQ ID NO: 1627 | 2hpf_pa-6hpf_pa | active | UACUGU | 1.93057 | 8.95E−06 |
| SEQ ID NO: 1628 | 2hpf_pa-6hpf_pa | active | GUUUUA | 1.48032 | 9.04E-06 |
| SEQ ID NO: 1629 | 2hpf_pa-6hpf_pa | repressed | AUGGC | 3.21133 | 1.95E−51 |
| SEQ ID NO: 1630 | 2hpf_pa-6hpf_pa | repressed | GAUGG | 2.93612 | 1.38E−41 |
| SEQ ID NO: 1631 | 2hpf_pa-6hpf_pa | repressed | GGAUG | 2.70231 | 3.39E−40 |
| SEQ ID NO: 1632 | 2hpf_pa-6hpf_pa | repressed | AUGGA | 2.64239 | 7.77E−39 |
| SEQ ID NO: 1633 | 2hpf_pa-6hpf_pa | repressed | CAUGG | 2.765 | 1.49E−35 |
| SEQ ID NO: 1634 | 2hpf_pa-6hpf_pa | repressed | AGGAUG | 4.57367 | 7.42E−34 |
| SEQ ID NO: 1635 | 2hpf_pa-6hpf_pa | repressed | CCAUG | 2.868 | 5.09E−29 |
| SEQ ID NO: 1636 | 2hpf_pa-6hpf_pa | repressed | AGAUG | 2.36363 | 3.35E−27 |
| SEQ ID NO: 1637 | 2hpf_pa-6hpf_pa | repressed | AUGGCG | 4.10916 | 5.29E−26 |
| SEQ ID NO: 1638 | 2hpf_pa-6hpf_pa | repressed | GAUGGC | 4.25741 | 8.73E−24 |
| SEQ ID NO: 1639 | 2hpf_pa-6hpf_pa | repressed | GAUGU | 2.14643 | 1.95E−23 |
| SEQ ID NO: 1640 | 2hpf_pa-6hpf_pa | repressed | AUGUG | 2.00103 | 2.73E−22 |
| SEQ ID NO: 1641 | 2hpf_pa-6hpf_pa | repressed | AUGGAG | 3.11691 | 4.79E−21 |
| SEQ ID NO: 1642 | 2hpf_pa-6hpf_pa | repressed | AAUGG | 2.2813 | 1.65E−20 |
| SEQ ID NO: 1643 | 2hpf_pa-6hpf_pa | repressed | AUGGG | 2.45779 | 1.92E−19 |
| SEQ ID NO: 1644 | 2hpf_pa-6hpf_pa | repressed | AAUGGC | 3.46761 | 8.85E−19 |
| SEQ ID NO: 1645 | 2hpf_pa-6hpf_pa | repressed | AUGAG | 2.04532 | 5.36E−18 |
| SEQ ID NO: 1646 | 2hpf_pa-6hpf_pa | repressed | AUCAUG | 3.3915 | 5.76E−18 |
| SEQ ID NO: 1647 | 2hpf_pa-6hpf_pa | repressed | GGAUGC | 3.54604 | 7.04E−18 |
| SEQ ID NO: 1648 | 2hpf_pa-6hpf_pa | repressed | UCAUG | 1.9889 | 1.58E−17 |
| SEQ ID NO: 1649 | 2hpf_pa-6hpf_pa | repressed | GGGGC | 2.2568 | 1.93E−17 |
| SEQ ID NO: 1650 | 2hpf_pa-6hpf_pa | repressed | CGAUG | 2.38862 | 2.11E−17 |
| SEQ ID NO: 1651 | 2hpf_pa-6hpf_pa | repressed | UGGAG | 1.74869 | 2.54E−17 |
| SEQ ID NO: 1652 | 2hpf_pa-6hpf_pa | repressed | AAAAUG | 2.85296 | 8.86E−17 |
| SEQ ID NO: 1653 | 2hpf_pa-6hpf_pa | repressed | GAUGC | 2.17126 | 1.03E−16 |
| SEQ ID NO: 1654 | 2hpf_pa-6hpf_pa | repressed | CAAUG | 2.17969 | 1.74E−16 |
| SEQ ID NO: 1655 | 2hpf_pa-6hpf_pa | repressed | GGAUGG | 3.57318 | 2.74E−16 |
| SEQ ID NO: 1656 | 2hpf_pa-6hpf_pa | repressed | AAAUG | 1.84436 | 3.33E−16 |
| SEQ ID NO: 1657 | 2hpf_pa-6hpf_pa | repressed | GAUGA | 1.96781 | 3.82E−16 |
| SEQ ID NO: 1658 | 2hpf_pa-6hpf_pa | repressed | GGGAUG | 3.35568 | 4.21E−16 |
| SEQ ID NO: 1659 | 2hpf_pa-6hpf_pa | repressed | UGGCG | 2.02691 | 5.33E−16 |
| SEQ ID NO: 1660 | 2hpf_pa-6hpf_pa | repressed | AGGGG | 2.15165 | 8.33E−16 |
| SEQ ID NO: 1661 | 2hpf_pa-6hpf_pa | repressed | GUCAUG | 3.31373 | 2.62E−15 |
| SEQ ID NO: 1662 | 2hpf_pa-6hpf_pa | repressed | GCCAUG | 3.46034 | 1.64E−14 |
| SEQ ID NO: 1663 | 2hpf_pa-6hpf_pa | repressed | GAUGGA | 2.88603 | 2.53E−14 |
| SEQ ID NO: 1664 | 2hpf_pa-6hpf_pa | repressed | AAGAUG | 3.02346 | 3.46E−14 |
| SEQ ID NO: 1665 | 2hpf_pa-6hpf_pa | repressed | AAAUGG | 2.90142 | 5.23E−14 |
| SEQ ID NO: 1666 | 2hpf_pa-6hpf_pa | repressed | ACCAUG | 4.00196 | 6.84E−14 |
| SEQ ID NO: 1667 | 2hpf_pa-6hpf_pa | repressed | GAUGUG | 2.57373 | 7.06E−14 |
| SEQ ID NO: 1668 | 2hpf_pa-6hpf_pa | repressed | AUGAGG | 2.93338 | 8.45E−14 |
| SEQ ID NO: 1669 | 2hpf_pa-6hpf_pa | repressed | AAUGU | 1.76328 | 9.36E−14 |
| SEQ ID NO: 1670 | 2hpf_pa-6hpf_pa | repressed | CCAUGG | 3.36043 | 1.96E−13 |
| SEQ ID NO: 1671 | 2hpf_pa-6hpf_pa | repressed | GGCGG | 1.8473 | 2.27E−13 |
| SEQ ID NO: 1672 | 2hpf_pa-6hpf_pa | repressed | CAUGGA | 2.6853 | 3.40E−13 |
| SEQ ID NO: 1673 | 2hpf_pa-6hpf_pa | repressed | UCAUGG | 2.88912 | 4.55E−13 |
| SEQ ID NO: 1674 | 2hpf_pa-6hpf_pa | repressed | ACAAUG | 3.239 | 4.66E−13 |
| SEQ ID NO: 1675 | 2hpf_pa-6hpf_pa | repressed | AUGGCA | 2.96657 | 6.47E−13 |
| SEQ ID NO: 1676 | 2hpf_pa-6hpf_pa | repressed | CAUGGC | 2.8435 | 9.17E−13 |
| SEQ ID NO: 1677 | 2hpf_pa-6hpf_pa | repressed | GGAGC | 1.67181 | 1.07E−12 |
| SEQ ID NO: 1678 | 2hpf_pa-6hpf_pa | repressed | CAUGGG | 3.62923 | 1.09E−12 |
| SEQ ID NO: 1679 | 2hpf_pa-6hpf_pa | repressed | GGCUG | 1.6482 | 2.38E−12 |
| SEQ ID NO: 1680 | 2hpf_pa-6hpf_pa | repressed | AUGUGG | 2.64704 | 2.39E−12 |
| SEQ ID NO: 1681 | 2hpf_pa-6hpf_pa | repressed | AGAUGU | 2.62137 | 2.45E−12 |
| SEQ ID NO: 1682 | 2hpf_pa-6hpf_pa | repressed | UAUGGA | 2.92827 | 5.07E−12 |
| SEQ ID NO: 1683 | 2hpf_pa-6hpf_pa | repressed | GGCGGC | 2.47226 | 7.75E−12 |
| SEQ ID NO: 1684 | 2hpf_pa-6hpf_pa | repressed | ACGAUG | 3.81139 | 7.76E−12 |
| SEQ ID NO: 1685 | 2hpf_pa-6hpf_pa | repressed | UAUGG | 2.00891 | 8.60E−12 |
| SEQ ID NO: 1686 | 2hpf_pa-6hpf_pa | repressed | CAUGA | 1.87985 | 1.14E−11 |
| SEQ ID NO: 1687 | 2hpf_pa-6hpf_pa | repressed | UGAUG | 1.7239 | 1.29E−11 |
| SEQ ID NO: 1688 | 2hpf_pa-6hpf_pa | repressed | AGGAU | 1.74254 | 1.59E−11 |
| SEQ ID NO: 1689 | 2hpf_pa-6hpf_pa | repressed | GAAAUG | 2.60792 | 2.41E−11 |
| SEQ ID NO: 1690 | 2hpf_pa-6hpf_pa | repressed | AGAUGG | 2.79207 | 3.28E−11 |
| SEQ ID NO: 1691 | 2hpf_pa-6hpf_pa | repressed | AUGGCU | 2.81575 | 3.78E−11 |
| SEQ ID NO: 1692 | 2hpf_pa-6hpf_pa | repressed | GGGGCG | 2.94665 | 5.02E−11 |
| SEQ ID NO: 1693 | 2hpf_pa-6hpf_pa | repressed | CAGGG | 1.86982 | 6.10E−11 |
| SEQ ID NO: 1694 | 2hpf_pa-6hpf_pa | repressed | GAGAUG | 2.8495 | 6.29E−11 |
| SEQ ID NO: 1695 | 2hpf_pa-6hpf_pa | repressed | AAAUGA | 2.24469 | 9.69E−11 |
| SEQ ID NO: 1696 | 2hpf_pa-6hpf_pa | repressed | GCAUGG | 2.86807 | 1.31E−10 |
| SEQ ID NO: 1697 | 2hpf_pa-6hpf_pa | repressed | UGUGGC | 2.46817 | 1.60E−10 |
| SEQ ID NO: 1698 | 2hpf_pa-6hpf_pa | repressed | AUGUC | 1.83966 | 1.68E−10 |
| SEQ ID NO: 1699 | 2hpf_pa-6hpf_pa | repressed | GGAGCG | 2.26138 | 2.33E−10 |
| SEQ ID NO: 1700 | 2hpf_pa-6hpf_pa | repressed | AUGGAA | 2.67177 | 2.67E−10 |
| SEQ ID NO: 1701 | 2hpf_pa-6hpf_pa | repressed | GGGGA | 1.78822 | 2.96E−10 |
| SEQ ID NO: 1702 | 2hpf_pa-6hpf_pa | repressed | CGAUGG | 3.07843 | 4.68E−10 |
| SEQ ID NO: 1703 | 2hpf_pa-6hpf_pa | repressed | GAGGG | 1.69558 | 4.79E−10 |
| SEQ ID NO: 1704 | 2hpf_pa-6hpf_pa | repressed | GCGAUG | 2.87911 | 4.98E−10 |
| SEQ ID NO: 1705 | 2hpf_pa-6hpf_pa | repressed | GGAGG | 1.6246 | 5.37E−10 |
| SEQ ID NO: 1706 | 2hpf_pa-6hpf_pa | repressed | AGCCAU | 2.50123 | 6.14E−10 |
| SEQ ID NO: 1707 | 2hpf_pa-6hpf_pa | repressed | GGCAUG | 3.33497 | 8.14E−10 |
| SEQ ID NO: 1708 | 2hpf_pa-6hpf_pa | repressed | CCAUGU | 2.96145 | 9.14E−10 |
| SEQ ID NO: 1709 | 2hpf_pa-6hpf_pa | repressed | GGGCUG | 2.32114 | 9.64E−10 |
| SEQ ID NO: 1710 | 2hpf_pa-6hpf_pa | repressed | GGGAU | 1.66753 | 1.14E−09 |
| SEQ ID NO: 1711 | 2hpf_pa-6hpf_pa | repressed | AAUGUG | 2.13271 | 1.74E−09 |
| SEQ ID NO: 1712 | 2hpf_pa-6hpf_pa | repressed | CGGGG | 1.85351 | 2.00E−09 |
| SEQ ID NO: 1713 | 2hpf_pa-6hpf_pa | repressed | GAUGCG | 2.92827 | 2.15E−09 |
| SEQ ID NO: 1714 | 2hpf_pa-6hpf_pa | repressed | UUAUGG | 2.60483 | 2.29E−09 |
| SEQ ID NO: 1715 | 2hpf_pa-6hpf_pa | repressed | UGGCGG | 2.36723 | 2.37E−09 |
| SEQ ID NO: 1716 | 2hpf_pa-6hpf_pa | repressed | AUGAU | 1.73 | 2.66E−09 |
| SEQ ID NO: 1717 | 2hpf_pa-6hpf_pa | repressed | GGGGG | 1.90988 | 2.91E−09 |
| SEQ ID NO: 1718 | 2hpf_pa-6hpf_pa | repressed | AUGGAC | 2.60734 | 3.35E−09 |
| SEQ ID NO: 1719 | 2hpf_pa-6hpf_pa | repressed | GCGGC | 1.6204 | 3.74E−09 |
| SEQ ID NO: 1720 | 2hpf_pa-6hpf_pa | repressed | AGCAUG | 2.62629 | 4.01E−09 |
| SEQ ID NO: 1721 | 2hpf_pa-6hpf_pa | repressed | CAAUGU | 2.5594 | 4.13E−09 |
| SEQ ID NO: 1722 | 2hpf_pa-6hpf_pa | repressed | GCGGCG | 2.1689 | 4.45E−09 |
| SEQ ID NO: 1723 | 2hpf_pa-6hpf_pa | repressed | AAUGA | 1.62306 | 4.77E−09 |
| SEQ ID NO: 1724 | 2hpf_pa-6hpf_pa | repressed | CAGGGG | 2.80441 | 4.79E−09 |
| SEQ ID NO: 1725 | 2hpf_pa-6hpf_pa | repressed | UCAGGG | 2.59621 | 8.74E−09 |
| SEQ ID NO: 1726 | 2hpf_pa-6hpf_pa | repressed | GCAUG | 1.73865 | 8.87E−09 |
| SEQ ID NO: 1727 | 2hpf_pa-6hpf_pa | repressed | AUGAUG | 2.43506 | 1.01E−08 |
| SEQ ID NO: 1728 | 2hpf_pa-6hpf_pa | repressed | GAAGGG | 2.46022 | 1.04E−08 |
| SEQ ID NO: 1729 | 2hpf_pa-6hpf_pa | repressed | GAAUG | 1.62628 | 1.08E−08 |
| SEQ ID NO: 1730 | 2hpf_pa-6hpf_pa | repressed | AUGAA | 1.59774 | 1.48E−08 |
| SEQ ID NO: 1731 | 2hpf_pa-6hpf_pa | repressed | UGAUGG | 2.36297 | 1.94E−08 |
| SEQ ID NO: 1732 | 2hpf_pa-6hpf_pa | repressed | CGAUGA | 2.7081 | 2.09E−08 |
| SEQ ID NO: 1733 | 2hpf_pa-6hpf_pa | repressed | GGAUGA | 2.24248 | 2.32E−08 |
| SEQ ID NO: 1734 | 2hpf_pa-6hpf_pa | repressed | AUGGCC | 2.8843 | 2.43E−08 |
| SEQ ID NO: 1735 | 2hpf_pa-6hpf_pa | repressed | UGUGG | 1.51231 | 2.54E−08 |
| SEQ ID NO: 1736 | 2hpf_pa-6hpf_pa | repressed | GGGCU | 1.67716 | 2.79E−08 |
| SEQ ID NO: 1737 | 2hpf_pa-6hpf_pa | repressed | AAUGAG | 2.20025 | 3.36E−08 |
| SEQ ID NO: 1738 | 2hpf_pa-6hpf_pa | repressed | AUGGGA | 2.35718 | 4.13E−08 |
| SEQ ID NO: 1739 | 2hpf_pa-6hpf_pa | repressed | GGCCG | 1.734 | 4.60E−08 |
| SEQ ID NO: 1740 | 2hpf_pa-6hpf_pa | repressed | AGGGGC | 2.67307 | 4.66E−08 |
| SEQ ID NO: 1741 | 2hpf_pa-6hpf_pa | repressed | AUGCG | 1.77826 | 5.48E−08 |
| SEQ ID NO: 1742 | 2hpf_pa-6hpf_pa | repressed | AGAAUG | 2.69974 | 5.51E−08 |
| SEQ ID NO: 1743 | 2hpf_pa-6hpf_pa | repressed | UGAGGG | 2.23854 | 6.11E−08 |
| SEQ ID NO: 1744 | 2hpf_pa-6hpf_pa | repressed | GAAUGU | 2.14391 | 6.29E−08 |
| SEQ ID NO: 1745 | 2hpf_pa-6hpf_pa | repressed | CGCUG | 1.45649 | 6.48E−08 |
| SEQ ID NO: 1746 | 2hpf_pa-6hpf_pa | repressed | GGGCG | 1.7679 | 7.22E−08 |
| SEQ ID NO: 1747 | 2hpf_pa-6hpf_pa | repressed | GCGGG | 1.62937 | 7.33E−08 |
| SEQ ID NO: 1748 | 2hpf_pa-6hpf_pa | repressed | CGGCC | 1.78591 | 7.33E−08 |
| SEQ ID NO: 1749 | 2hpf_pa-6hpf_pa | repressed | CAUGU | 1.61208 | 7.40E−08 |
| SEQ ID NO: 1750 | 2hpf_pa-6hpf_pa | repressed | AUGGGG | 2.98019 | 8.23E−08 |
| SEQ ID NO: 1751 | 2hpf_pa-6hpf_pa | repressed | CAGAUG | 2.13815 | 9.07E−08 |
| SEQ ID NO: 1752 | 2hpf_pa-6hpf_pa | repressed | GAGCG | 1.50122 | 9.92E−08 |
| SEQ ID NO: 1753 | 2hpf_pa-6hpf_pa | repressed | UGAGG | 1.52063 | 1.04E−07 |
| SEQ ID NO: 1754 | 2hpf_pa-6hpf_pa | repressed | GGGGGC | 2.51824 | 1.05E−07 |
| SEQ ID NO: 1755 | 2hpf_pa-6hpf_pa | repressed | GAGAU | 1.5943 | 1.23E−07 |
| SEQ ID NO: 1756 | 2hpf_pa-6hpf_pa | repressed | CGGGCU | 2.18857 | 1.28E−07 |
| SEQ ID NO: 1757 | 2hpf_pa-6hpf_pa | repressed | GAUGCU | 2.29279 | 1.36E−07 |
| SEQ ID NO: 1758 | 2hpf_pa-6hpf_pa | repressed | CGGGC | 1.71517 | 1.41E−07 |
| SEQ ID NO: 1759 | 2hpf_pa-6hpf_pa | repressed | CAUGC | 1.7225 | 1.55E−07 |
| SEQ ID NO: 1760 | 2hpf_pa-6hpf_pa | repressed | GCGCUG | 1.86316 | 1.66E−07 |
| SEQ ID NO: 1761 | 2hpf_pa-6hpf_pa | repressed | GAGGA | 1.40599 | 1.69E−07 |
| SEQ ID NO: 1762 | 2hpf_pa-6hpf_pa | repressed | AGGGA | 1.59869 | 2.31E−07 |
| SEQ ID NO: 1763 | 2hpf_pa-6hpf_pa | repressed | UGGAGC | 1.87258 | 2.40E−07 |
| SEQ ID NO: 1764 | 2hpf_pa-6hpf_pa | repressed | UGGCCG | 2.29621 | 2.46E−07 |
| SEQ ID NO: 1765 | 2hpf_pa-6hpf_pa | repressed | AGGGGG | 2.34033 | 2.75E−07 |
| SEQ ID NO: 1766 | 2hpf_pa-6hpf_pa | repressed | UGGAGG | 2.00803 | 2.87E−07 |
| SEQ ID NO: 1767 | 2hpf_pa-6hpf_pa | repressed | GGGAG | 1.58179 | 2.93E−07 |
| SEQ ID NO: 1768 | 2hpf_pa-6hpf_pa | repressed | GAGGGG | 2.2346 | 2.93E−07 |
| SEQ ID NO: 1769 | 2hpf_pa-6hpf_pa | repressed | GGAGGA | 1.77865 | 3.27E−07 |
| SEQ ID NO: 1770 | 2hpf_pa-6hpf_pa | repressed | AACAUG | 2.27139 | 3.37E−07 |
| SEQ ID NO: 1771 | 2hpf_pa-6hpf_pa | repressed | GCCCGG | 2.37617 | 3.57E−07 |
| SEQ ID NO: 1772 | 2hpf_pa-6hpf_pa | repressed | UCAUGA | 2.25917 | 3.93E−07 |
| SEQ ID NO: 1773 | 2hpf_pa-6hpf_pa | repressed | UCAGG | 1.55941 | 5.08E−07 |
| SEQ ID NO: 1774 | 2hpf_pa-6hpf_pa | repressed | UGGCC | 1.6734 | 5.11E−07 |
| SEQ ID NO: 1775 | 2hpf_pa-6hpf_pa | repressed | GAUGUC | 2.46498 | 5.79E−07 |
| SEQ ID NO: 1776 | 2hpf_pa-6hpf_pa | repressed | GGCAG | 1.52204 | 6.58E−07 |
| SEQ ID NO: 1777 | 2hpf_pa-6hpf_pa | repressed | AUGUGU | 1.86007 | 6.61E−07 |
| SEQ ID NO: 1778 | 2hpf_pa-6hpf_pa | repressed | ACAUG | 1.60377 | 6.76E−07 |
| SEQ ID NO: 1779 | 2hpf_pa-6hpf_pa | repressed | CGGAGC | 1.97939 | 7.35E−07 |
| SEQ ID NO: 1780 | 2hpf_pa-6hpf_pa | repressed | GUGAUG | 2.20003 | 8.32E−07 |
| SEQ ID NO: 1781 | 2hpf_pa-6hpf_pa | repressed | GAAGG | 1.50453 | 8.76E−07 |
| SEQ ID NO: 1782 | 2hpf_pa-6hpf_pa | repressed | CUCGGG | 2.44564 | 1.02E−06 |
| SEQ ID NO: 1783 | 2hpf_pa-6hpf_pa | repressed | CCGGC | 1.67695 | 1.04E−06 |
| SEQ ID NO: 1784 | 2hpf_pa-6hpf_pa | repressed | GAAGAU | 1.90416 | 1.14E−06 |
| SEQ ID NO: 1785 | 2hpf_pa-6hpf_pa | repressed | GGAUGU | 2.0715 | 1.20E−06 |
| SEQ ID NO: 1786 | 2hpf_pa-6hpf_pa | repressed | GCGCU | 1.43782 | 1.30E−06 |
| SEQ ID NO: 1787 | 2hpf_pa-6hpf_pa | repressed | CUCGG | 1.55785 | 1.33E−06 |
| SEQ ID NO: 1788 | 2hpf_pa-6hpf_pa | repressed | CCAUGA | 2.56763 | 1.55E−06 |
| SEQ ID NO: 1789 | 2hpf_pa-6hpf_pa | repressed | AUGUCG | 2.36227 | 1.65E−06 |
| SEQ ID NO: 1790 | 2hpf_pa-6hpf_pa | repressed | AUGAGC | 2.02631 | 1.80E−06 |
| SEQ ID NO: 1791 | 2hpf_pa-6hpf_pa | repressed | GAUGAU | 2.07475 | 1.91E−06 |
| SEQ ID NO: 1792 | 2hpf_pa-6hpf_pa | repressed | GGGUG | 1.60739 | 2.13E−06 |
| SEQ ID NO: 1793 | 2hpf_pa-6hpf_pa | repressed | CCGGGG | 2.44214 | 2.20E−06 |
| SEQ ID NO: 1794 | 2hpf_pa-6hpf_pa | repressed | GGCCA | 1.71199 | 2.39E−06 |
| SEQ ID NO: 1795 | 2hpf_pa-6hpf_pa | repressed | UCCAUG | 2.28871 | 2.54E−06 |
| SEQ ID NO: 1796 | 2hpf_pa-6hpf_pa | repressed | GAGGAU | 1.82443 | 2.72E−06 |
| SEQ ID NO: 1797 | 2hpf_pa-6hpf_pa | repressed | AGAUGA | 2.04646 | 2.81E−06 |
| SEQ ID NO: 1798 | 2hpf_pa-6hpf_pa | repressed | GAUGUU | 1.88645 | 2.81E−06 |
| SEQ ID NO: 1799 | 2hpf_pa-6hpf_pa | repressed | CAGGAU | 2.10103 | 2.93E−06 |
| SEQ ID NO: 1800 | 2hpf_pa-6hpf_pa | repressed | UGGGUG | 2.14009 | 3.09E−06 |
| SEQ ID NO: 1801 | 2hpf_pa-6hpf_pa | repressed | GCCAU | 1.57732 | 3.16E−06 |
| SEQ ID NO: 1802 | 2hpf_pa-6hpf_pa | repressed | GGCCGC | 2.29843 | 3.17E−06 |
| SEQ ID NO: 1803 | 2hpf_pa-6hpf_pa | repressed | GAUGAG | 2.01853 | 3.24E−06 |
| SEQ ID NO: 1804 | 2hpf_pa-6hpf_pa | repressed | GCUGAA | 1.6641 | 3.60E−06 |
| SEQ ID NO: 1805 | 2hpf_pa-6hpf_pa | repressed | GGGCA | 1.64495 | 3.86E−06 |
| SEQ ID NO: 1806 | 2hpf_pa-6hpf_pa | repressed | GCAAUG | 2.33691 | 4.18E−06 |
| SEQ ID NO: 1807 | 2hpf_pa-6hpf_pa | repressed | UGGCA | 1.59211 | 4.52E−06 |
| SEQ ID NO: 1808 | 2hpf_pa-6hpf_pa | repressed | UGGCUG | 1.77865 | 4.53E−06 |
| SEQ ID NO: 1809 | 2hpf_pa-6hpf_pa | repressed | UAUGAG | 2.40118 | 4.59E−06 |
| SEQ ID NO: 1810 | 2hpf_pa-6hpf_pa | repressed | CCCGC | 1.63272 | 4.59E−06 |
| SEQ ID NO: 1811 | 2hpf_pa-6hpf_pa | repressed | GUGCGG | 2.17754 | 4.64E−06 |
| SEQ ID NO: 1812 | 2hpf_pa-6hpf_pa | repressed | UGGGG | 1.58945 | 4.85E−06 |
| SEQ ID NO: 1813 | 2hpf_pa-6hpf_pa | repressed | AAUAUG | 2.1962 | 5.04E−06 |
| SEQ ID NO: 1814 | 2hpf_pa-6hpf_pa | repressed | AUGAAG | 1.89874 | 5.11E−06 |
| SEQ ID NO: 1815 | 2hpf_pa-6hpf_pa | repressed | GCCGC | 1.4977 | 5.18E−06 |
| SEQ ID NO: 1816 | 2hpf_pa-6hpf_pa | repressed | GGCAGC | 1.94427 | 5.92E−06 |
| SEQ ID NO: 1817 | 2hpf_pa-6hpf_pa | repressed | GGAGGG | 1.97522 | 5.92E−06 |
| SEQ ID NO: 1818 | 2hpf_pa-6hpf_pa | repressed | GGGGCC | 2.60545 | 5.97E−06 |
| SEQ ID NO: 1819 | 2hpf_pa-6hpf_pa | repressed | AUGAC | 1.60443 | 6.47E−06 |
| SEQ ID NO: 1820 | 2hpf_pa-6hpf_pa | repressed | CGGAG | 1.42399 | 6.67E−06 |
| SEQ ID NO: 1821 | 2hpf_pa-6hpf_pa | repressed | CCGGCC | 2.47828 | 7.17E−06 |
| SEQ ID NO: 1822 | 2hpf_pa-6hpf_pa | repressed | CGCUGU | 1.704 | 7.79E−06 |
| SEQ ID NO: 1823 | 2hpf_pa-6hpf_pa | repressed | AUGUCA | 2.10048 | 8.48E−06 |
| SEQ ID NO: 1824 | 2hpf_pa-6hpf_pa | repressed | GGAGA | 1.37949 | 8.70E−06 |
| SEQ ID NO: 1825 | 2hpf_pa-6hpf_pa | repressed | UGAUGU | 1.80468 | 9.29E−06 |
| SEQ ID NO: 1826 | 2hpf_pa-6hpf_pa | repressed | GGACGG | 2.11532 | 9.34E−06 |
| SEQ ID NO: 1827 | 2hpf_pa-6hpf_pa | repressed | GCAGA | 1.39548 | 9.52E−06 |
| SEQ ID NO: 1828 | 2hpf_pa-6hpf_pa | s1_activated | AGCACU | 2.66642 | 1.23E−10 |
| SEQ ID NO: 1829 | 2hpf_pa-6hpf_pa | s1_activated | GCACUU | 2.28426 | 9.77E−10 |
| SEQ ID NO: 1830 | 2hpf_pa-6hpf_pa | s1_activated | GUUUG | 1.40251 | 1.19E−09 |
| SEQ ID NO: 1831 | 2hpf_pa-6hpf_pa | s1_activated | UAGUG | 1.77143 | 3.12E−09 |
| SEQ ID NO: 1832 | 2hpf_pa-6hpf_pa | s1_activated | AAGAA | 1.57123 | 3.96E−09 |
| SEQ ID NO: 1833 | 2hpf_pa-6hpf_pa | s1_activated | UAAGAA | 2.46386 | 4.73E−09 |
| SEQ ID NO: 1834 | 2hpf_pa-6hpf_pa | s1_activated | UAUUG | 1.60393 | 5.92E−09 |
| SEQ ID NO: 1835 | 2hpf_pa-6hpf_pa | s1_activated | AGACU | 1.63756 | 7.91E−09 |
| SEQ ID NO: 1836 | 2hpf_pa-6hpf_pa | s1_activated | AGUUAC | 2.65643 | 9.99E−09 |
| SEQ ID NO: 1837 | 2hpf_pa-6hpf_pa | s1_activated | UUUUG | 1.30199 | 6.99E−08 |
| SEQ ID NO: 1838 | 2hpf_pa-6hpf_pa | s1_activated | UAGUGU | 2.11807 | 7.36E−08 |
| SEQ ID NO: 1839 | 2hpf_pa-6hpf_pa | s1_activated | UUAGUG | 2.06668 | 8.16E−08 |
| SEQ ID NO: 1840 | 2hpf_pa-6hpf_pa | s1_activated | UGUGUG | 1.60713 | 8.81E−08 |
| SEQ ID NO: 1841 | 2hpf_pa-6hpf_pa | s1_activated | GUGUG | 1.39552 | 1.04E−07 |
| SEQ ID NO: 1842 | 2hpf_pa-6hpf_pa | s1_activated | UUUUAG | 1.73709 | 2.21E−07 |
| SEQ ID NO: 1843 | 2hpf_pa-6hpf_pa | s1_activated | AAAGG | 1.54187 | 2.40E−07 |
| SEQ ID NO: 1844 | 2hpf_pa-6hpf_pa | s1_activated | GAGUUU | 1.66335 | 2.70E−07 |
| SEQ ID NO: 1845 | 2hpf_pa-6hpf_pa | s1_activated | UGUUU | 1.25757 | 2.95E−07 |
| SEQ ID NO: 1846 | 2hpf_pa-6hpf_pa | s1_activated | GUGUUU | 1.47612 | 3.19E−07 |
| SEQ ID NO: 1847 | 2hpf_pa-6hpf_pa | s1_activated | GUGUGU | 1.56218 | 3.78E−07 |
| SEQ ID NO: 1848 | 2hpf_pa-6hpf_pa | s1_activated | UGAAG | 1.39085 | 4.03E−07 |
| SEQ ID NO: 1849 | 2hpf_pa-6hpf_pa | s1_activated | GUGAAG | 1.81159 | 4.26E−07 |
| SEQ ID NO: 1850 | 2hpf_pa-6hpf_pa | s1_activated | GAAGA | 1.42564 | 4.75E−07 |
| SEQ ID NO: 1851 | 2hpf_pa-6hpf_pa | s1_activated | GUUUA | 1.33555 | 5.32E−07 |
| SEQ ID NO: 1852 | 2hpf_pa-6hpf_pa | s1_activated | UAAUUU | 1.74894 | 6.57E−07 |
| SEQ ID NO: 1853 | 2hpf_pa-6hpf_pa | s1_activated | UUACG | 1.65222 | 7.99E−07 |
| SEQ ID NO: 1854 | 2hpf_pa-6hpf_pa | s1_activated | GAAAG | 1.45616 | 8.01E−07 |
| SEQ ID NO: 1855 | 2hpf_pa-6hpf_pa | s1_activated | GAAGU | 1.44684 | 8.88E−07 |
| SEQ ID NO: 1856 | 2hpf_pa-6hpf_pa | s1_activated | CGUUUG | 1.70256 | 1.09E−06 |
| SEQ ID NO: 1857 | 2hpf_pa-6hpf_pa | s1_activated | AGUGU | 1.41657 | 1.29E−06 |
| SEQ ID NO: 1858 | 2hpf_pa-6hpf_pa | s1_activated | AGUUU | 1.31859 | 1.50E−06 |
| SEQ ID NO: 1859 | 2hpf_pa-6hpf_pa | s1_activated | AUAUC | 1.56684 | 1.51E−06 |
| SEQ ID NO: 1860 | 2hpf_pa-6hpf_pa | s1_activated | UUGAA | 1.3772 | 2.20E−06 |
| SEQ ID NO: 1861 | 2hpf_pa-6hpf_pa | s1_activated | GAAGUU | 1.75531 | 2.33E−06 |
| SEQ ID NO: 1862 | 2hpf_pa-6hpf_pa | s1_activated | UAAAG | 1.4145 | 2.85E−06 |
| SEQ ID NO: 1863 | 2hpf_pa-6hpf_pa | s1_activated | AGACUG | 1.86133 | 2.93E−06 |
| SEQ ID NO: 1864 | 2hpf_pa-6hpf_pa | s1_activated | UGUUUG | 1.52736 | 3.15E−06 |
| SEQ ID NO: 1865 | 2hpf_pa-6hpf_pa | s1_activated | UUGUU | 1.28544 | 3.38E−06 |
| SEQ ID NO: 1866 | 2hpf_pa-6hpf_pa | s1_activated | UUAAG | 1.50553 | 3.39E−06 |
| SEQ ID NO: 1867 | 2hpf_pa-6hpf_pa | s1_activated | UAUUU | 1.27171 | 3.42E−06 |
| SEQ ID NO: 1868 | 2hpf_pa-6hpf_pa | s1_activated | GUGAA | 1.39141 | 4.42E−06 |
| SEQ ID NO: 1869 | 2hpf_pa-6hpf_pa | s1_activated | AUUUU | 1.26051 | 4.63E−06 |
| SEQ ID NO: 1870 | 2hpf_pa-6hpf_pa | s1_activated | UUCUA | 1.57404 | 4.77E−06 |
| SEQ ID NO: 1871 | 2hpf_pa-6hpf_pa | s1_activated | GAAGAC | 1.98527 | 5.15E−06 |
| SEQ ID NO: 1872 | 2hpf_pa-6hpf_pa | s1_activated | UUUGA | 1.29958 | 5.18E−06 |
| SEQ ID NO: 1873 | 2hpf_pa-6hpf_pa | s1_activated | UGUUUA | 1.497 | 5.30E−06 |
| SEQ ID NO: 1874 | 2hpf_pa-6hpf_pa | s1_activated | AAGGCG | 2.11078 | 5.77E−06 |
| SEQ ID NO: 1875 | 2hpf_pa-6hpf_pa | s1_activated | UGAAGA | 1.69588 | 6.02E−06 |
| SEQ ID NO: 1876 | 2hpf_pa-6hpf_pa | s1_activated | GUGUU | 1.28715 | 6.97E−06 |
| SEQ ID NO: 1877 | 2hpf_pa-6hpf_pa | s1_activated | GUUACG | 2.58059 | 7.19E−06 |
| SEQ ID NO: 1878 | 2hpf_pa-6hpf_pa | s1_activated | UUAUU | 1.28328 | 7.64E−06 |
| SEQ ID NO: 1879 | 2hpf_pa-6hpf_pa | s1_activated | AUUAA | 1.42034 | 7.66E−06 |
| SEQ ID NO: 1880 | 2hpf_pa-6hpf_pa | s1_activated | GAGACU | 1.88431 | 7.81E−06 |
| SEQ ID NO: 1881 | 2hpf_pa-6hpf_pa | s1_activated | UUCUAC | 2.11697 | 9.18E−06 |
| SEQ ID NO: 1882 | 2hpf_pa-6hpf_pa | s1_activated | AUUGU | 1.39282 | 9.42E−06 |
| SEQ ID NO: 1883 | 2hpf_pa-6hpf_pa | s1_activated | GUUUAU | 1.54159 | 9.45E−06 |
| SEQ ID NO: 1884 | 2hpf_pa-6hpf_pa | s1_activated | AAGGG | 1.5709 | 9.75E−06 |
| SEQ ID NO: 1885 | 2hpf_pa-6hpf_pa | s1_activated | UGUGU | 1.29034 | 9.80E−06 |
| SEQ ID NO: 1886 | 2hpf_pa-6hpf_pa | s2_activated | CUCCC | 2.45651 | 2.41E−28 |
| SEQ ID NO: 1887 | 2hpf_pa-6hpf_pa | s2_activated | CCUCC | 2.2162 | 7.30E−27 |
| SEQ ID NO: 1888 | 2hpf_pa-6hpf_pa | s2_activated | CUCUC | 2.01395 | 6.49E−26 |
| SEQ ID NO: 1889 | 2hpf_pa-6hpf_pa | s2_activated | CCAUC | 2.25255 | 8.33E−25 |
| SEQ ID NO: 1890 | 2hpf_pa-6hpf_pa | s2_activated | UCCUC | 1.87788 | 1.65E−21 |
| SEQ ID NO: 1891 | 2hpf_pa-6hpf_pa | s2_activated | CCCCC | 2.61301 | 2.60E−21 |
| SEQ ID NO: 1892 | 2hpf_pa-6hpf_pa | s2_activated | UCUCC | 1.91056 | 3.89E−21 |
| SEQ ID NO: 1893 | 2hpf_pa-6hpf_pa | s2_activated | CCCUC | 2.14706 | 7.09E−21 |
| SEQ ID NO: 1894 | 2hpf_pa-6hpf_pa | s2_activated | CAUCC | 2.00996 | 3.01E−20 |
| SEQ ID NO: 1895 | 2hpf_pa-6hpf_pa | s2_activated | UCCCC | 2.1421 | 5.87E−20 |
| SEQ ID NO: 1896 | 2hpf_pa-6hpf_pa | s2_activated | UCCAC | 2.03838 | 2.30E−18 |
| SEQ ID NO: 1897 | 2hpf_pa-6hpf_pa | s2_activated | CUCCU | 1.86921 | 6.36E−18 |
| SEQ ID NO: 1898 | 2hpf_pa-6hpf_pa | s2_activated | CCCCU | 2.13693 | 6.90E−18 |
| SEQ ID NO: 1899 | 2hpf_pa-6hpf_pa | s2_activated | UCCCU | 1.99831 | 1.26E−17 |
| SEQ ID NO: 1900 | 2hpf_pa-6hpf_pa | s2_activated | UCAUC | 1.76424 | 3.63E−17 |
| SEQ ID NO: 1901 | 2hpf_pa-6hpf_pa | s2_activated | CCACC | 2.21428 | 6.70E−17 |
| SEQ ID NO: 1902 | 2hpf_pa-6hpf_pa | s2_activated | CUCCA | 1.83786 | 6.84E−16 |
| SEQ ID NO: 1903 | 2hpf_pa-6hpf_pa | s2_activated | CCCCCU | 2.89526 | 1.88E−15 |
| SEQ ID NO: 1904 | 2hpf_pa-6hpf_pa | s2_activated | CUCCCC | 2.9231 | 3.05E−15 |
| SEQ ID NO: 1905 | 2hpf_pa-6hpf_pa | s2_activated | CUCUCC | 2.46156 | 6.46E−15 |
| SEQ ID NO: 1906 | 2hpf_pa-6hpf_pa | s2_activated | CCUCU | 1.77141 | 2.81E−14 |
| SEQ ID NO: 1907 | 2hpf_pa-6hpf_pa | s2_activated | CCCUCC | 2.8844 | 4.28E−14 |
| SEQ ID NO: 1908 | 2hpf_pa-6hpf_pa | s2_activated | UCACC | 1.83095 | 2.32E−13 |
| SEQ ID NO: 1909 | 2hpf_pa-6hpf_pa | s2_activated | CCGCC | 1.95599 | 2.58E−13 |
| SEQ ID NO: 1910 | 2hpf_pa-6hpf_pa | s2_activated | CUCCCU | 2.55388 | 7.65E−13 |
| SEQ ID NO: 1911 | 2hpf_pa-6hpf_pa | s2_activated | CACCU | 1.88471 | 7.92E−13 |
| SEQ ID NO: 1912 | 2hpf_pa-6hpf_pa | s2_activated | CCUCCC | 2.71626 | 1.14E−12 |
| SEQ ID NO: 1913 | 2hpf_pa-6hpf_pa | s2_activated | GCUCC | 1.80779 | 1.52E−12 |
| SEQ ID NO: 1914 | 2hpf_pa-6hpf_pa | s2_activated | UCCAUC | 2.37703 | 1.58E−12 |
| SEQ ID NO: 1915 | 2hpf_pa-6hpf_pa | s2_activated | UCCCUC | 2.36767 | 2.71E−12 |
| SEQ ID NO: 1916 | 2hpf_pa-6hpf_pa | s2_activated | UCCAU | 1.77941 | 3.19E−12 |
| SEQ ID NO: 1917 | 2hpf_pa-6hpf_pa | s2_activated | CUCCAC | 2.65701 | 4.72E−12 |
| SEQ ID NO: 1918 | 2hpf_pa-6hpf_pa | s2_activated | AUUCC | 1.83736 | 8.76E−12 |
| SEQ ID NO: 1919 | 2hpf_pa-6hpf_pa | s2_activated | UCCACC | 2.71005 | 1.14E−11 |
| SEQ ID NO: 1920 | 2hpf_pa-6hpf_pa | s2_activated | ACCAUC | 2.65006 | 1.23E−11 |
| SEQ ID NO: 1921 | 2hpf_pa-6hpf_pa | s2_activated | UCCCCC | 2.76853 | 1.83E−11 |
| SEQ ID NO: 1922 | 2hpf_pa-6hpf_pa | s2_activated | GCUCUC | 2.24058 | 3.20E−11 |
| SEQ ID NO: 1923 | 2hpf_pa-6hpf_pa | s2_activated | GCCUC | 1.78369 | 3.57E−11 |
| SEQ ID NO: 1924 | 2hpf_pa-6hpf_pa | s2_activated | CUGCC | 1.75098 | 3.77E−11 |
| SEQ ID NO: 1925 | 2hpf_pa-6hpf_pa | s2_activated | UUCCUC | 1.98818 | 3.98E−11 |
| SEQ ID NO: 1926 | 2hpf_pa-6hpf_pa | s2_activated | CCUUU | 1.60096 | 4.05E−11 |
| SEQ ID NO: 1927 | 2hpf_pa-6hpf_pa | s2_activated | CGCUC | 1.6697 | 5.97E−11 |
| SEQ ID NO: 1928 | 2hpf_pa-6hpf_pa | s2_activated | AUCCC | 1.95484 | 6.89E−11 |
| SEQ ID NO: 1929 | 2hpf_pa-6hpf_pa | s2_activated | UCUCU | 1.54849 | 1.90E−10 |
| SEQ ID NO: 1930 | 2hpf_pa-6hpf_pa | s2_activated | CUCUCA | 2.30434 | 2.62E−10 |
| SEQ ID NO: 1931 | 2hpf_pa-6hpf_pa | s2_activated | CCCCCC | 3.37585 | 3.09E−10 |
| SEQ ID NO: 1932 | 2hpf_pa-6hpf_pa | s2_activated | CCAUCC | 2.51762 | 3.29E−10 |
| SEQ ID NO: 1933 | 2hpf_pa-6hpf_pa | s2_activated | UCCUCC | 2.0664 | 3.45E−10 |
| SEQ ID NO: 1934 | 2hpf_pa-6hpf_pa | s2_activated | UCUCCC | 2.439 | 3.73E−10 |
| SEQ ID NO: 1935 | 2hpf_pa-6hpf_pa | s2_activated | CAUCCU | 2.29829 | 5.54E−10 |
| SEQ ID NO: 1936 | 2hpf_pa-6hpf_pa | s2_activated | CACCG | 1.7637 | 7.07E−10 |
| SEQ ID NO: 1937 | 2hpf_pa-6hpf_pa | s2_activated | CACCC | 1.82488 | 9.48E−10 |
| SEQ ID NO: 1938 | 2hpf_pa-6hpf_pa | s2_activated | UCUCCU | 2.06499 | 1.23E−09 |
| SEQ ID NO: 1939 | 2hpf_pa-6hpf_pa | s2_activated | CCCUU | 1.74343 | 1.44E−09 |
| SEQ ID NO: 1940 | 2hpf_pa-6hpf_pa | s2_activated | UUCCC | 1.59498 | 1.49E−09 |
| SEQ ID NO: 1941 | 2hpf_pa-6hpf_pa | s2_activated | CCUCCU | 2.26062 | 1.50E−09 |
| SEQ ID NO: 1942 | 2hpf_pa-6hpf_pa | s2_activated | UCUCUC | 1.99929 | 2.72E−09 |
| SEQ ID NO: 1943 | 2hpf_pa-6hpf_pa | s2_activated | CCUGCC | 2.37967 | 2.76E−09 |
| SEQ ID NO: 1944 | 2hpf_pa-6hpf_pa | s2_activated | UUCUC | 1.47552 | 3.03E−09 |
| SEQ ID NO: 1945 | 2hpf_pa-6hpf_pa | s2_activated | CCUCCA | 2.28145 | 3.61E−09 |
| SEQ ID NO: 1946 | 2hpf_pa-6hpf_pa | s2_activated | UCAUCC | 2.09692 | 3.63E−09 |
| SEQ ID NO: 1947 | 2hpf_pa-6hpf_pa | s2_activated | CACCUG | 2.35813 | 3.85E−09 |
| SEQ ID NO: 1948 | 2hpf_pa-6hpf_pa | s2_activated | UGCUC | 1.56957 | 4.75E−09 |
| SEQ ID NO: 1949 | 2hpf_pa-6hpf_pa | s2_activated | CCCCUC | 2.36632 | 4.76E−09 |
| SEQ ID NO: 1950 | 2hpf_pa-6hpf_pa | s2_activated | CACUC | 1.66926 | 5.10E−09 |
| SEQ ID NO: 1951 | 2hpf_pa-6hpf_pa | s2_activated | CCCCGC | 2.73984 | 6.11E−09 |
| SEQ ID NO: 1952 | 2hpf_pa-6hpf_pa | s2_activated | CCAUCA | 2.32655 | 6.29E−09 |
| SEQ ID NO: 1953 | 2hpf_pa-6hpf_pa | s2_activated | CUUCC | 1.60669 | 6.63E−09 |
| SEQ ID NO: 1954 | 2hpf_pa-6hpf_pa | s2_activated | CACCA | 1.72385 | 6.86E−09 |
| SEQ ID NO: 1955 | 2hpf_pa-6hpf_pa | s2_activated | UCCUCU | 1.9885 | 6.88E−09 |
| SEQ ID NO: 1956 | 2hpf_pa-6hpf_pa | s2_activated | UUCCCC | 2.05199 | 7.13E−09 |
| SEQ ID NO: 1957 | 2hpf_pa-6hpf_pa | s2_activated | AAUCC | 1.73657 | 8.20E−09 |
| SEQ ID NO: 1958 | 2hpf_pa-6hpf_pa | s2_activated | CAUCA | 1.56572 | 8.49E−09 |
| SEQ ID NO: 1959 | 2hpf_pa-6hpf_pa | s2_activated | CUCAC | 1.63169 | 9.93E−09 |
| SEQ ID NO: 1960 | 2hpf_pa-6hpf_pa | s2_activated | AUCCU | 1.67139 | 2.28E−08 |
| SEQ ID NO: 1961 | 2hpf_pa-6hpf_pa | s2_activated | CCCCA | 1.84904 | 2.48E−08 |
| SEQ ID NO: 1962 | 2hpf_pa-6hpf_pa | s2_activated | CCUGC | 1.56154 | 2.53E−08 |
| SEQ ID NO: 1963 | 2hpf_pa-6hpf_pa | s2_activated | GCUCU | 1.50467 | 3.06E−08 |
| SEQ ID NO: 1964 | 2hpf_pa-6hpf_pa | s2_activated | UCCUU | 1.58577 | 3.32E−08 |
| SEQ ID NO: 1965 | 2hpf_pa-6hpf_pa | s2_activated | CCAGU | 1.66899 | 3.66E−08 |
| SEQ ID NO: 1966 | 2hpf_pa-6hpf_pa | s2_activated | CUCACC | 2.53805 | 3.88E−08 |
| SEQ ID NO: 1967 | 2hpf_pa-6hpf_pa | s2_activated | AUCAUC | 2.02338 | 4.21E−08 |
| SEQ ID NO: 1968 | 2hpf_pa-6hpf_pa | s2_activated | CUCCUC | 1.91521 | 5.36E−08 |
| SEQ ID NO: 1969 | 2hpf_pa-6hpf_pa | s2_activated | CUCUUC | 1.89334 | 5.81E−08 |
| SEQ ID NO: 1970 | 2hpf_pa-6hpf_pa | s2_activated | AUCCA | 1.62551 | 5.88E−08 |
| SEQ ID NO: 1971 | 2hpf_pa-6hpf_pa | s2_activated | UUCAUC | 1.8542 | 6.63E−08 |
| SEQ ID NO: 1972 | 2hpf_pa-6hpf_pa | s2_activated | GUAUUC | 2.55735 | 6.90E−08 |
| SEQ ID NO: 1973 | 2hpf_pa-6hpf_pa | s2_activated | CAUCCC | 2.31278 | 7.54E−08 |
| SEQ ID NO: 1974 | 2hpf_pa-6hpf_pa | s2_activated | CCAUCU | 2.23748 | 8.43E−08 |
| SEQ ID NO: 1975 | 2hpf_pa-6hpf_pa | s2_activated | AUCAGC | 1.92204 | 8.56E−08 |
| SEQ ID NO: 1976 | 2hpf_pa-6hpf_pa | s2_activated | CACUCC | 2.40295 | 9.17E−08 |
| SEQ ID NO: 1977 | 2hpf_pa-6hpf_pa | s2_activated | GCCCC | 1.88677 | 9.96E−08 |
| SEQ ID NO: 1978 | 2hpf_pa-6hpf_pa | s2_activated | CCUCUU | 2.02501 | 1.34E−07 |
| SEQ ID NO: 1979 | 2hpf_pa-6hpf_pa | s2_activated | CCCUUU | 2.01112 | 1.35E−07 |
| SEQ ID NO: 1980 | 2hpf_pa-6hpf_pa | s2_activated | CCCAG | 1.66361 | 1.40E−07 |
| SEQ ID NO: 1981 | 2hpf_pa-6hpf_pa | s2_activated | CCUCA | 1.56693 | 1.41E−07 |
| SEQ ID NO: 1982 | 2hpf_pa-6hpf_pa | s2_activated | UCCCCA | 2.4887 | 1.46E−07 |
| SEQ ID NO: 1983 | 2hpf_pa-6hpf_pa | s2_activated | UUCUCC | 1.82645 | 1.52E−07 |
| SEQ ID NO: 1984 | 2hpf_pa-6hpf_pa | s2_activated | CCUCG | 1.64181 | 1.66E−07 |
| SEQ ID NO: 1985 | 2hpf_pa-6hpf_pa | s2_activated | CUCCUU | 2.12969 | 1.79E−07 |
| SEQ ID NO: 1986 | 2hpf_pa-6hpf_pa | s2_activated | ACACC | 1.58541 | 1.88E−07 |
| SEQ ID NO: 1987 | 2hpf_pa-6hpf_pa | s2_activated | CCACCA | 2.60416 | 2.17E−07 |
| SEQ ID NO: 1988 | 2hpf_pa-6hpf_pa | s2_activated | CCCGU | 1.72184 | 2.45E−07 |
| SEQ ID NO: 1989 | 2hpf_pa-6hpf_pa | s2_activated | AUCCCU | 2.32149 | 2.49E−07 |
| SEQ ID NO: 1990 | 2hpf_pa-6hpf_pa | s2_activated | CCAGUU | 2.26815 | 2.58E−07 |
| SEQ ID NO: 1991 | 2hpf_pa-6hpf_pa | s2_activated | UCCGCC | 2.13513 | 2.83E−07 |
| SEQ ID NO: 1992 | 2hpf_pa-6hpf_pa | s2_activated | ACUCCC | 2.48775 | 3.16E−07 |
| SEQ ID NO: 1993 | 2hpf_pa-6hpf_pa | s2_activated | CCACCU | 2.56378 | 3.21E−07 |
| SEQ ID NO: 1994 | 2hpf_pa-6hpf_pa | s2_activated | CCCUG | 1.61794 | 3.49E−07 |
| SEQ ID NO: 1995 | 2hpf_pa-6hpf_pa | s2_activated | UGCCC | 1.75232 | 3.81E−07 |
| SEQ ID NO: 1996 | 2hpf_pa-6hpf_pa | s2_activated | CAUCU | 1.45278 | 4.47E−07 |
| SEQ ID NO: 1997 | 2hpf_pa-6hpf_pa | s2_activated | UCUUC | 1.4382 | 4.55E−07 |
| SEQ ID NO: 1998 | 2hpf_pa-6hpf_pa | s2_activated | GCCCCC | 2.8187 | 4.84E−07 |
| SEQ ID NO: 1999 | 2hpf_pa-6hpf_pa | s2_activated | CAUCCA | 2.10991 | 5.35E−07 |
| SEQ ID NO: 2000 | 2hpf_pa-6hpf_pa | s2_activated | AGCCC | 1.64918 | 5.96E−07 |
| SEQ ID NO: 2001 | 2hpf_pa-6hpf_pa | s2_activated | CCCCG | 1.71914 | 7.34E−07 |
| SEQ ID NO: 2002 | 2hpf_pa-6hpf_pa | s2_activated | UCACCC | 2.2038 | 7.96E−07 |
| SEQ ID NO: 2003 | 2hpf_pa-6hpf_pa | s2_activated | CUCAU | 1.50317 | 9.24E−07 |
| SEQ ID NO: 2004 | 2hpf_pa-6hpf_pa | s2_activated | UCUCA | 1.49336 | 9.49E−07 |
| SEQ ID NO: 2005 | 2hpf_pa-6hpf_pa | s2_activated | CCACA | 1.53346 | 1.12E−06 |
| SEQ ID NO: 2006 | 2hpf_pa-6hpf_pa | s2_activated | CCGUC | 1.55819 | 1.19E−06 |
| SEQ ID NO: 2007 | 2hpf_pa-6hpf_pa | s2_activated | CGGCCU | 2.46697 | 1.20E−06 |
| SEQ ID NO: 2008 | 2hpf_pa-6hpf_pa | s2_activated | UGCUCU | 1.85158 | 1.37E−06 |
| SEQ ID NO: 2009 | 2hpf_pa-6hpf_pa | s2_activated | AUCCUC | 2.01246 | 1.38E−06 |
| SEQ ID NO: 2010 | 2hpf_pa-6hpf_pa | s2_activated | UCUUCC | 2.02091 | 1.54E−06 |
| SEQ ID NO: 2011 | 2hpf_pa-6hpf_pa | s2_activated | AAGCCC | 2.27495 | 1.56E−06 |
| SEQ ID NO: 2012 | 2hpf_pa-6hpf_pa | s2_activated | UUCCU | 1.39662 | 1.64E−06 |
| SEQ ID NO: 2013 | 2hpf_pa-6hpf_pa | s2_activated | CUUCUC | 1.80505 | 1.64E−06 |
| SEQ ID NO: 2014 | 2hpf_pa-6hpf_pa | s2_activated | UCGGCC | 2.59831 | 1.81E−06 |
| SEQ ID NO: 2015 | 2hpf_pa-6hpf_pa | s2_activated | GCCGCC | 2.24737 | 2.13E−06 |
| SEQ ID NO: 2016 | 2hpf_pa-6hpf_pa | s2_activated | CUCUCU | 1.75143 | 2.16E−06 |
| SEQ ID NO: 2017 | 2hpf_pa-6hpf_pa | s2_activated | UCCGC | 1.50837 | 2.18E−06 |
| SEQ ID NO: 2018 | 2hpf_pa-6hpf_pa | s2_activated | CCUCUC | 1.85594 | 2.22E−06 |
| SEQ ID NO: 2019 | 2hpf_pa-6hpf_pa | s2_activated | UCACUC | 1.99626 | 2.22E−06 |
| SEQ ID NO: 2020 | 2hpf_pa-6hpf_pa | s2_activated | CAUCAU | 1.86531 | 2.27E−06 |
| SEQ ID NO: 2021 | 2hpf_pa-6hpf_pa | s2_activated | UCCACA | 1.98818 | 2.50E−06 |
| SEQ ID NO: 2022 | 2hpf_pa-6hpf_pa | s2_activated | CUGCCC | 2.17145 | 2.82E−06 |
| SEQ ID NO: 2023 | 2hpf_pa-6hpf_pa | s2_activated | CACCGU | 2.27774 | 2.86E−06 |
| SEQ ID NO: 2024 | 2hpf_pa-6hpf_pa | s2_activated | CCCCCG | 2.76471 | 2.97E−06 |
| SEQ ID NO: 2025 | 2hpf_pa-6hpf_pa | s2_activated | CUUCCC | 2.06061 | 2.98E−06 |
| SEQ ID NO: 2026 | 2hpf_pa-6hpf_pa | s2_activated | CCACCG | 2.4084 | 3.01E−06 |
| SEQ ID NO: 2027 | 2hpf_pa-6hpf_pa | s2_activated | CCCAU | 1.73567 | 3.02E−06 |
| SEQ ID NO: 2028 | 2hpf_pa-6hpf_pa | s2_activated | UUCCA | 1.49781 | 3.11E−06 |
| SEQ ID NO: 2029 | 2hpf_pa-6hpf_pa | s2_activated | CUCCCA | 2.44441 | 3.12E−06 |
| SEQ ID NO: 2030 | 2hpf_pa-6hpf_pa | s2_activated | AUUCCG | 2.52809 | 3.25E−06 |
| SEQ ID NO: 2031 | 2hpf_pa-6hpf_pa | s2_activated | CGCCUC | 2.15945 | 3.25E−06 |
| SEQ ID NO: 2032 | 2hpf_pa-6hpf_pa | s2_activated | AUCCAC | 2.06196 | 3.76E−06 |
| SEQ ID NO: 2033 | 2hpf_pa-6hpf_pa | s2_activated | ACCCC | 1.66796 | 3.76E−06 |
| SEQ ID NO: 2034 | 2hpf_pa-6hpf_pa | s2_activated | UCAGC | 1.38272 | 3.89E−06 |
| SEQ ID NO: 2035 | 2hpf_pa-6hpf_pa | s2_activated | UCCCUG | 2.02239 | 3.91E−06 |
| SEQ ID NO: 2036 | 2hpf_pa-6hpf_pa | s2_activated | UCCUCG | 1.9875 | 3.98E−06 |
| SEQ ID NO: 2037 | 2hpf_pa-6hpf_pa | s2_activated | CUCUU | 1.38097 | 3.98E−06 |
| SEQ ID NO: 2038 | 2hpf_pa-6hpf_pa | s2_activated | CUCCG | 1.52547 | 4.03E−06 |
| SEQ ID NO: 2039 | 2hpf_pa-6hpf_pa | s2_activated | ACUCC | 1.56645 | 4.30E−06 |
| SEQ ID NO: 2040 | 2hpf_pa-6hpf_pa | s2_activated | UUCAU | 1.34308 | 4.31E−06 |
| SEQ ID NO: 2041 | 2hpf_pa-6hpf_pa | s2_activated | ACUCUC | 2.01336 | 4.42E−06 |
| SEQ ID NO: 2042 | 2hpf_pa-6hpf_pa | s2_activated | CUCCAU | 1.89028 | 4.79E−06 |
| SEQ ID NO: 2043 | 2hpf_pa-6hpf_pa | s2_activated | GCCUCC | 2.11276 | 5.64E−06 |
| SEQ ID NO: 2044 | 2hpf_pa-6hpf_pa | s2_activated | ACACU | 1.41734 | 5.84E−06 |
| SEQ ID NO: 2045 | 2hpf_pa-6hpf_pa | s2_activated | UCCCA | 1.60026 | 6.42E−06 |
| SEQ ID NO: 2046 | 2hpf_pa-6hpf_pa | s2_activated | AUCACC | 2.14345 | 6.82E−06 |
| SEQ ID NO: 2047 | 2hpf_pa-6hpf_pa | s2_activated | GCGCUC | 1.81084 | 7.04E−06 |
| SEQ ID NO: 2048 | 2hpf_pa-6hpf_pa | s2_activated | CUUUCC | 1.73061 | 7.13E−06 |
| SEQ ID NO: 2049 | 2hpf_pa-6hpf_pa | s2_activated | GCCGC | 1.48887 | 7.48E−06 |
| SEQ ID NO: 2050 | 2hpf_pa-6hpf_pa | s2_activated | ACAUC | 1.41685 | 8.21E−06 |
| SEQ ID NO: 2051 | 2hpf_pa-6hpf_pa | s2_activated | GCUCCA | 1.93048 | 8.96E−06 |
| SEQ ID NO: 2052 | 2hpf_pa-6hpf_pa | s2_activated | UUUCC | 1.31252 | 9.86E−06 |
| SEQ ID NO: 2053 | 6hpf_pa | active | CCUCU | 2.051 | 7.57E−24 |
| SEQ ID NO: 2054 | 6hpf_pa | active | UUUCU | 1.60546 | 1.29E−20 |
| SEQ ID NO: 2055 | 6hpf_pa | active | UCCCC | 2.15456 | 2.49E−20 |
| SEQ ID NO: 2056 | 6hpf_pa | active | CCCUC | 2.1197 | 4.53E−20 |
| SEQ ID NO: 2057 | 6hpf_pa | active | CUUUU | 1.56044 | 1.26E−19 |
| SEQ ID NO: 2058 | 6hpf_pa | active | CCCCU | 2.19678 | 1.82E−19 |
| SEQ ID NO: 2059 | 6hpf_pa | active | CCCCC | 2.49394 | 7.92E−19 |
| SEQ ID NO: 2060 | 6hpf_pa | active | UUCUC | 1.73572 | 8.51E−19 |
| SEQ ID NO: 2061 | 6hpf_pa | active | UUUUC | 1.53362 | 1.16E−18 |
| SEQ ID NO: 2062 | 6hpf_pa | active | UCUCU | 1.7761 | 2.09E−18 |
| SEQ ID NO: 2063 | 6hpf_pa | active | CUCCC | 2.10854 | 4.79E−18 |
| SEQ ID NO: 2064 | 6hpf_pa | active | CUCUC | 1.81404 | 6.26E−18 |
| SEQ ID NO: 2065 | 6hpf_pa | active | UCCCU | 1.9482 | 3.30E−16 |
| SEQ ID NO: 2066 | 6hpf_pa | active | UUCCC | 1.8232 | 4.46E−16 |
| SEQ ID NO: 2067 | 6hpf_pa | active | UCCUC | 1.7133 | 3.14E−15 |
| SEQ ID NO: 2068 | 6hpf_pa | active | UCCCUC | 2.56173 | 3.90E−15 |
| SEQ ID NO: 2069 | 6hpf_pa | active | UUUCUC | 2.00106 | 3.93E−15 |
| SEQ ID NO: 2070 | 6hpf_pa | active | CCCCCU | 2.80558 | 2.50E−14 |
| SEQ ID NO: 2071 | 6hpf_pa | active | UUCCUC | 2.10946 | 2.40E−13 |
| SEQ ID NO: 2072 | 6hpf_pa | active | UUUUU | 1.41403 | 5.40E−13 |
| SEQ ID NO: 2073 | 6hpf_pa | active | ACUUU | 1.52728 | 2.22E−12 |
| SEQ ID NO: 2074 | 6hpf_pa | active | UCUCC | 1.65491 | 2.72E−12 |
| SEQ ID NO: 2075 | 6hpf_pa | active | CCUCUC | 2.31942 | 4.05E−12 |
| SEQ ID NO: 2076 | 6hpf_pa | active | CCUCC | 1.73876 | 7.65E−12 |
| SEQ ID NO: 2077 | 6hpf_pa | active | CUCUU | 1.58255 | 1.07E−11 |
| SEQ ID NO: 2078 | 6hpf_pa | active | UUCUU | 1.54051 | 1.32E−11 |
| SEQ ID NO: 2079 | 6hpf_pa | active | UUCUCU | 1.96923 | 2.89E−11 |
| SEQ ID NO: 2080 | 6hpf_pa | active | UCUCUC | 2.12699 | 3.63E−11 |
| SEQ ID NO: 2081 | 6hpf_pa | active | CCCCA | 2.02195 | 4.76E−11 |
| SEQ ID NO: 2082 | 6hpf_pa | active | UUUUCC | 1.83977 | 4.78E−11 |
| SEQ ID NO: 2083 | 6hpf_pa | active | CCCUU | 1.81447 | 4.98E−11 |
| SEQ ID NO: 2084 | 6hpf_pa | active | UUUCC | 1.47984 | 5.83E−11 |
| SEQ ID NO: 2085 | 6hpf_pa | active | CUCCCC | 2.55137 | 6.02E−11 |
| SEQ ID NO: 2086 | 6hpf_pa | active | CUCUCC | 2.19735 | 6.25E−11 |
| SEQ ID NO: 2087 | 6hpf_pa | active | UUCCU | 1.55415 | 7.67E−11 |
| SEQ ID NO: 2088 | 6hpf_pa | active | UUCCCC | 2.20022 | 7.82E−11 |
| SEQ ID NO: 2089 | 6hpf_pa | active | UCUUU | 1.4325 | 1.53E−10 |
| SEQ ID NO: 2090 | 6hpf_pa | active | UAUUU | 1.38104 | 2.28E−10 |
| SEQ ID NO: 2091 | 6hpf_pa | active | UUUUA | 1.34681 | 2.32E−10 |
| SEQ ID NO: 2092 | 6hpf_pa | active | CUUUUU | 1.66636 | 3.28E−10 |
| SEQ ID NO: 2093 | 6hpf_pa | active | UACUUU | 2.09656 | 3.61E−10 |
| SEQ ID NO: 2094 | 6hpf_pa | active | ACCCC | 1.93459 | 4.38E−10 |
| SEQ ID NO: 2095 | 6hpf_pa | active | CCAUC | 1.7171 | 4.79E−10 |
| SEQ ID NO: 2096 | 6hpf_pa | active | UUUAUU | 1.59245 | 5.04E−10 |
| SEQ ID NO: 2097 | 6hpf_pa | active | CUCUG | 1.53569 | 5.15E−10 |
| SEQ ID NO: 2098 | 6hpf_pa | active | UUUUUU | 1.62568 | 9.26E−10 |
| SEQ ID NO: 2099 | 6hpf_pa | active | AUUCC | 1.73822 | 1.10E−09 |
| SEQ ID NO: 2100 | 6hpf_pa | active | UCCCCA | 2.75716 | 1.29E−09 |
| SEQ ID NO: 2101 | 6hpf_pa | active | UUUCUU | 1.70248 | 1.48E−09 |
| SEQ ID NO: 2102 | 6hpf_pa | active | UUUCCC | 1.90461 | 1.51E−09 |
| SEQ ID NO: 2103 | 6hpf_pa | active | CCCCUC | 2.4122 | 1.66E−09 |
| SEQ ID NO: 2104 | 6hpf_pa | active | CCCCUU | 2.38141 | 1.90E−09 |
| SEQ ID NO: 2105 | 6hpf_pa | active | CCCAU | 1.97565 | 1.95E−09 |
| SEQ ID NO: 2106 | 6hpf_pa | active | CACCC | 1.80683 | 1.98E−09 |
| SEQ ID NO: 2107 | 6hpf_pa | active | CUCUCU | 1.96993 | 2.80E−09 |
| SEQ ID NO: 2108 | 6hpf_pa | active | UCUCCC | 2.3498 | 3.13E−09 |
| SEQ ID NO: 2109 | 6hpf_pa | active | UUUUCU | 1.61509 | 3.31E−09 |
| SEQ ID NO: 2110 | 6hpf_pa | active | CUUCC | 1.6058 | 6.87E−09 |
| SEQ ID NO: 2111 | 6hpf_pa | active | UAUUUU | 1.55497 | 7.52E−09 |
| SEQ ID NO: 2112 | 6hpf_pa | active | UCCAUC | 2.08748 | 9.47E−09 |
| SEQ ID NO: 2113 | 6hpf_pa | active | CUCUA | 1.7866 | 9.97E−09 |
| SEQ ID NO: 2114 | 6hpf_pa | active | CCCAG | 1.72485 | 1.19E−08 |
| SEQ ID NO: 2115 | 6hpf_pa | active | ACCCA | 1.81346 | 1.52E−08 |
| SEQ ID NO: 2116 | 6hpf_pa | active | ACCCCC | 2.64414 | 1.76E−08 |
| SEQ ID NO: 2117 | 6hpf_pa | active | UCCCCC | 2.43578 | 2.07E−08 |
| SEQ ID NO: 2118 | 6hpf_pa | active | CCACC | 1.77411 | 2.33E−08 |
| SEQ ID NO: 2119 | 6hpf_pa | active | CUCCAU | 2.10383 | 3.29E−08 |
| SEQ ID NO: 2120 | 6hpf_pa | active | CUCUGC | 1.90856 | 3.38E−08 |
| SEQ ID NO: 2121 | 6hpf_pa | active | CUCCU | 1.53191 | 3.40E−08 |
| SEQ ID NO: 2122 | 6hpf_pa | active | CCCUCU | 2.31988 | 3.56E−08 |
| SEQ ID NO: 2123 | 6hpf_pa | active | UUUUUC | 1.58268 | 3.74E−08 |
| SEQ ID NO: 2124 | 6hpf_pa | active | CUUUUC | 1.72009 | 4.45E−08 |
| SEQ ID NO: 2125 | 6hpf_pa | active | UCCUCC | 1.91101 | 4.79E−08 |
| SEQ ID NO: 2126 | 6hpf_pa | active | AUUUU | 1.31458 | 5.15E−08 |
| SEQ ID NO: 2127 | 6hpf_pa | active | UUUAU | 1.33165 | 5.35E−08 |
| SEQ ID NO: 2128 | 6hpf_pa | active | UUUAC | 1.40599 | 6.02E−08 |
| SEQ ID NO: 2129 | 6hpf_pa | active | UCCAC | 1.60968 | 6.42E−08 |
| SEQ ID NO: 2130 | 6hpf_pa | active | CCUCCC | 2.24746 | 7.30E−08 |
| SEQ ID NO: 2131 | 6hpf_pa | active | CUCAU | 1.556 | 7.78E−08 |
| SEQ ID NO: 2132 | 6hpf_pa | active | GCCUCU | 2.16197 | 8.22E−08 |
| SEQ ID NO: 2133 | 6hpf_pa | active | CCUUU | 1.47824 | 8.24E−08 |
| SEQ ID NO: 2134 | 6hpf_pa | active | UCACCC | 2.30825 | 1.11E−07 |
| SEQ ID NO: 2135 | 6hpf_pa | active | UACUU | 1.59033 | 1.14E−07 |
| SEQ ID NO: 2136 | 6hpf_pa | active | ACUUUU | 1.65743 | 1.15E−07 |
| SEQ ID NO: 2137 | 6hpf_pa | active | GCCUC | 1.61104 | 1.22E−07 |
| SEQ ID NO: 2138 | 6hpf_pa | active | UCUCA | 1.534 | 1.39E−07 |
| SEQ ID NO: 2139 | 6hpf_pa | active | AUUCUC | 2.11803 | 1.64E−07 |
| SEQ ID NO: 2140 | 6hpf_pa | active | GCUCU | 1.47024 | 2.05E−07 |
| SEQ ID NO: 2141 | 6hpf_pa | active | CCACUU | 2.4252 | 2.05E−07 |
| SEQ ID NO: 2142 | 6hpf_pa | active | CCUUUU | 1.78377 | 2.46E−07 |
| SEQ ID NO: 2143 | 6hpf_pa | active | AUCCCU | 2.321 | 2.51E−07 |
| SEQ ID NO: 2144 | 6hpf_pa | active | CUCUUU | 1.68446 | 2.89E−07 |
| SEQ ID NO: 2145 | 6hpf_pa | active | CCCCAU | 2.87806 | 3.06E−07 |
| SEQ ID NO: 2146 | 6hpf_pa | active | UAGUU | 1.51154 | 3.07E−07 |
| SEQ ID NO: 2147 | 6hpf_pa | active | CAUAU | 1.57074 | 3.12E−07 |
| SEQ ID NO: 2148 | 6hpf_pa | active | CCUCUA | 2.56323 | 3.22E−07 |
| SEQ ID NO: 2149 | 6hpf_pa | active | UAUUCC | 2.29506 | 3.45E−07 |
| SEQ ID NO: 2150 | 6hpf_pa | active | CUCGUU | 1.94036 | 3.92E−07 |
| SEQ ID NO: 2151 | 6hpf_pa | active | AUCCC | 1.71931 | 4.06E−07 |
| SEQ ID NO: 2152 | 6hpf_pa | active | CCAUUC | 2.36495 | 4.07E−07 |
| SEQ ID NO: 2153 | 6hpf_pa | active | UUAUU | 1.32352 | 4.40E−07 |
| SEQ ID NO: 2154 | 6hpf_pa | active | UCUCUU | 1.76562 | 4.45E−07 |
| SEQ ID NO: 2155 | 6hpf_pa | active | UCUUUU | 1.58139 | 4.56E−07 |
| SEQ ID NO: 2156 | 6hpf_pa | active | UCUUC | 1.43741 | 4.72E−07 |
| SEQ ID NO: 2157 | 6hpf_pa | active | CUGCC | 1.55421 | 4.99E−07 |
| SEQ ID NO: 2158 | 6hpf_pa | active | CUUUCU | 1.74444 | 5.37E−07 |
| SEQ ID NO: 2159 | 6hpf_pa | active | UUUUUA | 1.47352 | 5.70E−07 |
| SEQ ID NO: 2160 | 6hpf_pa | active | UUUUAU | 1.51489 | 5.98E−07 |
| SEQ ID NO: 2161 | 6hpf_pa | active | UCCCA | 1.67047 | 6.33E−07 |
| SEQ ID NO: 2162 | 6hpf_pa | active | UUAGUU | 1.80358 | 7.16E−07 |
| SEQ ID NO: 2163 | 6hpf_pa | active | CUCCCU | 2.01172 | 8.70E−07 |
| SEQ ID NO: 2164 | 6hpf_pa | active | CCCAA | 1.7592 | 9.75E−07 |
| SEQ ID NO: 2165 | 6hpf_pa | active | CCCAUU | 2.30271 | 1.15E−06 |
| SEQ ID NO: 2166 | 6hpf_pa | active | CUUCCC | 2.10699 | 1.23E−06 |
| SEQ ID NO: 2167 | 6hpf_pa | active | CUGCCU | 2.06551 | 1.31E−06 |
| SEQ ID NO: 2168 | 6hpf_pa | active | ACCUCU | 2.1196 | 1.36E−06 |
| SEQ ID NO: 2169 | 6hpf_pa | active | UUCUUU | 1.58599 | 1.44E−06 |
| SEQ ID NO: 2170 | 6hpf_pa | active | UCCUCU | 1.80252 | 1.45E−06 |
| SEQ ID NO: 2171 | 6hpf_pa | active | UUAUUU | 1.43757 | 1.51E−06 |
| SEQ ID NO: 2172 | 6hpf_pa | active | AUUCCU | 2.0122 | 1.75E−06 |
| SEQ ID NO: 2173 | 6hpf_pa | active | CUCAUC | 1.85904 | 2.53E−06 |
| SEQ ID NO: 2174 | 6hpf_pa | active | UCACC | 1.50701 | 2.71E−06 |
| SEQ ID NO: 2175 | 6hpf_pa | active | CCUCA | 1.49646 | 2.99E−06 |
| SEQ ID NO: 2176 | 6hpf_pa | active | UCUCCU | 1.78927 | 3.14E−06 |
| SEQ ID NO: 2177 | 6hpf_pa | active | UAAUUC | 1.97179 | 3.18E−06 |
| SEQ ID NO: 2178 | 6hpf_pa | active | UUCCCU | 1.88731 | 3.38E−06 |
| SEQ ID NO: 2179 | 6hpf_pa | active | AUCUC | 1.4891 | 3.53E−06 |
| SEQ ID NO: 2180 | 6hpf_pa | active | CAAUCC | 2.60518 | 3.95E−06 |
| SEQ ID NO: 2181 | 6hpf_pa | active | UGCCU | 1.53982 | 4.17E−06 |
| SEQ ID NO: 2182 | 6hpf_pa | active | CCCUCG | 2.21299 | 4.21E−06 |
| SEQ ID NO: 2183 | 6hpf_pa | active | UGCCC | 1.66695 | 5.01E−06 |
| SEQ ID NO: 2184 | 6hpf_pa | active | ACCACC | 2.35316 | 5.04E−06 |
| SEQ ID NO: 2185 | 6hpf_pa | active | UCCUU | 1.47329 | 5.14E−06 |
| SEQ ID NO: 2186 | 6hpf_pa | active | UCCUUU | 1.77148 | 5.20E−06 |
| SEQ ID NO: 2187 | 6hpf_pa | active | UCUGCC | 2.03234 | 5.54E−06 |
| SEQ ID NO: 2188 | 6hpf_pa | active | CCUGCC | 2.01313 | 5.62E−06 |
| SEQ ID NO: 2189 | 6hpf_pa | active | UCCAU | 1.48399 | 5.81E−06 |
| SEQ ID NO: 2190 | 6hpf_pa | active | CCCAC | 1.71062 | 5.90E−06 |
| SEQ ID NO: 2191 | 6hpf_pa | active | CAUUGU | 1.80787 | 6.22E−06 |
| SEQ ID NO: 2192 | 6hpf_pa | active | CAUAUU | 1.78718 | 6.39E−06 |
| SEQ ID NO: 2193 | 6hpf_pa | active | CGCUCU | 1.86084 | 9.26E−06 |
| SEQ ID NO: 2194 | 6hpf_pa | repressed | AUGGC | 3.09635 | 4.70E−47 |
| SEQ ID NO: 2195 | 6hpf_pa | repressed | GAUGG | 2.90232 | 2.10E−40 |
| SEQ ID NO: 2196 | 6hpf_pa | repressed | GGAUG | 2.66104 | 1.36E−38 |
| SEQ ID NO: 2197 | 6hpf_pa | repressed | AGGAUG | 4.7431 | 1.56E−36 |
| SEQ ID NO: 2198 | 6hpf_pa | repressed | AUGGA | 2.57657 | 2.84E−36 |
| SEQ ID NO: 2199 | 6hpf_pa | repressed | CAUGG | 2.60348 | 2.64E−30 |
| SEQ ID NO: 2200 | 6hpf_pa | repressed | AGAUG | 2.34868 | 1.01E−26 |
| SEQ ID NO: 2201 | 6hpf_pa | repressed | CCAUG | 2.75674 | 3.04E−26 |
| SEQ ID NO: 2202 | 6hpf_pa | repressed | GAUGGC | 4.25554 | 8.93E−24 |
| SEQ ID NO: 2203 | 6hpf_pa | repressed | AUGUG | 1.99973 | 2.94E−22 |
| SEQ ID NO: 2204 | 6hpf_pa | repressed | GAUGU | 2.11044 | 3.09E−22 |
| SEQ ID NO: 2205 | 6hpf_pa | repressed | AUGGAG | 3.15401 | 1.19E−21 |
| SEQ ID NO: 2206 | 6hpf_pa | repressed | CAAUG | 2.33897 | 2.83E−20 |
| SEQ ID NO: 2207 | 6hpf_pa | repressed | CGAUG | 2.52218 | 3.30E−20 |
| SEQ ID NO: 2208 | 6hpf_pa | repressed | AAUGG | 2.26354 | 4.68E−20 |
| SEQ ID NO: 2209 | 6hpf_pa | repressed | AUGGCG | 3.63114 | 6.28E−20 |
| SEQ ID NO: 2210 | 6hpf_pa | repressed | AAAUG | 1.95216 | 8.83E−20 |
| SEQ ID NO: 2211 | 6hpf_pa | repressed | UCAUG | 2.06033 | 1.12E−19 |
| SEQ ID NO: 2212 | 6hpf_pa | repressed | GAUGC | 2.29136 | 1.32E−19 |
| SEQ ID NO: 2213 | 6hpf_pa | repressed | GGAUGC | 3.67108 | 2.72E−19 |
| SEQ ID NO: 2214 | 6hpf_pa | repressed | GAUGA | 2.05592 | 1.24E−18 |
| SEQ ID NO: 2215 | 6hpf_pa | repressed | AUCAUG | 3.39001 | 5.88E−18 |
| SEQ ID NO: 2216 | 6hpf_pa | repressed | UGGAG | 1.7623 | 7.34E−18 |
| SEQ ID NO: 2217 | 6hpf_pa | repressed | AAUGGC | 3.2388 | 4.01E−16 |
| SEQ ID NO: 2218 | 6hpf_pa | repressed | AAGAUG | 3.18698 | 5.09E−16 |
| SEQ ID NO: 2219 | 6hpf_pa | repressed | AUGAG | 1.96588 | 7.95E−16 |
| SEQ ID NO: 2220 | 6hpf_pa | repressed | AUGGG | 2.2823 | 8.13E−16 |
| SEQ ID NO: 2221 | 6hpf_pa | repressed | AAAAUG | 2.77358 | 1.15E−15 |
| SEQ ID NO: 2222 | 6hpf_pa | repressed | ACCAUG | 4.22244 | 1.71E−15 |
| SEQ ID NO: 2223 | 6hpf_pa | repressed | ACAAUG | 3.45342 | 4.89E−15 |
| SEQ ID NO: 2224 | 6hpf_pa | repressed | AGGAU | 1.86826 | 7.96E−15 |
| SEQ ID NO: 2225 | 6hpf_pa | repressed | UGAUG | 1.83181 | 1.56E−14 |
| SEQ ID NO: 2226 | 6hpf_pa | repressed | AUGAGG | 2.98445 | 2.24E−14 |
| SEQ ID NO: 2227 | 6hpf_pa | repressed | AUGGCA | 3.08156 | 4.20E−14 |
| SEQ ID NO: 2228 | 6hpf_pa | repressed | AAAUGG | 2.90015 | 5.33E−14 |
| SEQ ID NO: 2229 | 6hpf_pa | repressed | AUGUGG | 2.78041 | 6.18E−14 |
| SEQ ID NO: 2230 | 6hpf_pa | repressed | GAUGGA | 2.83669 | 9.45E−14 |
| SEQ ID NO: 2231 | 6hpf_pa | repressed | GCGAUG | 3.30952 | 1.06E−13 |
| SEQ ID NO: 2232 | 6hpf_pa | repressed | UCAUGG | 2.94133 | 1.23E−13 |
| SEQ ID NO: 2233 | 6hpf_pa | repressed | GGAUGG | 3.28588 | 1.43E−13 |
| SEQ ID NO: 2234 | 6hpf_pa | repressed | CCAUGG | 3.35895 | 1.99E−13 |
| SEQ ID NO: 2235 | 6hpf_pa | repressed | GUCAUG | 3.12118 | 2.17E−13 |
| SEQ ID NO: 2236 | 6hpf_pa | repressed | UGGCG | 1.91066 | 3.42E−13 |
| SEQ ID NO: 2237 | 6hpf_pa | repressed | CAUGA | 1.94073 | 5.56E−13 |
| SEQ ID NO: 2238 | 6hpf_pa | repressed | AAAUGA | 2.40397 | 6.11E−13 |
| SEQ ID NO: 2239 | 6hpf_pa | repressed | GCCAUG | 3.23325 | 1.62E−12 |
| SEQ ID NO: 2240 | 6hpf_pa | repressed | GAUGUG | 2.4639 | 2.09E−12 |
| SEQ ID NO: 2241 | 6hpf_pa | repressed | GGGAUG | 2.98152 | 2.46E−12 |
| SEQ ID NO: 2242 | 6hpf_pa | repressed | AUGAU | 1.87295 | 2.66E−12 |
| SEQ ID NO: 2243 | 6hpf_pa | repressed | AUGGCU | 2.93428 | 2.82E−12 |
| SEQ ID NO: 2244 | 6hpf_pa | repressed | UGUGGC | 2.59925 | 5.42E−12 |
| SEQ ID NO: 2245 | 6hpf_pa | repressed | CGAUGG | 3.33351 | 5.87E−12 |
| SEQ ID NO: 2246 | 6hpf_pa | repressed | ACGAUG | 3.80972 | 7.85E−12 |
| SEQ ID NO: 2247 | 6hpf_pa | repressed | AAUGU | 1.69284 | 8.84E−12 |
| SEQ ID NO: 2248 | 6hpf_pa | repressed | CAUGGA | 2.55425 | 1.18E−11 |
| SEQ ID NO: 2249 | 6hpf_pa | repressed | AUGAUG | 2.69855 | 3.22E−11 |
| SEQ ID NO: 2250 | 6hpf_pa | repressed | AGAUGG | 2.79084 | 3.33E−11 |
| SEQ ID NO: 2251 | 6hpf_pa | repressed | CAUGGC | 2.68435 | 3.98E−11 |
| SEQ ID NO: 2252 | 6hpf_pa | repressed | UAUGGA | 2.80502 | 6.97E−11 |
| SEQ ID NO: 2253 | 6hpf_pa | repressed | AUGAA | 1.69581 | 7.92E−11 |
| SEQ ID NO: 2254 | 6hpf_pa | repressed | AGCAUG | 2.81265 | 1.01E−10 |
| SEQ ID NO: 2255 | 6hpf_pa | repressed | GAUGCG | 3.08959 | 1.39E−10 |
| SEQ ID NO: 2256 | 6hpf_pa | repressed | UAUGG | 1.92887 | 2.23E−10 |
| SEQ ID NO: 2257 | 6hpf_pa | repressed | GAAAUG | 2.51198 | 2.43E−10 |
| SEQ ID NO: 2258 | 6hpf_pa | repressed | CAAUGU | 2.67456 | 3.95E−10 |
| SEQ ID NO: 2259 | 6hpf_pa | repressed | AUGUC | 1.80621 | 7.23E−10 |
| SEQ ID NO: 2260 | 6hpf_pa | repressed | AAUGA | 1.65533 | 8.70E−10 |
| SEQ ID NO: 2261 | 6hpf_pa | repressed | ACAUG | 1.74976 | 1.56E−09 |
| SEQ ID NO: 2262 | 6hpf_pa | repressed | AUGCG | 1.87475 | 1.58E−09 |
| SEQ ID NO: 2263 | 6hpf_pa | repressed | AGAUGU | 2.3582 | 1.95E−09 |
| SEQ ID NO: 2264 | 6hpf_pa | repressed | CAUGC | 1.83125 | 2.67E−09 |
| SEQ ID NO: 2265 | 6hpf_pa | repressed | GAGAUG | 2.65837 | 2.70E−09 |
| SEQ ID NO: 2266 | 6hpf_pa | repressed | AUGGAC | 2.6062 | 3.40E−09 |
| SEQ ID NO: 2267 | 6hpf_pa | repressed | GCAAUG | 2.77387 | 4.18E−09 |
| SEQ ID NO: 2268 | 6hpf_pa | repressed | GGCGG | 1.66025 | 4.82E−09 |
| SEQ ID NO: 2269 | 6hpf_pa | repressed | CGAUGA | 2.7821 | 6.07E−09 |
| SEQ ID NO: 2270 | 6hpf_pa | repressed | GGAGC | 1.53364 | 7.75E−09 |
| SEQ ID NO: 2271 | 6hpf_pa | repressed | AGGGG | 1.79187 | 8.49E−09 |
| SEQ ID NO: 2272 | 6hpf_pa | repressed | CAUGGG | 3.03937 | 1.02E−08 |
| SEQ ID NO: 2273 | 6hpf_pa | repressed | GGCGGC | 2.20089 | 1.04E−08 |
| SEQ ID NO: 2274 | 6hpf_pa | repressed | CCAUGU | 2.80015 | 1.28E−08 |
| SEQ ID NO: 2275 | 6hpf_pa | repressed | GGCUG | 1.5147 | 1.34E−08 |
| SEQ ID NO: 2276 | 6hpf_pa | repressed | AGCCAU | 2.35306 | 1.59E−08 |
| SEQ ID NO: 2277 | 6hpf_pa | repressed | UGAUGG | 2.36193 | 1.96E−08 |
| SEQ ID NO: 2278 | 6hpf_pa | repressed | CAGGAU | 2.35012 | 2.32E−08 |
| SEQ ID NO: 2279 | 6hpf_pa | repressed | CAUGU | 1.63661 | 2.44E−08 |
| SEQ ID NO: 2280 | 6hpf_pa | repressed | AAUGUG | 2.03772 | 2.50E−08 |
| SEQ ID NO: 2281 | 6hpf_pa | repressed | AUGGAA | 2.44331 | 2.69E−08 |
| SEQ ID NO: 2282 | 6hpf_pa | repressed | GAGGA | 1.43017 | 3.45E−08 |
| SEQ ID NO: 2283 | 6hpf_pa | repressed | AACAUG | 2.3785 | 4.37E−08 |
| SEQ ID NO: 2284 | 6hpf_pa | repressed | UGGCGG | 2.23226 | 4.96E−08 |
| SEQ ID NO: 2285 | 6hpf_pa | repressed | GAAUG | 1.58989 | 6.01E−08 |
| SEQ ID NO: 2286 | 6hpf_pa | repressed | GGAUGA | 2.19839 | 6.18E−08 |
| SEQ ID NO: 2287 | 6hpf_pa | repressed | GGGAU | 1.58487 | 6.51E−08 |
| SEQ ID NO: 2288 | 6hpf_pa | repressed | AGAUGA | 2.22739 | 7.20E−08 |
| SEQ ID NO: 2289 | 6hpf_pa | repressed | UGGCCG | 2.34985 | 8.92E−08 |
| SEQ ID NO: 2290 | 6hpf_pa | repressed | GGAGG | 1.52455 | 1.16E−07 |
| SEQ ID NO: 2291 | 6hpf_pa | repressed | AUGCC | 1.92064 | 1.32E−07 |
| SEQ ID NO: 2292 | 6hpf_pa | repressed | AGAAUG | 2.61918 | 1.86E−07 |
| SEQ ID NO: 2293 | 6hpf_pa | repressed | UUAUGG | 2.36699 | 2.00E−07 |
| SEQ ID NO: 2294 | 6hpf_pa | repressed | UGAUGU | 1.96213 | 2.11E−07 |
| SEQ ID NO: 2295 | 6hpf_pa | repressed | GAGAU | 1.58071 | 2.20E−07 |
| SEQ ID NO: 2296 | 6hpf_pa | repressed | GAAGAU | 1.96784 | 2.36E−07 |
| SEQ ID NO: 2297 | 6hpf_pa | repressed | UGUGG | 1.46863 | 2.76E−07 |
| SEQ ID NO: 2298 | 6hpf_pa | repressed | UGGAGG | 2.00715 | 2.91E−07 |
| SEQ ID NO: 2299 | 6hpf_pa | repressed | GAGGAU | 1.91186 | 2.92E−07 |
| SEQ ID NO: 2300 | 6hpf_pa | repressed | CAUGAC | 2.76205 | 2.98E−07 |
| SEQ ID NO: 2301 | 6hpf_pa | repressed | AUGUGU | 1.88742 | 3.17E−07 |
| SEQ ID NO: 2302 | 6hpf_pa | repressed | GGGGA | 1.6246 | 3.18E−07 |
| SEQ ID NO: 2303 | 6hpf_pa | repressed | GUGAUG | 2.25143 | 3.18E−07 |
| SEQ ID NO: 2304 | 6hpf_pa | repressed | GGGGC | 1.71029 | 3.31E−07 |
| SEQ ID NO: 2305 | 6hpf_pa | repressed | AUGAC | 1.69346 | 3.39E−07 |
| SEQ ID NO: 2306 | 6hpf_pa | repressed | GAGGG | 1.55027 | 4.67E−07 |
| SEQ ID NO: 2307 | 6hpf_pa | repressed | AAUGAG | 2.0748 | 5.35E−07 |
| SEQ ID NO: 2308 | 6hpf_pa | repressed | GCAUGG | 2.40012 | 5.51E−07 |
| SEQ ID NO: 2309 | 6hpf_pa | repressed | GAUGAG | 2.10537 | 5.70E−07 |
| SEQ ID NO: 2310 | 6hpf_pa | repressed | CAGGGG | 2.50013 | 5.95E−07 |
| SEQ ID NO: 2311 | 6hpf_pa | repressed | CAGGG | 1.63912 | 6.95E−07 |
| SEQ ID NO: 2312 | 6hpf_pa | repressed | GAUGAU | 2.11993 | 7.88E−07 |
| SEQ ID NO: 2313 | 6hpf_pa | repressed | GGGGCG | 2.39738 | 8.07E−07 |
| SEQ ID NO: 2314 | 6hpf_pa | repressed | AUGGGA | 2.19907 | 8.42E−07 |
| SEQ ID NO: 2315 | 6hpf_pa | repressed | AUGGCC | 2.61275 | 1.05E−06 |
| SEQ ID NO: 2316 | 6hpf_pa | repressed | UCAUGA | 2.20442 | 1.06E−06 |
| SEQ ID NO: 2317 | 6hpf_pa | repressed | GUGGC | 1.6072 | 1.08E−06 |
| SEQ ID NO: 2318 | 6hpf_pa | repressed | GGCAG | 1.50986 | 1.11E−06 |
| SEQ ID NO: 2319 | 6hpf_pa | repressed | GGCAGC | 2.0244 | 1.15E−06 |
| SEQ ID NO: 2320 | 6hpf_pa | repressed | GCAUG | 1.61116 | 1.21E−06 |
| SEQ ID NO: 2321 | 6hpf_pa | repressed | GGAUGU | 2.07059 | 1.22E−06 |
| SEQ ID NO: 2322 | 6hpf_pa | repressed | GGAGGA | 1.73342 | 1.27E−06 |
| SEQ ID NO: 2323 | 6hpf_pa | repressed | GAUGUU | 1.91929 | 1.31E−06 |
| SEQ ID NO: 2324 | 6hpf_pa | repressed | UGGCAG | 2.09313 | 1.48E−06 |
| SEQ ID NO: 2325 | 6hpf_pa | repressed | GGAGCG | 1.91791 | 1.65E−06 |
| SEQ ID NO: 2326 | 6hpf_pa | repressed | GGGCUG | 2.0001 | 1.65E−06 |
| SEQ ID NO: 2327 | 6hpf_pa | repressed | GAUGUC | 2.39143 | 1.74E−06 |
| SEQ ID NO: 2328 | 6hpf_pa | repressed | UGGCC | 1.6371 | 1.74E−06 |
| SEQ ID NO: 2329 | 6hpf_pa | repressed | GCGAU | 1.65115 | 1.76E−06 |
| SEQ ID NO: 2330 | 6hpf_pa | repressed | GCCAU | 1.59175 | 1.88E−06 |
| SEQ ID NO: 2331 | 6hpf_pa | repressed | CAAUGG | 2.50013 | 1.89E−06 |
| SEQ ID NO: 2332 | 6hpf_pa | repressed | AUAUGA | 2.14671 | 2.16E−06 |
| SEQ ID NO: 2333 | 6hpf_pa | repressed | GAUGCU | 2.13553 | 2.48E−06 |
| SEQ ID NO: 2334 | 6hpf_pa | repressed | UGAGGG | 2.0549 | 2.50E−06 |
| SEQ ID NO: 2335 | 6hpf_pa | repressed | AAGAU | 1.53321 | 2.61E−06 |
| SEQ ID NO: 2336 | 6hpf_pa | repressed | CACGAU | 2.59629 | 2.78E−06 |
| SEQ ID NO: 2337 | 6hpf_pa | repressed | UGGAGA | 1.83193 | 2.83E−06 |
| SEQ ID NO: 2338 | 6hpf_pa | repressed | CAGAUG | 1.97591 | 2.99E−06 |
| SEQ ID NO: 2339 | 6hpf_pa | repressed | GGCCG | 1.61157 | 3.40E−06 |
| SEQ ID NO: 2340 | 6hpf_pa | repressed | UGGCA | 1.59107 | 4.64E−06 |
| SEQ ID NO: 2341 | 6hpf_pa | repressed | AUGUCG | 2.29179 | 4.68E−06 |
| SEQ ID NO: 2342 | 6hpf_pa | repressed | GAUGGG | 2.4371 | 4.79E−06 |
| SEQ ID NO: 2343 | 6hpf_pa | repressed | CCAUGA | 2.478 | 4.91E−06 |
| SEQ ID NO: 2344 | 6hpf_pa | repressed | GGCGAU | 2.5235 | 4.98E−06 |
| SEQ ID NO: 2345 | 6hpf_pa | repressed | AUGAAG | 1.89791 | 5.17E−06 |
| SEQ ID NO: 2346 | 6hpf_pa | repressed | GAUGAA | 2.01845 | 5.22E−06 |
| SEQ ID NO: 2347 | 6hpf_pa | repressed | GCGGG | 1.52066 | 5.58E−06 |
| SEQ ID NO: 2348 | 6hpf_pa | repressed | GAUGCC | 2.50013 | 6.01E−06 |
| SEQ ID NO: 2349 | 6hpf_pa | repressed | AUGGUG | 2.33095 | 6.18E−06 |
| SEQ ID NO: 2350 | 6hpf_pa | repressed | UGAGG | 1.43253 | 6.77E−06 |
| SEQ ID NO: 2351 | 6hpf_pa | repressed | GGGCU | 1.53158 | 7.74E−06 |
| SEQ ID NO: 2352 | 6hpf_pa | repressed | UGGGUG | 2.08567 | 7.76E−06 |
| SEQ ID NO: 2353 | 6hpf_pa | repressed | UGGAGC | 1.73806 | 8.35E−06 |

### ENUMERATED EMBODIMENTS

Embodiment 1: An mRNA molecule comprising a cis-regulatory element selected from the group consisting of SEQ ID NOs: 1-2353 and 2384, wherein the cis-regulatory element does not naturally exist in the mRNA molecule at a location of the cis-regulatory element.

Embodiment 2: The mRNA molecule of Embodiment 1, wherein the cis-regulatory element is a cis-regulatory element for the translational machinery of a mammal, optionally a human.

Embodiment 3: The mRNA molecule of Embodiment 2, wherein the cis-regulatory element comprises at least one sequence selected from the group consisting of SEQ ID NOS: 1-1156 and 2384.

Embodiment 4: The mRNA molecule of Embodiment 1, wherein the cis-regulatory element is a cis-regulatory element for the translational machinery of a fish, optionally a zebrafish.

Embodiment 5: The mRNA molecule of Embodiment 4, wherein the cis-regulatory element comprises at least one sequence selected from the group consisting of SEQ ID NOs: 1157-2353.

Embodiment 6: The mRNA of any one of Embodiments 1-5, further comprises a modified nucleobase that does not naturally exist at the location of the modified nucleobase.

Embodiment 7: The mRNA molecule of Embodiment 6, wherein the modified nucleobase comprises at least one of N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, and 4-acetylcytidine.

Embodiment 8: The mRNA molecule of any one of Embodiments 1-7, wherein the mRNA molecule encodes a therapeutic peptide or a therapeutic protein.

Embodiment 9: The mRNA molecule of Embodiment 8, wherein the therapeutic peptide or the therapeutic protein comprises a vaccine.

Embodiment 10: A method of modulating a translational efficiency of a messenger RNA (mRNA) molecule in a cell, the method comprising: modifying the mRNA molecule to comprise a cis-regulatory element selected from the group consisting of SEQ ID NOs: 1-2353 and 2384.

Embodiment 11: The method of Embodiment 10, wherein modifying the mRNA molecule comprises modifying a sequence of a DNA molecule encoding the mRNA molecule.

Embodiment 12: The method of any one of Embodiments 10-11, wherein the cis-regulatory element is a cis-regulatory element for the translational machinery of a mammal, optionally a human.

Embodiment 13: The method of Embodiment 12, wherein the cis-regulatory element comprises at least one sequence selected from the group consisting of SEQ ID NOs: 1-1156 and 2384.

Embodiment 14: The method of any one of Embodiment 10-11, wherein the cis-regulatory element is a cis-regulatory element for the translational machinery of a fish, optionally a zebrafish.

Embodiment 15: The method of Embodiment 14, wherein the cis-regulatory element comprises at least one sequence selected from the group consisting of SEQ ID NOs: 1157-2353.

Embodiment 16: The method of any one of Embodiments 10-15, wherein the mRNA molecule is further modified to comprise a modified nucleobase.

Embodiment 17: The method of Embodiment 16, wherein the modified nucleobase comprises at least one of N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

Embodiment 18: The method of any one of Embodiments 10-17, wherein the cis-regulatory element activates the translation of the mRNA molecule, and wherein a translational efficiency of the mRNA molecule is increased by about 50% or more in comparison to an mRNA molecule lacking the cis-regulatory element but otherwise has the same sequence.

Embodiment 19: The method of any one of Embodiments 10-17, wherein the cis-regulatory element represses the translation of the mRNA molecule, and wherein a translational efficiency of the mRNA molecule is decreased by about 30% or more in comparison to an mRNA molecule lacking the cis-regulatory element but otherwise has the same sequence.

Embodiment 20: The method of any one of Embodiments 10-19, wherein the cis-regulatory element activates or represses the translation of the mRNA molecule in the cell at a certain cellular stage.

Embodiment 21: The method of any one of Embodiments 10-20, wherein the cis-regulatory element is located in a 5′ untranslated region (5′-UTR), at a region including the start codon of the main open reading frame, within the open reading frame, or in a 3′ untranslated region (3′-UTR).

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. An mRNA molecule comprising a cis-regulatory element selected from the group consisting of SEQ ID NOs: 1-2353 and 2384, wherein the cis-regulatory element does not naturally exist in the mRNA molecule at a location of the cis-regulatory element.

2. The mRNA molecule of claim 1, wherein the cis-regulatory element is a cis-regulatory element for the translational machinery of a mammal, optionally a human.

3. The mRNA molecule of claim 2, wherein the cis-regulatory element comprises at least one sequence selected from the group consisting of SEQ ID NOs: 1-1156 and 2384.

4. The mRNA molecule of claim 1, wherein the cis-regulatory element is a cis-regulatory element for the translational machinery of a fish, optionally a zebrafish.

5. The mRNA molecule of claim 4, wherein the cis-regulatory element comprises at least one sequence selected from the group consisting of SEQ ID NOs: 1157-2353.

6. The mRNA of claim 1, further comprises a modified nucleobase that does not naturally exist at the location of the modified nucleobase.

7. The mRNA molecule of claim 6, wherein the modified nucleobase comprises at least one of N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, and 4-acetylcytidine.

8. The mRNA molecule of claim 1, wherein the mRNA molecule encodes a therapeutic peptide or a therapeutic protein.

9. The mRNA molecule of claim 8, wherein the therapeutic peptide or the therapeutic protein comprises a vaccine.

10. A method of modulating a translational efficiency of a messenger RNA (mRNA) molecule in a cell, the method comprising:
modifying the mRNA molecule to comprise a cis-regulatory element selected from the group consisting of SEQ ID NOs: 1-2353 and 2384.

11. The method of claim 10, wherein modifying the mRNA molecule comprises modifying a sequence of a DNA molecule encoding the mRNA molecule.

12. The method of claim 10, wherein the cis-regulatory element is a cis-regulatory element for the translational machinery of a mammal, optionally a human.

13. The method of claim 12, wherein the cis-regulatory element comprises at least one sequence selected from the group consisting of SEQ ID NOs: 1-1156 and 2384.

14. The method of claim 10, wherein the cis-regulatory element is a cis-regulatory element for the translational machinery of a fish, optionally a zebrafish.

15. The method of claim 14, wherein the cis-regulatory element comprises at least one sequence selected from the group consisting of SEQ ID NOs: 1157-2353.

16. The method of claim 10, wherein the mRNA molecule is further modified to comprise a modified nucleobase.

17. The method of claim 16, wherein the modified nucleobase comprises at least one of N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

18. The method of claim 10, wherein at least one of the following applies:
(a) the cis-regulatory element activates the translation of the mRNA molecule, and wherein a translational efficiency of the mRNA molecule is increased by about 50% or more in comparison to an mRNA molecule lacking the cis-regulatory element but otherwise has the same sequence,
(b) the cis-regulatory element represses the translation of the mRNA molecule, and wherein a translational efficiency of the mRNA molecule is decreased by about 30% or more in comparison to an mRNA molecule lacking the cis-regulatory element but otherwise has the same sequence.

19. (canceled)

20. The method of claim 10, wherein the cis-regulatory element activates or represses the translation of the mRNA molecule in the cell at a certain cellular stage.

21. The method of claim 10, wherein the cis-regulatory element is located in a 5′ untranslated region (5′-UTR), at a region including the start codon of the main open reading frame, within the open reading frame, or in a 3′ untranslated region (3′-UTR).


SEQUENCE LISTING (2384 sequences)

SEQ ID NO: 1-2354 - skipped (2354 entries)

SEQ ID NO: 2355 - RNA, 10 nt, synthetic construct
  source 1..10: mol_type = other RNA
gccrccatgg

SEQ ID NO: 2356 - RNA, 10 nt, synthetic construct
  source 1..10: mol_type = other RNA
gcaaacatgg

SEQ ID NO: 2357 - RNA, 10 nt, synthetic construct
  source 1..10: mol_type = other RNA
ttttgcatgg

SEQ ID NO: 2358 - RNA, 10 nt, synthetic construct
  source 1..10: mol_type = other RNA
gtattcatgt

SEQ ID NO: 2359 - RNA, 10 nt, synthetic construct
  source 1..10: mol_type = other RNA
tttgtcatgt

SEQ ID NO: 2360 - RNA, 10 nt, synthetic construct
  source 1..10: mol_type = other RNA
ataccaatga

SEQ ID NO: 2361 - RNA, 27 nt, synthetic construct
  source 1..27: mol_type = other RNA
aaatcaaaaacaaaaagaaaacgaaaa

SEQ ID NO: 2362 - RNA, 28 nt, synthetic construct
  source 1..28: mol_type = other RNA
aaagtcaaagacaaagagaaagcgaaag

SEQ ID NO: 2363 - RNA, 27 nt, synthetic construct
  source 1..27: mol_type = other RNA
aaattcaaatacaaatagaatcgaaat

SEQ ID NO: 2364 - RNA, 28 nt, synthetic construct
  source 1..28: mol_type = other RNA
aaactcaaacacaaacagaaaccgaaac

SEQ ID NO: 2365 - DNA, 25 bp, synthetic construct
  source 1..25: mol_type = other DNA
acagctcctcgcccttgctcaccat

SEQ ID NO: 2366 - DNA, 24 bp, synthetic construct
  source 1..24: mol_type = other DNA
ccctacacgacgctcttccgatct

SEQ ID NO: 2367 - DNA, 22 bp, synthetic construct
  source 1..22: mol_type = other DNA
gtaaacatggtgagcaagggcg

SEQ ID NO: 2368 - DNA, 20 bp, synthetic construct
  source 1..20: mol_type = other DNA
tgacgtggaagtcggtcaag

SEQ ID NO: 2369 - DNA, 20 bp, synthetic construct
  source 1..20: mol_type = other DNA
gtccagagacaaagtccggg

SEQ ID NO: 2370 - DNA, 20 bp, synthetic construct
  source 1..20: mol_type = other DNA
cacgaggaggaaccagtgac

SEQ ID NO: 2371 - DNA, 20 bp, synthetic construct
  source 1..20: mol_type = other DNA
ctgttgttgtgtgaagggcg

SEQ ID NO: 2372 - DNA, 20 bp, synthetic construct
  source 1..20: mol_type = other DNA
gctctcggtctcggaagaag

SEQ ID NO: 2373 - protein, 8 aa, synthetic construct
DYKDDDDK

SEQ ID NO: 2374 - DNA, 34 bp, synthetic construct
  source 1..34: mol_type = other DNA
gtgactggagttcagacgtgtgctcttccgatct

SEQ ID NO: 2375 - DNA, 23 bp, synthetic construct
  source 1..23: mol_type = other DNA
gcgtcatggtctttgtagtcctc

SEQ ID NO: 2376 - DNA, 29 bp, synthetic construct
  source 1..29: mol_type = other DNA
aatgatacggcgaccaccgagatctacac

SEQ ID NO: 2377 - DNA, 33 bp, synthetic construct
  source 1..33: mol_type = other DNA
acactctttccctacacgacgctcttccgatct

SEQ ID NO: 2378 - DNA, 24 bp, synthetic construct
  source 1..24: mol_type = other DNA
caagcagaagacggcatacgagat

SEQ ID NO: 2379 - DNA, 34 bp, synthetic construct; identical to SEQ ID NO: 2374
  source 1..34: mol_type = other DNA

SEQ ID NO: 2380 - RNA, 22 nt, synthetic construct
  source 1..22: mol_type = other RNA
taagtgctatttgttggggtag

SEQ ID NO: 2381 - RNA, 23 nt, synthetic construct
  source 1..23: mol_type = other RNA
aaagtgctatcaagttggggtag

SEQ ID NO: 2382 - RNA, 22 nt, synthetic construct
  source 1..22: mol_type = other RNA
taagtgcttctctttggggtag

SEQ ID NO: 2383 - RNA, 22 nt, synthetic construct
  source 1..22: mol_type = other RNA
tggaatgtaaagaagtatgtat

SEQ ID NO: 2384 - RNA, 124 nt, synthetic construct
  source 1..124: mol_type = other RNA
gtacgggccgcgttgtcgggggttttgtcccctttttcctcttttttttttccttctccgccccacccccttttctttttctttgctttttttttcctttctgtttttgttgttgttcttgcct